SEQUENCE LISTING (96 sequences)

SEQ ID NO: 1 - DNA, 400 bp, Mus musculus
  All n′s in this sequence represent unreadable nucleic acid.
cccacgcgtccggagagcggcagccatctccatgccaaggttgaacaaaatgccaggaatgttcttttctgctagcccaaaggattcgaaagaacacagccattctcttctagacnacaaaaagcagaaaaaaaggccaaagacttttggaatggacgtgaaaacatacctgagatcgatgatcccacatctggaatctgggatgaaatcggccaagtccaaagacatactttctgctgaagaagttatgcagtggtctcagtctctggaaaaatccttgccaaccagacaggtcnaaatgtctttggaagattctaaagtctgattcagtgaggaaaatattgaattctggttggcttgtgaggactatnanaaaacanaaactgatcttttgcatanc

SEQ ID NO: 2 - DNA, 148 bp, Mus musculus
  All n′s in this sequence represent unreadable nucleic acid.
cggctgctagaagacgacagaaggggactcactcgttttgagaagaccatganagcggcagccntctccatgccaaggttgaacaaaatgccaggaatgttcttttctgctancccaaaggattcgaaanaacacagccattctcttc

SEQ ID NO: 3 - DNA, 400 bp, Mus musculus
  All n′s in this sequence represent unreadable nucleic acid.
tacggctgcgagaagacnacagaaggggaancagctgcggtggccgcgggagtctgacaatgcaaagtgccatgttcctggctgtccngcacgactgcgtacccatggacaagagtgcangcaacggccccaaggtcnangagaagcgggagaaaatgaatcggacactcttnaangattggaanacccgtttgagctacttctngcagagnncctctgctcccgggaagcccnnaactggcaataaaacnaacagcatacttttatcnanccttctcctnangaagcgcanctctnggcataaacttttnatnanctgctggccngtatatatgggctggctgcattcanggcgttttttaaagtcccagttctntgaagaaaacattgaaattctggt

SEQ ID NO: 4 - DNA, 400 bp, Mus musculus
  All n′s in this sequence represent unreadable nucleic acid.
gcctgtggtggctccggaccccctcccttcctgctcatcctggtgtgtacggccccggagcacctgaaccagagaaacgccattcgggcatcttggggtgccatccgcgaagcccggggtttcagagtgcagacgctcttcctcctgggaaaacctagaagacagcagcttgctgacctgtcctcagagtcagcagcacacagggatatcttgcaggcctccttccaggattcctaccgcaacctcaccctcaagaccctcagtggactgaactgggtgaacaaatactgtcctatggcccgctacatcctcaagacggatgatgacgtgtatgtcaacgtcccananctggtgtcagagctgatacaaagaagggggccttcggacaatggcagaaggg

SEQ ID NO: 5 - DNA, 1081 bp, Mus musculus
  All n′s in this sequence represent unreadable nucleic acid.
cccacgcgtccgatctcctccagggccaccaagcacctctgaagagccatgttccaagctgccggagccgcccaggccaccccctctcatgaagccaaaggcagcagtggcagcagcacggtacagcggtctaagtcctttagcttgcgggctcaggtgaaggagacctgtgcagcctgccagaagactgtgtacccgatggagcggctggtggcagacaagctcattttccacaactcttgtttctgttgcaaacactgccacaccaaactcagcctgggcagttatgctgcaatgcacggtgaattttactgcagacctcactttcagcagctgtttaagagtaaaggcaactacgatgaagggtttggtcgtaaacagcacaaggagctctgggcccacaaggaggtggactcaggcaccaagacggcctgagacccctttaacacccattccctcccagcacatggcctcccgctgggcagtggaaaggagattaacccgggggcgcggggtgggagaggatgaggctccctcacacaggtttcaggcataaggctctgctccaggattccttacttttcccatgggaggttggcgttgggaaccagaattggaattttcaccatactgtgtcctttagtccacctcatctcaccccacggctccctgggaggcccacaagcccagcttccatacttaggtgcttttctccagcaaggagtcagcatgccctcctcagggtcccaagctccctcactgccacctgggccttgtgtacccccttgtctccccatctacctctgccccttagcctggtaatgagccacagagactggaagagggagagtgccatntactgggcctcatagatgccacctcgctgaggggggagggctggggaagaggcaagacagcctgcagccttcagggtctgggggtcccttgcaccacaaagctaaagctcttgctagagcctcagctgacagggtcggcagtagctatgctcctccatctgttgtgctgttctgttgtgatcaaccctcttttaaaaacatttaaacagcaaaaaaaaaaaaaa

SEQ ID NO: 6 - DNA, 743 bp, Mus musculus
  All n′s in this sequence represent unreadable nucleic acid.
attggtacgcctgcacgnaccggtccggaattcccgggtcgaccacgcgtccgcccacgcgtccgatctcctccagggggcaccaagcacctctgaagaacatgttccaagctgccggangcgcccangccaccccctctcntgaagcccaangcagcantggcagcagcacggtncagcggtctaagtcctttaacttgcgggctcaggtgaanganacctgtgcagcctgccngaaaactgtgtncccnatggancggctggtggcagacaanctcnttttccacaactcttgtttctgttgcaaacactgccacaccaaactcancctgggcagttatgctgcnatgcaccgtgaattttactgcagacctcactttcagcagctgtttnagaatnaaggcnactacnatgaaaggtttggtcgttaacagcacaanganctctgggcccncnaggaagtngactcaggcccnananaggctganaaccctttaacacccattccctcccnncacattgnctcccnctnggcagttggaaaagaaaataancccngggcnccgggttggganaagaaaaaggtcccccccnccggttttccggcntaagggtctcccccnnaatcccttcttttncccctgggaagtttgggtttgggaacccaaattggaatttccccctnccgtnttcctttnttccccnccnctcccccccnnggtccctgggaaggccccaanccn

SEQ ID NO: 7 - DNA, 743 bp, Mus musculus
  All n′s in this sequence represent unreadable nucleic acid.
gggggagtcttgagtcgcatcgcccgttncgtaagcttggatcctctanaacgggcgcccttttttttttgttttgctgtttaaatgtttttaaaaagaaggttgatcaccacagaacngcacnacngatggaggagcatanctnctgccgaccctgtcagctgangctctagcaanaactttagctttgtggtgcaagggaccccnnaccctgaaagctgcaggctgtcttgcctcttccccanccctcccccctcagcgaagtggcatctatgaagcccagtanatggcactctccctcntccnttctctgtggctcattaccangctaangggcngangttnatnggganacaaaggggtncacnatgcccangtggcantnaaggacttgggacctgaagaaggcatgctgactccttgctgganaaaaagcacctaanttggaactgggcttgtgggcctcccncggaaccntggggtgagatnaagtggactataggaccntatggtgaaaattccaattctggttcccaacccnacctcccatgggaaaaanttaggaatcctgcaaccaaacccttatgcctgaaaacctgttttnaagggancctcatcctcttcccanccccgngcccccnggggtttaaagggaaaaagggtttttttnaaagggggttccccnnggccccccnntggttnccctnaatttcccccccccctnntttgggccccnaaaaccn

SEQ ID NO: 8 - DNA, 174 bp, Mus musculus
ggattccggttaggaatgaaattagaagctgtagatctcatggagccacggttaatatgtgtagccacagttactcgaattattcaccatctcttgaggatacattttgatggttgggaagaagagtatgaccagtgggtagactgtgagtcccctgacctctatcctgtaggg

SEQ ID NO: 9 - DNA, 711 bp, Mus musculus
  All n′s in this sequence represent unreadable nucleic acid.
ggggctcagggaaggccgatcttccgggtggagggggaagcggcgtgactggagtggaaatttttcccaacacaacttctcggaggcaacatattggaagggactcggggaggccggagtccaaatggaagtggctgaaagaaacttctcgccctgctgattctgagccccgcgtcgtgccgcgcgccctcaattacctcatcgacctgtggtcttgacagaacattcttcacaatccaaaagaaaaagcagactggttcgggatgtttgacggctatgatagctgcagtgaggacacaagtagcagctccagctctgaggagagtgaagaagaagttgctcctttaccttccaatctcccaatcatcaagaataatggacaagtctacacatacccagatggtaaatctggcatggctacctgtgagatgtgtgggatggtcggtgtgcgagatgctttttactctaaaacgaaacgtttctgcagtgtttcctgttcaagaagttactcgtcaaactctaagaaggcaagcattctggccngacttcnngttacgggttngcctcccacnaagaaagccaaantccttccnaaacnaccnttagttggtaaattnnctgcctatgcccntntccanctaccttgcnnaatccnnccagacnaaancnggcnattctgccatctctgtggaaagggtccctgggggttac

SEQ ID NO: 10 - DNA, 356 bp, Mus musculus
  All n′s in this sequence represent unreadable nucleic acid.
tacggccgcganaagacgacagaagggtncgggctgcganaagacgacagaaagggggcctttctccgctggccccggcgcgcccggcagctcctccccggccatggcgttcactttcgcggccttctgctatatgctggcgctgctgctcaccgccgcgctcatcttcttcgccatctggcacatcatancntttgatgagctgaagaccgactacaagaaaccctatagaccagtgcaataccctgaacctcttgtccttccagaagtactcatccacgcgttcttctgtgtcatgtttctctgtgcgcagaattggctgaccctgggcctcaatatgccccttttggcatacc

SEQ ID NO: 11 - DNA, 400 bp, Mus musculus
  All n′s in this sequence represent unreadable nucleic acid.
tacggctgcgagaagacgacagaaggggagtgctcgccggtcccagcgttgccataccatcgagatggcatctcgtagccggagacccgaacacagcggaccgccggagctgttttatgaccagaatgaagcccggaaatacgttcgcaactcacggatgattgacatccagaccaanatgactgagcgagcgctggagctcctctgtttaccaganggtcagccttcttacctgttagacattggctgcggttctgggctgagtggaaattatatctcagaagaaggacactactgggtgggcattgacatcagccctgccatgttggatgccgccttggacgagatacanaaggggactgctgctangggactggccaaggctcctttcaaaccggcc

SEQ ID NO: 12 - DNA, 400 bp, Mus musculus
  All n′s in this sequence represent unreadable nucleic acid.
acggctgcgagaagacgacagaagggtacggctgcgagaagacgacagaagggtacggctgcganaagacgacagaagggtacggctgcgagaagacgacagaagggggaggaaactgtaaaagctatgctggggangcagcgcggancttgattcaccttcacctgctctggccacccgctgacccggggtttccggccgganagcagtcanatatgaacggacgggtggattatttagtcacggaggaagagatcaacctgacgagaggaccttcggggctgggcttcaacatcgtcngtgggacagatcaacagtatgtctccaacgacagtggcatctacntcagccgtatcaaanaagatggggctgcngcccangatgggcggctccaggaagg

SEQ ID NO: 13 - DNA, 1360 bp, Mus musculus
agctctagtccccagagatgtcgccactactgctgctgctgctgtgcctgctgctggggaatttggagcctgaggaggccaaactgatccgtgtccctcttcaacgaatccaccttggacacagaatcttaaacccactgaatggatgggaacagctggcagagctttctaggacctccacctctggtggcaacccctcctttgtgcctctctccaagttcatgaacacccagtattttggaactattggtttgggaacgcctcctcagaatttcaccgttgtctttgacacgggttcttccaacttgtgggttccgtccacgagatgtcatttcttcagtttggcatgctggtttcaccatcgctttaatcccaaggcctccagctccttcaggcccaatgggaccaagtttgccattcagtatgggaccgggcggctgagcggaatcctgagccaggacaatctgactatcggggggatccacgatgcttttgtgacatttggagaggctctgtgggagcccagcctgatctttgctttagcccactttgatgggatcctgggcctcggcttccccactctggctgtgggcggagttcagcctccgctggatgcgatggtggagcaagggctgctggagaaacccgtcttctccttttacctcaacagggattctgaagggtctgatgggggagagctggtcctagggggctcagaccccgctcactacgtacctcccctcaccttcataccagtcaccatccctgcctactggcaggtccacatggagagtgtgaaggtcggcacagggctgagcctctgtgcccagggctgcagtgccatcctagacacaggcacatccctcatcacaggacctagtgaggagatccgggccttgaataaagccattgggggatatcccttcctgaatgggcagtacttcattcagtgttccaagacgccaacgcttccccctgtctccttccaccttggtggagtctggtttaacctcacaggccaggactatgtcatcaagattcttcagagcgatgttggcctctgcctgttgggcttccaagccttggatatccccaagcctgcgggacccctctggatccttggggacgtctttttggggccctatgtggctgtctttgaccgtggggacaaaaacgtcggcccgcgcgtgggactggcgcgtgctcagtctcgttcaacagaccgggcagaaagaaggactacgcaggcgcagttcttcaaaagacgccctggttagggtacaagctcaccgggccacagcagctatgcttctttccaattaaacaaactaaaaaaaaaaaaaaaaaaaaaaaaaaaaaa

SEQ ID NO: 14 - DNA, 1712 bp, Mus musculus
tcttcgaaagccgggctgaggggaatcctggacaggggaatcctggacgtggagatcgtgagtcatggctgcttcccgagacgctgatgagatccacaaggacgttcagaactactatgggaatgtactgaagacatctgcagacctccagactaatgcttgtgtcacgcgagccaagccggtccccagctacatccgggaaagtctgcagaatgtacacgaagacgttagttcgaggtattatggctgtggtctgactgttcctgagcggctggaaaactgccgaattttggatctgggtagtgggagtggcagggattgctatgtgcttagccagctggttggtgagaagggacatgtcaccggaatagacatgactgaggtccaggtcgaagtggctaaaacctatcttgaacaccacatggaaaaatttggtttccaggcacccaatgtgacttttctccacggccgcatcgagaagttggcagaggctgggatccagagtgagagctatggtattgtcatatccaactgtgttatcaaccttgttcctgataaacaacaagtcctccaggaggtctatcgagtgctgaagcacggcggggagctctatttcagtgacgtctatgccagccttgaagtgccagaagacatcaagtcgcacaaagttttatggggggaatgcctgggaggcgctctgtactggaaggatcttgccatcattgcccaaaagattgggttctgccctccacgtttggtcactgccgatatcattactgttgaaaacaaggagctcgaaggggttcttggtgactgtcgctttgtgtctgccacatttcgcctcttcaaactccctaagacagagccagccgaaagatgccgagttgtttacaatggaggaatcaagggacatgaaaaggaactaattttcgatgcaaatttcacattcaaggaaggcgaagctgttgcagtggatgaggagacggcagctgtcctgaagaactcacgttttgctccggattttctcttcacacctgttgacgcctcgctgccagctccccaggggccgttctgagttagagacaaaggttctaatcagagatccattcaagcttgcagaggactctgacaagatgaagcccagacatgcacctgaaggcacgggaggctgctgtggcaagaggaaaaactgctagatctacagccagcgcggagcccaccgggctcaagagggtggctaaaggacagtcacagaggcttcttagcctgctcttcgccagtgcacagattatgtgaaggtggcaaagccaccacaagctagaccactgctaagaataagagtgacttttagaggatgttaattgaaggttcacagcaaatcgcctgcttttctatttctctatctcagagttctggtgccacctagtggtcagaagtagaacttggaagcccaaggtttactcaaagggccaaaggcatcatcaacgttgtgagaattatcttccttctggcctaccacaggacacctctgggttcttctctgtggttaccaggaagcacagtacttactaaatttatgctaaccatgacaaaagattgtcaactcaaatttgctaggagtattctttaggttgctgtctgcaatttttttctctgtaactgaatgaaaaagaaaacaaataaaaaataaatttgacttcgaaaaaaaaaaa

SEQ ID NO: 15 - DNA, 1539 bp, Mus musculus
cggacgcgtgggggagtctgtggagcccccggtcaaagacggcatcctctaccagcagcacgtaaagtttggcaagaaatgctggcgcaaagtgtgggctctgctgtatgcgggaggcccatcaggggtagctcggctagaaagctgggacgtgcgtgatggtggcctgggaccagcaggcgacaggtccacagggcccagccgtcgaggggaacgccgggtcatacgcttggctgactgtgtatctgtcctgcctgcggatggcgagagctgtcccagggacactggtgccttcctgattaccaccactgagcgaagccacctgttggctgcacagcaccgccagtcctgggtggaccccatctgtcagctggccttcccgggtaccggagaatgttcgtcaggatcaggacaggctgagaatccaaaaaggggctttgttcccatggaagaaaactctatctactcctcctggcaggaagtgaccgaatttccggtgatcgtgcagaagacagaagccacctcccgctgccagctgaaaggaccctacctcctggtgctgggccaagatgacatccaactgagggagacatccaagccccaggcctgttttagctggccctaccgtttcctgcgcaagtacggctctgacaagggtgtgttctcgtttgaggctggccgccgctgtgactcaggtgagggcctttttgccttcagtagcccgcgtgccccagacatatgtggggttgtggctgccgccattgcccgccagcgggagcgtcttccagagctggccatgtccccaccctgccccctgcctcgggccctctccctgccctccctagagccccctggagagcttcgggaggtggccccaggatttgagctgcccactcccagaaagctgcctctaactgatcccgggcctcaaagcctaccattgctgctcagccccaacacaagaaggaccggcatccggtctctatgcgtccgtgtgcaagcagaccagcaagcacacaggcacggcggagcatttctatgagaacgtgtgcatgctggaggccagccttgggctgaccaatgggggtcctgaagcccaagagggcccccttggtggccgcagcccccttgggcagccttatctaccataacactgaggatctgagttggccgggctcggcccaggacagcaatctggaagcccagtaccggaggctgctggaactggagctggatgaggccggaagcgccggccgctctggagcgcaggcaggcatcaaggccaagctggtgaccctgctgacccgtgaacggaagaagggccccgccccctgtgaccggccctgaaggcctgagcggccagccactgcaggacagaggtgatcacccaagaccaggaacaacttcgaacataacccgtctactctgacctgcagggacaagccaggtggcccggggaggagccacactctgccctacctcctccctcagactgtacagattgaacagtaataaagcttgcctatcaacttcaaaaaa

SEQ ID NO: 16 - DNA, 3599 bp, Mus abbotti
agagacagcgtgatcccggcctcccacggggcagcttttactgtctagggaagaaatccccaaagtccatggagtctgaagactctgtcaagcctcgctaggaaacctaggagttttagagggcacttggcaccggaagctagccgggtaggcggagcctcacctggattgagttcacagctgcctagacaggctcagactaggtgctgggcacctgggaggaggaggagacattagcagcaaaggctgttaacagaagtgcctgcctaggcttggaggcaagacgctgctgttcacagtgcgagacggaggtaggagtataatggctgtccaggtgctgcggcagatggtctacttcctactgagtctgttttctctggtgcaaggtgcacacagtggcagcccccgagaagacttccgcttctgtggccagcggaaccagacccaacagagcaccctccactatgatcaatcttcagagcctcacatctttgtgtggaacacagaggagaccctcacaattcgtgcccccttcctggcagccccagatattccccgcttcttcccagagcctagagggctctatcacttctgcctctactggagtcgccacactgggagactccacttgcgctatggcaagcatgactacctgcttagtagccaagcctccagactcctctgcttccagaaacaggagcagagcctgaagcagggagccccgctgatcgccacctctgtcagctcctggcagattccccagaacaccagcctgcctggggctccgagcttcatcttctccttccacaatgccccacacaaggtctcccacaatgcatctgtggacatgtgtgatctcaagaaggaattgcagcagcttagcaggtacctgcagcaccctcaaaaggctgccaagcggcccaccgcagcgttcatcagccagcagttacagagcctggagtcaaagctgacctctgtgagcttcctgggagacacattatcctttgaggaggaccgggtcaatgctacagtgtggaagctgccacccacagccggtctagaggatctgcatatccactcccagaaggaggaggagcagagtgaggtccaggcatactcgctgttgcttccccgggccgtattccagcagaccagaggccgtcgccgggatgacgccaagaggctcctggtagtagacttcagcagccaagctttgttccaggacaagaattctagccaagtcctgggtgagaaggtcttgggtattgtcgtgcagaacaccaaagtcaccaacctctcagatccggtggtactcaccttccagcaccagcctcagccaaaaaatgtgactctgcagtgcgtgttctgggttgaagacccggcatcaagcagcacagggagctggagcagtgcaggctgcgagacagtgagcagagacacacagacatcctgcctgtgcaaccacctgacctactttgcagtgctgatggtgtcatccacagaggtagaagccactcacaaacactacctcacgctcctgtcctacgtgggctgtgtcatctctgctctggcttgtgtcttcactatcgctgcctacctctgctccaggaggaagtcacgtgactacaccatcaaagtccacatgaacctgctgtccgctgtcttcctgctggacgtgagcttcctgctcagcgagcctgtggcactgacgggctccgaagcagcctgtcgcaccagtgccatgttcctgcacttctccctgcttgcctgcctctcctggatgggcctcgagggctacaatctctaccgactggtggtggaggtcttcggtacctatgtgcccggctatctgctcaagctgagcatcgtgggctggggttttcctgtcttcctggtcactctggtggcgttggtggatgtgaataactacggccccattatcctagctgtgcgccggactccggaacgtgtcacctacccctctatgtgctggatccgggactccctggtgagctatgtcaccaacctgggcctcttcagtctggtgttcctgttcaacctggctatgctggccaccatggtggtgcagatcctgcggcttcgcccgcacagccagaactggccccacgtgctgaccctgctgggcctcagcctggtccttggcctcccctgggccttggtcttcttttcctttgcttccggcaccttccagcttgtcatcctctacctcttcagcatcataacttcctaccaaggcttcctcatcttcctgtggtactggtccatgcggttccaggcccaaggcggcccctcccctctgaagaacaactcagacagcgccaaactccccatcagctccggcagcacctcctccagccgcatctaagccaccgccacacctcccctccgggaggacacatgcatggcgtccgctcacgatgtctgtggcccagtgctgtgcccacccagcctttgttggttagtggcatactagagaaggccctggtccttgaaggcgtagggctgttgctctggtaggtagatacctagcttgccttggggacgactctggtcctcaaagggctcagaagcacactgccattctgtttgtggggccgtttcagtctggagctaaggccttgtctttctggccacctctgggtccagctgttgctgctgggtgttgagacctgcagacccaagctggggttagatctcgaaggaggctgacacatccggcctgagacacagctaactgtcttgacttgctgctctgtctctgtggtcaccatgcagatcccgagggtggcactgggggtaaatgttctgggagaaggtttggaggcagagcaccttaggagctgagcatctcccccagcctttctgcaaaccctcctcttcattccccatccccaacccctcctctgtgttcccctaaccctccacctgaagcctggggtcctagaccaatgctgtgatttggggtggtagttcccagcagtttcctggtgccagctatcaacttctgtctgttgtgtgggctttggcctctgactcagggcaggtttctgtctgagccctctctccaagctgcctcacctttgctcgcacctcagagggacctccatctctcctgaagcctcctccctctggcaagtactgggatacagccaccctttcaacccagcactctgaagaccaagacagccccctctggtgacactggccaagcttgatctttttcctaagaagtggtcttcagatccccgcaggtcgctcagaagacactgggctgcctagtgtgaattctgtcctactaacgtacagtgagcagctcctcacccccacccccgcaaaagctctcaccaagtcctggagtgtcaggcagggggctggaaatccaggaggacttcctgcaaaaggcagcatttcatcttgacctcagccttcaggttggggagaatgttctttttaaataccagttcatttgtcttttgatattaaagctctttatagagagtctggaaactgtaggcgattgtcgagaagagaaataaaaatgagctgttatctaatgccatggcaaagcagcacaaaaa

SEQ ID NO: 17 - DNA, 399 bp, Mus musculus
  All n′s in this sequence represent unreadable nucleic acid.
agcgacatggccccgcccgcgctccaggcccagcctccaggcggctctcaactgaggttcctgctgttcctgctgctgttgctgctgctgctgtcatggccatcgcagggggacgccctggcaatgcctgaacagcgaccctccggccctgagtcccaactcaacgccgacgagctacggggtcgcttccaggacctgctgagccggctgcatgccanncatagcgagaggactctaactctgaaccaagtcctgaccatctgtcnggatactcantccacangtgagattggggtccacggccagctgctactccncgtcaaccgggcgtcgctgantcanggtctccccgaacctaccncgtgcancnagcgctgctcctgctgacnncnaangccg

SEQ ID NO: 18 - DNA, 400 bp, Mus musculus
  All n′s in this sequence represent unreadable nucleic acid.
cggacgcgtggggaaagcgggtggggatatacgtgtgctggtgccctacaattcgacaggcgtcttgggagggtcgaccaccttgcactgtactctgacttctaatgagaatgtgactatcactcaaataacctggatgaagaangattcaggtggaccccacgctcttgtggctgtcttccaccccaagaaggggcccaacatcaaagagccagagagggtgaaattcttggctgcccaacaggatctgaggaacgcatctctggccatctcgaacttaagtgtnnaagacgaangcatctatgaatgtcagattgccacattccccagaagcagtanaagcaccaatgcctggctgaangtgcaagcccgacctnanaacactgcanaagccctggac

SEQ ID NO: 19 - DNA, 2313 bp, Mus musculus
cgaattcctgggtcgacccacgcgcccgagagggtagacagaaaggcgggaaagggctgtgaggtcaaatggaccccatggaactgaacaacgtcagcatcgaacccgacggagacagctgcagcggggacagtattcaggacagctacaccggcatggaaaactccgacaaggacgccatgaacagccaatttgctaatgaagatgccgaaagtcagaagttcctgacaaatgggtttttagggaagaagaagctagccgattacgcggatgagcatcaccctggaatgacttcctttggaatgtcctcatttaacctgagcaacgccatcatgggcagtgggatcttaggcttgtcctatgccatggccaacaccgggatcatcctttttataatcatgctgcttactgtggcaatactctcgctctactcggttcaccttttgctgaagacagccaaggaaggagggtctctaatctatgaaaaattgggcgagaaagcatttggatggcctgggaaaattggagccttcatctctattacaatgcagaacattggagccatgtcaagctacctcttcatcattaagtacgaactgcctgaagtaatcagagcattcatgggacttgaagaaaacactggggaatggtacctcaacggcaactacctcgtcttatttgtgtccgtggggatcatcctcccgctctctctccttaaaaatttaggctaccttggctacaccagtggattttctctctcctgcatggtgtttttcgtcagtgtggtgatttacaaaaaattccaaattccctgccctctgcctgctctggatcacaacaacggaaatctgacgttcaacaacacacttccgattcacatgatctcgctgcctaatgactcggagagctcgggtgtgaacttcatgatggattacgctcaccacaacccagctgggctggatgagaagcaggtcgcaggccctcttcacagcaatggcgtggagtacgaagcccagggtgctgagaaatgccaaccaaaatactttgtgttcaattcccggacggcctatgcaatcccaatcctggcttttgcttttgtctgccaccctgaggtccttcccatctacagcgagcttaaagatcgatcccgcagaaagatgcagacggtgtccaacatttccatctcaggcatgctcgtcatgtaccttcttgcggccctctttggttatctgagcttctacggggacgttgaagacgagctgctgcatgcttacagcaaggtctacacatttgatacggctcttctcatggtgcgcctggcagtcctggtggcagtgacactgaccgtgcccatcgtgctgttcccgatccgtacttcggtgatcacactgctgtttccaaggaaacccttcagctggctgaagcatttcgggatcgctgcaatcatcatcgcactcaacaacatcctggtcatcctcgtgcctaccatcaaatacatctttggattcataggggcttcttctgccactatgctgattttcattcttccggctgcgttttatctcaagctcgtcaagaaagaacctctaagatcaccccagaagattggggctttggtcttccttgtgactggaattattttcatgatgggaagcatggcgctcattatactcgactggatctacaacccgccgaatcccaatcaccactaatcccggggagacgcgtctccactggaaacagctgaaattgtctgaaggacattttagttgtcttgattgggatgttagtctgaggaattagcaagattccaaagacgtttttctagctctatcagcacacattttaacccaggccgtgcagtgcagtgtgtgatgcccgagttgtgtttgcagcagctgtgcaagctgaagcctgttggctgcgtgtgttggtcagcagacaatagcctgtccccccatggtcactccacttctctccacccccagattaacaggtaattctactctcagaacatcagacaaagacctcctggttgggatacttgtggaagagaaaattatgggttttgttgggaatggttttgttgggaatggtgaaggatgcattaaaaattctgtgcgaagtatcatcagttacggccatctctcactctacaccaacactaagggtcggttgactagctgaggcagggggatatcttgggctgtccctgtgaggatcatgacgtatgacggttgccagtatagagtacttcatttcaatactcaaggaatagtttgcccaacctgcttattacaccgagttagtgaa

SEQ ID NO: 20 - DNA, 3408 bp, Mus musculus
  All n′s in this sequence represent unreadable nucleic acid.
gcagctaccggtccggaattcccgggtcgacccacgcgtcccacggctgcgagaagatcacagaagggggcggtgacgcaggaccaggactcgcgcgtccagcggagaagcaggagaagccggcgaccttgcgctctcagcctgatccctgtcttggcggcctgaacattcgcagctggagagatggcgttcgtgaagagtggatggttacttcggcagagcaccattctgaaacgctggaagaagaattggttcgacctgtggtcagacggtcacctgatctactacgatgatcagactcggcagagcatagaggataaggtccacatgcccgtggactgcatcaatatccgcacggggcatgagtgccgggacatccagcctccagatgggaagcccagagactgtctgctgcagatcgtttgccgagacgggaagaccatcagtctctgtgcagagagcacagacgattgcctggcatggaagtttacactgcaggattccagaacaaacacagcttacgttggttcagcaatcctgtctgaagagactgcagtggccgcgtccccgcctccctacgcaacctatgctacaccnacccctgaggtctacggctatggtccatacagcggcgcataccccgcaggaactcaagttgtctatgccgccaacgggcaggcatatgcagtgccataccagtacccgtatgcaggagtttatggacaacagcctgccaaccaagtcatcatccgcgagcggtaccgagacaatgacagtgacctggctctgggcatgctcgccggggcagccaccggcatggccctgggctctctgttctgggtcttctagagccttcaacattttctgtgcatagcttctgttagtcctgtgtgcagtaatttgatttgcagggcatttctgtttgtgacaagtgtctttcataataatttaaatagttcttttgaaggtggtaatctaataattgtgactgacctgcatggtaccacaaagaaagcccgaggtatgctgtgagtgagagcctgagtccttccgggtactagcttgcaccaagtctttcttagggacttttggatggctttatgtaaacacacccagttaaatgggcaatttccgtccagttaggtgcagtgttgaattaagggatggctttccttgctatgccaatactaatactgctgatggaggaagatgtgtgcaagtgtggtgaggagagtcacagcttctttaactgtggattctcttctagacccctgctgcgtgttaccctaggagctgtgggctggtggctcctgcaagactatggtgtgaggaccctgtaacgtacctcttggagcacttaggtaccccttgaagctcctaggtatcaccagcaggattggctgctcaggatgcagagggccaccccctccctttaaaaattacgctccagtaatctgcccagttttattttcttgttattcttctgtttgcttttcctggggatgattggcattagtctggagttaggaattgattcgagtgccggtgggtggaggcatgcagggagctgtccagcgacctgctctcagtgtttgttttaggtatattgattgccagctcaggctgcagagagcctatagagactatttttctacttgtaaagaaagtatggtgaggggaattgaggagagccttgtttgaatgttcctgcctcaggcctcctggggcccacactgcgtggtcctggggaggcttttcctcagcagtggaagggaggccccgtggctgtccagagtctcaggttttgagtgagaaatgggattgggtagagcatctcagggatttgttctaatccctcatgttatggggatccagccgtgttctcagtccagacccgctcacctcagaagagcttaaaacatttctggtccccaaatgtgtggcactctgagaagctcacaatctggctttctaacgaaaatttgtatttctaaaattagagaatacatgttccacgcatttaaaatttatgttctttcatgttttaaagctcccaaatccagctttgtgactggcatattttagtttcaaacagtaccccggcacaaaggtgggatggcacagtgaaggccccccgccctctactttgcatagtcttgtttctccagggtgctcccaggaagcattcattctgactttgctcagcccagtgcatgcgtgctgccttgccgccgtgctgctgggtagctctttcttggtcagatcaagtcttcaacagatctccatgtgagacagttgccaagtagatgaggtggtgcccatagtgctttctcgatactccttggggacctgttgacacctgcccatttccagctgacatttgtttttctgtcatctctgatagatgggatatgtgacaacatggtacggacgccgttcagtgtcgctttaataagcatgatgctgattttacatcctgtgctgtatgactgccatttgctcacagtgtcaccattgctaaagctccgtgctttacttacaaaacactaaaaccagtggttagtgtttcacagtgattttaattttagagttagttactggcattcctaaagccatagagtactgagtcacatccctgaagtacttttgaaacagaattgtctcctactgtcccatgggtgtgccctgcctgtctcctggccccaatggggctagctgtaccaggcagccatagttgagcctgatcattcctgtcaccagtttgacttgattatatacccagaatggaatacattcttgggcatctcagttcctcagccctgatcctcatagacgccaccctttcgatggcttttgcggcgtcacttgtacctcagtgagtcctgcgattcttgagttagaggggacgacttgtccagcattgaggaacatgtctcctccactgagacttaaatgatgatgcagggctggaagaggctggctgctgacactgcatcgtggctgatgtcattgctctcctagttctttgatttaagaacctttcatatggaaggcctgaggctccctcagatcgtcccttgccaagaaggcctggcttaggtcattagtgcccacagtagccttctggagtgtagcaagttcctgcgtttgagacagaatggttcagatttattttctacatctgttgttgaccccatgcaccctctcattttgccttccagtctacgtagatgaaagatgaaaggcagaggatgcagacagtcttctttgtgattgcttctgttattctgttgcatctaccgagcccgttttctccctgtctgtgcatacagtatgtttataagtgaacttgttaaaatattaaatgatcactaaccctaaaaaaaaaaaaaaaaaaaaaaaaaaaaa

SEQ ID NO: 21 - DNA, 878 bp, Mus musculus
  All n′s in this sequence represent unreadable nucleic acid.
aaatctcagctgtgcagccttctagaacctcatgtctacaaagtgacaaactttcctcaggcacaatccatcatacagtggatcaatcagtcagagtcagaggaagaactagtcaaaattacctcattttctgaattcattaacaccttaaagaaaacccacaaatacctaatggaagagagtttcaaaactgagcccccagaaagagtggaagaagcaaagagaatggctacatatgaagtcaccacagctctcagctccttcttgaagtacctcagagaaacacagcagccagacatgcagctgttgctactctccattgctactggtgtaggctatcagttggtaaacagtatctttcagcatcttctggggtgtgatgagttaaacttcctcttggatcaaacggaaaataacgaacataaataccaagaactgaaaaatatttgcaattacagagcccaggcattcttggtgctcacagccctaagagccacagttgaaatcacagatgtttctacagaagagaaaggacaacgtttgacattaatacaacaacatatggggtcactgttgtctgaagaagttgcacatgtcctcacaaaacatggagaacatcatgactgggaaaggctggagaatgatttgagattactcattgagggggactataaagccaccacccattccttacaaatggatgaagtaaaaaaacaattgcaaagtttntgccatgaaaagaaacagacttataaacaacaaggtaatgaaaacagaacaaaagaaatgatagaaaatggacatttcctggacttactccaacgtttaggcctagacaattactatccaaaaaaaaaaaaaaaaaaaaaaaaaaaaaa

SEQ ID NO: 22 - DNA, 731 bp, Mus musculus
  All n′s in this sequence represent unreadable nucleic acid.
actagactctggtagagaaaaaagtgcctcggagtggctgctgaacaacctcatgactcaccagagtgtggagcttttcanaaaaactcacttntaaacgtcaggaaacagctcattcgttcntctaacttatttgtgatgcaagtggaaatagatgtntatacagctcttaaaaagtggatgttccttcagctggtgccntcctggaatggggtctttgaaaacagcttttgacaaaaacagatgtctggttttccaagtggaaagaagactttgaaaggacgactttccntgaaactgaacagggaaaaccagntcgcgcccntgtnccgacatttaaggctacagtacattatcagtgatctggcttctgcaaggatcattgagcaggattctctggtaccttcagaatggctggcggcagtgtataaacagcagtggctggctatgctacgggctgaacaagacagtgaagtggcgcctcaagaaatcaataaagaagancttgagggaaacagcatgaggtgtggtcgaaagcttgccaaagatggtgngtactgctggcgctggacacgcttcaatttcggctttgacctccttgtgncttacaccaatcgatacatcattttccaacgccatacnctgaaccagccatgttattggatctgtcagcttacacctcgaaggagcatagcatttaaatgcccttggcttctttgacagtagtggga

SEQ ID NO: 23 - DNA, 400 bp, Mus musculus
  All n′s in this sequence represent unreadable nucleic acid.
gacatttaaggctacagtacattatcagtgatctggcttctgcaaggatcattgagcaggattctctggtaccttcagaatggctggcggcagtgtataaacagcagtggctggctatgctacgggctgaacaagacagtgaagtggggcctcaagaaatcaataaagaagaacttgagggaaacagcatgaggtgtggtcgaaagcttgccaaagatggtgagtactgctggcgctggacacgcttcaatttcggctttgacctccttgtgacttacaccaatcgatacatcattttccaacgccatacnctgaaccagccatgttattggatctgtcagcttacacctcgaaggancatancatttaaatgcccttggcttctttgacagtagtggga

SEQ ID NO: 24 - DNA, 2649 bp, Mus musculus
tgagcgcaacgcaattaatgtgagttagctcattcattaggcaccccaggctttacactttatgcttccggctcgtatgttgtgtggaattgtgagcggataacaatttcacacaggaaacagctatgaccatgattacgccaagctctaatacgactcactatagggaaagctggtacgcctgcaggtaccggtccggaattcccgggtcgacccacgcgtccgacggctgcgagaagacgacagaagggggcaccacagaggaccccttcaacctaaggaaacacccaggcttcgataggaccatgctgcagaggtggcagaaaagggagatcagcaactttgaatacctcatgtacctcaacacactggccggaaggacctacaatgactacatgcagtatcccgtgtttccctgggtcctcgctgactacacctcagagatgttgaacttgacgaatcccaagactttccgggatctttctaagccaatgggggctcagaccaaggaaaggaagttgaagtttacccagaggtttaaagatgttgaaaagattgaaggagacatgaccgtgcagtgccactactacacccactattcctcagccatcattgtcgcttcctacttggtccgaatgccaccattcacgcaggccttctgctccttacagggcggaagctttgatgtggctgatagaatgttccacagtgtaaagagcacgtgggagtctgcctccaaagagaacatgagcgatgtcagggagctgacacctgaattcttctacctgcccgagtttttaaccaactgtaatgcagtggagtttggctgcatgcaggatggaacgacactgggggatgtgcagcttcctccctgggctgacggggatccgaggaaattcatcagcttgcacagacaggctctggaaagtgacttcgtcagcagcaacctccaccactggatagacctaatttttgggtataagcagcaggggccggctgctgtagaggcagtgaacactttccacccctacttctacggtgatagaatagacctgggcagcatcactgacccgctgatcaagagcaccatcctgggcttcatcagcaactttggacaggtgcccaagcagatcttcactaaaccccacccatccagaaacaccacagggaaaaacccagggcctggaaaggatgcttccacccctgtaggcctcccaggccactcacagtccttcctccacagcctgccagcactgagaccctctcaggtcacagtcaaagatatgtaccttttctctctagggtcggaatcccccaaaggggccatcggccacatcgtccctactgagaagtcaatcctggcagtggagaagaacaagctgctgatgccccctctctggaacaggaccttcagctggggctttgatgacttcagttgctgcctggggagctacggctctgacaagatcctgatgacctttgagaacctggctgcctggggtccctgtctgtgcgctgtatgcccttcccccacgatgatcgtcacatccggggccagcgcagtggtgtgcatctgggagctgagcctggtcaaaggtcgcccgagaggtctgaaactccgacaggccttgtatggacacactcaggcggtcacatgtctgacagcctctgtcaccttcagcctcctggtgagcggatcccaggatcgcacttgtatcctgtgggacctggaccacctctctcgtgtggcctgcctgcctgtccaccgggaaggcatctcagccattgccatcagtgatgtctcgggaaccattgtctcctgtgccggagcccacttgtccctgtggaatgtcaatggacagcccctggccagtattaccacagcctggggcccagaaggaaccataacgtgctgctgcatagtagaggggccagcgtgggatgcaagccacgtgatcatcacggggagtaaggacggaatggttcggatttggaagacagaggacgtgaagatgcctgttcccaggcaggcagtgatggaggagccctccacggagcccctaagccccagaggtcacaagtgggccaagaatcttgccctgagccgagagctggatgtcagtgttgctctgagtggcaagcccagcaaggcaagtcctgctgtgacagctctggccatcactaggaaccagagcaagctcttggttggcgatgagaagggcgaatcttctgctggtctgctgatgggtaggagacaaggagctggagggaccgacctgaaagcctggagccctggggtcggcagcaacaggctacaggcacaagatgatgtgtagcctgggctgcttaaccagagcaagttttgggggggctccactccacacagttctcaaggagtccctgatggtttgcaccgtgtaccctaaacatgtctgtagtctatgggacttctgtaagaaggatcttggtagacactgatgctggaaactgacgctgcatgggaaatttctacgtggctcacttcaccaaggcttattgcactgggaaaagaagagggggtgggtattggttcatggaaaccaccccactgtctttattttattaaaactccattttccgaaaaaaaaaaaaaaaaaaaaaaaaaaaaaa

SEQ ID NO: 25 - DNA, 499 bp, Mus musculus
cccattacttccacctcccatgaggactgcacctgcggcagcgattttatgtgaacttggattactgtatggaaatggaggagtgagaaagtgtgggaatcataaaagaaggcagaaccaaaagacagagggatcttgggccatgagtcacagcaaggaaagcctcccaccaaacatctgcactggactcctacaggaacaagaaatgaactatcgtgttcagcccctgagacttgggcttaacatgattttcacattgtctaccctacctaatagagcagagatgtaaatattattcttattttagaggtgtgatgcctcagctgcaatgggtgagaactactcctcatttattctcttccaaggcaataaaagagaatggaccaaagacagtctgtcatcacatctagtcaaaagagctaatgtcgcagtacaactcttcaaaagaaaaagaaaaaacaagaaaaaagtaataaacagatgtgttctgcttgaaaaa

SEQ ID NO: 26 - DNA, 686 bp, Mus musculus
  All n′s in this sequence represent unreadable nucleic acid.
cggggtcgcgcgggaattaagtgagtccattcttgcacaccgagaaaatatgcgacagatgataagaagtttttctgaaccctttgggaagagacttgctcagtatctctgatggtaganggagagctcataatcgtagaggacataatgatggtgaagattctttgactcatacagatgtcagctctttccagacaatggaccaaatggtgtctaatatgagaaactatatgcagaaattagaaagaaacttcggtcaactttcagtggatccaaatggacattcattttgttcttcctcagttatgacttattccaaaataggagatgaaccgccaaaggtttttcatgcctcaactcaaactcgtcgagctccaggaagaataaaggaaaccaggaaagcnatgagagattctgacagtgggactagaaaaaatggctattggtcntcatatccatgaaccgagctcntgttcnttaataaagtccnnnganccnagaaagactgggagatgaaaaaaggtccacccaggaanttcatcccttatgaaatgaaaattgatgccccctgccttttgaatgaaggaattggccaaattnaaggtttttgaaattttcaaaccccggaacaacccaatcctnggaaaacccctnnaaatgaaaaaattttttgggcccttaaaaan

SEQ ID NO: 27 - DNA, 651 bp, Mus musculus
  All n′s in this sequence represent unreadable nucleic acid.
caaaannacntgtgtttagcaaataaaaactgcccagtgggacaancgtcgccgggaatcgctgtcagtactgccganttcanaaatgcctggctgttggganggncaaagaaatggnncgcacagacagnnnaaaaggccggagaagncgnntgcccncgaaaccgaagaaccacanganccctctcccccttcgcccccgttccangcgaacctgacnancaaangaanggaganganacccagcananccngcaantctatganctcctgactggctccanggaaancatcccgggcngggcagaaaaaatccctgggcnncgcagaactgccccaagccgaccaagaactgcnnnntgaancagcnntctnagaactgnttgtccnnccannagcanacagaagatgaacatcgacatntctgccttctcctgcatngcngccctgggctatggncacnnaaaaacacggcncngggaaccggaaaanggaanaangncnnccngatngntgantgncgcggagacacgngactttcnacnanggggggggnanncgccccccttnnttgttccnactgggggggggaacnncccnaaattcgngcccctnngggcccnaggggggggggggnccnttttctttctn

SEQ ID NO: 28 - DNA, 683 bp, Mus musculus
  All n′s in this sequence represent unreadable nucleic acid.
gggtctgtttggactgcagaagcaagggggtgatgtagcccatccttccctttggagatgctgagggtgtttcttcctgcacccacagccagggggatgccactcctccctccggcttgacctgtttctctgccgctacctccctccccgtctcattccgttgtctgtggatggtcattgcagtttaagagcagaacagatcttttactttggccgcttgaaaagctagtgtacctcctctcagtgttttggactccatctctcntcctccantaccttgcttcttactgataattttgctggaattcctaacttttcaatgacattttttttaactactatattgattgtcctttaaaaaagaaaagtgcatatttatccaaaatgtgtatttcttatacgcttttctttgttataccatttcctcagcttatctcttttatatttgtagganaaactcccatgttatggaatcccactgtatgatttataaacagacaatatgtngagtgccttttgcaanaaaaagggtgtgtttgaaatcatccggantcagccangaaactgtctccaaaggaaacnccnacctctctgttccttgctgttntgctgatcatccgccanaaggtgcttccccccgaattttggtttggttntgtttnccgaaaaattttttccgnttn

SEQ ID NO: 29 - DNA, 624 bp, Mus musculus
  All n′s in this sequence represent unreadable nucleic acid.
aatgcacctccttgtattcccactttcgtantcatttcggttctgatcttgtcaaacccagcctgaccgcttctgacgccgggatggcctcgttactanacttttctttttaaggaantgctgtttttttttganggttttcaaaacattttgaaaagcatttacttttttgaccacganccatganttttcaaaaaaatccggggttgtgtgggtttttggtttttgttttaatttttggttgcgttgccttttttttttaatggggttggccccatgaaatgggtgccccactcacttctctganatcgaacngactgtgaatccgctctttgtcngaanctgaacaanctgtggcttttttccaactccgtgtgacntttctgaatgttatgtggtaagaccccgcgggtgtggcancaactgccctggaaccccancccctgcntccatctgtngctgntgcgccccacantaaaacnttcanacntccctgaaaagttcttgaaaaaanttaattanattgtcccnnttttactgggaaaaaattanccntactccnnccaattttnttcttncnantgggctgaaagaaatccttgttccccanttttcctttgggcn

SEQ ID NO: 30 - DNA, 641 bp, Mus abbotti
  All n′s in this sequence represent unreadable nucleic acid.
atcccagaccaggcaacaaggtcctttgaggtctataatgaaagatctgcattctgatgacaatgaggaggaatcagatgaagtggaggataacgacaatgactctgaaatggagaggcctgtnaatagaggangcagccgaagtcgcagagttagcttaagtgatggcagcgatagtgaaagcagttctgcttcttcacccctacatcacgaacctccaccacccttactaaaaaccaacaacaaccagattcttgaagtgaaaagtccnataaagcaaagcaaatcagataagcaaataaagaatggtgaatgtgacaaggcatacctagatgaactggtngagcttcccagaangttnatgacattgagagaaagacacattctgcagcagatcgtgaaccttatngaagaaactgggacactttcatatcaccaacacaacatttgattttgatctttgctcgctggacaaaaccacagtccgttaactacagagttcctggaaacatctggaacatcctgaagatataaccactggatgcctcnagaactaatgtgtttnnnnnnnnnnngggttnnnnnntttgggtgtgaatttttgttcccgttgtttaaatgaaaaccccccnaatgatgcn

SEQ ID NO: 31 - DNA, 400 bp, Mus musculus
  All n′s in this sequence represent unreadable nucleic acid.
gcgagccgacggagnggctctacggtggatagcgtgttccggaacctatccctcgaattagccgagtcaggcagagagggggcggggngtgcttccgcccttgctaggaggggctgcattgcaggggagacccagcggcagattctgtcacagacgagggagaaggcgtgaggagacaaagccgtcacatccgcgacagcttccttcagcagcctcctcctctccagtccagagccgacccccgagcccctgaggcatcccttccgtcttcggaacaccctagtcattcattgctaacaggggatcatgagggaccctgtaagtagccagtacagctcctttcttttctggangatgcccatcccacaactggatctgtcngagctggaaggcctgggcc

SEQ ID NO: 32 - DNA, 400 bp, Mus musculus
  All n′s in this sequence represent unreadable nucleic acid.
acggctgcgagaagacgacagaagggaagctaagggctggtcgctgcgtctgaacgccgggttggacgaaccgctgtgcgcccttggcggacgtnagcggaaagaagatggcggtgcaggtggtgcaagctgtgcangcggttcatcttgantctgacgctttcctantttgtctcaaccatgctctgagcacagaaaangangaagtgatgggtctgtgtatagggganttgaatgatgacataaggagtgactccnaatttacatacactggaacggaaatgcgcacagtccnagaaaagatggataccatcagaattgttcatatccattctgtcatcatcttgcggcgttctgacaaganaaangaccgtgtnnaaatttctccanancanctgtc

SEQ ID NO: 33 - DNA, 400 bp, Mus musculus
  All n′s in this sequence represent unreadable nucleic acid.
gatggcggtctccacaggagttaaagttcctcgtaattttcgcttgttggaagaacttgaagaaggacaaaaaggagtaggtgatggtactgttagctggggccttgaagatgatgaagacatgacacttacaaggtggacaggcatgattattgggccaccaaggacaaactatgaaaacagaatatatagcctgaaagtagaatgtggatctaaatacccagaagctcctccatcagttagatttgtaacaaaaattaatatgaatgggatcaataattccagtggaatggtggatgcacggagcataccagtattagcaaaatggcaaaattcctatagcattaaagtcatacttcaaganctaagactcttatgatgtccaaagaaaatatgaanc

SEQ ID NO: 34 - DNA, 400 bp, Mus musculus
  All n′s in this sequence represent unreadable nucleic acid.
acggctgcgagaagacgacagaagggggagcttctgcgtcccagggactccagtacaccaccatggcggattctgagcgtctctcggcccccggctgctggttagcctgcaccagcttctcgcgcaccaaaaagggaattctcctgtttgctganattatactgtgcctggtgatcttgatttgcttcagtgcatctacaacatcggcctactcctccctgtcggtgattgagatgatctgtgctgctgtcttacttgtcttctacacgtgtgacctgcactccaagatatcattcatcaactggccttggactgacttcttcagatccctcataacaaccatcctgtacctgatcacctccattgttgtccttgtagaaagaagaagcagctccagagt

SEQ ID NO: 35 - DNA, 400 bp, Mus musculus
  All n′s in this sequence represent unreadable nucleic acid.
tacggctgcgagaagacgacagaaggggctccgccttcttccccacctccggctgccggcgacaccggtctttgcagtccgggaccccgtgcgatcgttagcccggtgcacgatgcccccgaaaaagggaggagatggaattaaaccgcctccaattattggaagatttggaacctcactgaaaattggtatcgttggattgccaaatgttgggaaatctaccttcttcaatgtattaaccaatagtcaggcttcancagaaaacttcccattctgcactattgatcctaatgagagcagagtgcctgtgccagatgagaggttcgactttctttgccagtaccataaaccancaagcaagatcctgctttcctaaatgtaatggatattgctggccttg

SEQ ID NO: 36 - DNA, 400 bp, Mus musculus
  All n′s in this sequence represent unreadable nucleic acid.
acggctgcgagaagacgacngaaggggtcctttttcctgcgcggcgtttcagtccctcttgagttgggctgtcgtcnantcgcggcganaccggcgcaccgcagccatgacagaagctgatgtnaatccgaangcctatcccctcncanatncccacctcaccaagaanctgctggaccttgttcnacagtcttgttnctacaancagcttcngaaaggaccnatgaaccnccaaaaccctcaacagaagcatctctganttcattgtgatgcngcananctgaaccttggagatctcctgcacctccctctnctgtcgaaaaaaanaatgtccctacctntttgtncnnccaacaggctttangaaaggcctntgggtttccagccntcatccctgtct

SEQ ID NO: 37 - DNA, 394 bp, Mus musculus
  All n′s in this sequence represent unreadable nucleic acid.
gaaaggcgcctagataccgcgatacttgcgggtatcctggagctggactctcctgttcttgccacatttgactgaagaaggacaacggggtcaaggtttgaggactgacagattctanacccaggcttcctcngcccccanaaagccaccanacctcacatggaaccaaaggccccatgcccanccgccgtcccctcagagganangaaattccgtgttcttgtnggcgtcactggcagcgtggccgctctgaagctgcctctcctgntatctannctgttggacgttcctgngatgtggaagcgccgttctgacccggttctccacattgacctgcggatgtgggctgacctcatgctagtggctcccctcnatgcnnacactctgnggaagt

SEQ ID NO: 38 - DNA, 315 bp, Mus musculus
  All n′s in this sequence represent unreadable nucleic acid.
acggctgcgagaagacgacngaangggggcaggatggctcggtggtacagttcaagatcaanangcataccccactgagcaagctgatgaagcctactgtgagaggcagggcttgtcnatnangcagattcgattccggtttgatggacaaccaatcaatgaaacagacactccaccccanctggagatggaggatgaggacaccnttgatgtattccagcagcagacaggaggatcancctcccgagggagcgtcccacacccaaccgttgtcctgacctgtgctattgagcagtgaccatgctnccacaccca

SEQ ID NO: 39 - DNA, 400 bp, Mus musculus
  All n′s in this sequence represent unreadable nucleic acid.
acggctgcgagaagacgacagaaggggagagcatcatggcgctaagcggtcgactggcattggccgcgctcagactgtggggtccgggaggatgtatctganctgcttcggaccanancctgcagtagggtggtggtcatggtgggggccggcatcancacacccagtggcatcccggacttcagatccccaggggagcggcctctacagcaaccttcagcagtatgacatcccgtaccctgaagccatctttgaacttggctttttctttcacaaccccaagccctttttcatgttggccaaggactgtnccctgngcactacaggccaatgtcactcactacttcctgaagctcctccacgacanggaccgcttctgcgctctntacacanaaatcga

SEQ ID NO: 40 - DNA, 349 bp, Mus musculus
  All n′s in this sequence represent unreadable nucleic acid.
ggccatcaancgtgctgttggtcatggccgactcctcaaagatggacccctctaccgcctcaacactaaggcagccagtgctgaagggaaagagggctgtgagtcgctctcgtgtttacctcccgtgtcactgcttccacacganaaagacaagccggttgctgaaccaatccccatctgtagcttctgtcttggtacaaaagaacagaaccgggagaagcagcccgaggaactcgtctcctgcgcggactgcggcaatancggtcatccatcgtgtttaaagttctccccanagctnacagtgagagtgaagccttacngtggcttncattgantgtaaaacatcact

SEQ ID NO: 41 - DNA, 397 bp, Mus musculus
cccacgcgtccgatctcctccagggccaccaagcacctctgaagagccatgttccaagctgccggagccgcccaggccaccccctctcatgaagccaaaggcagcagtggcagcagcacggtacagcggtctaagtcctttagcttgcgggctcaggtgaaggagacctgtgcagcctgccagaagactgtgtacccgatggagcggctggtggcagacaagctcattttccacaactcttgtttctgttgcaaacactgccacaccaaactcagcctgggcagttatgctgcaatgcacggtgaattttactgcagacctcactttcagcagctgtttaagagtaaaggcaactacgatgaaggttttcgtaaacagcacaaggactctgggccac

SEQ ID NO: 42 - protein, 158 aa, Mus musculus
MFQAAGAAQATPSHEAKGSSGSSTVQRSKSFSLRAQVKETCAACQKTVYPMERLVADKLIFHNSCFCCKHCHTKLSLGSYAAMHGEFYCRPHFQQLFKSKGNYDEGFGRKQHKELWAHKEVDSGTKTADPFNTHSLPAHGLPLGSGKEINPGARGGRG

SEQ ID NO: 43 - DNA, 1360 bp, Mus musculus; identical to SEQ ID NO: 13

SEQ ID NO: 44 - protein, 419 aa, Mus musculus
MSPLLLLLLCLLLGNLEPEEAKLIRVPLQRIHLGHRILNPLNGWEQLAELSRTSTSGGNPSFVPLSKFMNTQYFGTIGLGTPPQNFTVVFDTGSSNLWVPSTRCHFFSLACWFHHRFNPKASSSFRPNGTKFAIQYGTGRLSGILSQDNLTIGGIHDAFVTFGEALWEPSLIFALAHFDGILGLGFPTLAVGGVQPPLDAMVEQGLLEKPVFSFYLNRDSEGSDGGELVLGGSDPAHYVPPLTFIPVTIPAYWQVHMESVKVGTGLSLCAQGCSAILDTGTSLITGPSEEIRALNKAIGGYPFLNGQYFIQCSKTPTLPPVSFHLGGVWFNLTGQDYVIKILQSDVGLCLLGFQALDIPKPAGPLWILGDVFLGPYVAVFDRGDKNVGPRVGLARAQSRSTDRAERRTTQAQFFKRRPG

SEQ ID NO: 45 - DNA, 3338 bp, Mus musculus
  All n′s in this sequence represent unreadable nucleic acid.
cggtgacgcaggaccaggactcgcgcgtccagcggagaagcaggagaagccggcgaccttgcgctctcagcctgatccctgtcttggcggcctgaacattcgcagctggagagatggcgttcgtgaagagtggatggttacttcggcagagcaccattctgaaacgctggaagaagaattggttcgacctgtggtcagacggtcacctgatctactacgatgatcagactcggcagagcatagaggataaggtccacatgcccgtggactgcatcaatatccgcacggggcatgagtgccgggacatccagcctccagatgggaagcccagagactgtctgctgcagatcgtttgccgagacgggaagaccatcagtctctgtgcagagagcacagacgattgcctggcatggaagtttacactgcaggattccagaacaaacacagcttacgttggttcagcaatcctgtctgaagagactgcagtggccgcgtccccgcctccctacgcaacctatgctacaccnacccctgaggtctacggctatggtccatacagcggcgcataccccgcaggaactcaagttgtctatgccgccaacgggcaggcatatgcagtgccataccagtacccgtatgcaggagtttatggacaacagcctgccaaccaagtcatcatccgcgagcggtaccgagacaatgacagtgacctggctctgggcatgctcgccggggcagccaccggcatggccctgggctctctgttctgggtcttctagagccttcaacattttctgtgcatagcttctgttagtcctgtgtgcagtaatttgatttgcagggcatttctgtttgtgacaagtgtctttcataataatttaaatagttcttttgaaggtggtaatctaataattgtgactgacctgcatggtaccacaaagaaagcccgaggtatgctgtgagtgagagcctgagtccttccgggtactagcttgcaccaagtctttcttagggacttttggatggctttatgtaaacacacccagttaaatgggcaatttccgtccagttaggtgcagtgttgaattaagggatggctttccttgctatgccaatactaatactgctgatggaggaagatgtgtgcaagtgtggtgaggagagtcacagcttctttaactgtggattctcttctagacccctgctgcgtgttaccctaggagctgtgggctggtggctcctgcaagactatggtgtgaggaccctgtaacgtacctcttggagcacttaggtaccccttgaagctcctaggtatcaccagcaggattggctgctcaggatgcagagggccaccccctccctttaaaaattacgctccagtaatctgcccagttttattttcttgttattcttctgtttgcttttcctggggatgattggcattagtctggagttaggaattgattcgagtgccggtgggtggaggcatgcagggagctgtccagcgacctgctctcagtgtttgttttaggtatattgattgccagctcaggctgcagagagcctatagagactatttttctacttgtaaagaaagtatggtgaggggaattgaggagagccttgtttgaatgttcctgcctcaggcctcctggggcccacactgcgtggtcctggggaggcttttcctcagcagtggaagggaggccccgtggctgtccagagtctcaggttttgagtgagaaatgggattgggtagagcatctcagggatttgttctaatccctcatgttatggggatccagccgtgttctcagtccagacccgctcacctcagaagagcttaaaacatttctggtccccaaatgtgtggcactctgagaagctcacaatctggctttctaacgaaaatttgtatttctaaaattagagaatacatgttccacgcatttaaaatttatgttctttcatgttttaaagctcccaaatccagctttgtgactggcatattttagtttcaaacagtaccccggcacaaaggtgggatggcacagtgaaggccccccgccctctactttgcatagtcttgtttctccagggtgctcccaggaagcattcattctgactttgctcagcccagtgcatgcgtgctgccttgccgccgtgctgctgggtagctctttcttggtcagatcaagtcttcaacagatctccatgtgagacagttgccaagtagatgaggtggtgcccatagtgctttctcgatactccttggggacctgttgacacctgcccatttccagctgacatttgtttttctgtcatctctgatagatgggatatgtgacaacatggtacggacgccgttcagtgtcgctttaataagcatgatgctgattttacatcctgtgctgtatgactgccatttgctcacagtgtcaccattgctaaagctccgtgctttacttacaaaacactaaaaccagtggttagtgtttcacagtgattttaattttagagttagttactggcattcctaaagccatagagtactgagtcacatccctgaagtacttttgaaacagaattgtctcctactgtcccatgggtgtgccctgcctgtctcctggccccaatggggctagctgtaccaggcagccatagttgagcctgatcattcctgtcaccagtttgacttgattatatacccagaatggaatacattcttgggcatctcagttcctcagccctgatcctcatagacgccaccctttcgatggcttttgcggcgtcacttgtacctcagtgagtcctgcgattcttgagttagaggggacgacttgtccagcattgaggaacatgtctcctccactgagacttaaatgatgatgcagggctggaagaggctggctgctgacactgcatcgtggctgatgtcattgctctcctagttctttgatttaagaacctttcatatggaaggcctgaggctccctcagatcgtcccttgccaagaaggcctggcttaggtcattagtgcccacagtagccttctggagtgtagcaagttcctgcgtttgagacagaatggttcagatttattttctacatctgttgttgaccccatgcaccctctcattttgccttccagtctacgtagatgaaagatgaaaggcagaggatgcagacagtcttctttgtgattgcttctgttattctgttgcatctaccgagcccgttttctccctgtctgtgcatacagtatgtttataagtgaacttgttaaaatattaaatgatcactaaccctaaaaaaaaaaaaaaaaaaaaaaaaaaaaa

SEQ ID NO: 46 - protein, 221 aa, Mus musculus
  All Xaa′s in this sequence represent unreadable amino acid.
MAFVKSGWLLRQSTILKRWKKNWFDLWSDGHLIYYDDQTRQSIEDKVHMPVDCINIRTGHECRDIQPPDGKPRDCLLQIVCRDGKTISLCAESTDDCLAWKFTLQDSRTNTAYVGSAILSEETAVAASPPPYATYATXTPEVYGYGPYSGAYPAGTQVVYAANGQAYAVPYQYPYAGVYGQQPANQVIIRERYRDNDSDLALGMLAGAATGMALGSLFWVF

SEQ ID NO: 47 - DNA, 2396 bp, Mus musculus
caccacagaggaccccttcaacctaaggaaacacccaggcttcgataggaccatgctgcagaggtggcagaaaagggagatcagcaactttgaatacctcatgtacctcaacacactggccggaaggacctacaatgactacatgcagtatcccgtgtttccctgggtcctcgctgactacacctcagagatgttgaacttgacgaatcccaagactttccgggatctttctaagccaatgggggctcagaccaaggaaaggaagttgaagtttacccagaggtttaaagatgttgaaaagattgaaggagacatgaccgtgcagtgccactactacacccactattcctcagccatcattgtcgcttcctacttggtccgaatgccaccattcacgcaggccttctgctccttacagggcggaagctttgatgtggctgatagaatgttccacagtgtaaagagcacgtgggagtctgcctccaaagagaacatgagcgatgtcagggagctgacacctgaattcttctacctgcccgagtttttaaccaactgtaatgcagtggagtttggctgcatgcaggatggaacgacactgggggatgtgcagcttcctccctgggctgacggggatccgaggaaattcatcagcttgcacagacaggctctggaaagtgacttcgtcagcagcaacctccaccactggatagacctaatttttgggtataagcagcaggggccggctgctgtagaggcagtgaacactttccacccctacttctacggtgatagaatagacctgggcagcatcactgacccgctgatcaagagcaccatcctgggcttcatcagcaactttggacaggtgcccaagcagatcttcactaaaccccacccatccagaaacaccacagggaaaaacccagggcctggaaaggatgcttccacccctgtaggcctcccaggccactcacagtccttcctccacagcctgccagcactgagaccctctcaggtcacagtcaaagatatgtaccttttctctctagggtcggaatcccccaaaggggccatcggccacatcgtccctactgagaagtcaatcctggcagtggagaagaacaagctgctgatgccccctctctggaacaggaccttcagctggggctttgatgacttcagttgctgcctggggagctacggctctgacaagatcctgatgacctttgagaacctggctgcctggggtccctgtctgtgcgctgtatgcccttcccccacgatgatcgtcacatccggggccagcgcagtggtgtgcatctgggagctgagcctggtcaaaggtcgcccgagaggtctgaaactccgacaggccttgtatggacacactcaggcggtcacatgtctgacagcctctgtcaccttcagcctcctggtgagcggatcccaggatcgcacttgtatcctgtgggacctggaccacctctctcgtgtggcctgcctgcctgtccaccgggaaggcatctcagccattgccatcagtgatgtctcgggaaccattgtctcctgtgccggagcccacttgtccctgtggaatgtcaatggacagcccctggccagtattaccacagcctggggcccagaaggaaccataacgtgctgctgcatagtagaggggccagcgtgggatgcaagccacgtgatcatcacggggagtaaggacggaatggttcggatttggaagacagaggacgtgaagatgcctgttcccaggcaggcagtgatggaggagccctccacggagcccctaagccccagaggtcacaagtgggccaagaatcttgccctgagccgagagctggatgtcagtgttgctctgagtggcaagcccagcaaggcaagtcctgctgtgacagctctggccatcactaggaaccagagcaagctcttggttggcgatgagaagggcgaatcttctgctggtctgctgatgggtaggagacaaggagctggagggaccgacctgaaagcctggagccctggggtcggcagcaacaggctacaggcacaagatgatgtgtagcctgggctgcttaaccagagcaagttttgggggggctccactccacacagttctcaaggagtccctgatggtttgcaccgtgtaccctaaacatgtctgtagtctatgggacttctgtaagaaggatcttggtagacactgatgctggaaactgacgctgcatgggaaatttctacgtggctcacttcaccaaggcttattgcactgggaaaagaagagggggtgggtattggttcatggaaaccaccccactgtctttattttattaaaactccattttccgaaaaaaaaaaaaaaaaaaaaaaaaaaaaaa

SEQ ID NO: 48 - protein, 693 aa, Mus musculus
TTEDPFNLRKHPGFDRTMLQRWQKREISNFEYLMYLNTLAGRTYNDYMQYPVFPWVLADYTSEMLNLTNPKTFRDLSKPMGAQTKERKLKFTQRFKDVEKIEGDMTVQCHYYTHYSSAIIVASYLVRMPPFTQAFCSLQGGSFDVADRMFHSVKSTWESASKENMSDVRELTPEFFYLPEFLTNCNAVEFGCMQDGTTLGDVQLPPWADGDPRKFISLHRQALESDFVSSNLHHWIDLIFGYKQQGPAAVEAVNTFHPYFYGDRIDLGSITDPLIKSTILGFISNFGQVPKQIFTKPHPSRNTTGKNPGPGKDASTPVGLPGHSQSFLHSLPALRPSQVTVKDMYLFSLGSESPKGAIGHIVPTEKSILAVEKNKLLMPPLWNRTFSWGFDDFSCCLGSYGSDKILMTFENLAAWGPCLCAVCPSPTMIVTSGASAVVCIWELSLVKGRPRGLKLRQALYGHTQAVTCLTASVTFSLLVSGSQDRTCILWDLDHLSRVACLPVHREGISAIAISDVSGTIVSCAGAHLSLWNVNGQPLASITTAWGPEGTITCCCIVEGPAWDASHVIITGSKDGMVRIWKTEDVKMPVPRQAVMEEPSTEPLSPRGHKWAKNLALSRELDVSVALSGKPSKASPAVTALAITRNQSKLLVGDEKGESSAGLLMGRRQGAGGTDLKAWSPGVGSNRLQAQDDV

SEQ ID NO: 49 - DNA, 2363 bp, Mus musculus
  All n′s in this sequence represent unreadable nucleic acid.
cagaggcaggcaggacaggcactgcasgcactgagagccttgcagtcagaagggaagcacasacaggaagaggcagtgaggagaaaaggagcasaactgagacaagcaatggagatccctgaggagtgctggccaacggctgaagtgtccctaaaagacatcactgaaataatggagagacatctcagtcacatggaacggaccctgtctcacagtcaaaagctctcagatggagacctggtaagatgggcatctggagggctggtcctgcagggaatttataagaccaaccacccaagaagcctgattcagaagagagaagagctactcagtgtccctaagcagttctcacttgctggcccagtacatggcacagagataaaaacaaaggaattttcatcttttcaagaacaagccatgtttacacagactatagagagggtgggtttcagttcaacccccttagttaaaggagaaggttggggacttagtctagaagctggtatgggtcacaacaaacagacagaatctgaagataactaccagtcccattcaaagcaaacttatttttgctcagccaggttcagctacatcccattggccacctgccattttcacatcaatgatcttgaactctcccaggctgctctccaggaactaaaaagtattgaagaaatcctggagcagactacacaccaccgagatggactacccttactgaggcacagggctaaaaactttttccacaggtttggctctcatgctaaccaaggccctgtgcacctggggggaatctactgctggaaagccatttcagaaggtttcaaaagtgagcacttggctgatgtaaagcagcaagcagaagagtctttgaatatttacattatgggcagttatagtggctttggagttaaagttggtgcgagtgtaaatataacaaattcaaaatcaaaaacagcattttacagtaaaactcatctaaactcgcaaaccaaggtacaactatctgtagccaagataggtggaccagcagaagcagatggaattgcccagtggacagctggccttgtagctagcaatcaaacctggtctgttattgataggaaactgcagttggtacctatttgggacattatcctgtccagtcacagaactgaatttaagaatgctcttcaactggctaactgcctcaaagaccactacactgctctgactgaactagctgcccagattcaagaaggggaagaatttctgactgctagaaaagaagctaagcttttcctaaagaatgtgaaaggctgggaggtttctgatcctgaagaacagcttaggaagttagtagattttatgcaaacattgagtcaaaaaataaaaagttatgacatttggattaacacatgcctcatagattgggatctgcaaaattttctaataaacattgtcaacttctgcaaaaattcacccacttataaaactcactttattaaatctcagttgtgcagccttctagaacctcatgtctacaaagtgacaaactttcctcaggcacaatccatcatacagtggatcaatcagtcagagtcagaggaagaactagtcaaaattacctcattttctgaattcattaacaccttaaagaaaacccacaaatacctaatggaagagagtttcaaaactgagcccccagaaagagtggaagaagcaaagagaatggctacatatgaagtcaccacagctctcagctccttcttgaagtacctcagagaaacacagcagccagacatgcagctgttgctactctccattgctactggtgtaggctatcagttggtaaacagtatttttcagcatcttctggggtgtgatgagttaaacttcctcttggatcaaatggaaaataacgaacataaataccaagaactgaaaaatatttgcaattacagagcccaggcattcttggtgctcacagccctaagagccacagttgaaatcacagatgtttctacagaagagaaaggacaacgtttgacattaatacaacaacatatggggtcactgttgtctgaagaagttgcacatgtcctcacaaaacatggagaacatcatgactgggaaaggctggagaatgatttgagattactcattgagggggactataaagccaccacccattccttacaaatggatgaagtaaaaaaacaattgcaaagtttntgccatgaaaagaaacagacttataaacaacaaggtaatgaaaacagaacaaaagaaatgatagaaaatggacatttcctggacttactccaacgtttaggcctagacaattactatccaaaaaaaaaaaaaaaaaaaaaaaaaaaaaa

SEQ ID NO: 50 - protein, 778 aa, Mus musculus
  All Xaa′s in this sequence represent unreadable amino acid.
QRQAGQALXALRALQSEGKHXQEEAVRRKGAXLRQAMEIPEECWPTAEVSLKDITEIMERHLSHMERTLSHSQKLSDGDLVRWASGGLVLQGIYKTNHPRSLIQKREELLSVPKQFSLAGPVHGTEIKTKEFSSFQEQAMFTQTIERVGFSSTPLVKGEGWGLSLEAGMGHNKQTESEDNYQSHSKQTYFCSARFSYIPLATCHFHINDLELSQAALQELKSIEEILEQTTHHRDGLPLLRHRAKNFFHRFGSHANQGPVHLGGIYCWKAISEGFKSEHLADVKQQAEESLNIYIMGSYSGFGVKVGASVNITNSKSKTAFYSKTHLNSQTKVQLSVAKIGGPAEADGIAQWTAGLVASNQTWSVIDRKLQLVPIWDIILSSHRTEFKNALQLANCLKDHYTALTELAAQIQEGEEFLTARKEAKLFLKNVKGWEVSDPEEQLRKLVDFMQTLSQKIKSYDIWINTCLIDWDLQNFLINIVNFCKNSPTYKTHFIKSQLCSLLEPHVYKVTNFPQAQSIIQWINQSESEEELVKITSFSEFINTLKKTHKYLMEESFKTEPPERVEEAKRMATYEVTTALSSFLKYLRETQQPDMQLLLLSIATGVGYQLVNSIFQHLLGCDELNFLLDQMENNEHKYQELKNICNYRAQAFLVLTALRATVEITDVSTEEKGQRLTLIQQHMGSLLSEEVAHVLTKHGEHHDWERLENDLRLLIEGDYKATTHSLQMDEVKKQLQSXCHEKKQTYKQQGNENRTKEMIENGHFLDLLQRLGLDNYYP

SEQ ID NO: 51 - DNA, 1712 bp, Mus musculus; identical to SEQ ID NO: 14

SEQ ID NO: 52 - protein, 336 aa, Mus musculus
MAASRDADEIHKDVQNYYGNVLKTSADLQTNACVTRAKPVPSYIRESLQNVHEDVSSRYYGCGLTVPERLENCRILDLGSGSGRDCYVLSQLVGEKGHVTGIDMTEVQVEVAKTYLEHHMEKFGFQAPNVTFLHGRIEKLAEAGIQSESYGIVISNCVINLVPDKQQVLQEVYRVLKHGGELYFSDVYASLEVPEDIKSHKVLWGECLGGALYWKDLAIIAQKIGFCPPRLVTADIITVENKELEGVLGDCRFVSATFRLFKLPKTEPAERCRVVYNGGIKGHEKELIFDANFTFKEGEAVAVDEETAAVLKNSRFAPDFLFTPVDASLPAPQGPF

SEQ ID NO: 53 - DNA, 3599 bp, Mus musculus; identical to SEQ ID NO: 16

SEQ ID NO: 54 - protein, 687 aa, Mus musculus
MAVQVLRQMVYFLLSLFSLVQGAHSGSPREDFRFCGQRNQTQQSTLHYDQSSEPHIFVWNTEETLTIRAPFLAAPDIPRFFPEPRGLYHFCLYWSRHTGRLHLRYGKHDYLLSSQASRLLCFQKQEQSLKQGAPLIATSVSSWQIPQNTSLPGAPSFIFSFHNAPHKVSHNASVDMCDLKKELQQLSRYLQHPQKAAKRPTAAFISQQLQSLESKLTSVSFLGDTLSFEEDRVNATVWKLPPTAGLEDLHIHSQKEEEQSEVQAYSLLLPRAVFQQTRGRRRDDAKRLLVVDFSSQALFQDKNSSQVLGEKVLGIVVQNTKVTNLSDPVVLTFQHQPQPKNVTLQCVFWVEDPASSSTGSWSSAGCETVSRDTQTSCLCNHLTYFAVLMVSSTEVEATHKHYLTLLSYVGCVISALACVFTIAAYLCSRRKSRDYTIKVHMNLLSAVFLLDVSFLLSEPVALTGSEAACRTSAMFLHFSLLACLSWMGLEGYNLYRLVVEVFGTYVPGYLLKLSIVGWGFPVFLVTLVALVDVNNYGPIILAVRRTPERVTYPSMCWIRDSLVSYVTNLGLFSLVFLFNLAMLATMVVQILRLRPHSQNWPHVLTLLGLSLVLGLPWALVFFSFASGTFQLVILYLFSIITSYQGFLIFLWYWSMRFQAQGGPSPLKNNSDSAKLPISSGSTSSSRI

SEQ ID NO: 55 - DNA, 3590 bp, Mus musculus
ctcagggaaggccgatcttccgggtggagggggaagcggcgtgactggagtggaaatttttcccaacacaacttctcggaggcaacatattggaagggactcggggaggccggagtccaaatggaagtggctgaaagaaacttctcgccctgctgattctgagccccgcgtcgtgccgcgcgccctcaattacctcatcgacctgtggtcttgacagaacattcttcacaatccaaaagaaaaagcagactggttcgggatgtttgacggctatgatagctgcagtgaggacacaagtagcagctccagctctgaggagagtgaagaagaagttgctcctttaccttccaatctcccaatcatcaagaataatggacaagtctacacatacccagatggtaaatctggcatggctacctgtgagatgtgtgggatggtcggtgtgcgagatgctttttactctaaaacgaaacgtttctgcagtgtttcctgttcaagaagttactcgtcaaactctaagaaggcaagcattctggccagacttcagggtaagcctccaacaaagaaagcaaaagtccttcaaaaacaacctttagttgctaaattagctgcctatgcccagtatcaagctaccttgcaaaatcaagcaaagacaaaagcaggcaattctgcaatctctgtggaaggcttcagctggggtaactacatcaacagcaacagcttcatagcagctccagtggcctgttttaagcatgcacctatggggacctgctggggtgatatctcggaaaatgtaaggatagaagttcccaatacagactgcagtctacctaccaaagtcttctggattgctggaattataaaattagcaggttataatgcccttttgagatatgaaggatttgaaaatgattcttctctggacttctggtgcaatatatgtgggtctgatattcatccagttggttggtgtgcagctagtggaaaacctctcgttcctcctagaactgttcaacataaatatacaaactggaaagcttttctagtaaaaagacttactggtgccaaaacacttcctcctgatttctcacaaaaggtttctgagagtatgcaatatcctttcaaaccttgcatgagagtagaagtagttgacaagaggcatttatgtcgaacaagagtagcagtggtggaaagtgtaattggaggacgactacggctggtgtatgaagagagtgaagatggaacagacgacttctggtgccacatgcacagccccttaatccaccatattggatggtcaagaagcataggccatcgattcaagagatcagatattacgaagaaacaggacggacatttcgatacacctccacacttatttgctaaggtaaaagaagtagaccagagtggagaatggttcaaagaaggaatgaaattggaagctatagacccattaaatctttctacaatatgtgttgccaccattagaaaggtgctggctgatggattcctgatgattgggattgatggctcagaagcagcagatggatctgactggttctgttatcatgcaacctctccttccattttccctgtgggtttctgtgaaattaacatgatagaactgactccacccagaggttacacaaaacttccttttaaatggtttgactacctcagggaaaccggctccattgcagcaccagtaaaactatttaataaggatgttccaaaccacggattccggttaggaatgaaattagaagctgtagatctcatggagccacggttaatatgtgtagccacagttactcgaattattcaccatctcttgaggatacattttgatggttgggaagaagagtatgaccagtgggtagactgtgagtcccctgacctctatcctgtagggtggtgtcagttaactggatatcaactacagcctccagcatcacagtcatcaagagaaagccaatcagcttcttcaaaacagaagaaaaaggctaagtctcagcaatacaaaggacataagaaaatgaccacgtcacagctgaaggaggagctgctggacggggaggactatagcttcctccatggagcatctgaccaggaaagcaatggctctgccaccgtctacatcaaacaagagccatgaggcgactcggaaaccatgggcaggcggggctgtttacaggactgatttggaatcagccagctgtatagcgggctattctactgggacattttgctaaacacagaaaaaagttcagttccagatttttcaggtgggggggaaactattttggtgggggggcaatttttcaatttataaagacggacaatttttgtgttgtatttgaagcttttgaaagaattttgtaatattttccaagtttggatttatgtgcattgttaacaagaactgaaattataacttttttggtaagataaaagtttaggtagcaggattgaaggaaatgattaagaaggatatagttgtaaatgcacatgaactgtcattacaaatgaaccttcttggtacctgttgggagattttttggattttagagttaggccagtcacatctccagcttcctttgctgcgaaaatatgcaactgaacccctcacagagggctcatcaccatgtagatcacggaaggggtcattaattgtgctcgctgacgttttattgcagcccatttaactgtttgtacagaaactttttcattctgctaaaatttatttggagttgtatatgaaactaggagaactctggatacttttatatgctctccttcacgtaaagatgattaaaattgtctaacactaaagtgttaaactggaatggttgacaaagtacacaagatctcagtctacataaagggttgggggaaattagtattttcctaagtttattctgttttccttgctagaaaaatcccataaaagggatattcttgtcagacctctgccatttcttccatctgtgagagagcaacgtgatgtaaccacacctgagagcaggtactgtcctgtgttacaaaagaatcacatggaaaattgttaaccaaaataagtgtagatttcagaatcattccagctctactcttaactgtcctgaatttgttagaaactgatttgaaagaaatgtttcttaaaattccatacacacacacacacatattatgaattgtctttaattgtagtcagaatttattataacaatatttgatttggaccattttaaacattccctattttaaaattcatacggcttccttaagaagtagaatgaaagggtaaattggtgacatgtttgctctctgcattttctaaccttctaccgaattgtgactgactcagagagctctagcatttaccagtgaggttcagaaactaaactctcaggaattcattgcatctgttgtagaagtgcttctgggtctgagcctggcctcctcagagtggtaatactgcccacttcctctggaaaataggcagggctaatgagaaactaatcaaatgattaacctctgcggcccttcagcctttggaatgctaaaaaaaaaaaaaaaaaaaaaaaaaaaaa

SEQ ID NO: 56 - protein, 631 aa, Mus musculus
MFDGYDSCSEDTSSSSSSEESEEEVAPLPSNLPIIKNNGQVYTYPDGKSGMATCEMCGMVGVRDAFYSKTKRFCSVSCSRSYSSNSKKASILARLQGKPPTKKAKVLQKQPLVAKLAAYAQYQATLQNQAKTKAGNSAISVEGFSWGNYINSNSFIAAPVACFKHAPMGTCWGDISENVRIEVPNTDCSLPTKVFWIAGIIKLAGYNALLRYEGFENDSSLDFWCNICGSDIHPVGWCAASGKPLVPPRTVQHKYTNWKAFLVKRLTGAKTLPPDFSQKVSESMQYPFKPCMRVEVVDKRHLCRTRVAVVESVIGGRLRLVYEESEDGTDDFWCHMHSPLIHHIGWSRSIGHRFKRSDITKKQDGHFDTPPHLFAKVKEVDQSGEWFKEGMKLEAIDPLNLSTICVATIRKVLADGFLMIGIDGSEAADGSDWFCYHATSPSIFPVGFCEINMIELTPPRGYTKLPFKWFDYLRETGSIAAPVKLFNKDVPNHGFRVGMKLEAVDLMEPRLICVATVTRIIHRLLRIHFDGWEEEYDQWVDCESPDLNPVGWCQLTGYQLQPPASQSSRESQSASSKQKKKAKSQQYKGHKKMTTSQLKEELLDGEDYSFLHGASDQESNGSATVYIKQEP

SEQ ID NO: 57 - DNA, 1650 bp, Mus musculus
ggggcttcactctccagccttggagagcagtcagggaaggcctccaggaggaagcagctacctgtggcagggcagagcagtgcctgtaccagtctcccggaacttcagaactgcggtggccatggagtctgtggagcccccggtcaaagacggcatcctctaccagcagcacgtaaagtttggcaagaaatgctggcgcaaagtgtgggctctgctgtatgcgggaggcccatcaggggtagctcggctagaaagctgggacgtgcgtgatggtggcctgggaccagcaggcgacaggtccacagggcccagccgtcgaggggaacgccgggtcatacgcttggctgactgtgtatctgtcctgcctgcggatggcgagagctgtcccagggacactggtgccttcctgattaccaccactgagcgaagccacctgttggctgcacagcaccgccagtcctgggtggaccccatctgtcagctggccttcccgggtaccggagaatgttcgtcaggatcaggacaggctgagaatccaaaaaggggctttgttcccatggaagaaaactctatctactcctcctggcaggaagtgaccgaatttccggtgatcgtgcagaagacagaagccacctcccgctgccagctgaaaggaccctacctcctggtgctgggccaagatgacatccaactgagggagacatccaagccccaggcctgttttagctggccctaccgtttcctgcgcaagtacggctctgacaagggtgtgttctcgtttgaggctggccgccgctgtgactcaggtgagggcctttttgccttcagtagcccgcgtgccccagacatatgtggggttgtggctgccgccattgcccgccagcgggagcgtcttccagagctggccatgtccccaccctgccccctgcctcgggccctctccctgccctccctagagccccctggagagcttcgggaggtggccccaggatttgagctgcccactcccagaaagctgcctctaactgatcccgggcctcaaagcctaccattgctgctcagccccaacacaagaaggaccggcatccggtctctatgcgtccgtgtgcaagcagaccagcaagcacacaggcacggcggagcatttctatgagaacgtgtgcatgctggaggccagccttgggctgaccaatgggggtcctgaagcccaagagggcccccttggtggccgcagcccccttgggcagccttatctaccataacactgaggatctgagttggccgggctcggcccaggacagcaatctggaagcccagtaccggaggctgctggaactggagctggatgaggccggaagcgccggccgctctggagcgcaggcaggcatcaaggccaagctggtgaccctgctgacccgtgaacggaagaagggccccgccccctgtgaccggccctgaaggcctgagcggccagccactgcaggacagaggtgatcacccaagaccaggaacaacttcgaacataacccgtctactctgacctgcagggacaagccaggtggcccggggaggagccacactctgccctacctcctccctcagactgtacagattgaacagtaataaagcttgcctatcaacttcaaaaaa

SEQ ID NO: 58 - protein, 359 aa, Mus musculus
MESVEPPVKDGILYQQHVKFGKKCWRKVWALLYAGGPSGVARLESWDVRDGGLGPAGDRSTGPSRRGERRVIRLADCVSVLPADGESCPRDTGAFLITTTERSHLLAAQHRQSWVDPICQLAFPGTGECSSGSGQAENPKRGFVPMEENSIYSSWQEVTEFPVIVQKTEATSRCQLKGPYLLVLGQDDIQLRETSKPQACFSWPYRFLRKYGSDKGVFSFEAGRRCDSGEGLFAFSSPRAPDICGVVAAAIARQRERLPELAMSPPCPLPRALSLPSLEPPGELREVAPGFELPTPRKLPLTDPGPQSLPLLLSPNTRRTGIRSLCVRVQADQQAHRHGGAFLERVHAGGQPWADQWGS

SEQ ID NO: 59 - DNA, 1750 bp, Mus musculus
ggcctccgacatattgcccgcaggagctgcggcggtgagcggagagcgccgggggaaggagatgggaggacgaagaggtcccaacaggacatcttactatcgaaacccgctctgtgagccaggatcatcaggggcctctggtggaggccactcttctagtgcatccgtgagcagcgtccgttctcgaagcaggaccacttctgggacgggcctctccagccctccgctggccgcgcagaccgttgtgcctctacagcactgcaagatccccgagctgcccgtccaggccagcattctgttcgagctacagctcttcttctgtcagctcatagccctgttcgtgcactacatcaacatctataagacagtctggtggtatccaccctcgcacccaccctcccacacttccctgaacttccacctgatcgacttcaacttgctgatggtgaccgccattgttctgggccgccgattcatcgggtccatcgtgaaggaggcttctcagagggggaaaggtctccctcttccgctccatcctgctgttcctcacccgcttcacgttctcacggcgacaggctggagtctgtgccggtccctcatccacctcttcaggacctactccttcctgaacctcctgttcctctgctatccgtttgggatgtacattccgttcctgcagctgaactatgatcttcgcaagacgaacctcttcacccacatggcttccatgggaccccgagaggcagtcagtggcctggcaaggagtcgggactactttctgacactgcgggagacttggaagcagcatacacgacagctatatggcccggaagccatgcccacccatgcctgctgcttgtcacccagcctcattcgcaatgaagttgagttcctcaaaatggacttcaattggcgaatgaaggaagtacttgtcagctccatgctgagtgcctactatgtggcctttgtacctgtgtggtttgtgaagaatacacattactatgacaagcgctggtcctgtgagctcttcctgctggtgtccatcagtacctccgtgatcctcatgcagcacttgttgcctgccagctactgtgacctgctgcacaaggccgccgcccacctggcgtgctggcagaaggtggacccagctctgtgctccaatgtgctgcagccaccgtggactgaagagtgcatgtggccacagggtgtgctggtgaaacacagcaagaatgtctacaaagcagtgggccactacaacgtggcatcccctccgatgtctcccacttccgcttccatttctttttcagcaaccccctgcggatcctcaacatccttttgctgctggagggtgccgtcattgtctaccagctgtactccttaatgtcctcagagaagtggcaccagaccatctccctggccctcatcctcttcagcaactactatgccttcttcaagctgctccgggaccgcctggtattgggcaaagctactcctactcagccagcccccagaggacctggaccaccggttctcctgagtcccagggtgatcctccaaacaacatccgggcttcacccaggggcttcctggccaagggctcttggggttgggggggagggatacaaaaaacaaaaacaaaaaacaacaaaaaaaaaccaacccaccagagctttgtatttttgttacgtactgtttctttctttgataattgatgtggtaagaaaaaagtcttatttttatactccaaaaa

SEQ ID NO: 60 - protein, 439 aa, Mus musculus
MGGRRGPNRTSYYRNPLCEPGSSGASGGGHSSSASVSSVRSRSRTTSGTGLSSPPLAAQTVVPLQHCKIPELPVQASILFELQLFFCQLIALFVHYINIYKTVWWYPPSHPPSHTSLNFHLIDFNLLMVTAIVLGRRFIGSIVKEASQRGKGLPLPLHPAVPHPLHVLTATGWSLCRSLIHLFRTYSFLNLLFLCYPFGMYIPFLQLNYDLRKTNLFTHMASMGPREAVSGLARSRDYFLTLRETWKQHTRQLYGPEAMPTHACCLSPSLIRNEVEFLKMDFNWRMKEVLVSSMLSAYYVAFVPVWFVKNTHYYDKRWSCELFLLVSISTSVILMQHLLPASYCDLLHKAAAHLACWQKVDPALCSNVLQPPWTEECMWPQGVLVKHSKNVYKAVGHYNVASPPMSPTSASISFSATPCGSSTSFCCWRVPSLSTSCTP

SEQ ID NO: 61 - DNA, 3071 bp, Mus musculus
gtgaaagcagcagtgcgcctctgctcccttcagagcacagcctggtgtcaaggtccaggttccaccggctgctgctgtcaccgcaggggagtctagcccctcccagaaggagacacagaagaatggccatctcaactggtttgttcctgctgctggggctccttggccagccctgggcaggggctgctgctgattcacaggctgtggtgtgcgaggggactgcctgctatacagcccattggggcaagctgagtgccgctgaagcccagcatcgctgcaatgagaatggaggcaatcttgccaccgtgaagagtgaggaggaggcccggcatgttcagcaagccctgactcagctcctgaagaccaaggcacccttggaagcaaagatgggcaaattctggatcgggctccagcgagagaagggcaactgtacgtaccatgatttgccaatgaggggcttcagctgggtgggtggtggagaggacacagcttattcaaactggtacaaagccagcaagagctcctgtatctttaaacgctgtgtgtccctcatactggacctgtccttgacacctcaccccagccatctgcccaagtggcatgagagtccctgtgggacccccgaagctccaggtaacagcattgaaggtttcctgtgcaagttcaacttcaaaggcatgtgtaggccactggcgctgggtggtccagggcgggtgacctataccacccctttccaggccactacctcctctctggaggctgtgccttttgcctctgtagccaatgtagcttgtggggatgaagctaagagtgaaacccactatttcctatgcaatgaaaagactccaggaatatttcactggggcagctcaggcccactctgtgtcagccccaagtttggttgcagtttcaacaacgggggctgccagcaggattgcttcgaaggtggcgatggctccttccgctgcggctgccggcctggatttcgactgctggatgatctagtaacttgtgcctccaggaacccctgcagctcaaacccatgcacaggaggtggcatgtgccattctgtaccactcagtgaaaactacacttgccgttgtcccagcggctaccagctggactctagccaagtgcactgtgtggatatagatgagtgccaggactccccctgtgcccaggattgtgtcaacactctagggagcttccactgtgaatgttgggttggttaccaacccagtggccccaaggaagaggcctgtgaagatgtggatgagtgtgcagctgccaactcgccctgtgcccaaggctgcatcaacactgatggctctttctactgctcctgtaaagagggctatattgtgtctggggaagacagtacccagtgtgaggatatagatgagtgttcggacgcaaggggcaatccatgtgattccctgtgcttcaacacagatggttccttcaggtgtggctgcccgccaggctgggagctggctcccaatggggtcttttgtagcaggggcactgtgttttctgaactaccagccaggcctccccaaaaggaagacaacgatgacagaaaggagagtactatgcctcctactgaaatgcccagttctcctagtggctctaaggatgtctccaacagagcacagacaacaggtctcttcgtccaatcagatattcccactgcctctgttccactagaaatagaaatccctagtgaagtatctgatgtctggttcgagttgggcacatacctccccacgacctccggccacagcaagccgacacatgaagattctgtgtctgcacacagtgacaccgatgggcagaacctgcttctgttttacatcctggggacggtggtggccatctcactcttgctggtgctggccctagggattctcatttatcataaacggagagccaagaaggaggagataaaagagaagaagcctcagaatgcagccgacagctattcctgggttccagagcgagcagagagccaagccccggagaatcagtacagcccaacaccagggacagactgctgaagactatgtggccttagagacagctgccactaccttcagagctaccttcttagatgagggggaagccacatcattctgaatgacttgactggactctcagcaaaaaaattgtgcaccttccacttaagaacctggtggcttgggataggcaggtattttcttggtgcctttgatatgtctgggggtgaaagctgtgtgttggtttgtcattgtggggagttttgtggatattgacagacctcactcaaacacccttttcaaatccaatagcaactggttcctctggttcctaattagggggaaaggagtcagaggggtgggacagggtggggggatggggcttcaaagttttttcttatcacttgatttatcatcgaaggagttactggtgctaattacaatggaaacagttcctttccatcacaggacagacacacctcaatcctccatggggtcaacaactatatacccccagtgaccccttaggcaaggacttgttgagaactgcatcacattttgacctgttctcaacagtacccatctatttcaggtgggatctctggacctttcctccttcccatcttgtctgcaatgtggcaaatggcttctttttgcatttttactccgcccccaccccaagctgaagttcatttgcagatcagcgattaagtctgaattgtgtggtggtcagtcttgtttccttttgtcaggggttattgtaaatgttagtaatttcgcctcaagccctcagtaagaacataaatattttaaaatatgtgcgtttgaaatctgtttcatgcatcctggaactgtgggatgctcaggcaagagtgactttagtctttcagtgaatgttgcccagaatgtgggtagggaaggctcacaggttactctcctccttagagctacaacataacattctgaggggagtcacagggttgcctttaaaaagtgggagctatgtcatgctttgagctttctgttaagcacctctcctaataaactctgaaaaaa

SEQ ID NO: 62 - protein, 644 aa, Mus musculus
MAISTGLFLLLGLLGQPWAGAAADSQAVVCEGTACYTAHWGKLSAAEAQHRCNENGGNLATVKSEEEARHVQQALTQLLKTKAPLEAKMGKFWIGLQREKGNCTYHDLPMRGFSWVGGGEDTAYSNWYKASKSSCIFKRCVSLILDLSLTPHPSHLPKWHESPCGTPEAPGNSIEGFLCKFNFKGMCRPLALGGPGRVTYTTPFQATTSSLEAVPFASVANVACGDEAKSETHYFLCNEKTPGIFHWGSSGPLCVSPKFGCSFNNGGCQQDCFEGGDGSFRCGCRPGFRLLDDLVTCASRNPCSSNPCTGGGMCHSVPLSENYTCRCPSGYQLDSSQVHCVDIDECQDSPCAQDCVNTLGSFHCECWVGYQPSGPKEEACEDVDECAAANSPCAQGCINTDGSFYCSCKEGYIVSGEDSTQCEDIDECSDARGNPCDSLCFNTDGSFRCGCPPGWELAPNGVFCSRGTVFSELPARPPQKEDNDDRKESTMPPTEMPSSPSGSKDVSNRAQTTGLFVQSDIPTASVPLEIEIPSEVSDVWFELGTYLPTTSGHSKPTHEDSVSAHSDTDGQNLLLFYILGTVVAISLLLVLALGILIYHKRRAKKEEIKEKKPQNAADSYSWVPERAESQAPENQYSPTPGTDC

SEQ ID NO: 63 - DNA, 499 bp, Mus musculus; identical to SEQ ID NO: 25

SEQ ID NO: 64 - protein, 89 aa, Mus musculus
MRTAPAAAILCELGLLYGNGGVRKCGNHKRRQNQKTEGSWAMSHSKESLPPNICTGLLQEQEMNYRVQPLRLGLNMIFTLSTLPNRAEM

SEQ ID NO: 65 - DNA, 3857 bp, Mus musculus
agagggtagacagaaaggcgggaaagggctgtgaggtcaaatggaccccatggaactgaacaacgtcagcatcgaacccgacggagacagctgcagcggggacagtattcaggacagctacaccggcatggaaaactccgacaaggacgccatgaacagccaatttgctaatgaagatgccgaaagtcagaagttcctgacaaatgggtttttagggaagaagaagctagccgattacgcggatgagcatcaccctggaatgacttcctttggaatgtcctcatttaacctgagcaacgccatcatgggcagtgggatcttaggcttgtcctatgccatggccaacaccgggatcatcctttttataatcatgctgcttactgtggcaatactctcgctctactcggttcaccttttgctgaagacagccaaggaaggagggtctctaatctatgaaaaattgggcgagaaagcatttggatggcctgggaaaattggagccttcatctctattacaatgcagaacattggagccatgtcaagctacctcttcatcattaagtacgaactgcctgaagtaatcagagcattcatgggacttgaagaaaacactggggaatggtacctcaacggcaactacctcgtcttatttgtgtccgtggggatcatcctcccgctctctctccttaaaaatttaggctaccttggctacaccagtggattttctctctcctgcatggtgtttttcgtcagtgtggtgatttacaaaaaattccaaattccctgccctctgcctgctctggatcacaacaacggaaatctgacgttcaacaacacacttccgattcacatgatctcgctgcctaatgactcggagagctcgggtgtgaacttcatgatggattacgctcaccacaacccagctgggctggatgagaagcaggtcgcaggccctcttcacagcaatggcgtggagtacgaagcccagggtgctgagaaatgccaaccaaaatactttgtgttcaattcccggacggcctatgcaatcccaatcctggcttttgcttttgtctgccaccctgaggtccttcccatctacagcgagcttaaagatcgatcccgcagaaagatgcagacggtgtccaacatttccatctcaggcatgctcgtcatgtaccttcttgcggccctctttggttatctgagcttctacggggacgttgaagacgagctgctgcatgcttacagcaaggtctacacatttgatacggctcttctcatggtgcgcctggcagtcctggtggcagtgacactgaccgtgcccatcgtgctgttcccgatccgtacttcggtgatcacactgctgtttccaaggaaacccttcagctggctgaagcatttcgggatcgctgcaatcatcatcgcactcaacaacatcctggtcatcctcgtgcctaccatcaaatacatctttggattcataggggcttcttctgccactatgctgattttcattcttccggctgcgttttatctcaagctcgtcaagaaagaacctctaagatcaccccagaagattggggctttggtcttccttgtgactggaattattttcatgatgggaagcatggcgctcattatactcgactggatctacaacccgccgaatcccaatcaccactaatcccggggagacgcgtctccactggaaacagctgaaattgtctgaaggacattttagttgtcttgattgggatgttagtctgaggaattagcaagattccaaagacgtttttctagctctatcagcacacattttaacccaggccgtgcagtgcagtgtgtgatgcccgagttgtgtttgcagcagctgtgcaagctgaagcctgttggctgcgtgtgttggtcagcagacaatagcctgtccccccatggtcactccacttctctccacccccagattaacaggtaattctactctcagaacatcagacaaagacctcctggttgggatacttgtggaagagaaaattatgggttttgttgggaatggttttgttgggaatggtgaaggatgcattaaaaattctgtgcgaagtatcatcagttacggccatctctcactctacaccaacactaagggtcggttgactagctgaggcagggggatatcttgggctgtccctgtgaggatcatgacgtatgacggttgccagtatagagtacttcatttcaatactcaaggaatagtttgcccaacctgcttattacaccgagttagtgaattttcattctttttattttttggtatggcaaatgttcaaacatctcactaaacatagaggggggttatttagtttatcatgaaacaactaccaaaaagtaatggtttttaacatctgccttttcatgttgtttctgatatctttgttccactttgtctttgaacaaggcttcccctctcgtcttccgttctcactcttcgtaattttcagaaaaaccacagtccgtgtgggagacacactaccccagtattgtttgatacatctctatttgataaacattcagtgcaggaaactgtgattttgctatatgtttgtgtacataatctcattctgcagttatcagaacgttgacatatgggacattggatttttattttttacatatgtaggttttttttcttcacagacaaaatgtttatatcatcagggtggggcagggaattaagctggtgggctcaaaaatccatgggtacttatctgtccattggagacatctaaaaattaaagtcaaagttgtgcatagttcagtaatgctcttcactgtttacaagactataatcatctcagcaaagtagcgaaggaagggtttgtaaataattttcagtgctaacaatggtctttgaatttaacatgtctagaatagggtttagttcattttagtttacatcggaacttggtgacattcatttgccattaatagaacatcgtgtggttaggaataaatgcaccaacatgacccaaaaccacattgctcacatccatggacgcttacacgggatggagttcagactccgcccactgcctagaatctctggcattgacttctttttttcaattactggttgtatattttacatttcagtaaaaagccaagtctgaaatgcaagtatcctacaggtagtgatatactgtcacaccaaagcagtttttcaacagtgtgcctaagaaacatatatttgtttactgtgtcattttctggaccttaagactatacaatggtgtgctttgtgatagaaattgagcattactagaaactaccataacttcggagttattatcatttgggagggaaaaaaatcaccaaataggatctttaagatgtctttctattttcattcatccaagaaataaatcaagatagaaagcagaaagggtgtgctgcaaatattaaattattattttatctgaacatattaatgtgaatataaaagattaatccaaaaatattggaaacctgtgaaatctgtacgtatttcggatctttacacttagagcagctaacctggaagtatccacttgtgtagttagatattcaagagcaatatcacagtgccaaagacttcattcacacagcaccgatcatttgtgtggccgttttcaggattagagttcacacagactccagggcagatactcagaataccgttttctggtaaatttaacccatttgtaaacagatacaaattttattttcttacatcatttataagacggttcaatgtactggggggattttttttattacagactgtgtattggtatataataaataaacttttcaaatgaaaaaaa

SEQ ID NO: 66 - protein, 547 aa, Mus musculus
MDPMELNNVSIEPDGDSCSGDSIQDSYTGMENSDKDAMNSQFANEDAESQKFLTNGFLGKKKLADYADEHHPGMTSFGMSSFNLSNAIMGSGILGLSYAMANTGIILFIIMLLTVAILSLYSVHLLLKTAKEGGSLIYEKLGEKAFGWPGKIGAFISITMQNIGAMSSYLFIIKYELPEVIRAFMGLEENTGEWYLNGNYLVLFVSVGIILPLSLLKNLGYLGYTSGFSLSCMVFFVSVVIYKKFQIPCPLPALDHNNGNLTFNNTLPIHMISLPNDSESSGVNFMMDYAHHNPAGLDEKQVAGPLHSNGVEYEAQGAEKCQPKYFVFNSRTAYAIPILAFAFVCHPEVLPIYSELKDRSRRKMQTVSNISISGMLVMYLLAALFGYLSFYGDVEDELLHAYSKVYTFDTALLMVRLAVLVAVTLTVPIVLFPIRTSVITLLFPRKPFSWLKHFGIAAIIIALNNILVILVPTIKYIFGFIGASSATMLIFILPAAFYLKLVKKEPLRSPQKIGALVFLVTGIIFMMGSMALIILDWIYNPPNPNHH

SEQ ID NO: 67 - DNA, 1070 bp, Mus musculus
agcgacatggccccgcccgcgctccaggcccagcctccaggcggctctcaactgaggttcctgctgttcctgctgctgttgctgctgctgctgtcatggccatcgcagggggacgccctggcaatgcctgaacagcgaccctccggccctgagtcccaactcaacgccgacgagctacggggtcgcttccaggacctgctgagccggctgcatgccaaccagagccgagaggactcgaactcagaaccaagtcctgacccagctgtccggatactcagtccagaggtgagattggggtcccacggccagctgctactccgcgtcaaccgggcgtcgctgagtcagggtctccccgaagcctaccgcgtgcaccgagcgctgctcctgctgacgccgacggcccgcccctgggacatcactaggcccctgaagcgtgcgctcagcctccggggaccccgtgctcccgcattacgcctgcgcctgacgccgcctccggacctggctatgctgccctctggcggcacgcagctggaactgcgcttacgggtagccgccggcagggggcgccgaagcgcgcatgcgcacccaagagactcgtgcccactgggtccggggcgctgctgtcacttggagactgtgcaggcaactcttgaagacttgggctggagcgactgggtgctgtccccgcgccagctgcagctgagcatgtgcgtgggcgagtgtccccacctgtatcgctccgcgaacacgcatgcgcagatcaaagcacgcctgcatggcctgcagcctgacaaggtgcctgccccgtgctgtgtcccctccagctacaccccggtggttcttatgcacaggacagacagtggtgtgtcactgcagacttatgatgacctggtggcccggggctgccactgcgcttgagcaccgggccctgctcctcacctacactccccttcaaggatgctatttatatttgtatttattaatattattaatttattggggtcgggctgggtggatggattgtgtatttatttaaaactctgctaataaaggtgagcttggtttcaaaa

SEQ ID NO: 68 - protein, 298 aa, Mus musculus
MAPPALQAQPPGGSQLRFLLFLLLLLLLLSWPSQGDALAMPEQRPSGPESQLNADELRGRFQDLLSRLHANQSREDSNSEPSPDPAVRILSPEVRLGSHGQLLLRVNRASLSQGLPEAYRVHRALLLLTPTARPWDITRPLKRALSLRGPRAPALRLRLTPPPDLAMLPSGGTQLELRLRVAAGRGRRSAHAHPRDSCPLGPGRCCHLETVQATLEDLGWSDWVLSPRQLQLSMCVGECPHLYRSANTHAQIKARLHGLQPDKVPAPCCVPSSYTPVVLMHRTDSGVSLQTYDDLVAR

SEQ ID NO: 69 - protein, 134 aa, Mus musculus
AKYVCLANKNCPVDKRRRNRCQYCRFQKCLAVGMVKEVVRTDSLKGRRGRLPSKPKSPQEPSPPSPPFQANPDYQMSGDDTQHIQQFYDLLTGSMEIIRGWAEKIPGFADLPKADQDLLFESAFLELFVLRLAY

SEQ ID NO: 70 - protein, 140 aa, Mus musculus
  All Xaa′s in this sequence represent unreadable amino acid.
SQTRQQGPLRSIMKDLHSDDNEEESDEVEDNDNDSEMERPVNRGXSRSRRVSLSDGSDSESSSASSPLHHEPPPPLLKTNNNQILEVKSPIKQSKSDKQIKNGECDKAYLDELVELPRXXMTLRERHILQQIVNLXEETG

SEQ ID NO: 71 - protein, 89 aa, Mus musculus; identical to SEQ ID NO: 64

SEQ ID NO: 72 - DNA, 3121 bp, Homo sapiens
gaaagcagcagtgcgcctctgctcccttcagagcacagcctggtgtcaaggtccaggttccaccggctgctgctgtcaccgcaggggagtctagcccctcccagaaggagacacagaagaatggccatctcaactggtttgttcctgctgctggggctccttggccagccctgggcaggggctgctgctgattcacaggctgtggtgtgcgaggggactgcctgctatacagcccattggggcaagctgagtgccgctgaagcccagcatcgctgcaatgagaatggaggcaatcttgccaccgtgaagagtgaggaggaggcccggcatgttcagcaagccctgactcagctcctgaagaccaaggcacccttggaagcaaagatgggcaaattctggatcgggctccagcgagagaagggcaactgtacgtaccatgatttgccaatgaggggcttcagctgggtgggtggtggagaggacacagcttattcaaactggtacaaagccagcaagagctcctgtatctttaaacgctgtgtgtccctcatactggacctgtccttgacacctcaccccagccatctgcccaagtggcatgagagtccctgtgggacccccgaagctccaggtaacagcattgaaggtttcctgtgcaagttcaacttcaaaggcatgtgtaggccactggcgctgggtggtccagggcgggtgacctataccacccctttccaggccactacctcctctctggaggctgtgccttttgcctctgtagccaatgtagcttgtggggatgaagctaagagtgaaacccactatttcctatgcaatgaaaagactccaggaatatttcactggggcagctcaggcccactctgtgtcagccccaagtttggttgcagtttcaacaacgggggctgccagcaggattgcttcgaaggtggcgatggctccttccgctgcggctgccggcctggatttcgactgctggatgatctagtaacttgtgcctccaggaacccctgcagctcaaacccatgcacaggaggtggcatgtgccattctgtaccactcagtgaaaactacacttgccgttgtcccagcggctaccagctggactctagccaagtgcactgtgtggatatagatgagtgccaggactccccctgtgcccaggattgtgtcaacactctagggagcttccactgtgaatgttgggttggttaccaacccagtggccccaaggaagaggcctgtgaagatgtggatgagtgtgcagctgccaactcgccctgtgcccaaggctgcatcaacactgatggctctttctactgctcctgtaaagagggctatattgtgtctggggaagacagtacccagtgtgaggatatagatgagtgttcggacgcaaggggcaatccatgtgattccctgtgcttcaacacagatggttccttcaggtgtggctgcccgccaggctgggagctggctcccaatggggtcttttgtagcaggggcactgtgttttctgaactaccagccaggcctccccaaaaggaagacaacgatgacagaaaggagagtactatgcctcctactgaaatgcccagttctcctagtggctctaaggatgtctccaacagagcacagacaacaggtctcttcgtccaatcagatattcccactgcctctgttccactagaaatagaaatccctagtgaagtatctgatgtctggttcgagttgggcacatacctccccacgacctccggccacagcaagccgacacatgaagattctgtgtctgcacacagtgacaccgatgggcagaacctgcttctgttttacatcctggggacggtggtggccatctcactcttgctggtgctggccctagggattctcatttatcataaacggagagccaagaaggaggagataaaagagaagaagcctcagaatgcagccgacagctattcctgggttccagagcgagcagagagccaagccccggagaatcagtacagcccaacaccagggacagactgctgaagactatgtggccttagagacagctgccactaccttcagagctaccttcttagatgagggggaagccacatcattctgaatgacttgactggactctcagcaaaaaaattgtgcaccttccacttaagaacctggtggcttgggataggcaggtattttcttggtgcctttgatatgtctgggggtgaaagctgtgtgttggtttgtcattgtggggagttttgtggatattgacagacctcactcaaacacccttttcaaatccaatagcaactggttcctctggttcctaattagggggaaaggagtcagaggggtgggacagggtggggggatggggcttcaaagttttttcttatcacttgatttatcatcgaaggagttactggtgctaattacaatggaaacagttcctttccatcacaggacagacacacctcaatcctccatggggtcaacaactatatacccccagtgaccccttaggcaaggacttgttgagaactgcatcacattttgacctgttctcaacagtacccatctatttcaggtgggatctctggacctttcctccttcccatcttgtctgcaatgtggcaaatggcttctttttgcatttttactccgcccccaccccaagctgaagttcatttgcagatcagcgattaagtctgaattgtgtggtggtcagtcttgtttccttttgtcaggggttattgtaaatgttagtaatttcgcctcaagccctcagtaagaacataaatattttaaaatatgtgcgtttgaaatctgtttcatgcatcctggaactgtgggatgctcaggcaagagtgactttagtctttcagtgaatgttgcccagaatgtgggtagggaaggctcacaggttactctcctccttagagctacaacataacattctgaggggagtcacagggttgcctttaaaaagtgggagctatgtcatgctttgagctttctgttaagcacctctcctaataaactctgaaaaaataaaaaaaaaaaaaaaaaaaaaaaaaaaaaaaaaaaaaaaaaaaaaaaaaaa

SEQ ID NO: 73 - protein, 644 aa, Homo sapiens; identical to SEQ ID NO: 62

SEQ ID NO: 74 - DNA, 27 bp, Artificial Sequence
  Description of Artificial Sequence Primer
tggtgtcgacgcagagtagcggccgct

SEQ ID NO: 75 - DNA, 14 bp, Artificial Sequence
  Description of Artificial Sequence Primer
ggcccgggccggcc

SEQ ID NO: 76 - DNA, 17 bp, Artificial Sequence
  Description of Artificial Sequence Primer
tcgaggccgcccgggcc

SEQ ID NO: 77 - DNA, 18 bp, Artificial Sequence
  Description of Artificial Sequence Primer
cagccctcactccttctc

SEQ ID NO: 78 - DNA, 18 bp, Artificial Sequence
  Description of Artificial Sequence Primer
ggtggggtctttcattcc

SEQ ID NO: 79 - DNA, 18 bp, Artificial Sequence
  Description of Artificial Sequence Primer
ttcagcaagccctgactc

SEQ ID NO: 80 - DNA, 18 bp, Artificial Sequence
  Description of Artificial Sequence Primer
gccaccttcgaagcaatc

SEQ ID NO: 81 - DNA, 18 bp, Artificial Sequence
  Description of Artificial Sequence Primer
gagcggtacaggagaatg

SEQ ID NO: 82 - DNA, 18 bp, Artificial Sequence
  Description of Artificial Sequence Primer
gcccacccaaccaaatca

SEQ ID NO: 83 - DNA, 18 bp, Artificial Sequence
  Description of Artificial Sequence Primer
accgcgttcttctgtaac

SEQ ID NO: 84 - DNA, 18 bp, Artificial Sequence
  Description of Artificial Sequence Primer
cagctaacagcaggatcc

SEQ ID NO: 85 - DNA, 15 bp, Artificial Sequence
  Description of Artificial Sequence Primer
gacccaggctgcccc

SEQ ID NO: 86 - DNA, 15 bp, Artificial Sequence
  Description of Artificial Sequence Primer
ggtactcttgttgag

SEQ ID NO: 87 - DNA, 34 bp, Artificial Sequence
  Description of Artificial Sequence Primer
gagggtaccaagcttcgtggtccgcaaccagaag

SEQ ID NO: 88 - DNA, 36 bp, Artificial Sequence
  Description of Artificial Sequence Primer
ctcagatctgaattcggcctgtccctcggtgaggag

SEQ ID NO: 89 - DNA, 35 bp, Artificial Sequence
  Description of Artificial Sequence Primer
gacaagatctcagctgaatagcgacccaccctgtg

SEQ ID NO: 90 - DNA, 41 bp, Artificial Sequence
  Description of Artificial Sequence Primer
gcatctagagcggccgctcaggcctgtccctcggtgaggag

SEQ ID NO: 91 - DNA, 37 bp, Artificial Sequence
  Description of Artificial Sequence Primer
gcatctagagcggccgcgaacgctgcttagatctcct

SEQ ID NO: 92 - protein, 131 aa, Mus musculus
  All Xaa′s in this sequence represent unreadable amino acid.
EVFLNPLGRDLLSISDGRXRAIHRRGHNDGEDSLTHTDVSSFQTMDQMVSNMRNYMQKLERNTGQLSVDPNGHSFCSSSVMTYSKIGDEPPKVFHASTQTRRAPGRIKETRKAMRDSDSGTRKHGYWSSYP

SEQ ID NO: 93 - protein, 131 aa, Mus musculus
RSFSEPFGRDLLSISDGRGRAHNRRGHNDGEDSLTHTDVSSFQTMDQMVSNMRNYMQKLERNFGQLSVDPNGHSFCSSSVMTYSKIGDEPPKVFQASTQTRRAPGGIKETRKAMRDSDSGLEKMAIGHHIH

SEQ ID NO: 94 - protein, 159 aa, Mus musculus
  All Xaa′s in this sequence represent unreadable amino acid.
AKYVCLANKNCPVDKRRRNRCQYCRFQKCLAVGMVKEXVRTDSLKGRRGRLPSKPKSPQEPSPPSPPVSLISALVRAHVDSNPAMTSLDYSRFQANPDYQMSGDDTQHIQQFYDLLTGSMEIIRGWAXXIPGFADLPKADQDLLFESAFLELFVLRLAY

SEQ ID NO: 95 - protein, 140 aa, Mus musculus
SQTRQQGPLRSIMKDLHSDDNEEESDEVEDNDNDSEMERPVNRGGSRSRRVSLSDGSDSESSSASSPLHHEPPPPLLKTNNNQILEVKSPIKQSKSDKQIKNGECDKAYLDELVELHRRLMTLRERHILQQIVNLIEETG

SEQ ID NO: 96 - protein, 652 aa, Mus musculus
MATSMGLLLLLLLLLTQPGAGTGADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPNRLPKWSEGPCGSPGSPGSNIEGFVCKFSFKGMCRPLALGGPGQVTYTTPFQTTSSSLEAVPFASAANVACGEGDKDETQSHYFLCKEKAPDVFDWGSSGPLCVSPKYGCNFNNGGCHQDCFEGGDGSFLCGCRPGRRLLDDLVTCASRNPCSSSPCRGGATCVLGPHGKNYTCRCPQGYQLDSSQLDCVDVDECQDSPCAQECVNTPGGFRCECWVGYEPGGPGEGACQDVDECALGRSPCAQGCTNTDGSFHCSCEEGYVLAGEDGTQCQDVDECVGPGGPLCDSLCFNTQGSFHCGCLPGWVLAPNGVSCTMGPVSLGPPSGPPDEEDKGEKEGSTVPRAATASPTRGPEGTPKATPTTSRPSLSSDAPITSAPLKMLAPSGSSGVWREPSIHHATAASGPQEPAGGDSSVATQNNDGTDGQKLLLFYILGTVVAILLLLALALGLLVYRKRRAKREEKKEKKPQNAADSYSWVPERAESRAMENQYSPTPGTDC

(19) United States
(12) Patent Application Publication
(10) Pub. No.: US 2002/0064855 A1
(43) Pub. Date: May 30, 2002

(54) Genes that regulate hematopoietic blood forming stem cells and uses thereof

(76) Inventors: Ihor Lemischka, Princeton, NJ (US); Kateri Moore, Princeton, NJ (US)

Correspondence Address: KLAUBER & JACKSON, 411 HACKENSACK AVENUE, HACKENSACK, NJ, 07601

(21) Appl. No.: 09/789,919

(22) Filed: Feb. 21, 2001

(30) Foreign Application Priority Data
Aug. 20, 1999 (US) PCT/US99/19052

(51) Int. Cl. C12N 9/02; C12N 9/64; C07H 21/04; C12P 21/02; C12N 5/08

(52) U.S. Cl. 435/226; 435/69.1; 435/372; 435/320.1; 536/23.2; 435/189

(57) ABSTRACT

The present invention provides an isolated nucleic acid derived from an isolated hematopoietic stem cell. The present invention additionally provides an isolated hematopoietic stem cell specific protein or a portion thereof encoded by the provided nucleic acid. In addition, the present invention provides a method for generating a stem cell/progenitor cell from a primitive hematopoietic cell. Also, the present invention farther provides a method for identifying the presence in a sample of a compound that modulates hematopoietic stem cell activity. The present invention additionally provides a molecularly defined primitive hematopoietic stem cell. Finally, the present invention provides a method of ex vivo expansion of hematopoietic stem cells.

### GOVERNMENTAL SUPPORT

This invention was made with the support of National Institutes of Health Grant No. RO1 CA45339-09 and American Cancer Society No. DHP-144/01. The United States government may have certain rights to this invention.

Throughout this application, various publications are referenced by number. Full citations for these publications may be found listed at the end of the specification and preceding the claims. The disclosures of these publications in their entireties are hereby incorporated by reference into this application in order to more fully describe the state of the art. A Sequence Listing is provided.

### FIELD OF THE INVENTION

The present invention relates to hematopoietic stem cells and the stem cell and support cell genes that support stem cell replication and differentiation.

### BACKGROUND OF THE INVENTION

The adult hematopoietic system is organized as a hierarchy of cells with decreasing self-renewal and multilineage differentiation potential. This is accompanied by progressively larger numbers of more mature cells and an increasing tendency to be in active cell cycle (Lemischka, I. R., 1992; Morrison, S. J., et al. 1995). Collectively, the properties of this hierarchical system result in the balanced, lifelong production of at least eight distinct cell lineages. A population of stem cells establishes the entire hierarchy; therefore, in order to understand the fundamental mechanisms responsible for normal hematopoiesis it is ultimately necessary to understand the biology of the stem cells.

Most information concerning the biology of stem cells has been obtained from the mouse model. In this system, the most critical, characteristic property of the stem cell population has been defined; that is, its ability to reconstitute a normal blood system in a transplanted host. A number of variations on the basic transplantation assay have been described (Harrison, D. E. 1980; Spangrude, G. J., et al. 1995). All of these systems, together with the appropriate donor vs. host or clonotypic markers have rigorously defined the most primitive stem cells and have provided a description of their developmental properties. Perhaps the most striking characteristics of this cell population come from retroviral “marking” studies (Leminschka, I. R. 1992). These studies clearly show that a single stem cell clone is both necessary and sufficient, not only to sustain lifelong, multilineage hematopoiesis in one primary recipient but in numerous secondary animals. This illustrates the remarkable proliferative potential of the stem cell and directly demonstrates stem cell self-renewal. A major advance in mouse stem cell biology was the development of strategies which facilitate the substantial enrichment of stem cell activity (Bauman, J. G., et al. 1988; Spangrude, G. J., et al. 1988; Jordan C. T. , et al 1990). Purification procedures enabled the first direct approaches to unravel the mechanisms responsible for the unique biological properties of the stem cell population. A key observation was that the phenotypically defined stem/progenitor cell population is heterogeneous with respect to in vivo functional properties (Fleming, W. G., et al 1993; Li, C. L. and Johnson G. R. 1992; Spangrude, G. J. and Johnson, G. R. 1990, Jones R., et al. 1990; Uchida, N., et al. 1993). In addition to the in vivo repopulating cells, other primitive progenitor cells are often contained in a purified population (Weilbaecher, K., 1991; Trevisan, M. and Iscove, N. N. 1995; Ogawa, M. 1993). These can be assayed in a variety of in vitro systems. Whether all of these in vivo and in vitro activities represent discrete cell subpopulations or whether there is a continuum of functional potential is still an unanswered question. Recent studies have suggested distinct physical properties for functionally different activities within the primitive population Morrison, S. J., and Weissman, I. L. 1994); Morrison, S. J. et al. 1997; Jones, R. M., el al. 1996). A second set of observations revealed an inverse correlation between a tendency for active cell cycling and primitive, uncommitted developmental potential in BM (Spangrude, G. J., and Johnson, G. R. 1990). In fetal liver a higher proportion of primitive stem cells is actively cycling (Fleming, W. H., et al. 1993). Moreover, it has been shown that fetal stem cells are more potent than adult stem cells in LTRA (Jordan, C. T., et al. 1995; Pawliuk, R., et al 1996). These are exciting observations because they suggest that rapid stem cell cycling can be compatible with the maintenance of primitive in vivo activity. Very recent studies suggest that the adult BM stem cell compartment may in fact be cycling at a very slow rate (Bradford, G. B., et al. 1997). Clearly, stem cell cycle regulation is a critical area for investigation. Stem cell purification has facilitated studies aimed at ex vivo maintenance or expansion of the most primitive, transplantable stem cell. Most culture systems strongly favor a differentiation process (Van der Sluijs, J. P., et al. 1993; Traycoff, C. M., et al. 1996; Peters, S. O., et al. 1995; Knobel, K. M., et al. 1994). However, several recent reports have been encouraging. It has been shown that colonies grown in defined cytokines can retain not only myeloid and erythroid but also lymphoid potentials (Ball, T. C., et al. 1995). Moreover, the short-term (2-3 weeks) maintenance of LTRA has been demonstrated in suspension cultures supported by IL6, IL 11, together with ckit ligand (KL) or flk2/flt3 ligand (FL) (Yonemura, Y. H., et al. 1997). A recent report has shown that colonies initiated in cytokine-supplemented semisolid cultures retain LTRA (Trevisan, M., et al. 1996). The studies described herein have developed a stromal cell line supported system which quantitatively maintains LTRA for an extended (4-7 weeks) time (Moore, F. A., et al. 1997).

In the human system it is clearly not possible to do the same kind of extensive marking and transplantation assays. However, several xenograft model systems have been developed to assess the in vivo behavior of human stem cells (Traycoff, C., et al. 1994; Turner, C., et al. 1996; Cashman, J., et al. 1997). Some of these experiments can be done quantitatively in limiting dilution (Bhatia, M., et al. 1997). A very recent study has demonstrated a common proviral integration site in granulocyte macrophages and T-cells derived from beige/nude/XID mice 7-11 months after engraftment with genetically transduced human stem cells (Nolta, J. A., et al. 1996). This important study paves the way for precise in vivo clonal analyses. The largest amount of functional information about human stem/progenitor cells has been obtained in vitro using a wide range of stromal cell and cytokine supported culture systems. It is not possible herein to describe and properly accredit all of the important studies, however several advances deserve mention. The long-term culture-initiating cell (LTCIC) assay measures the in vitro production of colony forming cells (CFC) after periods of at least five weeks in culture (Sutherland, H. S., et al. 1989). The cells producing these CFC derive from a population of cells which, at least to some extent, probably overlaps with the most primitive compartment. The maintenance and expansion of primitive functional abilities in this culture system has recently been documented (Petzer, A. L., et al. 1996). A variation on this assay system, the extended LTCIC (ELTCIC) has been suggested to measure an even more primitive cell population in BM and CB (Hao, Q. L., et al. 1996). A very exciting prospect for the near future will be the integration of the various in vivo xenograft assays with the in vitro LTCIC and ELTCIC systems. Some very recent efforts have suggested that the NODSCID xenograft system and the LTCIC assay may measure distinct stem/progenitor subsets (Larochelle, A. J., et al. 1997). Clearly, much more work needs to be done, however, it may be anticipated that the ELTCIC system will provide the “bridge” in this continuum. Collectively, and including the various strictly cytokine-driven systems, the above studies illustrate the current possibility to accurately and quantitatively reveal the majority (if not all) functional entities in the human stem/progenitor cell hierarchy. The physical characterization and purification of human stem/progenitor cells has proceeded along lines which are parallel to the mouse system. Indeed, because of clinical impetus, it can be argued that they are further advanced. Thus, as measured in the range of assays discussed above, the consensus physical phenotype of the most primitive portion of the human stem/progenitor hierarchy is CD34+Lin−CD38− (Terstappen, L. W. M. M., et al. 1991). The CD34+Lin−−CD38+ subset contains less primitive, more committed cellular entities. Other studies have shown that, similar to the mouse, low level expression of Thy1 (CD90) is a feature of the primitive human stem cell (CD34+Lin−CD90+) (Baum, C. M., et al. 1992; Craig, W., et al. 1993). Most CD90+ cells in this compartment are CD38−. Therefore, the consensus phenotype can be described as CD34+Lin−CD90+ (Craig, W., et al. 1993). Two potential differences with the murine system can now be highlighted. First, a very recent and elegant study has shown that the most primitive mouse stem cell may in fact be CD34−/lo (Osawa, M., et al. 1996). Whether this is a genuine difference or whether it reflects the ability to perform more accurate long-term engraftment studies in the mouse remains to be determined. Second, it has been suggested that in the mouse, CD38 expression is a positive indicator for primitive stem cell function in a purified population (Randall, T. D., et al. 1996). As in the mouse, human stem/progenitor cells have been identified and purified from various sources. These include: adult BM (Baum, C. M., et al. 1992), CB (DiGiusto, D. L., et al 1996), fetal liver (Craig, W., et al. 1993) and peripheral blood stem cells after various mobilization protocols (Murray, L., et al. 1994). Similar to the data obtained in the murine system, comparative studies reveal that, in general, the basic and fundamental functional properties of stem/progenitor cells are shared regardless of the tissue source. There are however, significant functional and physical differences.

Numerous insights into hematopoietic molecular control mechanisms have come from gene-targeting studies in mice. Mutations in specific genes, most notably, those encoding transcription or DNA-binding proteins, have profound cell-intrinsic, global or lineage-specific effects on hematopoietic development (Shivdasani, R. A., and Orkin, S. H. 1996; Orkin, S. H. 1996). In the latter cases, it is tempting to speculate that the phenotypes result from defects in the commitment process. However, malfunctions in the commitment decision to “set up” a program of differentiation are difficult to distinguish from malfunctions in the differentiation program itself. Two gene products, AML1 (CBF2) and SCL (tal-1) appear to be necessary global regulators of hematopoiesis (Wang, W., et al. 1996; Okuda, T., et al. 1996; Porcher, C., et al. 1996; Robb, L. , et al. 1996). Whether these molecules act to specify a hematopoietic stem cell or by other means is an open question. Interestingly, both of these molecules play roles in leukemic transformation. A very important gain of function study documents the apparent ability of HOXB4 to increase primitive cell numbers without significant impairment of differentiation abilities (Sauvageau, G., et al. 1995). Together with observations that HOXA9 is translocated in myeloid leukemia (Nakamura, T., et al. 1996; Borrow, J., et al. 1996), these studies suggest an important hematopoietic role for homeobox proteins. Without question, the above and other studies have identified important regulators of hematopoiesis. However, in almost all cases these regulators were first identified in other systems. The opposite approach is to directly search for stem cell regulators in stem cells, Graf, L., and Torok-Storb, B. 1995; Yang, Y., et al. 1996). The present invention solves these problems.

### SUMMARY OF THE INVENTION

The human hematopoietic stem/progenitor cell population has been extensively characterized according to physical and antigenic criteria as well as in a variety of in vitro and in vivo assay systems. Collectively the human studies have revealed similarities to the hierarchical stem/progenitor cell organization defined in the murine system. In spite of significant strides in the identification of cytokines which can act on stem cells, it has not been possible to define a system where undifferentiated expansion of the most primitive stem cell population occurs. Similarly, it has not been possible to direct differentiation along lineage-specific pathways. These limitations, which also apply to the murine system, have hampered the elucidation of regulatory mechanisms which mediate the most fundamental aspect of stem cell biology; that is, the decision to self-renew or commit to differentiation. As a consequence, very little is known about the molecular biology of the most primitive hematopoietic stem cell in any organism. It was hypothesized that the regulation of primitive stem cells will be mediated at least in part by the products of genes which are uniquely or predominantly expressed in these cells. One precedent for an important, differentially-expressed molecule is the flk2/flt3 receptor tyrosine kinase. There presumably are other important and differentially expressed gene products. Therefore, it is an object of the present invention to identify these molecules and address their functional roles. Specifically, an aspect of identification of gene expression patterns specific to primitive human stem cells is the molecular phenotype of the human stem cell. The present invention describes methods to define the profile of genes specifically expressed in undifferentiated human stem/progenitor cell populations.

A primary focus of the present invention is on primitive cells isolated from normal bone marrow (BM) samples. The present invention further comprehends use of other sources of stem cells, such as umbilical cord blood (CB).

The methods of the present invention combine diverse technical approaches and sophisticated bioinformatic analyses.

This invention further provides methods to identify genes whose expression can be modulated by cytokine or stromal-dependent culture and/or by cell-cycle status.

Another object of the present invention is to provide methods for the functional characterization of human stem cell-specific gene products. An aspect of this invention is a method to facilitate the functional characterization of specifically expressed gene products as candidate regulators of a variety of stem/progenitor cell processes. In particular, a provided method uses an in vitro system which approximates many characteristic properties of normal stem cells to analyze positive and negative regulation of proliferation, cell-cycle parameters, apoptosis and commitment.

It is a further goal of the present invention to provide a necessary (and usually missing) component for stem cell gene-expression screens; that is, the ability to quickly assess the function of extensive panels of genes.

It is also an object of the present invention to provide a method for the functional identification of stem cell regulators. An aspect of this invention is a facile screening method for “categorizing” large populations of specifically-expressed molecules according to their potential roles in a variety of stem/progenitor cell processes. Gain of function as well as loss of function approaches are contemplate by the present invention. This method does not rely on any prior knowledge of nucleotide or predicted protein sequence.

Accordingly, the present invention provides an isolated nucleic acid derived from an isolated hematopoietic stem cell, the isolated nucleic acid comprising the following characteristics: (1) specifically expressed in the hematopoietic stem cell; and (2) encoding a hematopoietic stem cell-specific protein.

The present invention additionally provides an isolated hematopoietic stem cell specific protein or a portion thereof encoded by the provided nucleic acid. The present invention further still provides a nucleic acid probe capable of specifically hybridizing with the provided nucleic acid under standard hybridization conditions.

Also, the present invention provides an antibody capable of specifically binding to the provided protein without substantially cross-reacting with a non-stem cell specific protein or homologs thereof under conditions permissive to antibody binding. Additionally, the present invention provides a cell capable of producing the provided antibody.

In addition, the present invention provides a method for identifying the presence of a primitive hemopoietic stem cell in a sample comprising nucleic acids specifically expressed in hematopoietic stem cells. Further still, the present invention provides a method for generating a stem cell/progenitor cell from a primitive hematopoietic cell in a sample.

The present invention further provides a method for identifying the presence in a sample of a compound that modulates hematopoietic stem cell activity.

The present invention even further still provides a method for identifying primitive hematopoietic stem cell-specific nucleic acids.

Also the present invention additionally provides a molecularly defined primitive hematopoietic stem cell.

Yet additionally, the present invention provides a method for treating a condition in a subject comprising administering to the subject a therapeutically effective amount of a provided pharmaceutical composition. The present invention provides a method of introducing an exogenous nucleic acid into a hematopoietic stem cell. Finally, the present invention provides a method of ex vivo expansion of hematopoietic stem cells. The expanded cells are available to receive exogenous genes, including by retroviral or other vectors which require a round of replication. Alternatively, the cells are available for transplantation either autologously or heterologously.

### BRIEF DESCRIPTION OF THE FIGURES

FIG. 1. Schematic “flow of information” strategy.

FIG. 2. Mammalian stem cell system (black circle=strong hybridization signal, gray circle=detectable hybridization signal, and open circle=no detectable hybridization signal). The Smc-34 cDNA is a completely novel sequence with a predicted leucine zipper and several potential membrane spanning domains.

FIGS. 3A-3B. (right panel), the control, non-subtracted RDA cDNA population (38−) contains b-actin sequences which are missing in the two subtracted RDA populations, 38− and 38−)38+. A differentially expressed gene (HDD-2, described below) is enriched in the 38− RDA population and at least retained in the 38−)38+ RDA population (FIG. 3A, left panel). Two, bidirectional, RDA CDNA populations (38− and the converse 34− )38) were used to probe (See FIG. 3) duplicate arrays of a subtracted 38− library (FIG. 3).

FIGS. 4A-4E. FIG. 4A: 34B4 (SEQ.ID.No.: 69) is closely related to a gene encoding TINUR The sequence homologies and restricted expression pattern of 34A5 is shown in FIGS. 4B and 4C.

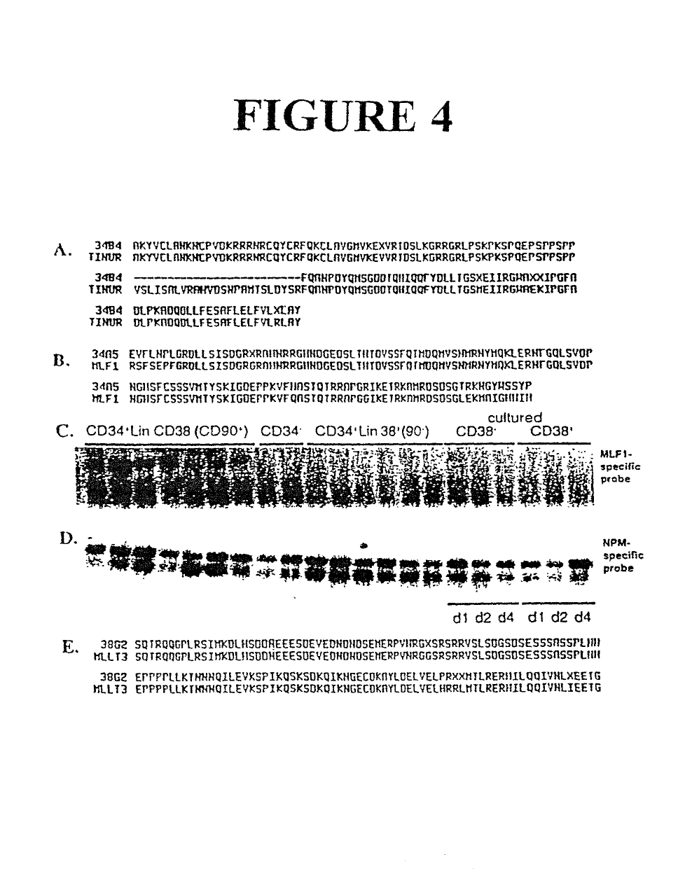

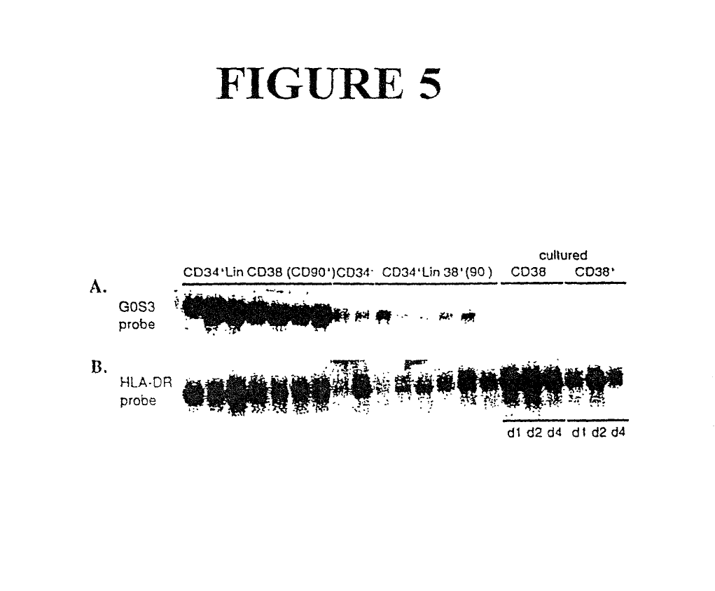

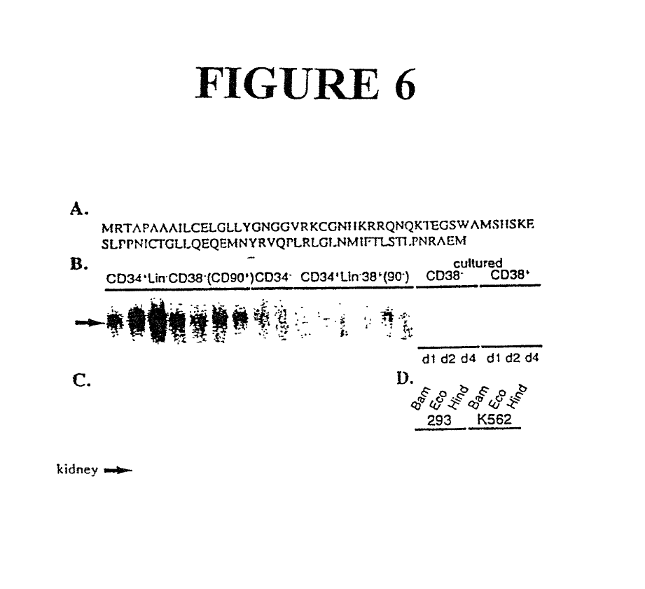

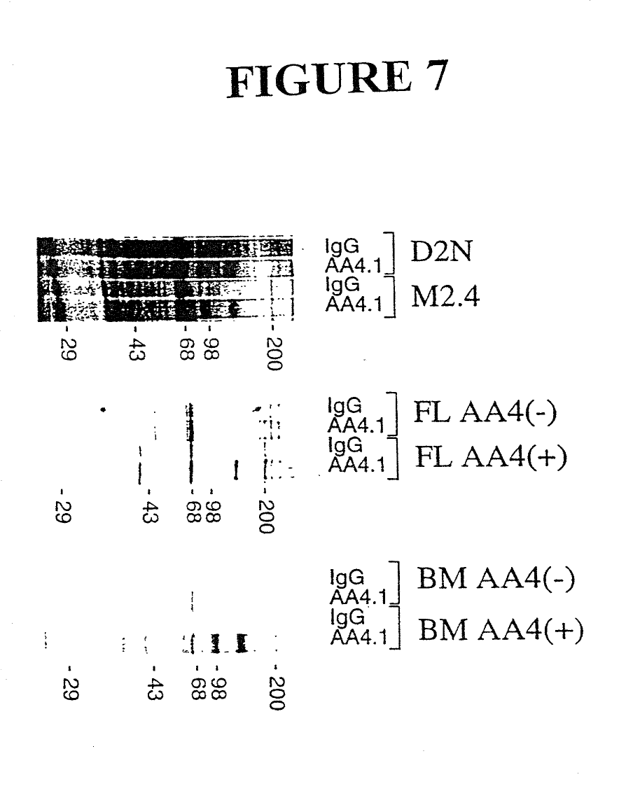

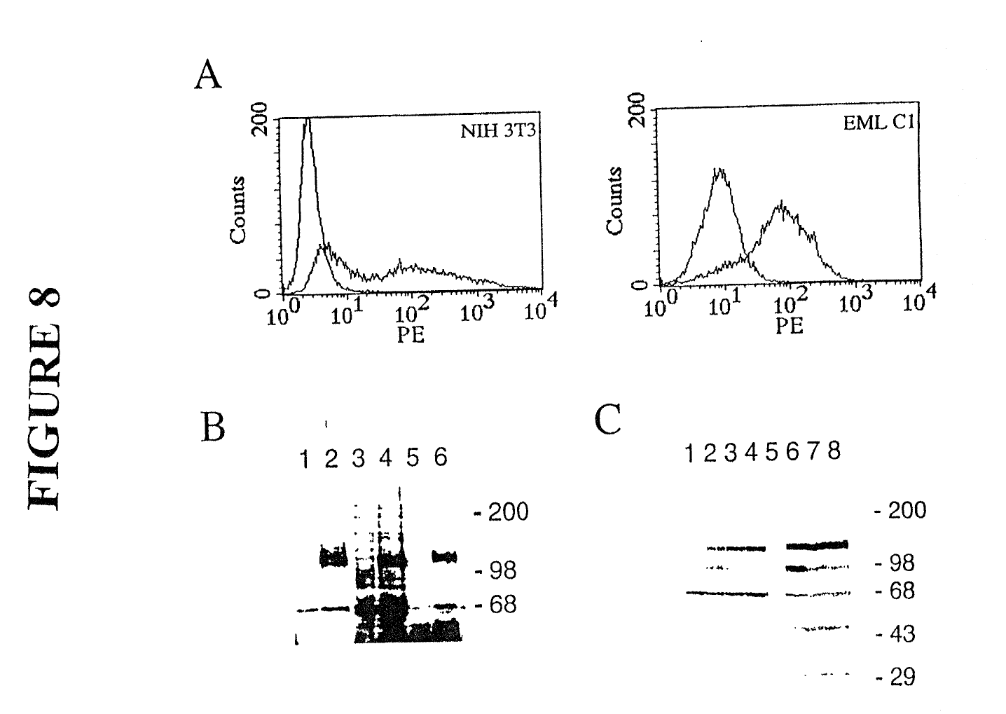

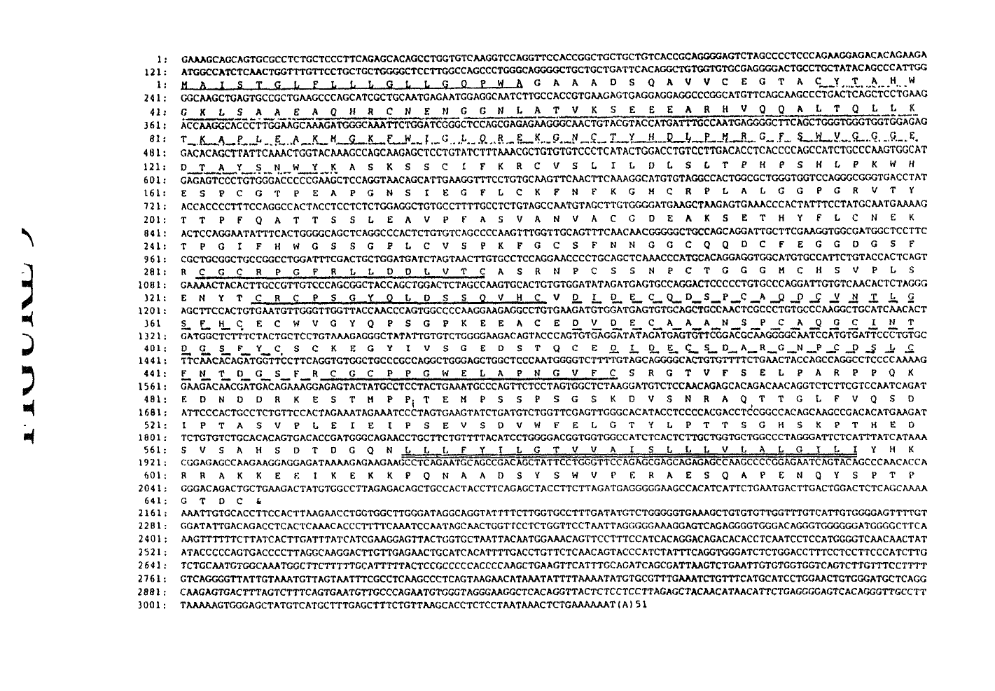

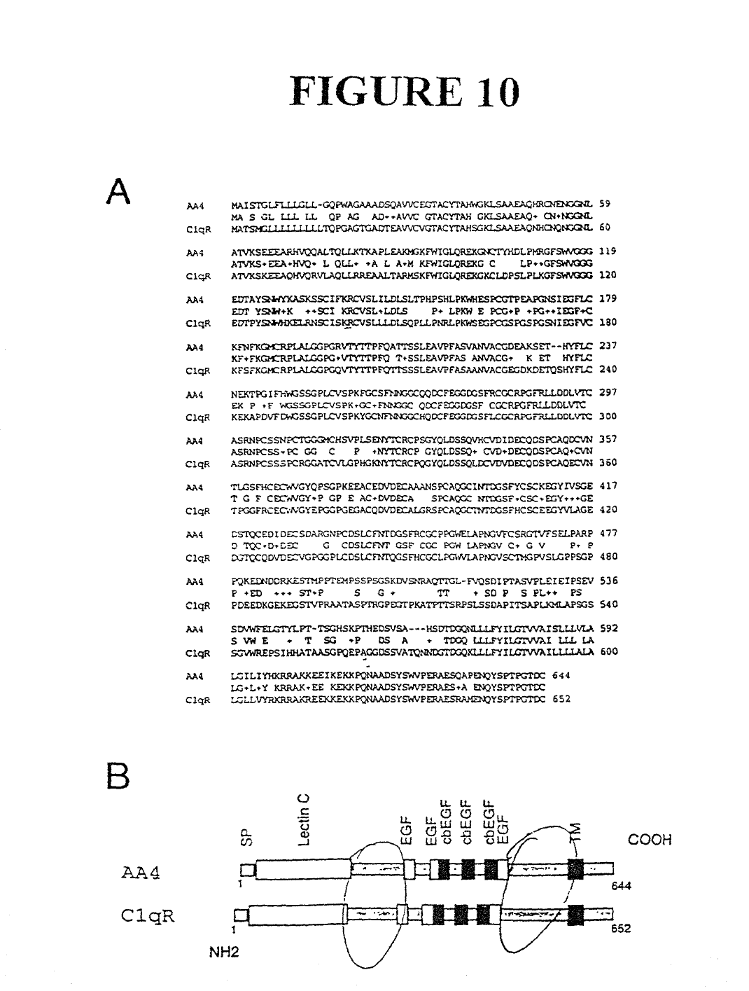

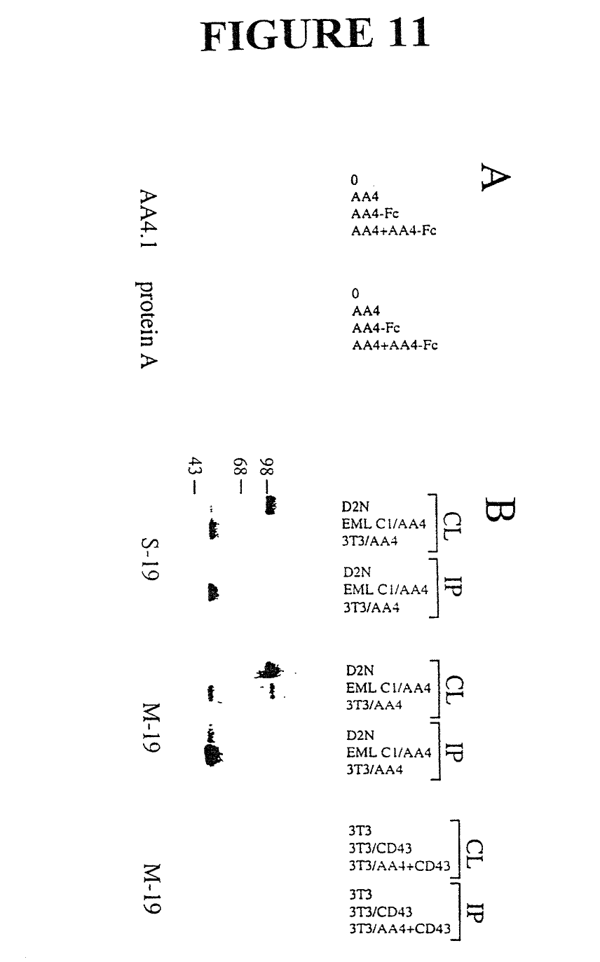

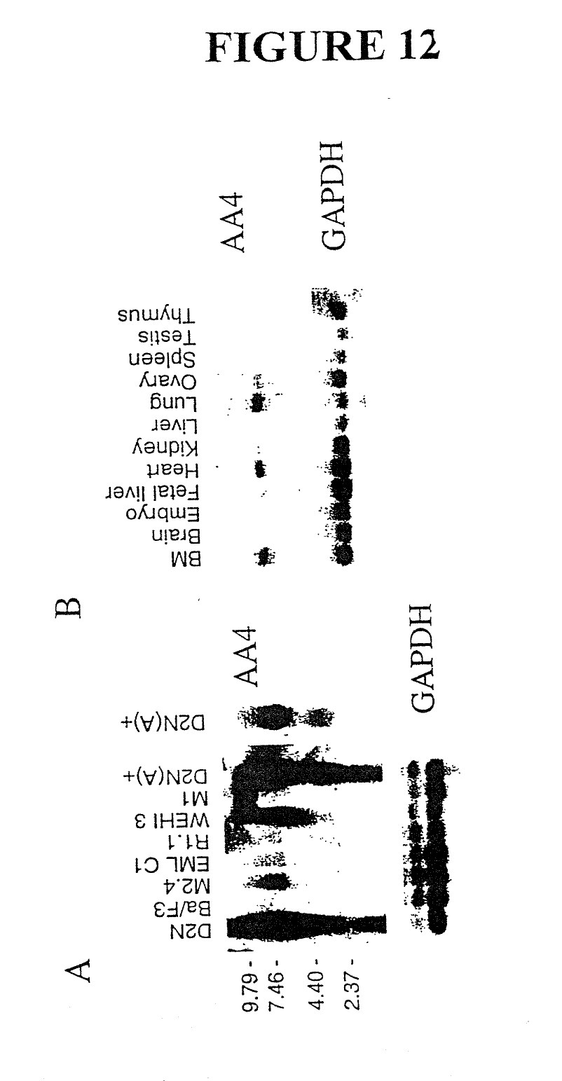

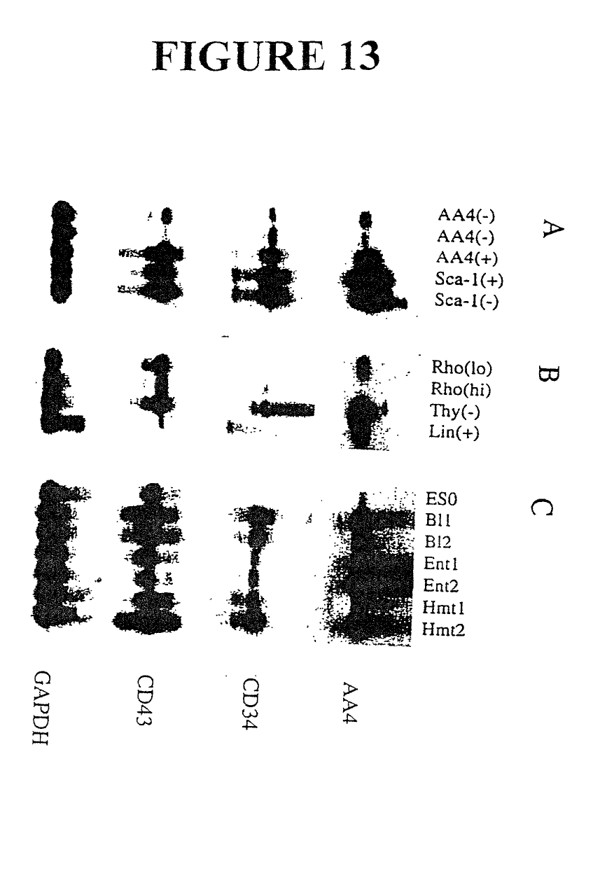

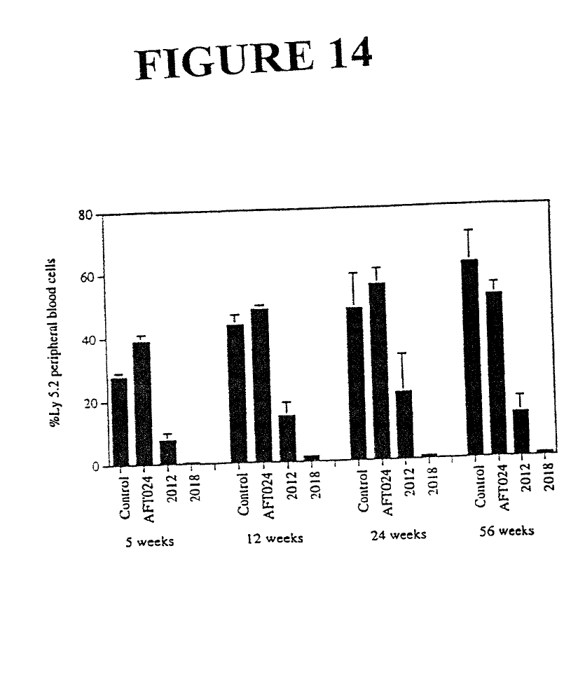

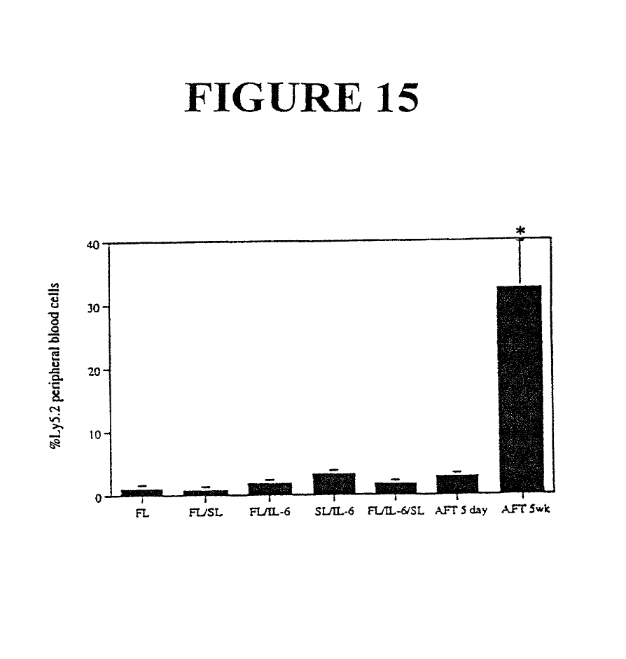

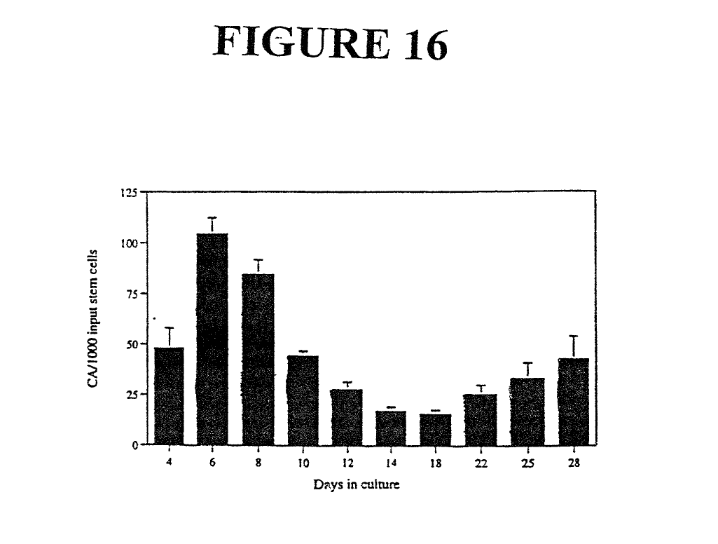

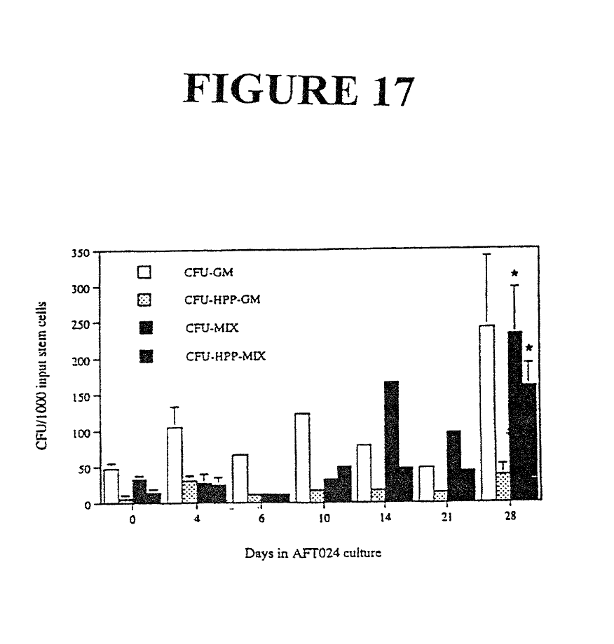

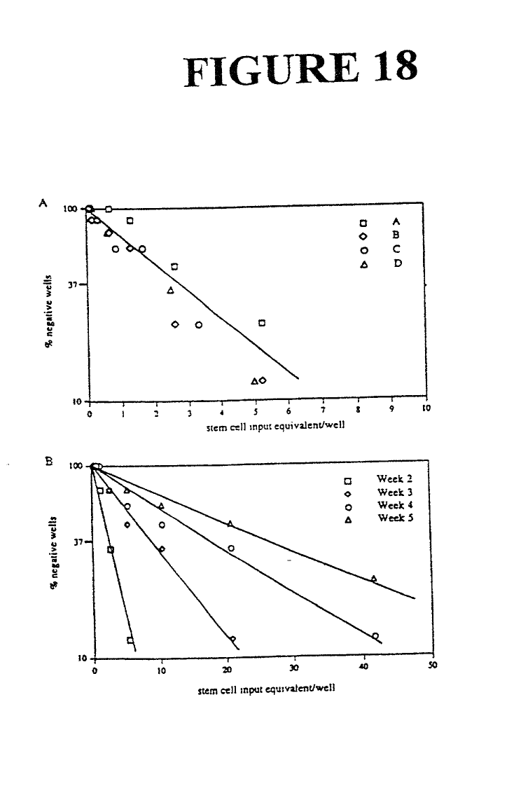

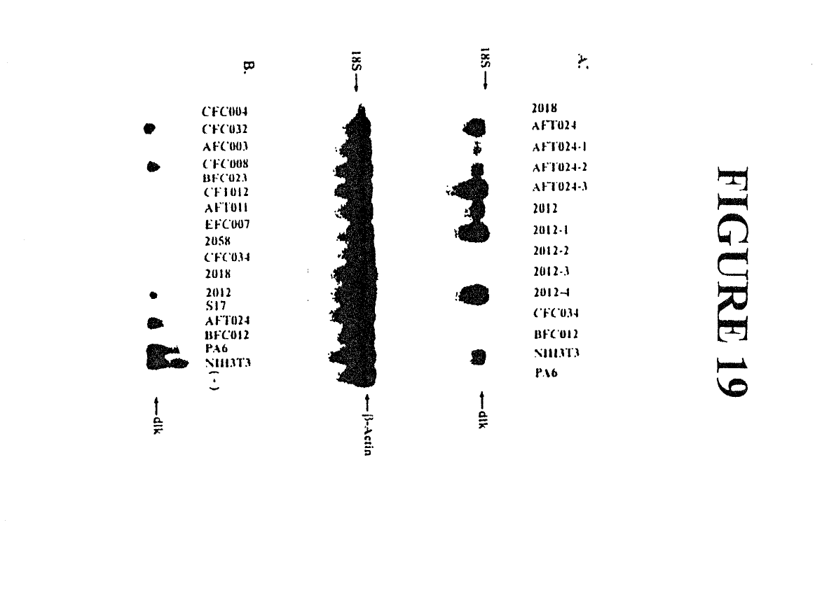

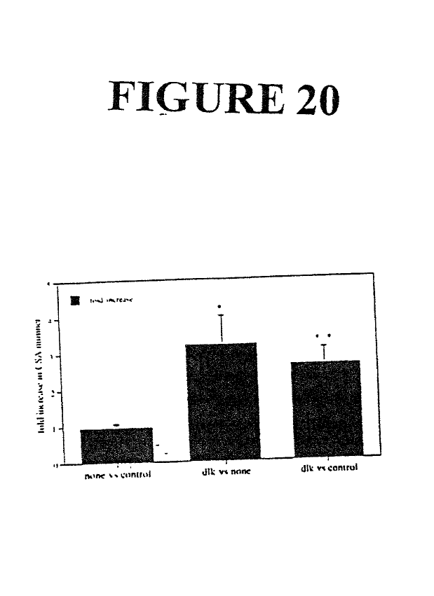

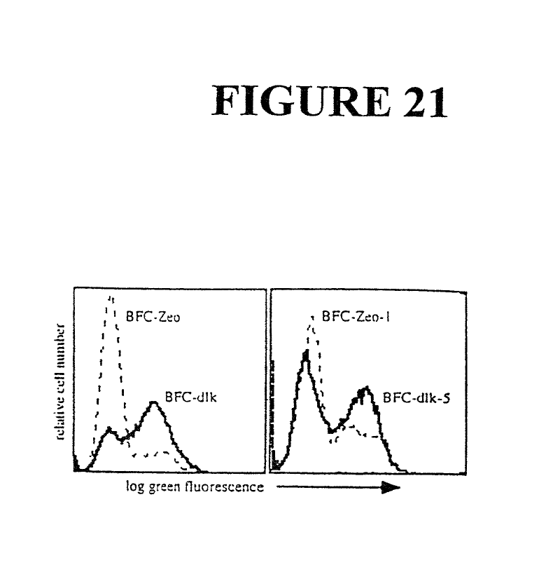

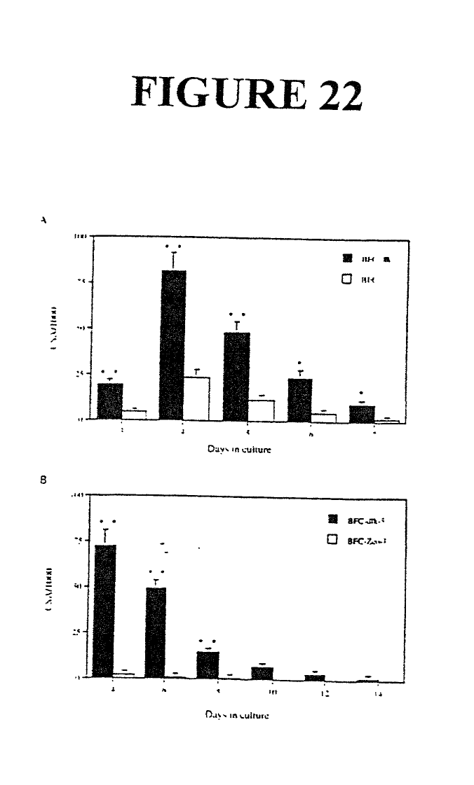

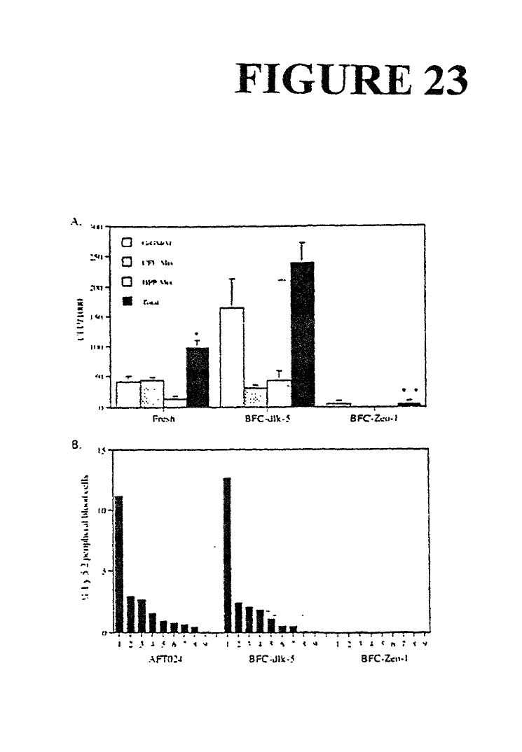

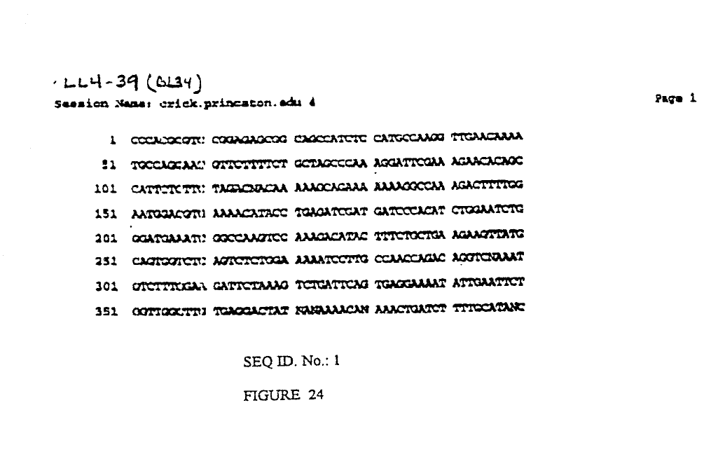

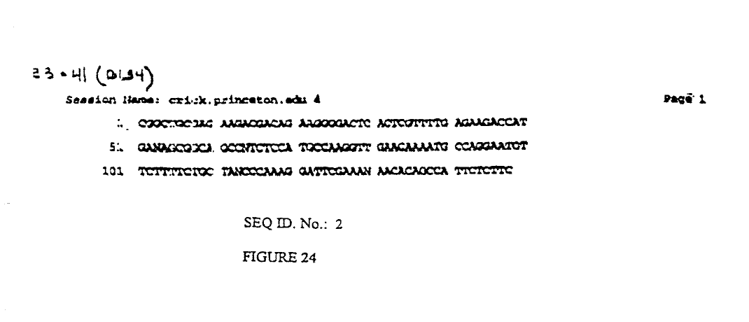

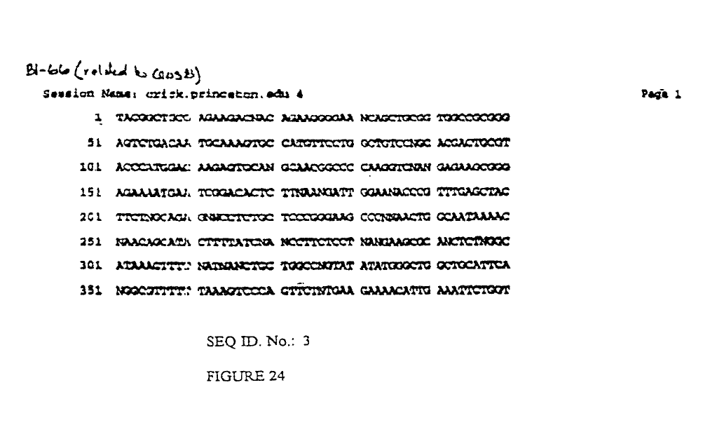

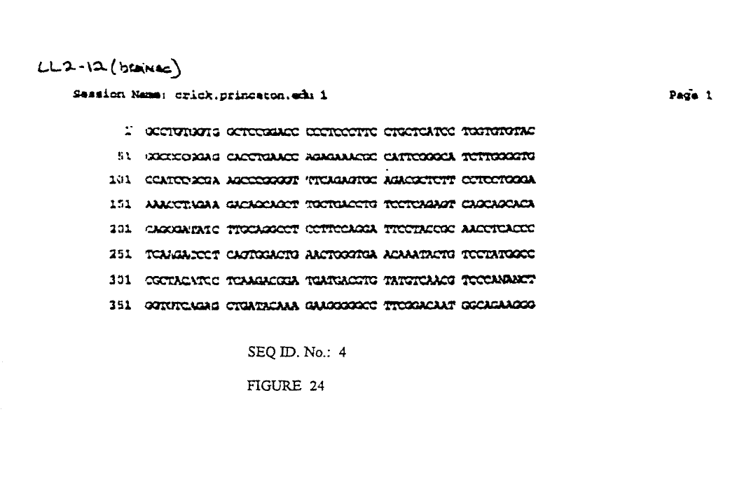

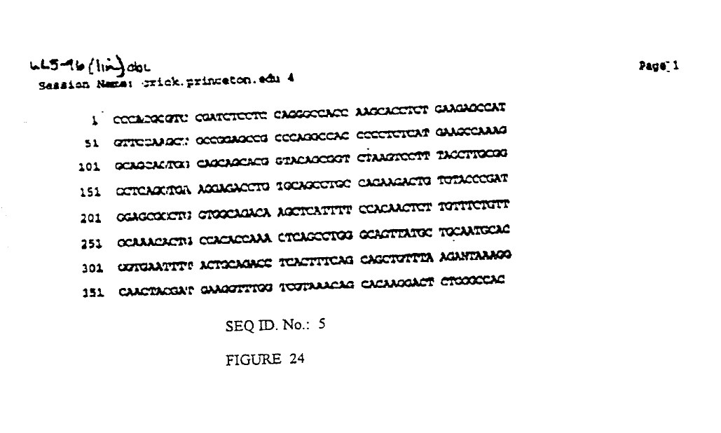

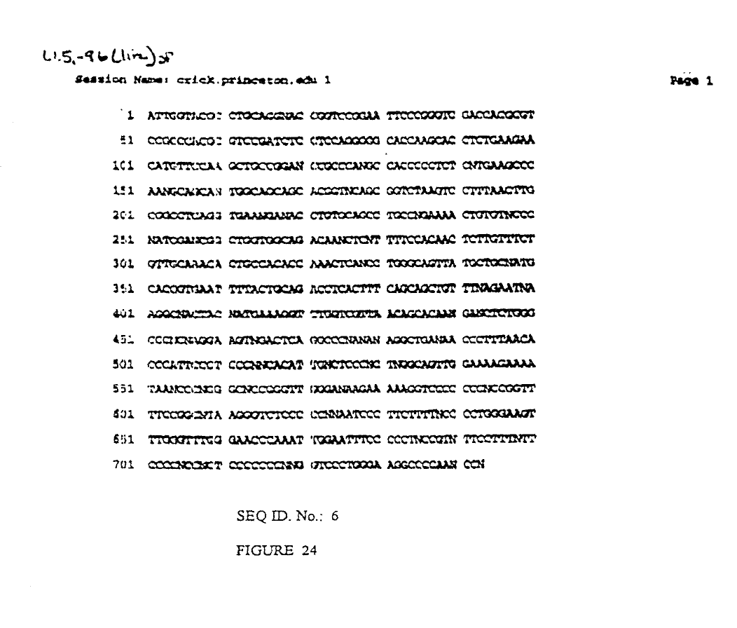

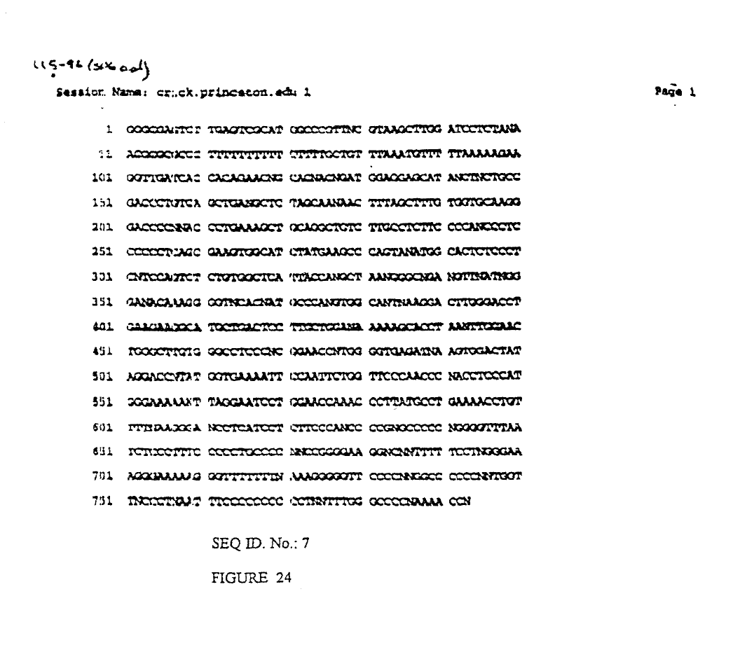

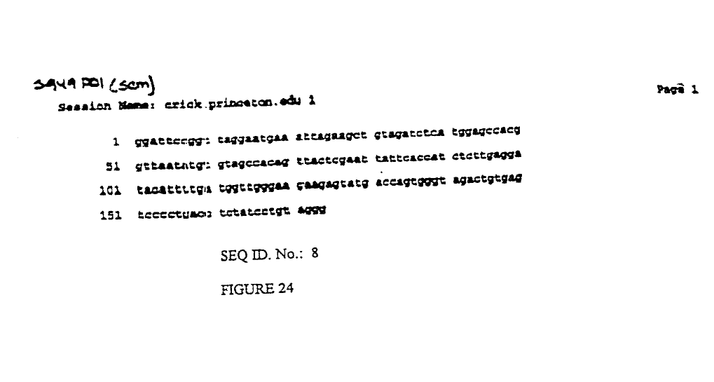

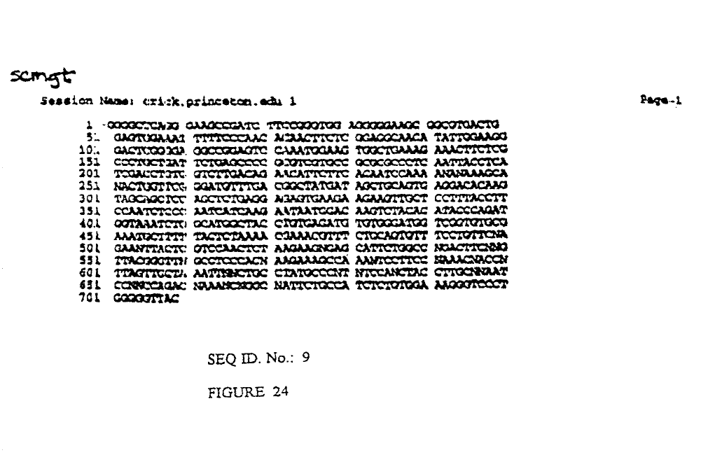

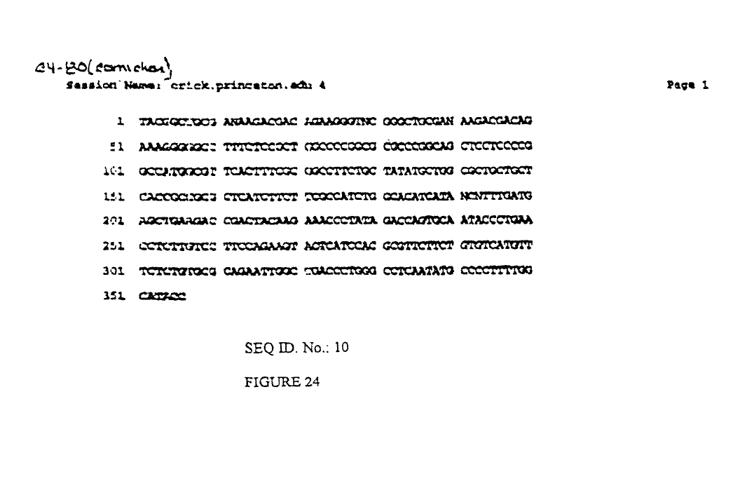

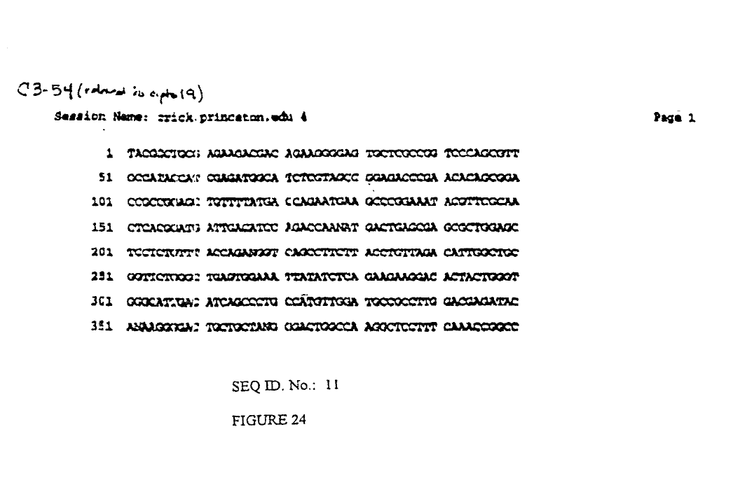

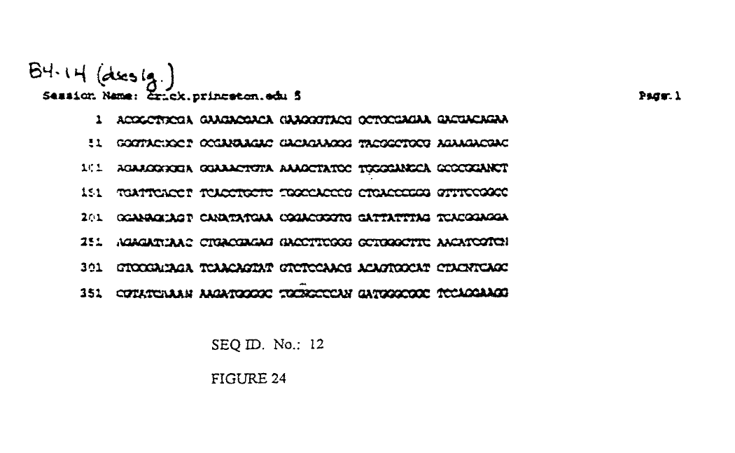

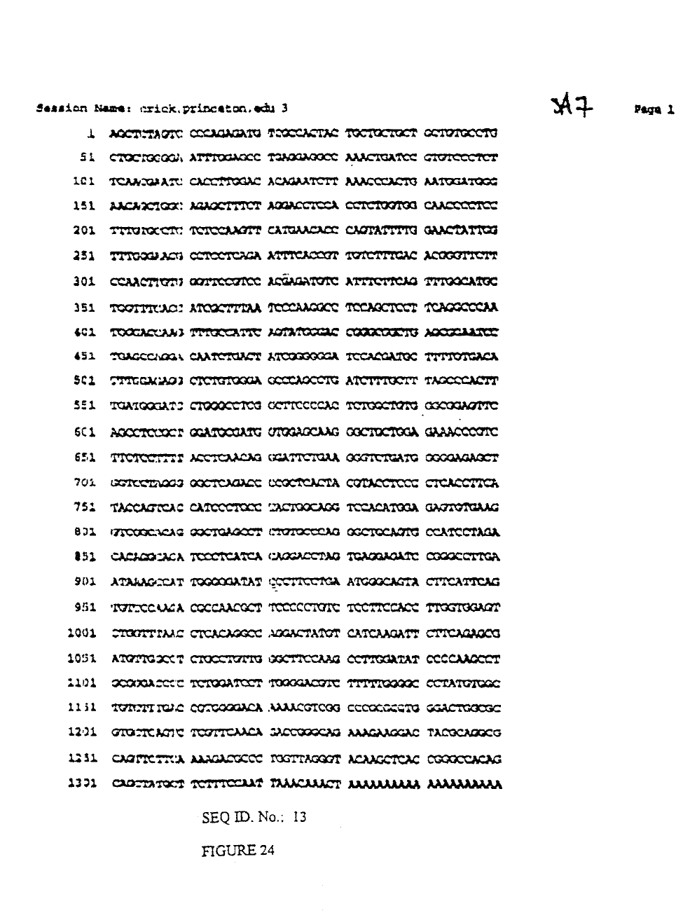

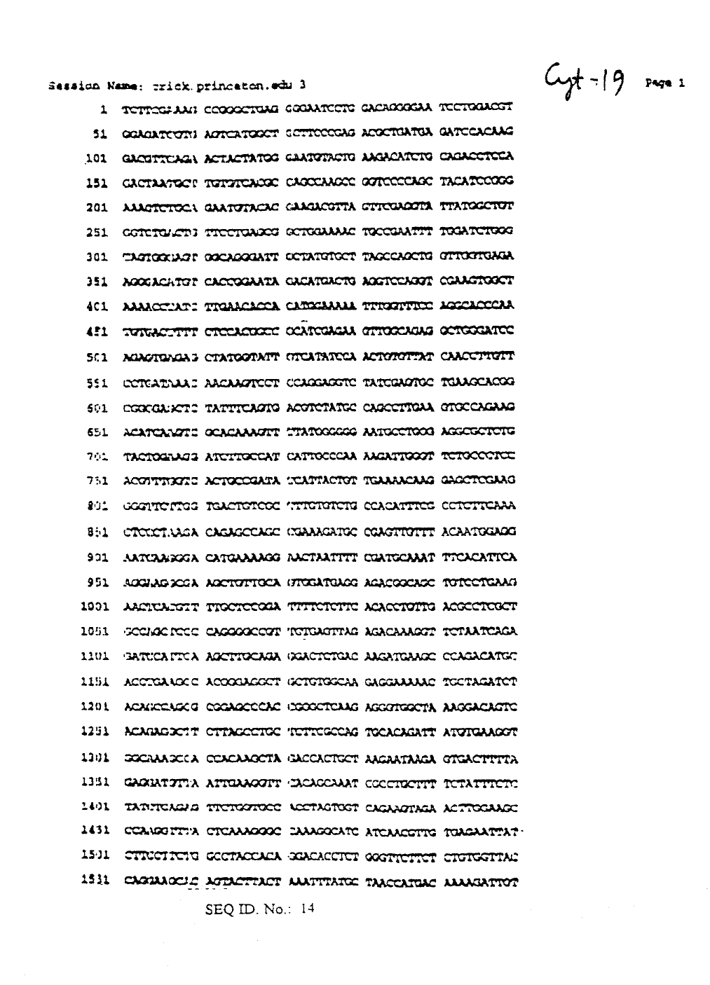

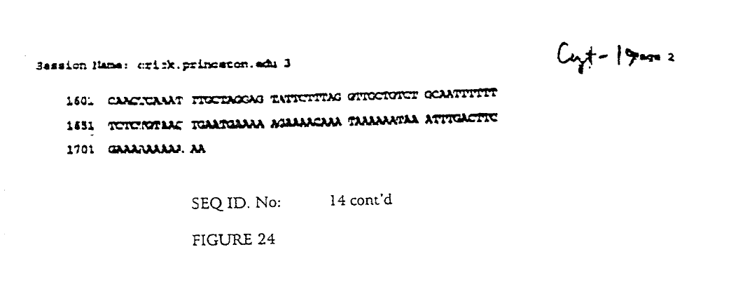

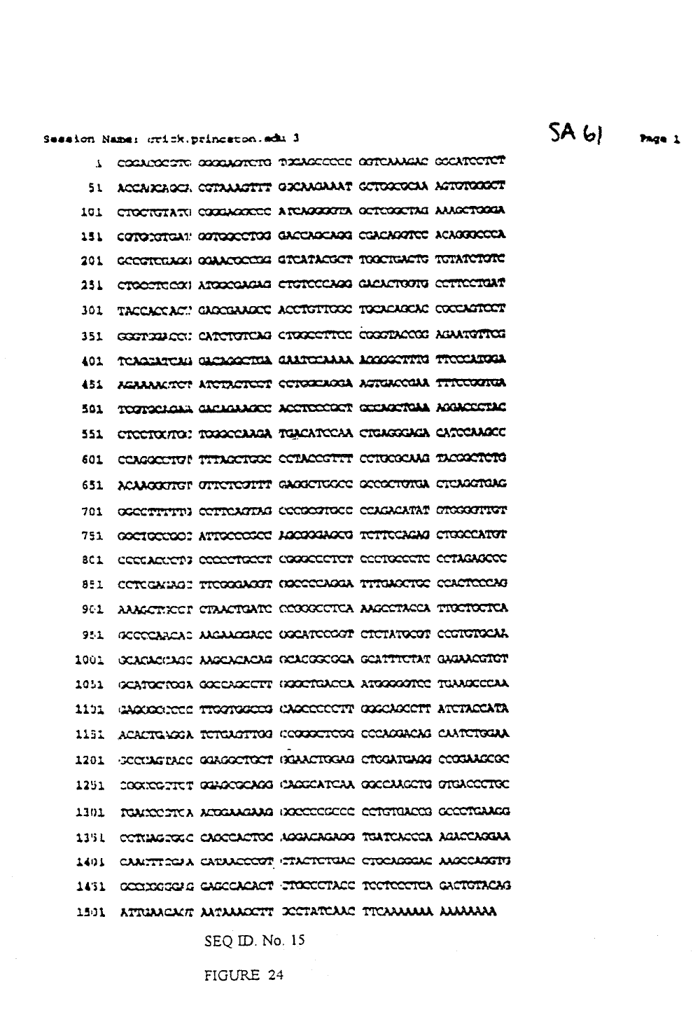

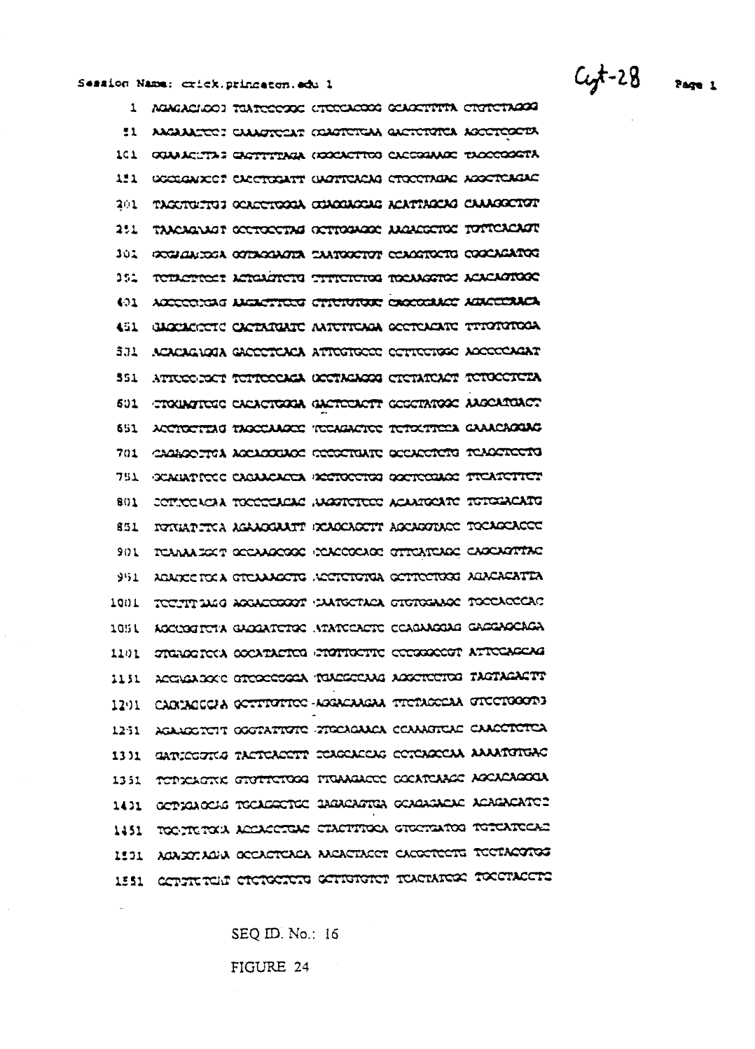

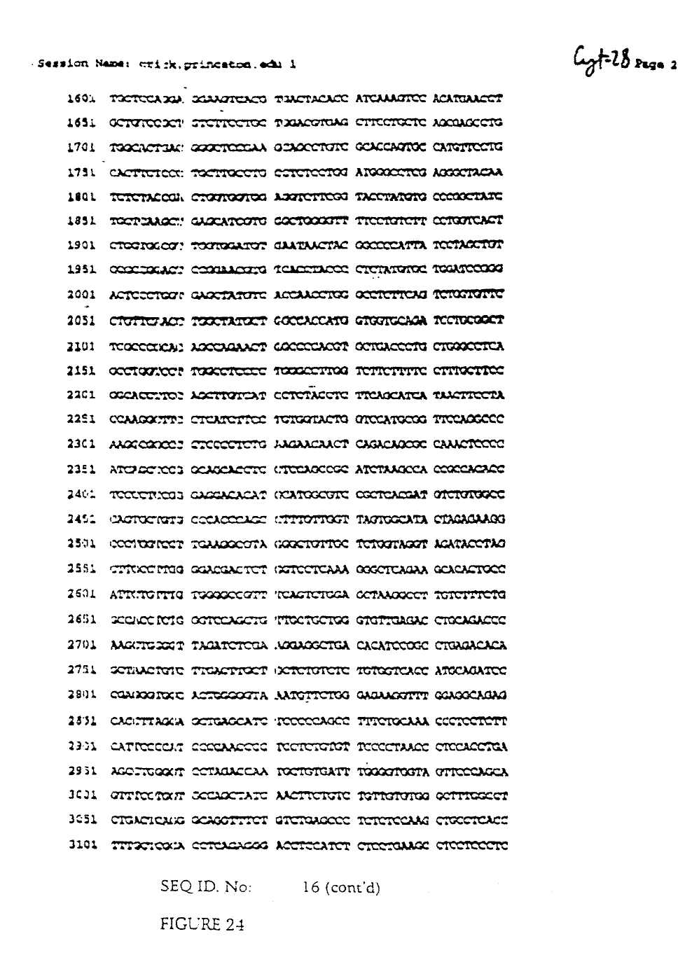

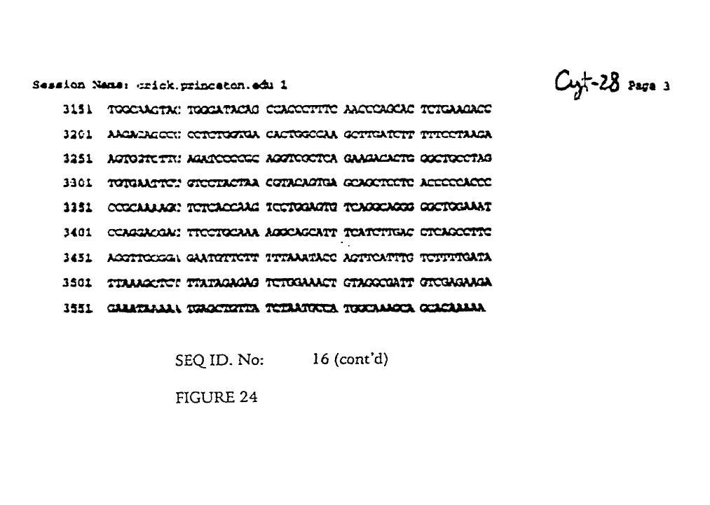

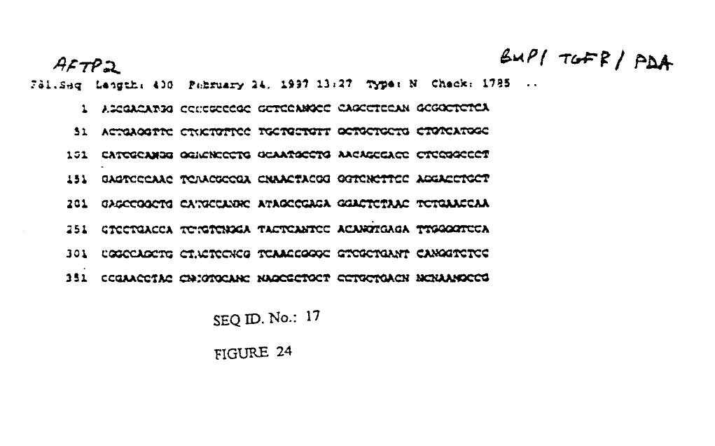

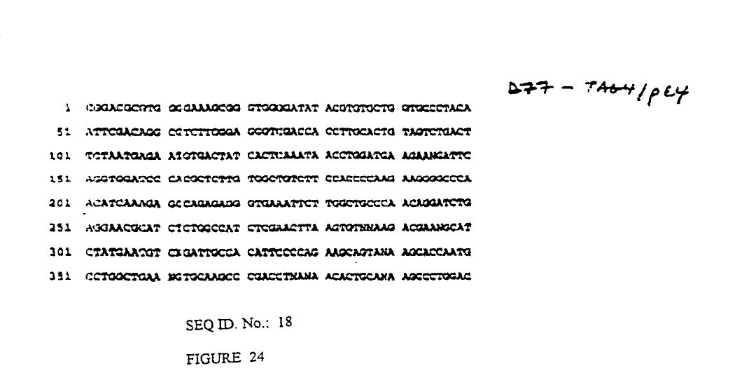

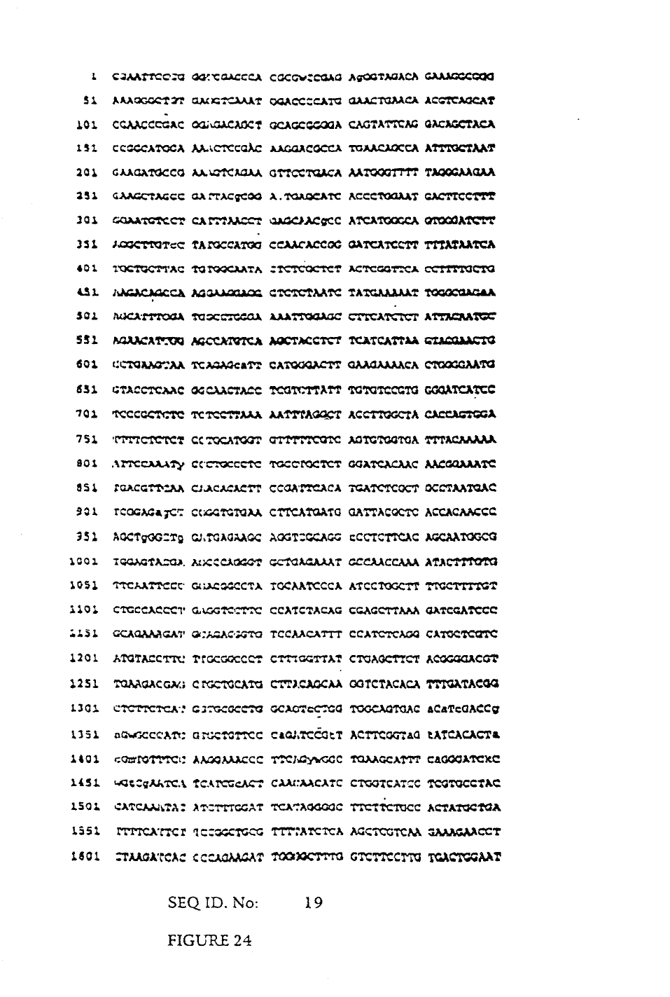

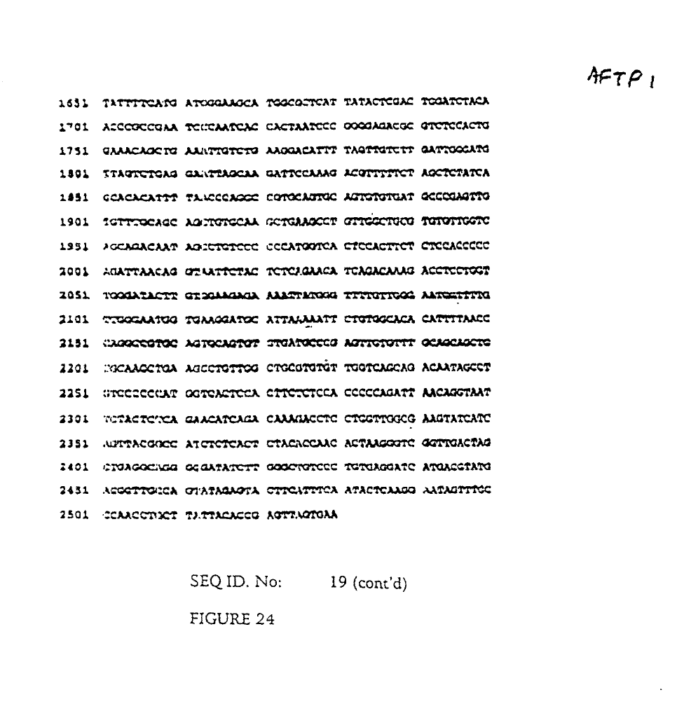

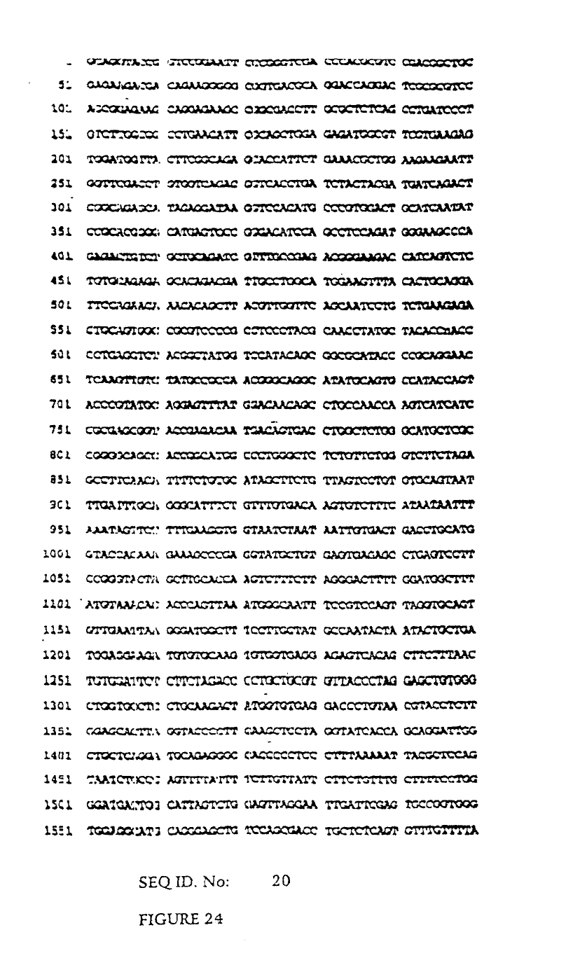

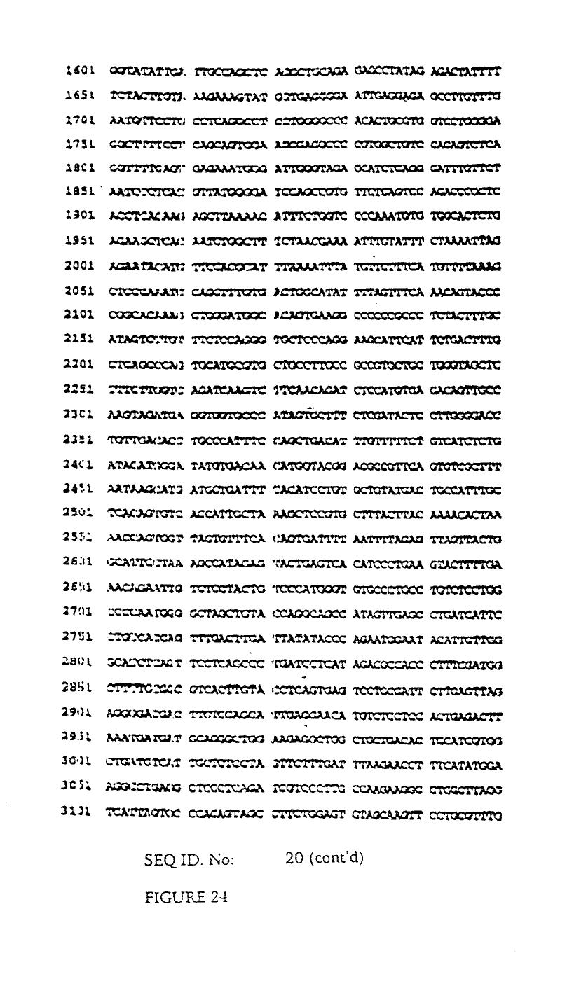

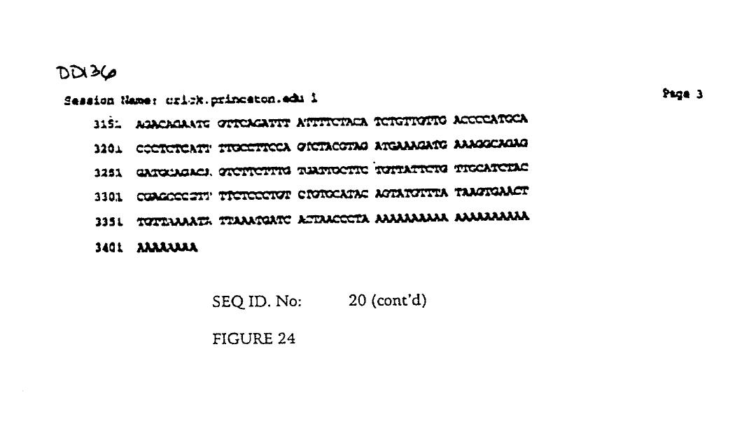

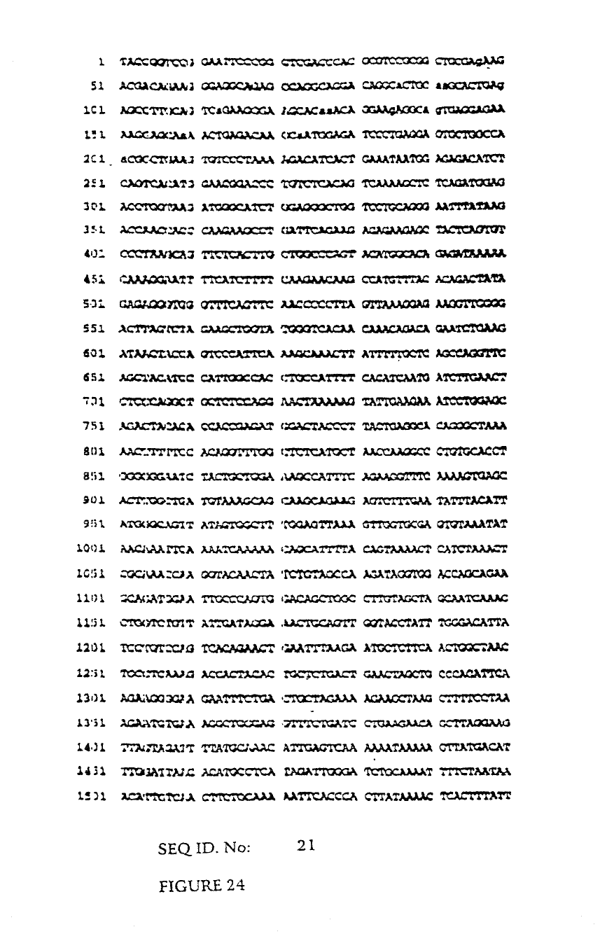

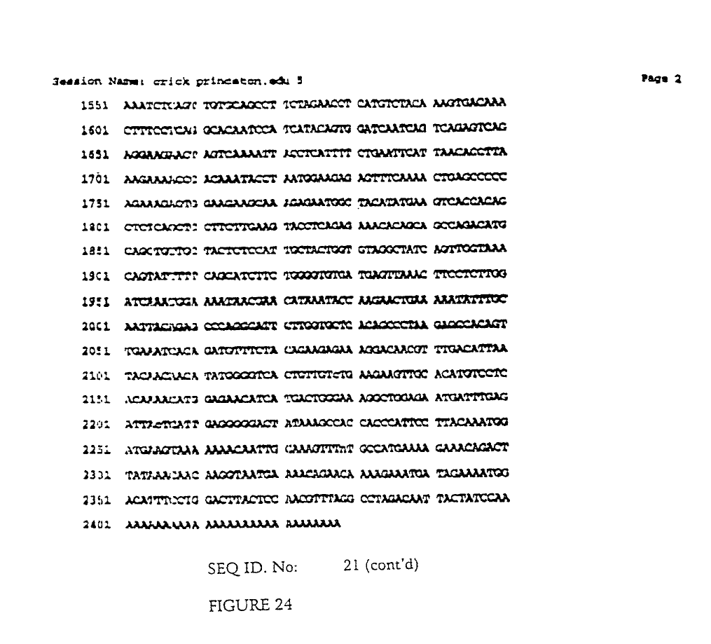

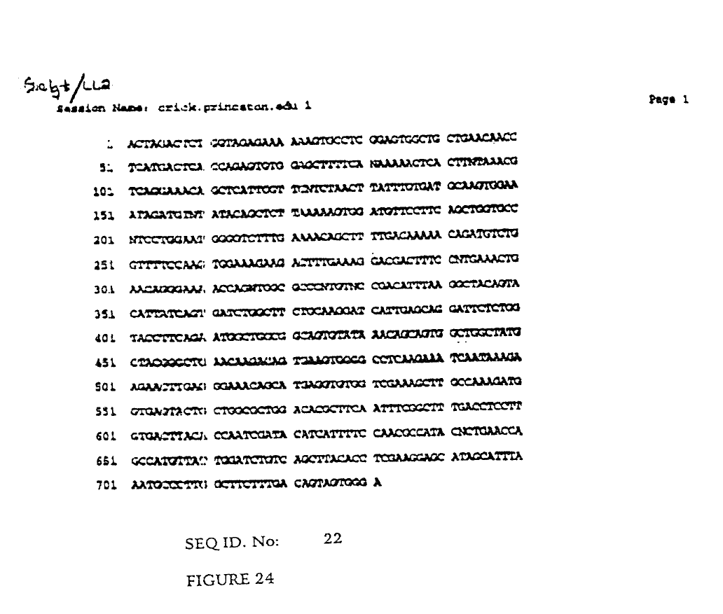

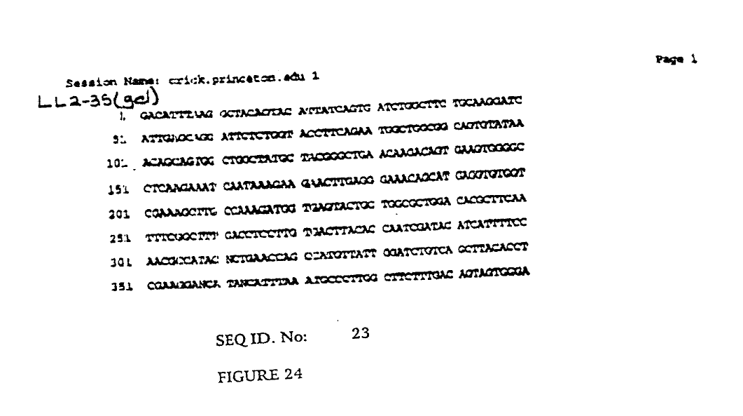

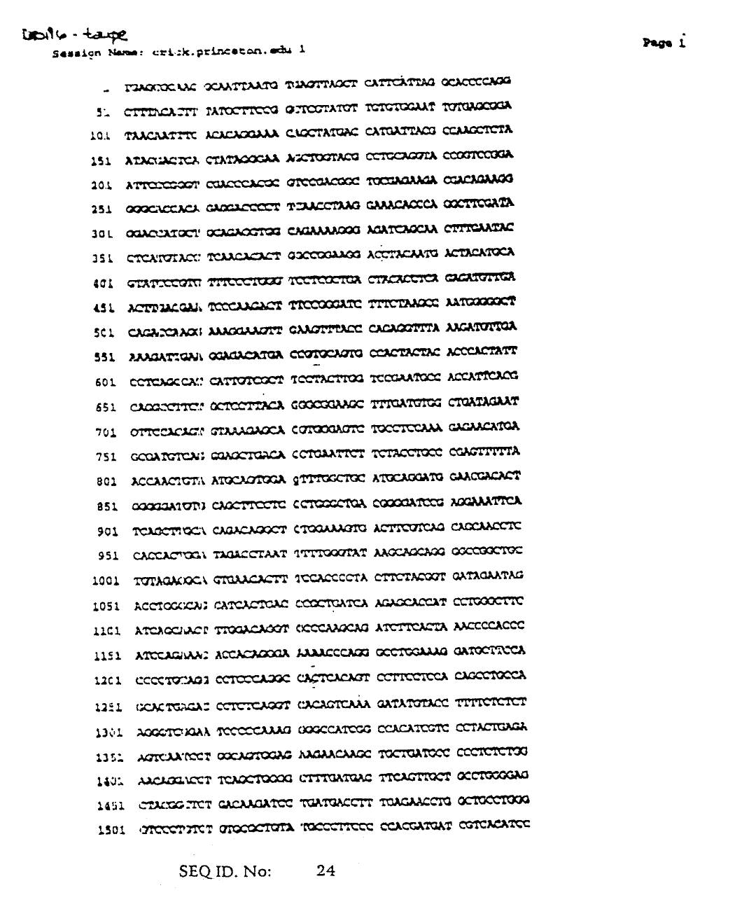

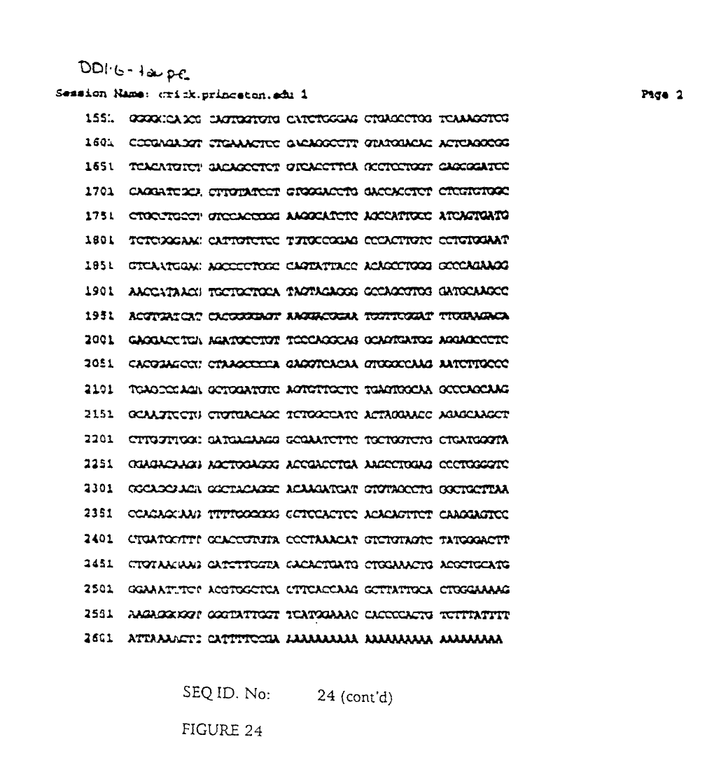

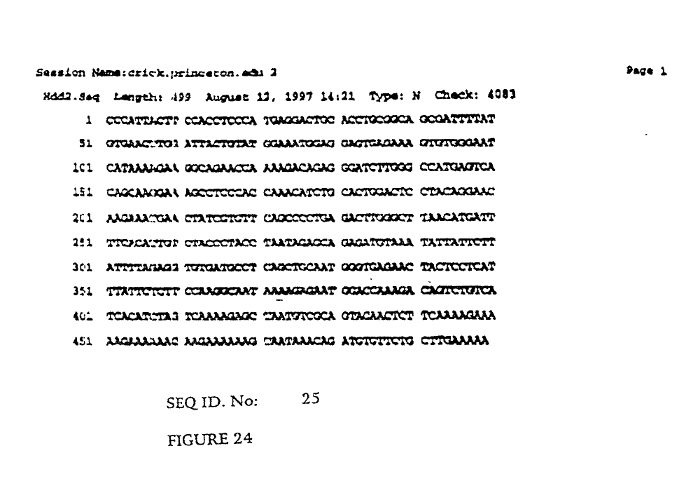

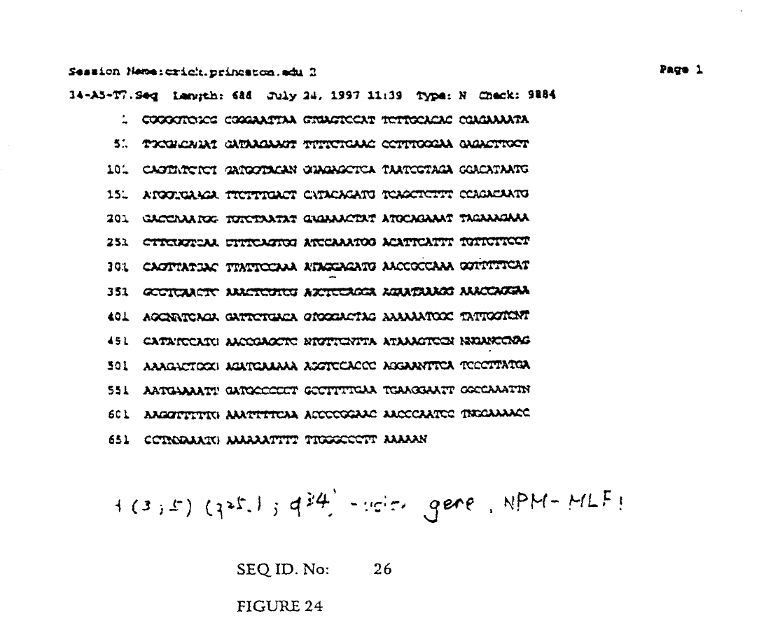

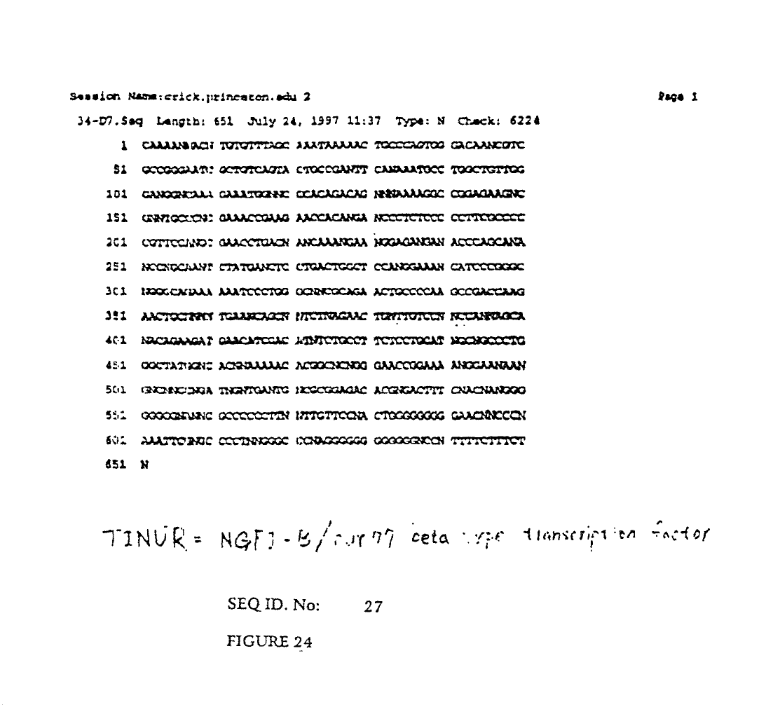

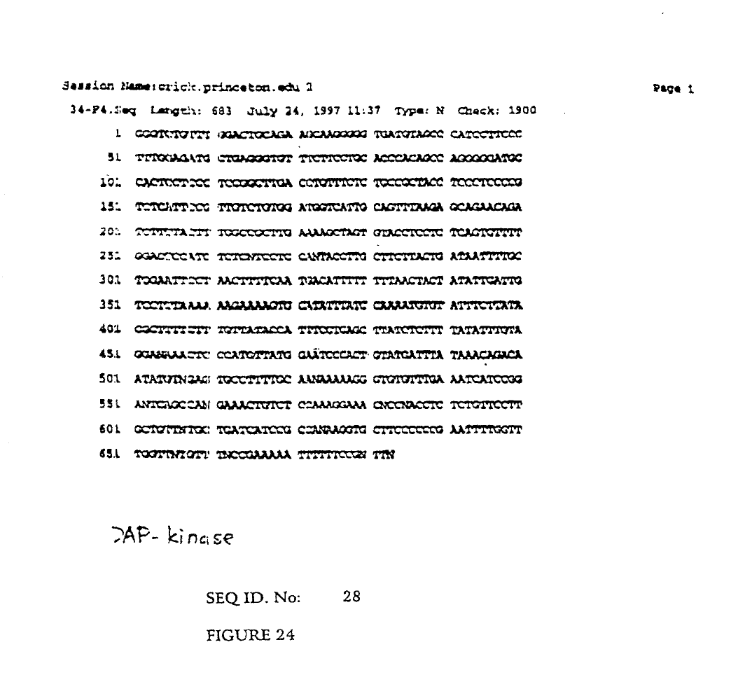

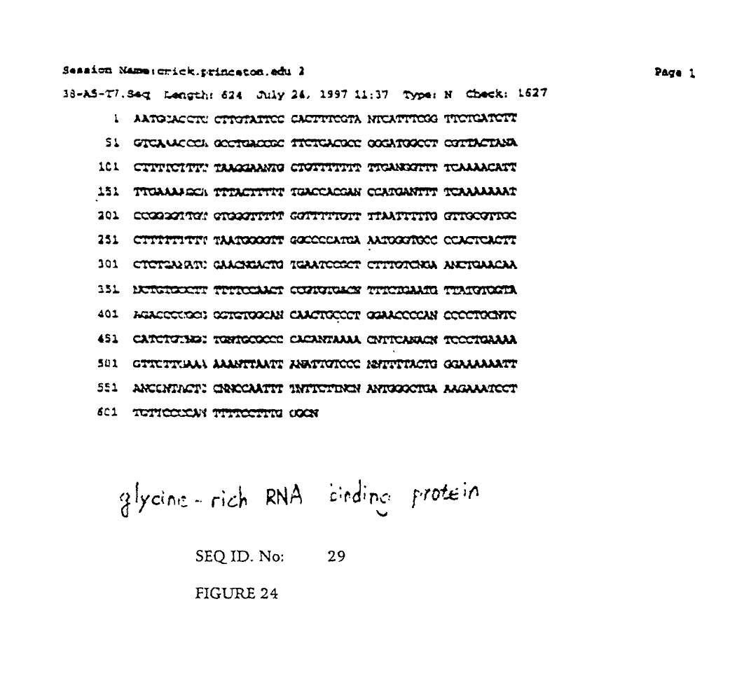

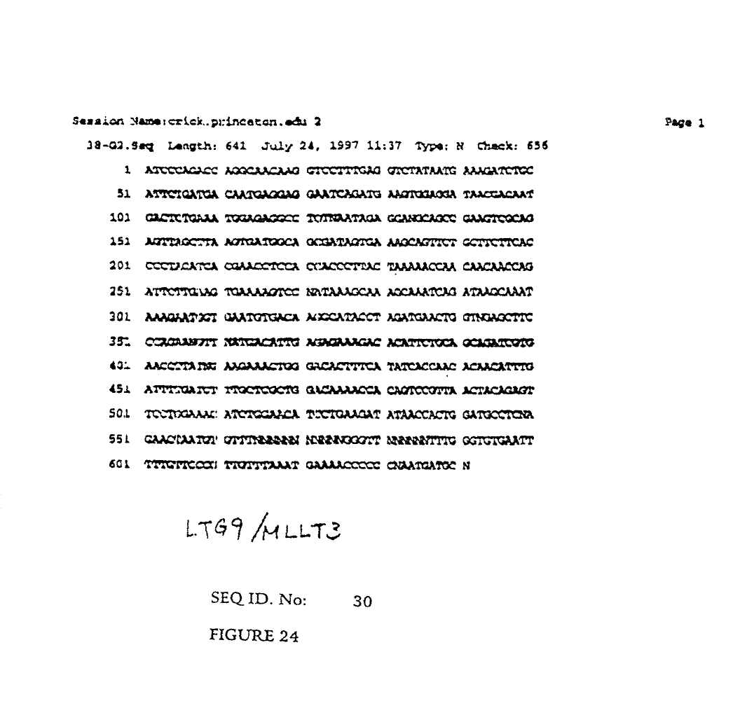

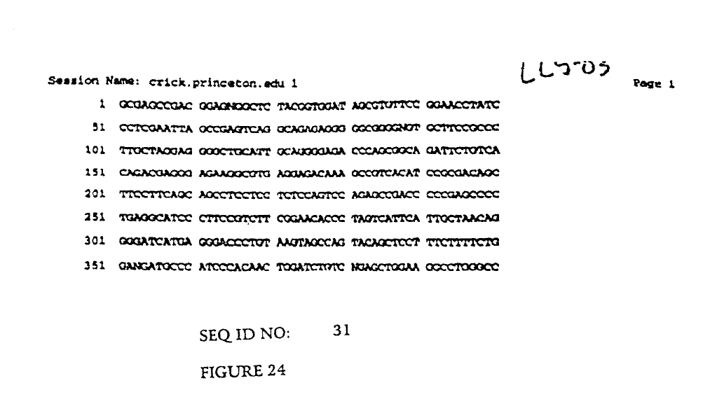

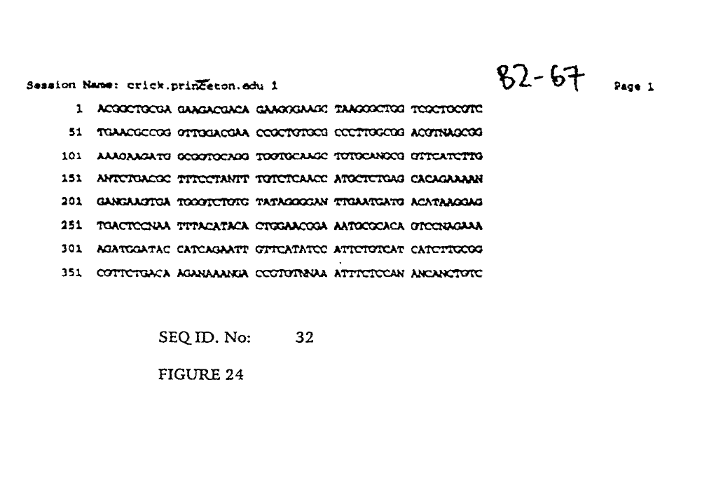

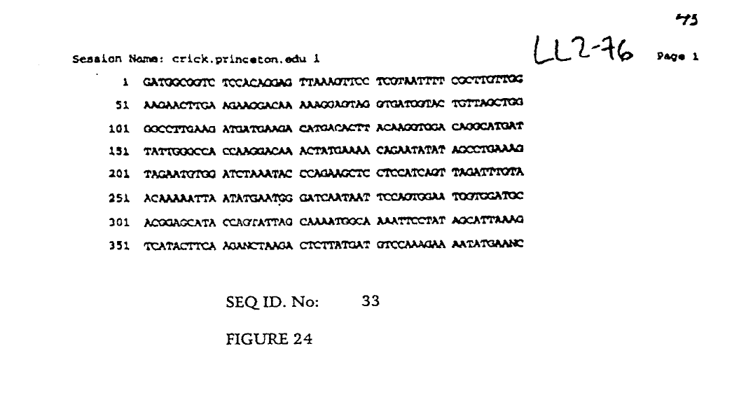

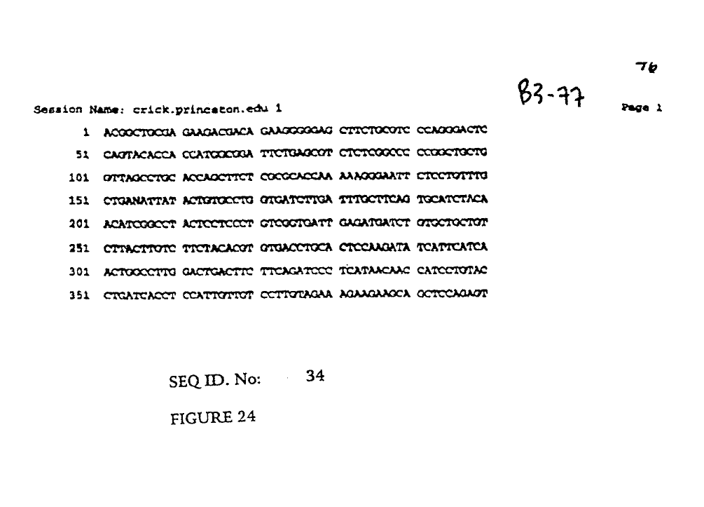

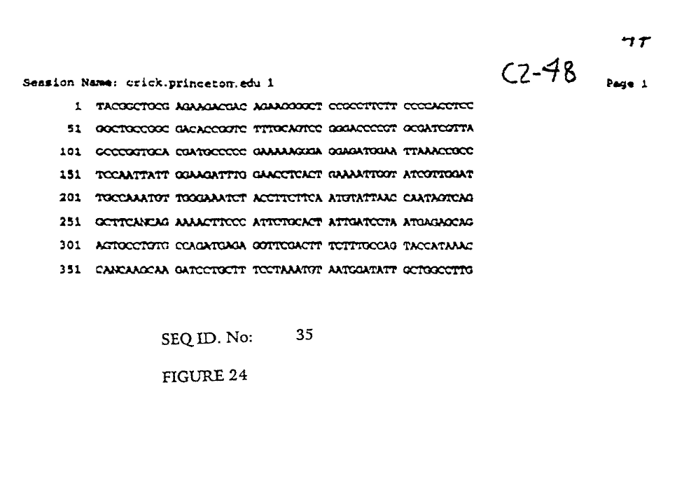

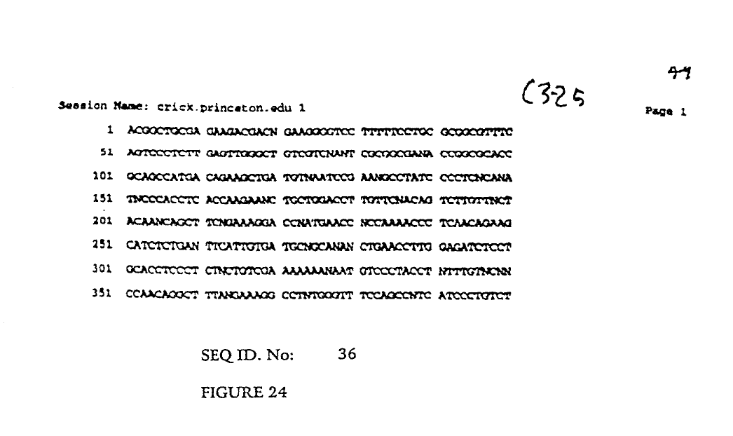

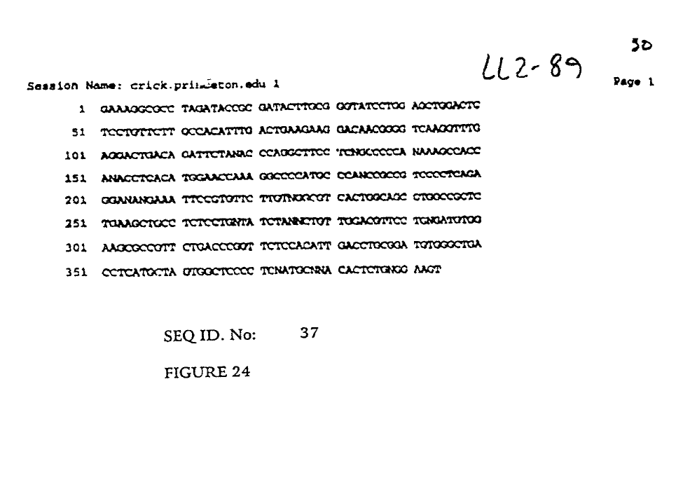

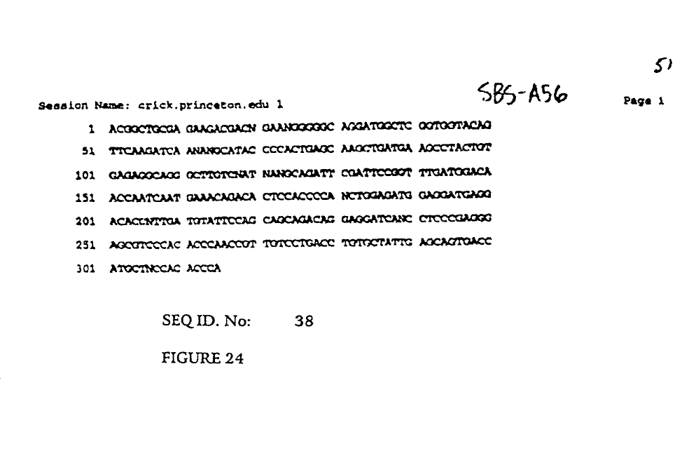

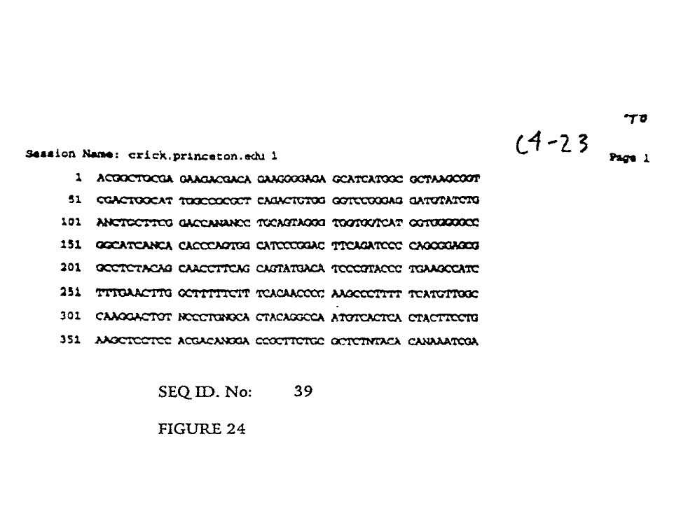

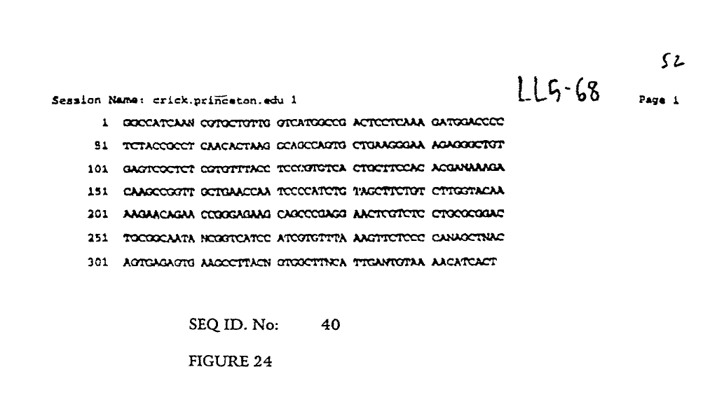

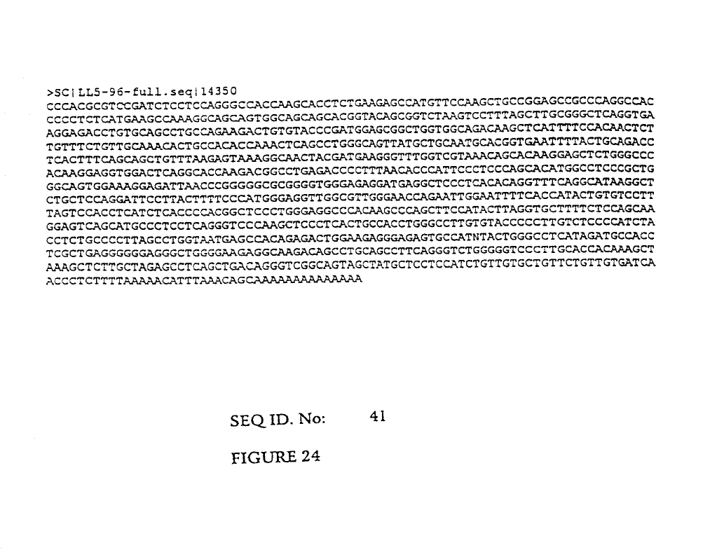

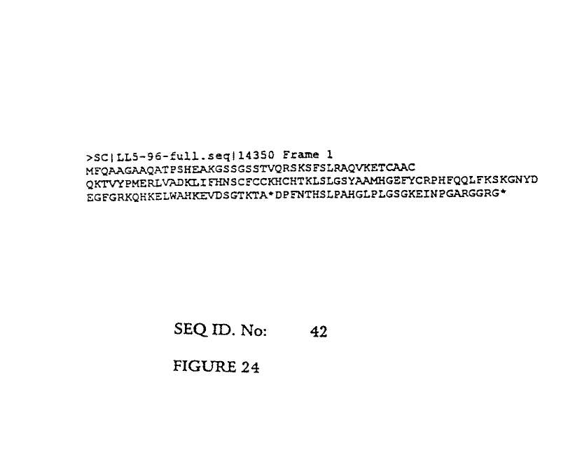

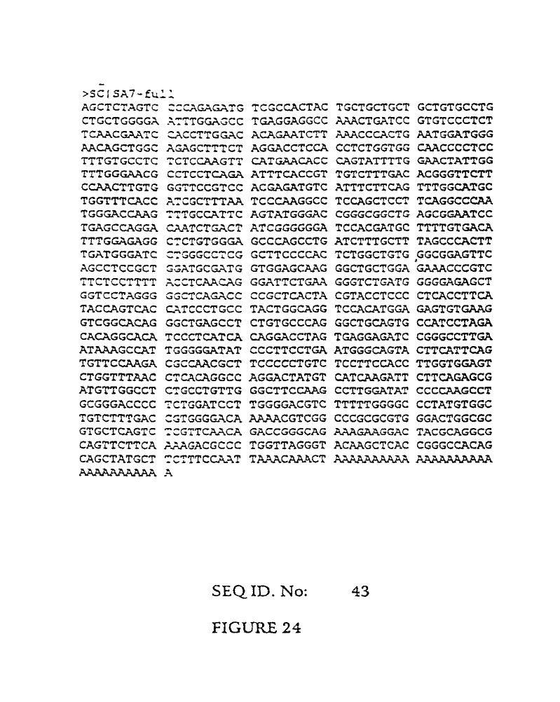

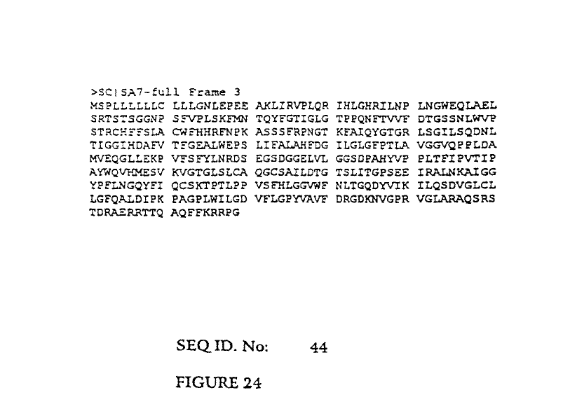

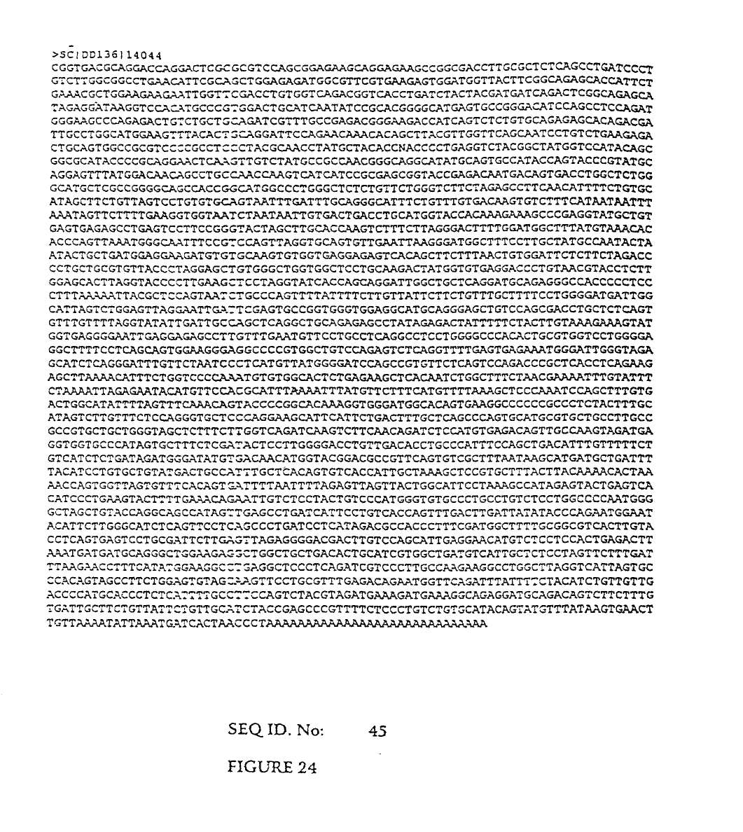

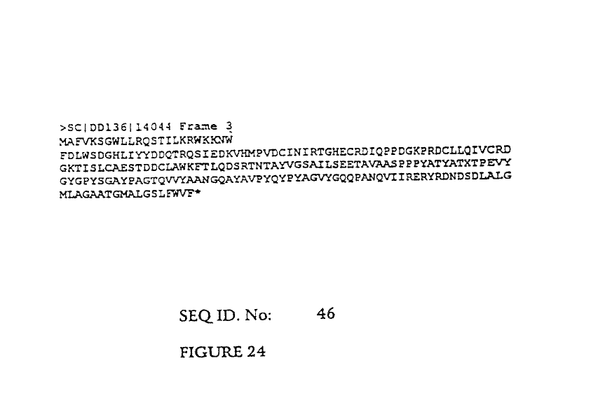

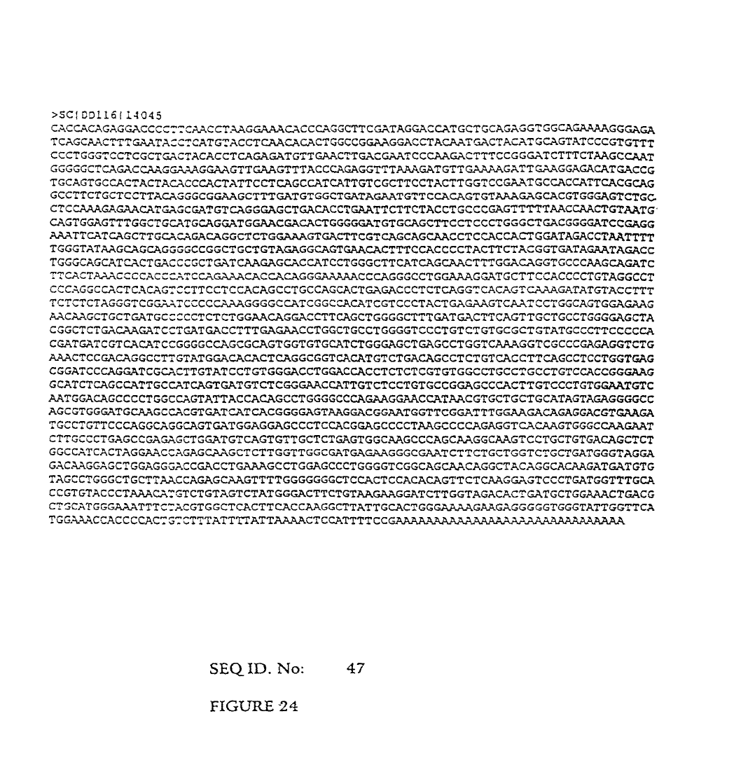

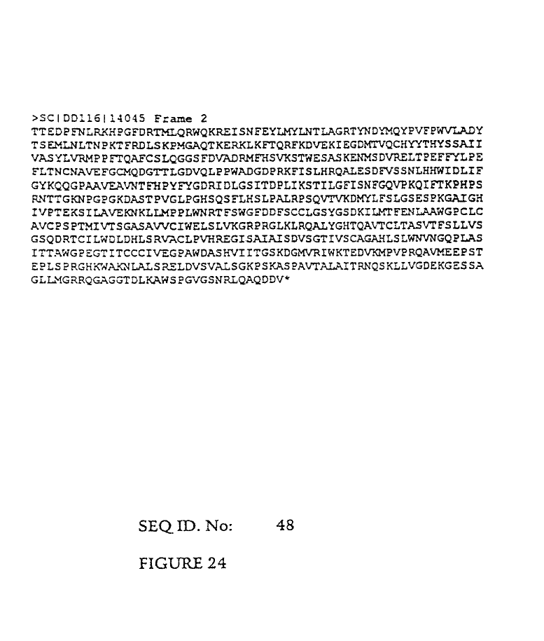

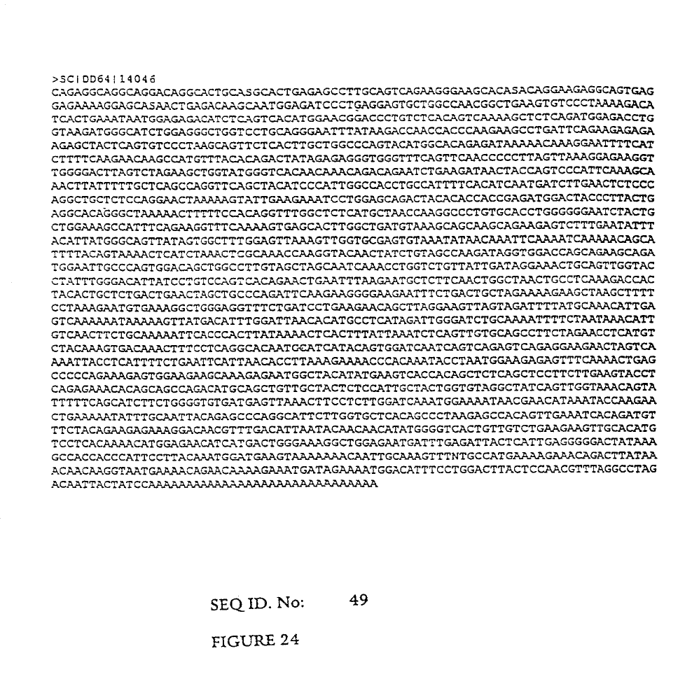

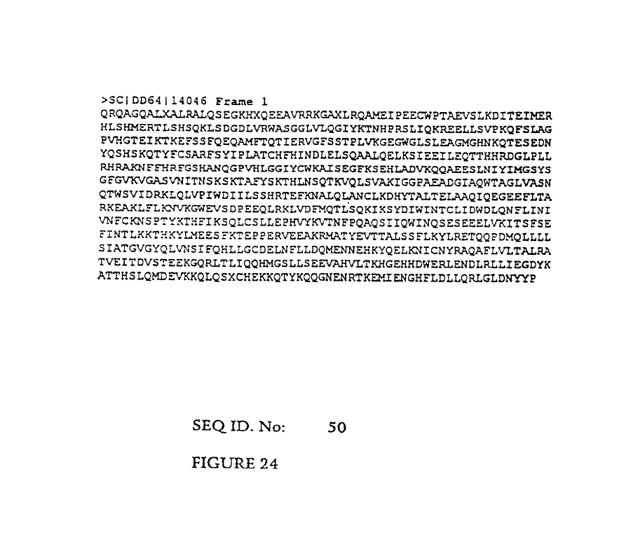

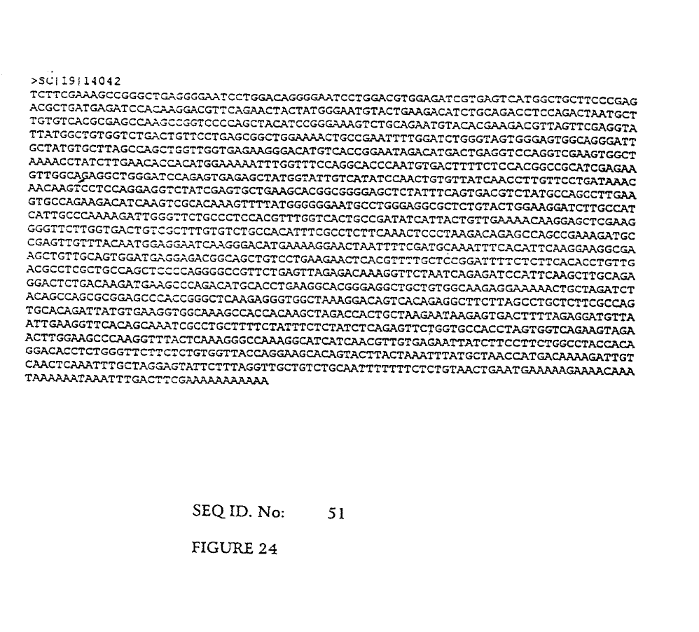

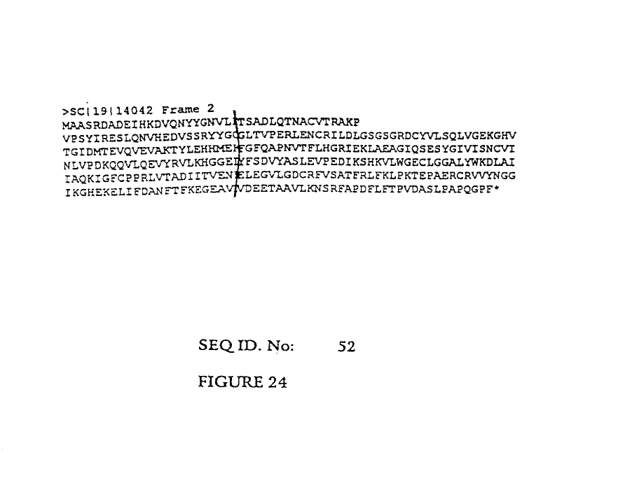

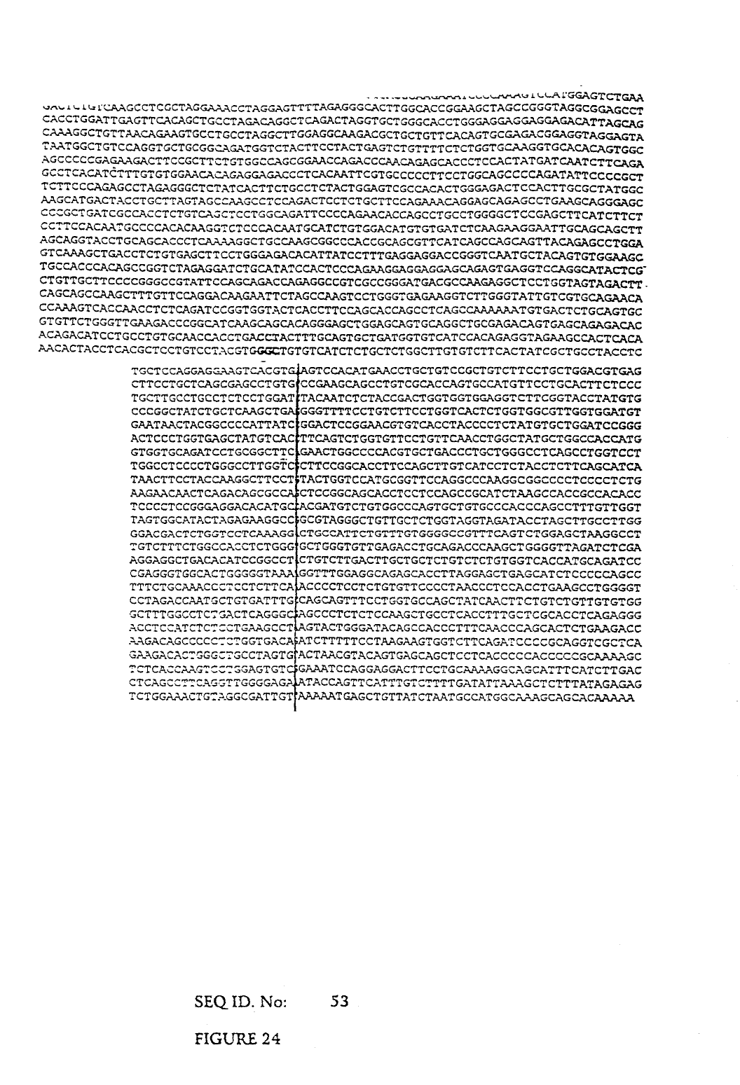

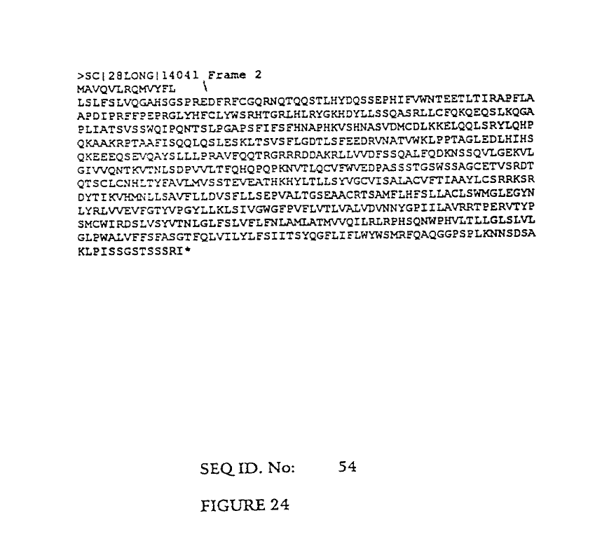

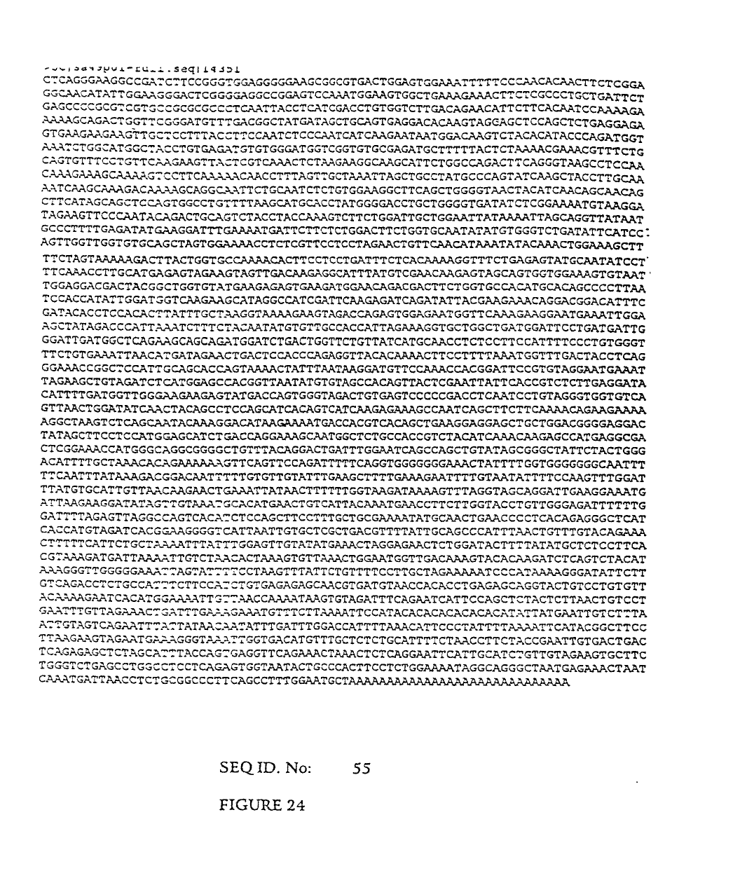

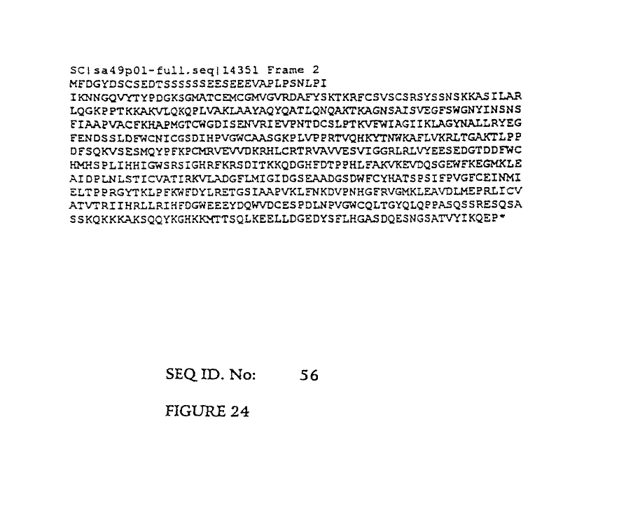

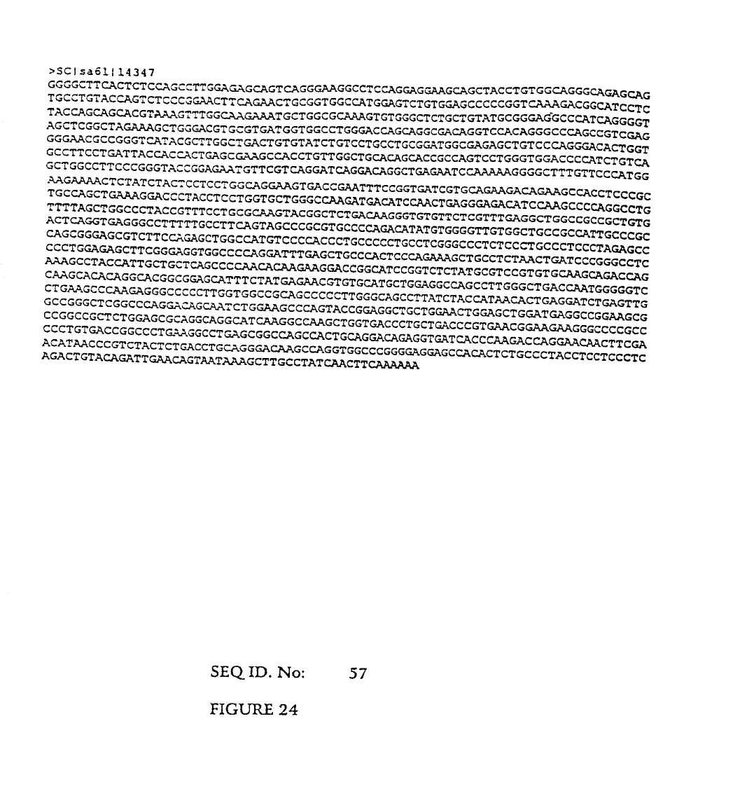

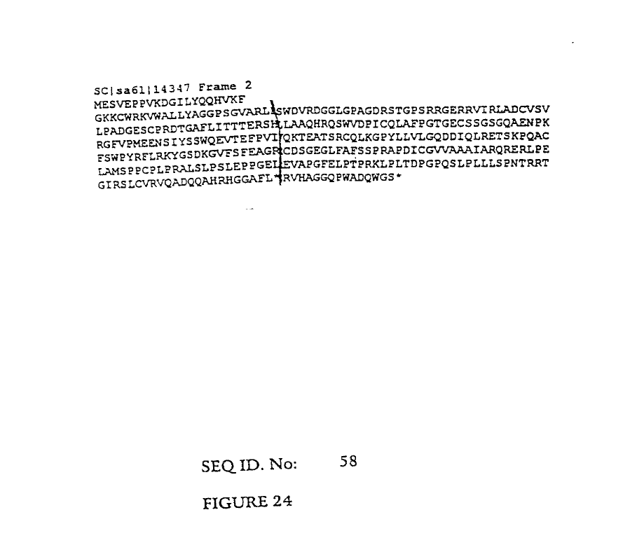

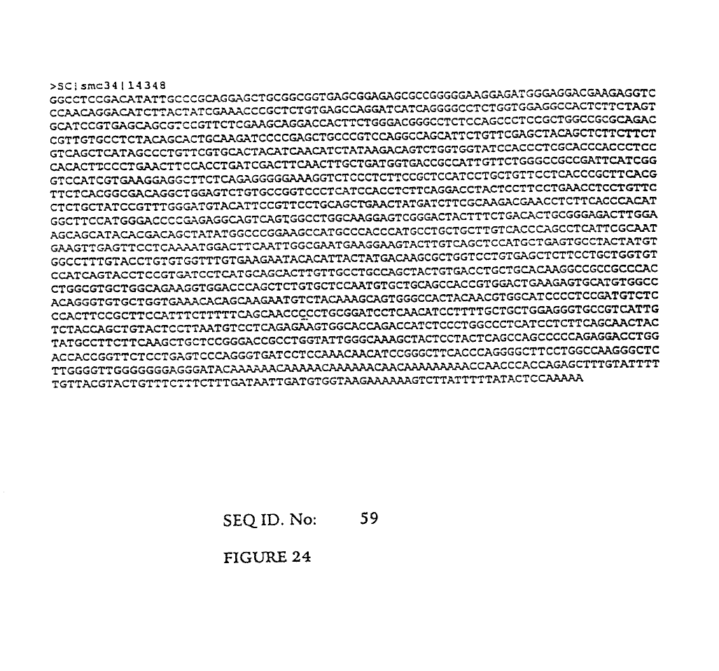

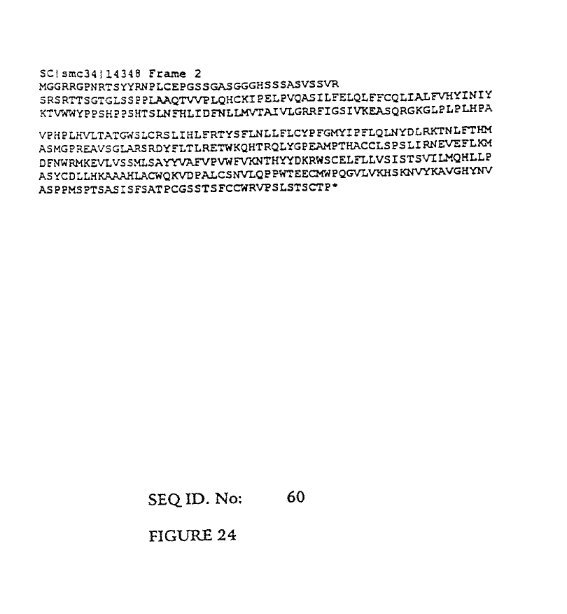

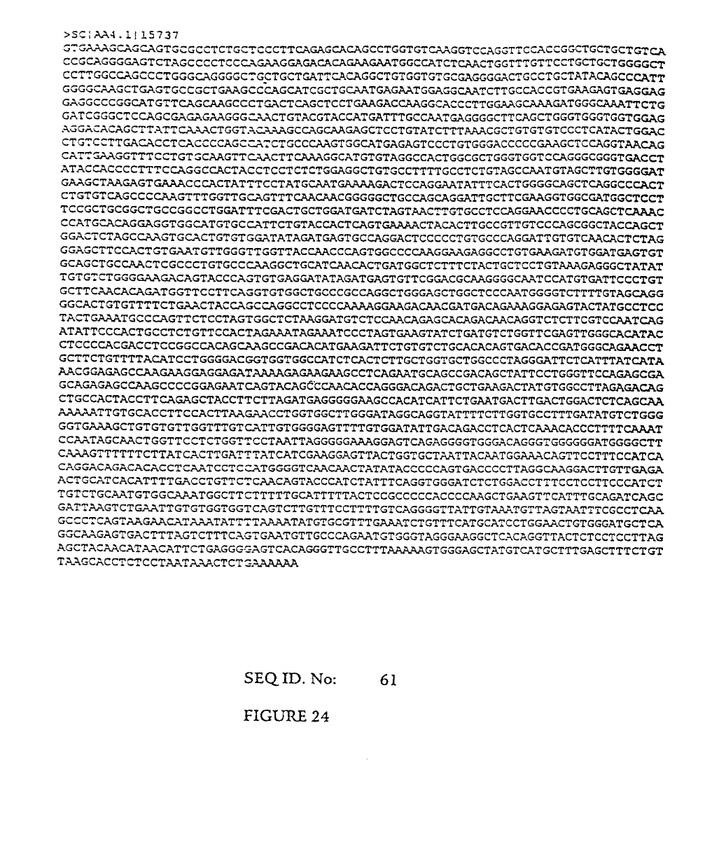

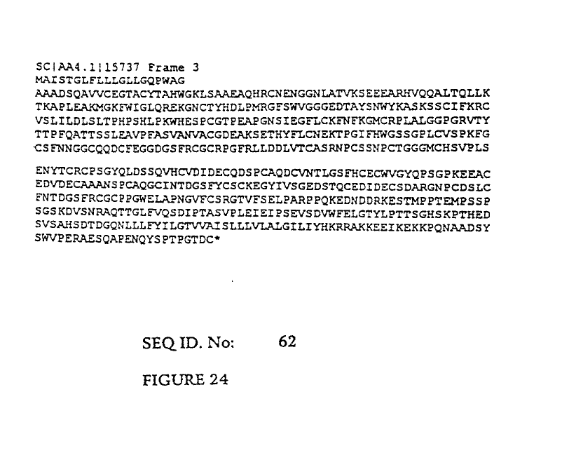

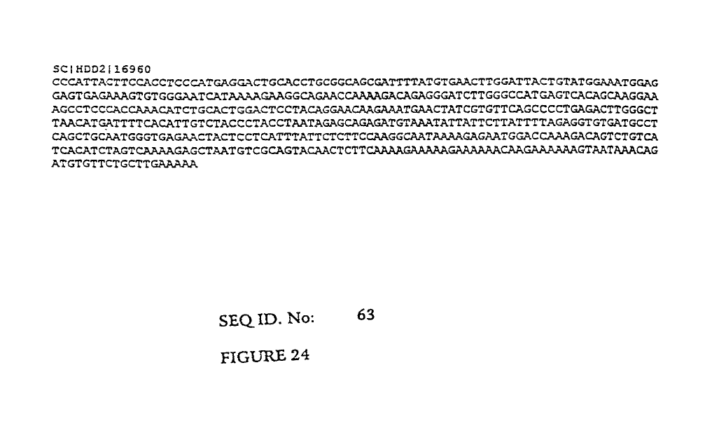

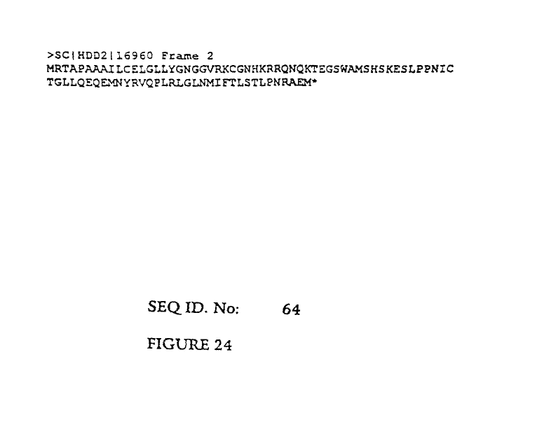

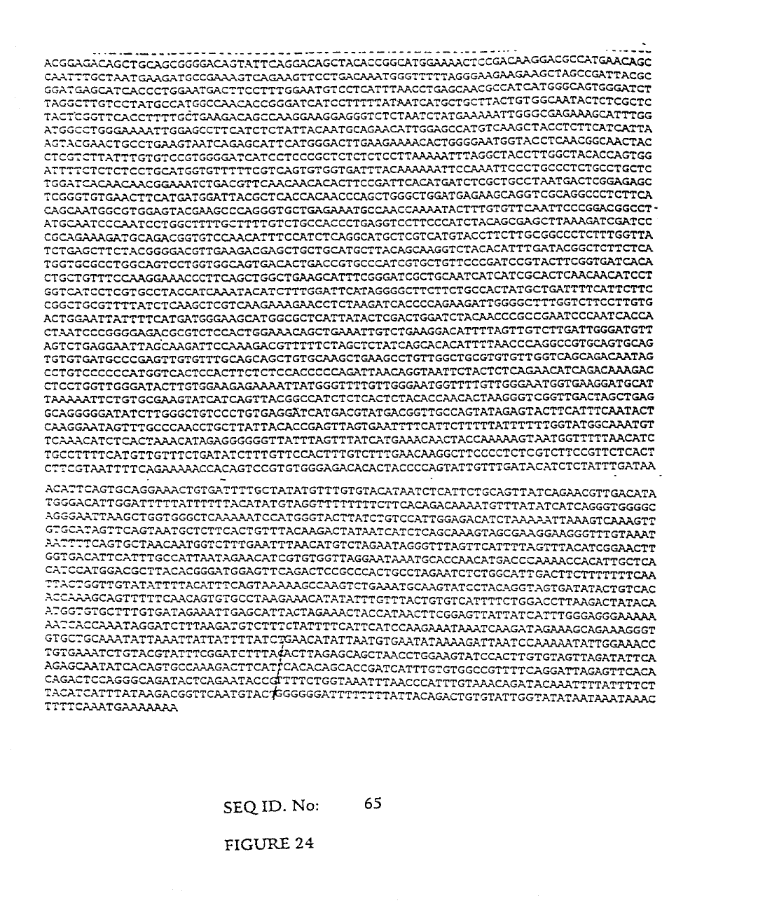

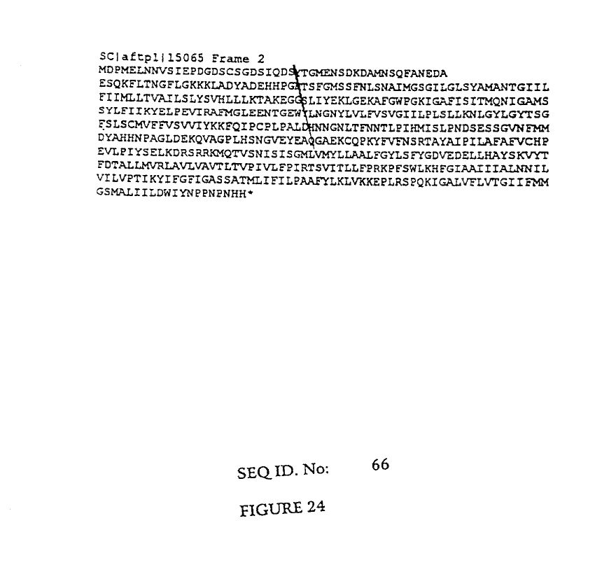

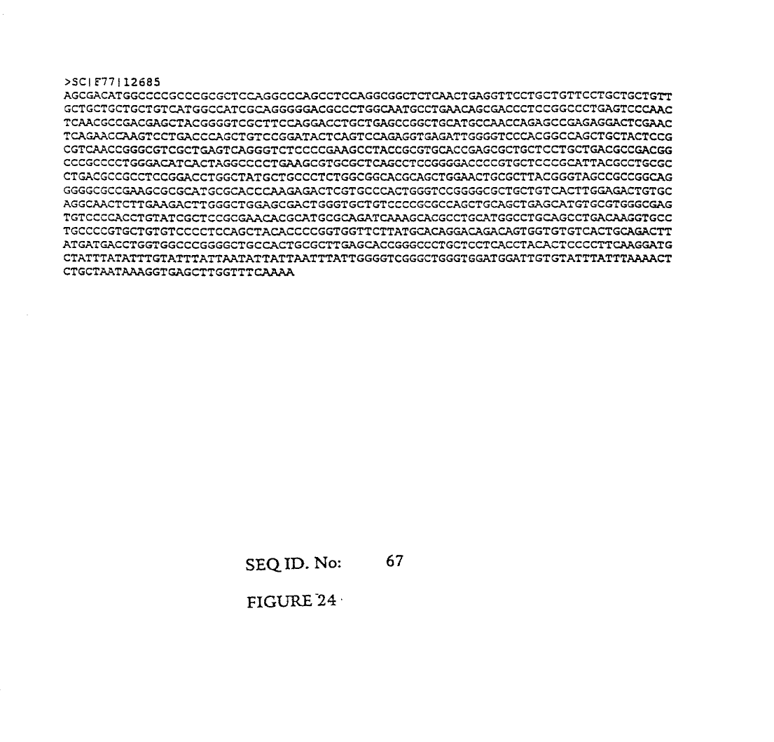

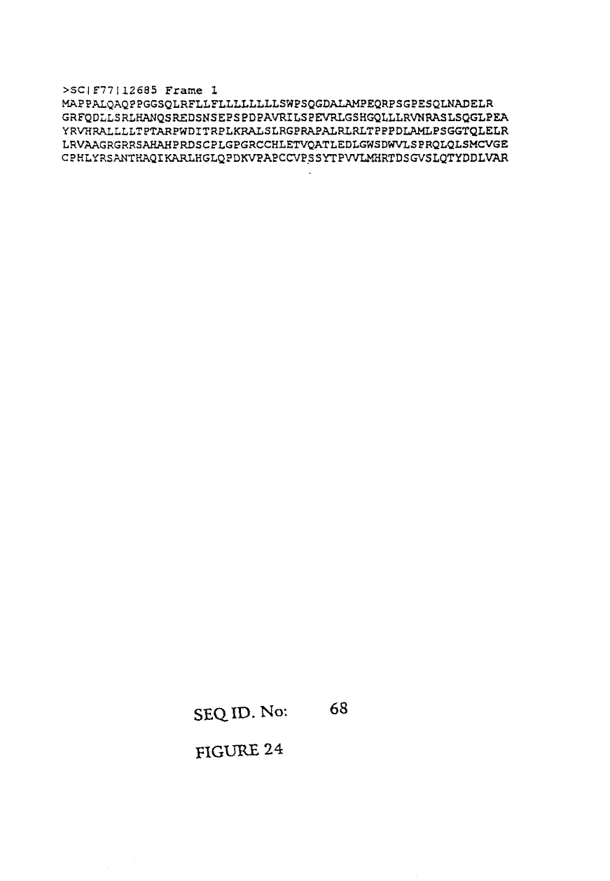

In FIG. 4C and 4D (and also 5a, 5b, and 6b) there are twenty-one samples of capfinder-amplified cDNA from various hematopoietic populations. From left to right these are: four CD34+Lin− populations, three CD34+Lin−CD90+ populations, two CD34− populations, four CD34+Lin−CD38+ (obtained from the same BMs as the CD38− samples in lanes 1-4), two CD34+Lin− samples (obtained from the same BMs as the CD90+ samples in lanes 5 and 6), three CD34+Lin− populations obtained after 1, 2 or 4 days of culture and finally their three CD34+Lin−CD38+ counterparts. The 38G2 CDNA (SEQ.ID.No.: 70) is closely homologous to the LTG9/MLLT3 gene located on 9q22 and involved in t(9;11) leukemia (Iida, S., et al. 1993) (FIG. 4E).

FIGS. 5A-5B. G0S3, a fos-related gene (Heximer, S., et al. 1996) (FIG. 5A) and (2) HLA-DR (FIG. 5B).

FIGS. 6A-6D. FIG. 6A. HDD-2 is about 500 bp. It contains a short open reading frame of 89 amino acids (SEQ.ID.No.: 71). The predicted peptide sequence is shown.

FIG. 6B. The expression profile of HDD-2 demonstrates that it is stem cell restricted.

FIG. 6C. HDD-2 hybridization to a dot blot with numerous human pA+ mRNA samples (Clontech). HDD-2 hybridization is only visible in kidney (the other “spots” are background).

FIG. 6D. Genomic Southern blot confirmed that HDD-2 corresponds to a single-copy human gene.

FIG. 7. Immunoprecipitation analysis of protein extracts using rat IgG2b isotype control antibody (IgG) or AA4.1 mAb. Protein extracts were prep-ared from D2N cells; M2.4 cells; AA4-depleted fetal liver cells (FLAA4−); AA4enriched fetal liver cells (FLAA4+); AA4-depleted bone marrow cells (BMAA4−); AA4-enriched bone marrow cells (BMAA4+). Indicated on the right are positions of molecular weight markers.

FIGS. 8A-8C. AA4 expression in retrovirus infected cells.

FIG. 8A. Flow cytometry analysis of NIH 3T3 and EML C1 cells using PE-conjugated AA4.1 antibody before and after infection with REBNA/AA4.

FIG. 8B. Immunoprecipitation of biotin-labeled surface proteins using rat IgG2b isotype control antibody (lanes 1, 3, 5) or AA4.1 mAb (lanes 2, 4, 6). Cellular extracts were prepared from D2N cells (lanes 1 and 2), REBNA/AA4 infected NIH 3T3 fibroblasts (lanes 3 and 4), and REBNA/AA4 infected EML C1 cells (lanes 5 and 6).

FIG. 8C. Immunoprecipitation of cellular extracts using AA4. 1. REBNA/AA4 NIH 3T3 cells (lanes 2-4) and REBNA/AA4 EML Cl cells (lanes 6-8) were labeled with 35S-methionine and chased with nonradioactive media for 10 min. (lanes 3 and 7) or 20 min. (lanes 4 and 8). REBNAIGFP NIH 3T3 (lane 1) and REBNA/GFP EML C1 cells (lane 5) are shown as controls. Indicated on the right are positions of molecular weight markers.

FIG. 9. Nucleotide sequence (SEQ.ID.No.: 72) and the deduced amino acid sequence of AA4 (SEQ.ID.No.: 73). Amino acid sequences is numbered 1 with initiator methionine. Underlined is the putative signal peptide in AA4. The transmembrane domain is double underlined. Light grey line indicates C-type lectin carbohydrate recognition domain. Dark grey lines show EGF-like repeats, while broken grey lines indicate calcium-binding EGF motifs.

FIGS. 10A-10B. Comparison of the primary structures of AA4 and human C1qR.

FIG. 10A. Alignment of amino acid sequences of AA4 (SEQ.ID.No.: 73) and C1qR.

FIG. 10B. Comparison of the domain structures of AA4 and C1qR. The proteins contain N-terminal signal peptides (SP), a C-type lectin recognition domain, six EGF-like domains, including three calcium-binding (cb) EGF-like domains, and a transmembrane domain (TM).

FIG. 11. In normal tissues and transformed cell lines, a 7 kb RNA species hybridizes with the cloned cDNA (FIGS. 11A and 11B). In addition to the 7 kb species, poly(A)− RNA from D2N cells contains a minor band corresponding to a 3.2 kb mRNA (FIG. 11A, lane 8).

FIGS. 12A-12B. Northern blot analysis of AA4 expression in transformed cell lines FIG. 12A and normal mouse tissues FIG. 12B. Indicated on the left are positions of 9.44-0.24 kb RNA molecular weight markers. Hybridization with D2N poly(A)+ RNA is shown after a 2 hr and overnight exposures. Hybridizations with GAPDH are shown as controls for equal loading.

FIGS. 13A-13C. RT-PCR of cDNAs prepared from murine fetal liver, (A) bone marrow-derived hematopoietic cells (B), or differentiating ES cells (C).

FIG. 13A. Lanes 1 and 2, AA4− cells; lane 3, AA4+ cells; lane 4, AA4+Sca-1+c-Kit+LinlO cells; lane 5, AA4+Sca-1−c−Kit+LinIO cells.

FIG. 13B. Lane 1, RholOSca-1+Thy-llOLincells; lane 2, RhohiSca-1+Thy-llOLin− cells; lane 3, Sca-1+Thy-1−Lin− cells; lane 4, Lin+ cells.

FIG. 13C. ESO, undifferentiated ES cells; BL1 and 2, blast cell colonies; ENT1 and 2, differentiated endothelial cells; HMT1, 2 and 3, differentiated hematopoietic cells. The cDNAs probes used for hybridization are indicated on the right.

FIG. 14. AFT024 maintains in vivo repopulating stem cells. The ability of 3 different stromal cell lines to support highly purified fetal liver stem cells was studied. Freshly purified day 14 fetal liver cells were transplanted directly (10³ AA4.1⁺, lin^{-/lo}, Sca-1⁺, c-kit⁺ cells plus 10⁶ Ly5.1 competitor BM per mouse; n=6) into Ly5.1 congenic mice (Control). Additional stem cells from the same purification were also used to initiate Dexter-LTC over irradiated AFT024, 2012, and 2018 stromal cell monolayers (10⁴ cells/10 cm dish). After 4 weeks of culture, 10% aliquots of each culture (10³ stem cell equivalents) were transplanted into Ly5.1 recipients (n=8) together with 10⁶ competitor Ly5.1 BM cells. Peripheral blood cells from mice were analyzed for the presence of Ly5.2⁺ cells at 5, 12, 24, and 56 weeks after transplant. Error bars represent SEM.

FIG. 15. Long-term culture on AFT024 maintains greater levels of repopulating stem cell activity than short-term cytokine- or short-term AFT024-supported cultures. The levels of stem cell activity maintained in short-term cytokine-supported and short-term AFT024-supported cultures were compared to those maintained in long-term AFT024 coculture. Purified fetal liver cells were cultured for 5 days in suspension with cytokines or on an AFT024 monolayer (3000/well-12-well tray). Additional cells from the same purification were seeded onto AFT024 monolayers (3000/6 cm dish) and maintained in Dexter-LTC for 5 weeks. At completion of both culture periods the cells were harvested, mixed with Ly5.1 BM and used to transplant mice. Each mouse received 20% of each culture (600 stem cell equivalents) and 4×10⁵ competitor BM cells (4 mice/culture). Peripheral blood cells from mice were analyzed for the presence of Ly5.2⁺ cells at 15 weeks after transplant. FL 1.0±0.57; FL/SL 0.75±0.25; FL/IL-6 1.8±0.14; SL/IL-6 3.2±0.46; FL/IL-6/SL 1.7±0.21; AFT024 5 days 2.8±0.11; AFT024 5 weeks 32.2±7.4 * (p<0.004) Students t-test. See Methods for culture conditions and cytokine concentrations. FL, flk2/flt3-ligand; SL, steel factor. Error bars represent SEM.

FIG. 16. Time course of cobblestone area formation on AFT024. The formation of stromal dependent CA derived from purified fetal liver stem cells was studied in AFT024 cocultures. Characteristic clusters of at least 50 cells were scored as CA over 28 days of culture. Results are expressed as the mean number of CA/1000 input stem cells from 3 separate fetal liver purifications (300-600 cells/well in 12-well trays). Error bars represent the SEM. The frequency of CA after 28 days is approximately 1 for every 20 input stem cells.

FIG. 17. High-proliferative potential multilineage clonogenic progenitors are selectively expanded on AFT024. The clonogenic progenitor content of stem cells maintained in AFT024 supported Dexter-LTC was determined. Enriched fetal liver stem cells were seeded onto AFT024 monolayers, at various time points, an individual well was harvested and the cells placed into semi-solid clonogenic progenitor assay (CFU-C). Colonies were scored at 8-14 days. Colonies were designated as HPP upon reaching a size ≧1 mm after 8 days. CFU numbers at days 0, 4, and 28 are averaged from 3-5 individual stem cell purifications. Error bars represent the SEM for these experiments. Other time points are individual determinations. CFU are normalized to 1000 input stem cells in the stromal cocultures for comparison to day 0 progenitors. *CFU-Mix (p=0.01) and *CFU-HPP-Mix (p=0.001) are significantly expanded at day 28 compared to day 0, (Student's T-test).

FIGS. 18A-18B. Cobblestone area-initiating cells are expanded on AFT024.

FIG. 18A. A quantitative estimate of the number of 28 day cultured stem cell equivalents required to form a CA after replating on secondary AFT024 monolayers was determined. Four different AFT024 cocultures from separate fetal liver purifications were studied (A, B, C, and D). In limiting-dilution assay, the frequency of stem cell equivalents required to form a CA in another 7 days was 1 in 4 (3.56±0.64, r²=0.96).

FIG. 18 B. CA maintenance in one of the 4 above cultures was followed for an additional 4 weeks. The frequency of stem cell equivalents maintaining CA was determined. At 37% negative wells the frequencies were: 2 weeks 1 in 3, 3 weeks 1 in 10, 4 weeks 1 in 19, and at 5 weeks 1 in 29.

FIGS. 19A-19B. Dlk expression analysis in stromal cell lines.

FIG. 19A. (Top) A 1.6 kb dlk transcript is visualized in the parental AFT024 and 2012 cell lines and their subclones, but not in 2018, CFC034 and BFC012. (Bottom) The same filter hybridized with a b-actin probe.

FIG. 19B. RT-PCR analysis of 14 fetal liver-derived stromal cell lines and other cell lines.

FIG. 20. Cobblestone area formation by hematopoietic stem cells in the presence of soluble dlk protein. Data are from 4 experiments; 2 each with adult BM cells (Sca-1⁺, c-kit⁺, lin^{lo/-}) and day 14 fetal liver cells (AA4.1⁺, Sca-1⁺, c-kit⁺, lin^{lo/-}). Results are expressed as the ratio/fold-increase in CSA number for fourteen data points each (bars represent, none vs control; dlk vs none; dlk vs control) for the 4 different experiments. Error bars represent SEM. *P=0.01 comparing dlk vs none to none vs control, **P=0.001 comparing dlk vs control to none vs control (Student's t-test).

FIG. 21. Membrane bound dlk expression in transfected BFC012 cells. Full length dlk cDNA was transfected into BFC012 cells. (Left) A flow diagram of dlk expression in transfected BFC012 populations (BFC-dlk) and cells transfected with the selection plasmid alone (BFC-Zeo). (Right) Expression of dlk in a cloned line (BFC-dik-5) derived from the expressing population and a control clone (BFC-Zeo-1).

FIG. 22A. CSA formation by hematopoietic stem cells in the presence of membrane-bound dlk.

FIG. 22A. Bars labeled BFC are from 5 groups (nontransfected BFC012 cells, two control pZeo transfected BFC012 populations, and two clones derived from the pZeo transfected populations). Bars labeled BFC-dlk are from three groups shown to express transfected dlk; one dlk-transfected BFC012 population (BFC-dlk) and two individual transfected clones (BFC-dlk-1 and BFC-dlk-5). Error bars represent SEM. ** P<0.001 days 3, 4, and 5; *P<0.01 days 6 and 7 (Student's t-test).

FIG. 22B. A clone derived from the dlk transfected populations of BFC012 cells (BFC-dlk-5) and a clone derived from pZeo transfected populations (BFC-Zeo-1) were used for CSA assay with purified fetal liver stem cells. CSA/1000 input stem cells are expressed as the mean of three individual experiments, error bars represent the SEM. **P<0.001 at days 4, 6, and 8 (Student's t-test).

FIGS. 23A-23B. HPP multilineage clonogenic progenitors and ill vivo repopulating stem cells are maintained in short-term dlk-expressing cocultures.

FIG. 23A. Fetal liver stem cells were purified as described and assayed for their progenitor content immediately after purification and after culture on BFC-dlk-5, and BFC-Zeo-1. At day 4 the cultures were used for clonogenic progenitor (3 experiments) and transplantation assay (2 experiments). Bars represent data from 3 experiments with day 0 cells (Fresh) and day 4 14 cocultured cells (BFC-dlk-5 and BFC-Zeo-1), error bars represent SEM. *P=0.01 for total CFU-C from fresh stem cells compared to total CFU-C from BFC-dlk-5 cocultures at day 4; ** P=0.001 for total CFU-C from BFC-dlk-5 compared to total CFU-C from BFC-Zeo-1 (Student's t-test). FIG. 23 B. Analysis of in vivo repopulating ability of purified fetal liver stem cells cocultured for 4 days on BFC-dlk-5, BFC-Zeo-1, and AFT024 monolayers. Results are from nine individual mice in two experiments (4-5 mice in each experiment) at 10 weeks after transplantation. P=0.05 for BFC-dlk-5 vs BFC-Zeo-1 (Student's t-test).

FIG. 24. Genes and predicted proteins isolated from primitive stem cells by the techniques of the present invention. (Sequence Identification Numbers are indicated on the figure).

### DETAILED DESCRIPTION

The present invention provides an isolated nucleic acid derived from an isolated hematopoietic stem cell, the isolated nucleic acid comprising the following characteristics: (1) specifically expressed in the hematopoietic stem cell; and (2) encoding a hematopoietic stem cell-specific protein.

An embodiment of this invention further comprises the following characteristic: capable of hybridizing under standard conditions with a sequence selected from the group consisting of SEQ.ID.No.: 1, SEQ.ID.No.: 2, SEQ.ID.No.: 3, SEQ.ID.No.: 4, SEQ.ID.No.: 5, SEQ.ID.No.: 6, SEQ.ID.No.: 7, SEQ.ID.No.: 8, SEQ.ID.No.: 9, SEQ.ID.No.: 10, SEQ.ID.No.: 11, SEQ.ID.No.: 12, SEQ.ID.No.: 13, SEQ.ID.No.: 14, SEQ.ID.No.: 15, SEQ.ID.No.: 16, SEQ.ID.No.: 17, SEQ.ID.No.: 18, SEQ.ID.No.: 19, SEQ.ID.No.: 20, SEQ.ID.No.: 21, SEQ.ID.No.: 22, SEQ.ID.No.: 23, SEQ.ID.No.: 24, SEQ.ID.No.: 25, SEQ.ID.No.: 26, SEQ.ID.No.: 27, SEQ.ID.No.: 28, SEQ.ID.No.: 29, SEQ.ID.No.: 30, SEQ.ID.No.: 31, SEQ.ID.No.: 32, SEQ.ID.No.: 33, SEQ.ID.No.: 34, SEQ.ID.No.: 35, SEQ.ID.No.: 36, SEQ.ID.No.: 37, SEQ.ID.No.: 38, SEQ.ID.No.: 39, SEQ.ID.No.: 40, SEQ.ID.No.: 41, SEQ.ID.No.: 42, and SEQ.ID.No.: 43, SEQ.ID.No.: 45, SEQ.ID.No.: 47, SEQ.ID.No.: 49, SEQ.ID.No.: 51, SEQ.ID.No.: 53, SEQ.ID.No.: 55, SEQ.ID.No.: 57, SEQ.ID.No.: 59, SEQ.ID.No.: 61, SEQ.ID.No.: 63, SEQ.ID.No.: 65, SEQ.ID.No.: 67, SEQ.ID.No.: 72 or a portion thereof. A portion thereof, in a preferred embodiment of this invention is the 5′ end region or the 3′ end region of the nucleic acid. In another preferred embodiment, a portion thereof is at least a 8-18 nucleotide portion of the coding region. In yet another preferred embodiment, a portion therof is at least a 8-18 nucleotide portion of a non-coding regulatory region or a binding region such as a stem-cell specific promoter or enhancer region. According to still another embodiment of the provided isolated nucleic acid further comprises the characteristic of encoding a protein capable of modulating hematopoietic stem cell activity. According to this invention modulating hematopoietic stem cell activity includes up-regulating, down-regulating or otherwise changing the activity of the hematopoietic stem cell. Such activity is contemplated as inducing differentiation or inhibiting differentiation of the cell. However, directing differentiation toward one or another daughter cell type is also within the scope of a preferred embodiment of this invention. Other preferred embodiments include but are not limited to modulation of transcription, translation, gene splicing, transport, proteolytic processing, replication, expression of cell surface markers and transplantation. According to still another embodiment of the present invention, the activity is selected from the group consisting of hematopoietic stem cell differentiation and hematopoietic stem cell replication. According to yet another embodiment of this invention, the protein is selected from the group consisting of a growth factor, a transcription factor, a splicing factor, a capping factor, a transport protein, a translation factor, and a replication factor. In one preferred embodiment of this invention, the provided nucleic acid comprises the nucleotide sequence of SEQ.ID.No.: 72, an analog thereof, or a portion thereof. According to another preferred embodiment of this invention, the hematopoietic stem cell is a primitive hematopoietic stem cell. In one embodiment of this invention, the primitive hematopoietic stem cell is selected from the group consisting of an umbilical cord cell , a bone marrow cell and a fetal liver cell. In a preferred embodiment of this invention, the primitive hematopoietic stem cell is selected from the group consisting of a AFT024 cell, a 2012 cell and a 2018 cell.

The present invention further provides a composition comprising the provided nucleic acid, wherein the nucleic acid comprises one selected from the group consisting of SEQ.ID.No.: 1, SEQ.ID.No.: 2, SEQ.ID.No.: 3, SEQ.ID.No.: 4, SEQ.ID.No.: 5, SEQ.ID.No.: 6, SEQ.ID.No.: 7, SEQ.ID.No.: 8, SEQ.ID.No.: 9, SEQ.ID.No.: 10, SEQ.ID.No.: 11, SEQ.ID.No.: 12, SEQ.ID.No.: 13, SEQ.ID.No.: 14, SEQ.ID.No.: 15, SEQ.ID.No.: 16, SEQ.ID.No.: 17, SEQ.ID.No.: 18, SEQ.ID.No.: 19, SEQ.ID.No.: 20, SEQ.ID.No.: 21, SEQ.ID.No.: 22, SEQ.ID.No.: 23, SEQ.ID.No.: 24, SEQ.ID.No.: 25, SEQ.ID.No.: 26, SEQ.ID.No.: 27, SEQ.ID.No.: 28, SEQ.ID.No.: 29, SEQ.ID.No.: 30, SEQ.ID.No.: 31, SEQ.ID.No.: 32, SEQ.ID.No.: 33, SEQ.ID.No.: 34, SEQ.ID.No.: 35, SEQ.ID.No.: 36, SEQ.ID.No.: 37, SEQ.ID.No.: 38, SEQ.ID.No.: 39, SEQ.ID.No.: 40, SEQ.ID.No.: 41, SEQ.ID.No.: 42, SEQ.ID.No.: 43, SEQ.ID.No.: 45, SEQ.ID.No.: 47, SEQ.ID.No.: 49, SEQ.ID.No.: 51, SEQ.ID.No.: 53, SEQ.ID.No.: 55, SEQ.ID.No.: 57, SEQ.ID.No.: 59, SEQ.ID.No.: 61, SEQ.ID.No.: 63, SEQ.ID.No.: 65, SEQ.ID.No.: 67, SEQ.ID.No.: 72 or a portion thereof. According to one embodiment of this invention, the the nucleic acid is selected from the group consisting of DNA, RNA and cDNA. Another embodiment of this invention is a vector comprising the provided nucleic acid. According to yet another embodiment, the vector comprises viral or plasmid DNA. A further embodiment of this invention is an expression vector comprising the provided nucleic acid and a regulatory element. A still further embodiment of this invention is a host vector system which comprises the expression vector in a suitable host. In a preferred embodiment of this invention, the suitable host is selected from the group consisting of a bacterial cell, a eukaryotic cell, a mammalian cell and an insect cell.

The present invention additionally provides an isolated hematopoietic stem cell specific protein or a portion thereof encoded by the provided nucleic acid. According to one embodiment of this invention is the provided protein further comprising the characteristic of being capable of modulating hematopoietic stem cell activity. According to this invention modulating hematopoietic stem cell activity includes up-regulating, down-regulating or otherwise changing the activity of the hematopoietic stem cell. Such activity is contemplated as inducing differentiation or inhibiting differentiation of the cell. However, directing differentiation toward one or another daughter cell type is also within the scope of a preferred embodiment of this invention. Other preferred embodiments include but are not limited to modulation of transcription, translation, gene splicing, transport, proteolytic processing, replication, expression of cell surface markers and transplantation. According to still another embodiment of the present invention, the activity is selected from the group consisting of hematopoietic stem cell differentiation and hematopoietic stem cell replication. According to yet another embodiment of this invention, the protein is selected from the group consisting of a growth factor, a transcription factor, a splicing factor, a capping factor, a transport protein, a translation factor, and a replication factor. According to one embodiment the activity is selected from the group consisting of hematopoietic stem cell differentiation and hematopoietic stem cell replication. According to another embodiment, the protein is selected from the group consisting of a growth factor, a transcription factor, a splicing factor, a capping factor, a transport protein, a translation factor, and a replication factor. According to still another embodiment, the protein has substantially the same amino acid sequence as one selected from the group consisting of SEQ.ID.No.: 42, SEQ.ID.No.: 44, SEQ.ID.No.: 46, SEQ.ID.No.: 48, SEQ.ID.No.: 50, SEQ.ID.No.: 52, SEQ.ID.No.: 54, SEQ.ID.No.: 56, SEQ.ID.No.: 58, SEQ.ID.No.: 60, SEQ.ID.No.: 62, SEQ.ID.No.: 64, SEQ.ID.No.: 66, SEQ.ID.No.: 68, SEQ.ID.No.: 70, SEQ.ID.No.: 71, and SEQ.ID.No.: 73.

The present invention further still provides a nucleic acid probe capable of specifically hybridizing with the provided nucleic acid under standard hybridization conditions.

Also, the present invention provides an antibody capable of specifically binding to the provided protein without substantially cross-reacting with a non-stem cell specific protein or homologs thereof under conditions permissive to antibody binding. Additionally, the present invention provides a cell capable of producing the provided antibody.

In addition, the present invention provides a method for identifying the presence of a primitive hemopoietic stem cell in a sample comprising nucleic acids specifically expressed in hematopoietic stem cells comprising (a) contacting the sample with the provided antibody under conditions permissive to the formation of an antibody complex; (b) detecting the presence of the complex formed in step (a), the presence of a complex formed indicating the presence of a primitive hemopoietic stem cell in the sample. According to one embodiment of this invention, the antibody is labeled with a detectable marker. In a preferred embodiment, the detectable marker is selected from the group consisting of a radioactive isotope, enzyme, magnetic bead, dye, flourescent marker and biotin.

Further still, the present invention provides a method for generating a stem cell/progenitor cell from a primitive hematopoietic cell in a sample comprising contacting the sample with the provided protein . Another embodiment of this invention, provides a method for generating a stem cell/progenitor cell from a primitive hematopoietic cell in a sample comprising contacting the sample with the provided nucleic acid. According to an preferred embodiment, the nucleic acid is in an expression vector. According to another preferred embodiment the nucleic acid is introduced into the cell under conditions permissive to the expression of the nucleic acid.

The present invention further provides a method for identifying the presence in a sample of a compound that modulates hematopoietic stem cell activity comprising: (a) contacting the hematopoietic stem cell with the sample; (b) determining the hematopoietic stem cell activity; and (c) comparing the hematopoietic stem cell activity determined in step (b) with the activity determined in the absence of the compound an increase or decrease in hematopoietic stem cell activity indicating the presence in the sample of a compound that modulates hematopoietic stem cell activity. According to one embodiment, the activity is selected from the group consisting of gene expression, replication, differentiation, transplantation, and self regeneration. The present invention also still further provides a compound identified by the method of this invention, previously unknown.

The present invention even further still provides a method for identifying primitive hematopoietic stem cell-specific nucleic acids, comprising: (a) creating a primitive hematopoietic stem cell cDNA library and a non-primitive stem cell immune cell cDNA library; and (b) subtracting the two libraries, thereby identifying primitive stem cell specific nucleic acids. According to one embodiment is (i) contacting the nucleic acids of the stem cell and non-stem cell libraries with each other under conditions permissive to hybridization, thereby forming hybrid complexes; (ii) separating the hybrid complexes formed in step (b) from the nucleic acids which did not form complexes; and (iii) isolating the nucleic acids which did not form complexes, thereby identifying hematopoietic stem cell specific nucleic acids. In still another embodiment, step (ii) further comprising amplification of the nucleic acids. Yet another embodiment is step (iii) further comprising ampliciation of the nucleic acids which did not form complexes. Even still another embodiment is further comprising displaying the amplified DNA on a chromatography gel. A further embodiment is step (b) comprising differential display of the two libraries, thereby identifying primitive stem cell specific nucleic acids. Also yet another embodiment is step (b) comprising representation difference analysis of the two libraries, thereby identifying primitive stem cell specific nucleic acids. Yet even another embodiment is further comprising cloning the stem cell specific nucleic acid. According to a preferred embodiment, the stem cell is selected from the group consisting of AF024, 2012, and 2018. The present invention further provides a nucleic acid identified by the provided method.

The present invention additionally provides a composition comprising the provided compound and a carrier. An embodiment of this invention is a pharmaceutical composition comprising the provided compound and a pharmaceutically acceptable carrier.

Also the present invention additionally provides a primitive hematopoietic stem cell specifically expressing one selected from the group consisting of: SEQ.ID.No.: 1, SEQ.ID.No.: 2, SEQ.ID.No.: 3, SEQ.ID.No.: 4, SEQ.ID.No.: 5, SEQ.ID.No.: 6, SEQ.ID.No.: 7, SEQ.ID.No.: 8, SEQ.ID.No.: 9, SEQ.ID.No.: 10, SEQ.ID.No.: 11, SEQ.ID.No.: 12, SEQ.ID.No.: 13, SEQ.ID.No.: 14, SEQ.ID.No.: 15, SEQ.ID.No.: 16, SEQ.ID.No.: 17, SEQ.ID.No.: 18, SEQ.ID.No.: 19, SEQ.ID.No.: 20, SEQ.ID.No.: 21, SEQ.ID.No.: 22, SEQ.ID.No.: 23, SEQ.ID.No.: 24, SEQ.ID.No.: 25, SEQ.ID.No.: 26, SEQ.ID.No.: 27, SEQ.ID.No.: 28, SEQ.ID.No.: 29, SEQ.ID.No.: 30, SEQ.ID.No.: 31, SEQ.ID.No.: 32, SEQ.ID.No.: 33, SEQ.ID.No.: 34, SEQ.ID.No.: 35, SEQ.ID.No.: 36, SEQ.ID.No.: 37, SEQ.ID.No.: 38, SEQ.ID.No.: 39, SEQ.ID.No.: 40, SEQ.ID.No.: 41, SEQ.ID.No.: 42, and SEQ.ID.No.: 43, SEQ.ID.No.: 45, SEQ.ID.No.: 47, SEQ.ID.No.: 49, SEQ.ID.No.: 51, SEQ.ID.No.: 53, SEQ.ID.No.: 55, SEQ.ID.No.: 57, SEQ.ID.No.: 59, SEQ.ID.No.: 61, SEQ.ID.No.: 63, SEQ.ID.No.: 65, SEQ.ID.No.: 67, SEQ.ID.No.: 72 or a portion thereof. An embodiment of this invention is a primitive hematopoietic stem cell specifically expressing a nucleic acid identified by the provided method.

Yet additionally, the present invention provides a method for treating a condition in a subject comprising administering to the subject a therapeutically effective amount of the provided pharmaceutical composition. According to an embodiment of this invention, the condition is an immune system condition. In a further embodiment of this invention, the condition is leukemia.

The present invention provides a method of introducing an exogenous nucleic acid into a hematopoietic stem cell comprising contacting the stem cell with the provided composition.

Finally, the present invention provides a method of ex vivo expansion of hematopoietic stem cells comprising contacting the cell with the provided composition. According to an embodiment of this invention, the ex vivo expanded hematopoietic stem cells are available for therapeutic use. The expanded cells are available to receive exogenous genes, including by retroviral or other vectors which require a round of replication. Alternatively, the cells are available for transplantation either autologously or heterologously.

As used herein, the term, a sequence is conserved if there is substantial homology of sequence between multiple gene species.

As used herein, the terms, “hybridization” and “binding” in the context of probes, primers and denatured DNA are used interchangeably. Probes which are hybridized or bound to denatured DNA are aggregated to complementary sequences in the polynucleotide. Whether or not a particular probe remains aggregated with the polynucleotide depends on the degree of complementarity, the length of the probe, and the stringency of the binding conditions. The higher the stringency, the higher must the degree of complementarity, and/or the longer the probe.

As used herein, the term, probe, refers to an oligonucleotide designed to be sufficiently complementary to a sequence in a denatured nucleic acid to be probed, in relation to its length, to be bound under selected stringency conditions. Primers may vary in length. Preferably such primers should be sufficiently long to hybridize to the modified RNAs in a specific and stable manner. A semi-random primer as the term is used herein, encompasses a class of primers wherein either a discrete portion of the primer is random, while another discrete portion is conserved as well as primers which have nucleotide preferences at particular positions within a sequence. For example, the discrete portion-type primer may have a predetermined adaptor sequence at its 5′ end and a random sequence at its 3′ end. Alternatively, several preferred primers have nucleotide preferences at specific positions within the primers while other positions are random. A degenerate primer as the term is used herein, encompasses a cocktail or mixture of primers wherein one or more of the possible triplet nucleotide sequences encoding an amino acid is incorporated into the primer sequence. For example, Serine may be encoded by six separate triple sequences (AGU, AGC, UCU, UCC, UCA, and UCG). Thus, a “degenerate” primer may reflect the degeneracy of the nucleotide triplet code. Alternatively, a randomized primer, as the term is used herein, encompasses a primer wherein, the nucleotide at one or more positions may be randomized in order to yield a triplet sequence encoding an alternative or a random amino acid at the position.

An end region as the term is used herein, consists of the end nucleotide and a portion of the region including as much as that half of the entire sequence. For example, the “3′ end region” or “3′ region” of a primer may include the 3′ half of the primer.

A preferred method of hybridization is blot hybridization. See Sambrook et al. 1989 Molecular Cloning: A Laboratory Manual 2nd Ed. for additional details regarding blot hybridization. Using this method, separated amplification products are transferred onto a solid matrix, such as a filter. The probe, which is detectable, either directly or indirectly, is hybridized to the solid matrix under appropriate conditions. The matrix is washed to remove excess probe. Thereafter the probe which specifically hybridized to the solid matrix can be detected.

The probe can be DNA or RNA and can be made detectable by any of the many labeling techniques readily available and known to the skilled artisan. Such methods include, but are not limited to, radio-labeling, digoxygenin-labeling, and biotin-labeling. A well-known method of labeling DNA is ³²P using DNA polymerase, Kienow enzyme or polynucleotide kinase. In addition, there are known non-radioactive techniques for signal amplification including methods for attaching chemical moieties to pyrimidine and purine rings (Dale, R. N. K. et al, 1973 Proc. Natl. Acad. Sci. USA 70:2238-42), methods which allow detection by chemiluminescence (Barton, S. K. et al, 1992 J. Am. Chem. Soc. 114:8736-40) and methods utilizing biotinylated nucleic acid probes (Johnson, T. K. et al, 1983 Anal. Biochem. 133:125-131; Erickson, P. F. et al, 1982 J. Immunol. Methods 51:241-49; Matthaei, F. S. et al, 1986 Anal. Biochem. 157-123-28) and methods which allow detection by fluorescence using commercially available products. Non-radioactive labeling kits are also commercially available.

A basic description of nucleic acid amplification is described in Mullis, U.S. Pat. No. 4,683,202, which is incorporated herein by reference. The amplification reaction uses a template nucleic acid contained in a sample, two primer sequences and inducing agents. The extension product of one primer when hybridized to the second primer becomes a template for the production of a complementary extension product and vice versa, and the process is repeated as often as is necessary to produce a detectable amount of the sequence.

The inducing agent may be any compound or system which will function to accomplish the synthesis of primer extension products, including enzymes. Suitable enzymes for this purpose include, for example, E.coli DNA polymerase 1, thermostable Taq DNA polymerase, Klenow fragment of E.coli DNA polymerase I, T4 DNA polymerase, other available DNA polymerases, reverse transcriptase and other enzymes which will facilitate combination of the nucleotides in the proper manner to form amplification products. The oligonucleotide primers can be synthesized by automated instruments sold by a variety of manufacturers or can be commercially prepared based upon the nucleic acid sequence of this invention.

The degree of hybridization depends on the degree of complementarity, the length of the nucleic acid molecules being hybridized, and the stringency of the conditions in a reaction mixture. Stringency conditions are affected by a variety of factors including, but not limited to temperature, salt concentration, concentration of the nucleic acids, length of the nucleic acids, sequence of the nucleic acids and viscosity of the reaction mixture. More stringent conditions require greater complementarity between the nucleic acids in order to achieve effective hybridization.

“Hybridization” and “binding” in the context of probes, primers and denatured DNA are used interchangeably. Probes which are hybridized or bound to denatured DNA are aggregated to complementary sequences in the polynucleotide. Whether or not a particular probe remains aggregated with the polynucleotide depends on the degree of complementarity, the length of the probe, and the stringency of the binding conditions. The higher the stringency, the higher must the degree of complementarity, and/or the longer the probe.

“Probe” refers to an oligonucleotide designed to be sufficiently complementary to a sequence in a denatured nucleic acid to be probed, in relation to its length, to be bound under selected stringency conditions.

Primers may vary in length. Preferably such primers should be sufficiently long to hybridize to the modified RNAs in a specific and stable manner.

A semi-random primer as the term is used herein, encompasses a class of primers wherein either a discrete portion of the primer is random, while another discrete portion is conserved as well as primers which have nucleotide preferences at particular positions within a sequence. For example, the discrete portion-type primer may have a predetermined adaptor sequence at its 5′ end and a random sequence at its 3′ end. Alternatively, several preferred primers have nucleotide preferences at specific positions within the primers while other positions are random.

A degenerate primer as the term is used herein, encompasses a cocktail or mixture of primers wherein one or more of the possible triplet nucleotide sequences encoding an amino acid is incorporated into the primer sequence. For example, Serine may be encoded by six separate triple sequences (AGU, AGC, UCU, UCC, UCA, and UCG). Thus, a “degenerate” primer may reflect the degeneracy of the nucleotide triplet code. Alternatively, a randomized primer, as the term is used herein, encompasses a primer wherein, the nucleotide at one or more positions may be randomized in order to yield a triplet sequence encoding an alternative or a random amino acid at the position.

Solid matrices are available to the skilled artisan. Solid phases useful to serve as a matrix for the present invention include but are not limited to polystyrene, polyethylene, polypropylene, polycarbonate, or any solid plastic material in the shape of test tubes, beads, microparticles, dip-sticks, plates or the like. Additionally matrices include, but are not limited to membranes, 96-well microtiter plates, test tubes and Eppendorf tubes. Solid phases also include glass beads, glass test tubes and any other appropriate shape made of glass. A functionalized solid phase such as plastic or glass which has been modified so that the surface carries carboxyl, amino, hydrazide, or aldehyde groups can also be used. In general such matrices comprise any surface wherein a ligand-binding agent can be attached or a surface which itself provides a ligand attachment site.

As used herein, “pharmaceutically acceptable” refers to molecular entities and compositions that are physiologically tolerable and do not typically produce an allergic or similar untoward reaction, such as gastric upset, dizziness and the like, when administered to a human. A pharmaceutically acceptable carrier encompasses any of the standard pharmaceutically accepted carriers, such as phosphate buffered saline solution, water emulsions such as an oil/water emulsion or a triglyceride emulsion, various types of wetting agents, tablets, coated tablets and capsules. Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients. The invention also provides for pharmaceutical compositions together with suitable diluents, preservatives, solubilizers, emulsifiers and adjuvants. Other embodiments of the compositions of the invention incorporate particulate forms, protective coatings, protease inhibitors or permeation enhancers for various routes of administration, including but not limited to intravenous, intramuscular, parenteral, pulmonary, nasal and oral.

As used herein, an “effective amount” is the amount required to achieve a clinically significant effect. For example a significant reduction of infection, or reduction of cell growth or reduction of tumor size is a reduction of preferably of at least 30 percent, more preferably of at least 50 percent, most preferably of at least 90 percent. Accordingly, the effective amount will vary with the subject being treated, as well as the condition to be treated. For the purposes of this invention, the methods of administration are to include, but are not limited to administration cutaneously, subcutaneously, intravenously, parenterally, orally, topically, or by aerosol.

The present invention further contemplates therapeutic compositions useful in practicing the therapeutic methods of this invention. A subject therapeutic composition includes, in admixture, a pharmaceutically acceptable excipient (carrier) and one or more of a polypeptide analog or fragment of the provided peptide or peptide composition, a peptidomimetic composition thereof as described herein as an active ingredient. A cocktail of the provided pharmaceutical composition in various combinations is also contemplated.

The preparation of therapeutic compositions which contain polypeptides, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic polypeptide-, analog- or active fragment-containing compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of inhibition desired. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

As used herein, the term “synthetic amino acid” means an amino acid which is chemically synthesized and is not one of the 20 amino acids naturally occurring in nature. As used herein, the term “biosynthetic amino acid” means an amino acid found in nature other than the 20 amino acids commonly described and understood in the art as “natural amino acids.”

As used herein, amino acid residues are preferred to be in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxy terminus of a polypeptide. Abbreviations for amino acid residues are used in keeping with standard polypeptide nomenclature delineated in J. Biol. Chem., 243:3552-59 (1969).

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues.

Amino acids with nonpolar R groups include: Alanine, Valine, Leucine, Isoleucine, Proline, Phenylalanine, Tryptophan and Methionine. Amino acids with uncharged polar R groups include: Glycine, Serine, Threonine, Cysteine, Tyrosine, Asparagine and Glutamine. Amino acids with charged polar R groups (negatively charged at Ph 6.0) include: Aspartic acid and Glutamic acid. Basic amino acids (positively charged at pH 6.0) include: Lysine, Arginine and Histidine (at pH 6.0). Amino acids with phenyl groups include: Phenylalanine, Tryptophan and Tyrosine. Particularly preferred substitutions are: Lys for Arg and vice versa such that a positive charge may be maintained; Glu for Asp and vice versa such that a negative charge may be maintained; Ser for Thr such that a free -OH can be maintained; and Gln for Asn such that a free NH2 can be maintained. Amino acids can be in the “D” or “L” configuration. Use of peptidomimetics may involve the incorporation of a non-amino acid residue with non-amide linkages at a given position.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most comrnon amino acid in biochemical catalysis). Pro may be introduced because of its particularly planar structure, which induces-turns in the protein's structure.

As used herein, “pM” means picomolar, “nM” means nanmolar, “uM, means micromolar, “mM” means millimolar, “ul” or “μl” mean microliter, “ml” means milliliter, “l” means liter.

The following examples are presented in order to more fully illustrate the preferred embodiments of the invention. They should in no way be construed, however, as limiting the broad scope of the invention. While the invention is described and illustrated herein by references to various specific material, procedures and examples, it is understood that the invention is not restricted to the particular material combinations of material, and procedures selected for that purpose. Numerous variations of such details can be implied as will be appreciated by those skilled in the art.

### EXAMPLES

### Example 1

### Molecules Specifically Expressed in Murine Stem/Progenitor Cells

Protein tyrosine kinases and phosphatases expressed in murine hematopoietic stem cells have been previously identified (Matthews, W., et al. 1991; Matthews, W., et al. 1991; Dosil, M., et al. 1996). These molecules play important roles in hematopoiesis and development (Dosil, M., et al. 1996; Mackarehtschian, K., et al. 1995; Shalaby, F., et al. 1995; Kabrun, N. , et al. 1997). The present invention contemplates an even more global approach in order to identify molecules specifically expressed in the murine stem/progenitor cell hierarchy. For most of these studies purified (AA4.1+Lin−/loSca+ckit+) fetal liver cells were used. This population is approximately 1,000-fold enriched for in vivo repopulating activity (LTRA) measured by competitive repopulation (Ly5.1/Ly5.2 congenic system) (Moore, K. A., et al. 1997). All myeloid and lymphoid lineages in primary and in secondary recipients are repopulated by these cells. This degree of enrichment is comparable to the current “state of the art.” Other primitive members of the stem/progenitor cell hierarchy share this same cell surface phenotype. These include: (1) LTCIC or cobblestone area forming cells (Ploemacher, R. E., et al. 1989; Ploemacher, R. E., et al. 1991), (2) CFU-blast progenitors , (3) HPP-CFC progenitors (Lowry, P. A., et al. 1995) and (4) stromal-dependent B-lymphoid progenitors (Whitlock, C. A., and Müller-Sieburg, C. E. 1990). The AA4.1+Lin−/loSca-ckit+ subset is depleted of LTRA but contains significant in vitro progenitor activity. In contrast, no stem/progenitor cell activity is found in the AA4.1 subset (Jordan, C. T., et al. 1990). Short-term (5-7 days) cytokine cultures of stem cells were used to generate committed progenitor populations at the expense of LTRA (Traycoff, C. M., et al. 1996; Peters, S. O., et al. 1995; Knobel, K. M., et al. 1994; Yonemura, Y., et al. 1996). In summary, several cell populations were defined which represent the beginning, the middle and the end points of the hematopoietic hierarchy. This sets the stage for a comparative analysis of gene expression patterns. A goal of the present invention is to complement the physical and functional phenotypes of stem/progenitor cells with profiles of uniquely expressed genes. It was hypothesized that some of these gene products contribute to the unique biological properties of primitive stern/progenitor cells and therefore are regulators of self-renewal, proliferation, commitment and other processes.

There are a number of ways to compare gene expression profiles which are available to one of skill in the art. It is possible to do this by exhaustive sequencing of representative cDNA collections obtained from stem cell and mature cell sources followed by “electronic subtraction”. This approach has several drawbacks. Most importantly, the number of sequences which must be obtained is prohibitive. For a homogeneously pure population this number is on the order of 50,000 (based on approximately 10-20,000 expressed genes in an average cell type and a statistical calculation). In practice, even the most purified stem cell population is heterogeneous. Stem cell enrichment values are only meaningful in relation to an unenriched standard and cannot be converted into an absolute stem cell number. It has been documented that cell populations with the same cell-surface phenotypes can differ in biological activity . The unique properties of stem cells also suggest caution when extrapolating from expressed gene numbers in other cells. In short, the extent of sequencing necessary to ensure complete coverage of gene expression in stem cells is not possible to estimate. Normalization procedures (Uchida, N., et al. 1995; Patanjali, S. R., et al. 1991; Soares, M. B., et al. 1994) designed to “equalize” the mRNA abundance classes are not advisable because they obliterate potentially important quantitative expression differences. Additionally, a high-throughput sequencing effort is not applicable to numerous libraries. Comparisons of gene expression in diverse sources of stem cells will provide valuable information. An elegant technique, Serial Analysis of Gene Expression (SAGE) permits the rapid acquisition of thousands of DNA sequence “tags” (Zhang, L., et al. 1997). This technology was not considered herein because the size of each sequence “tag” is very small (10 base pair, bp). Therefore, SAGE is only informative in two extremes; exact nucleotide matches or no matches to sequences in the databases. This limits database comparison to the same species from which the “tags” originate. A key component of the strategy presented herein relies on broad bioinformatic database comparisons. In addition, even with a specific “tag” one still needs to obtain a full-length cDNA clone for functional studies. Analysis of gene expression can be done in single progenitor cells after these form a colony “start” (Brady, G., et al. 1990; Brady, G., and Iscove, N. N. 1993). Replating of the sibling cells in a “start” colony allows the approximation of the lineage potential present in the starting cell. This technique suffers from several drawbacks. First, it is limited to 3′, non-coding ends, thus preventing protein database comparisons. Second, the technique relies on cell growth, thus it is not suitable for analysis of quiescent cells. Moreover, approaches to allow colony-formation by true stem cells with a retention of primitive properties are in their infancy (Ball, T. C., et al. 1995; Trevisan, M., et al. 1996). Third, the technique does not take into account stochastic models of stem cell behavior (Ogawa, M. 1993). To accurately reveal physical and functional properties of stem cells it is wise to analyze populations where stochastic differences would average out. Single-cell derived cDNA populations could however, provide valuable “adjunct” material for more refined gene expression screens. There are many techniques to physically identify differentially expressed genes. For these and other reasons, most notably the technical and economic ease with which physical pre-enrichments of cDNA libraries can be achieved, the studies described herein began with cDNA libraries which are highly enriched in differentially expressed sequences. The integration of individual techniques served to overcome the inherent limitations of each technique. Three strategies were successfully employed: (1) Differential Display (DD) (Liang, P., et al. 1994; Bauer, D., et al. 1993), (2) Representational Difference Analysis (RDA) (Braun, B. S., et al. 1995; Hubank, M., and Schatz, D. G. 1994; Diatchenko, L., et al. 1996) and (3) standard subtractive hybridization (Li, W. -B., et al. 1994; Harrison, S. M., et al. 1995). The latter underlies the present invention. A key feature is that the differentially expressed cDNAs have a high probability of being full-length. This facilitates a rapid transition to functional studies. The two former techniques were utilized because of “visual” nature (DD) and the ability to generate representative, differentially expressed probe populations (RDA) in a rapid manner.

Murine Stem Cell Gene Expression Profiles.

As a first step, a series of high quality, representative cDNA libraries were generated. The cDNA populations were directionally cloned into the pSport-1 or pSport-2 plasmids (BRL-Gibco). The most important libraries originate from purified stem cells. In one case, enough AA4.1+Lin−/loSca+ckit+ cells were purified to allow construction of a non-based library using standard methodologies. This library contains ˜4×10⁶ independent recombinants (average cDNA insert size of 1-2 kilobases, kb). A second library was constructed using a new PCR-based technology called cap-finder (Clontech) designed to yield full-length cDNA copies. cDNAs ranging from 1 to 4 kb were commonly attained using this technique. For cap-finder procedures the purified cells were processed into DNAse-I digested, poly-A+ mRNA according to microscale procedures routinely in use. The synthesis of cDNA was done with an aliquot of mRNA corresponding to approximately 20,000 cell equivalents. It has not been necessary to use less material, therefore this is not a lower limit. A Not I restriction site was included in the 3′ reverse transcriptase primer (cap-finder, version 2) to facilitate directional cloning. An aliquot of the cDNA was amplified for varying PCR cycle numbers, and analyzed by Southern blots (pseudo-Northerns) for the presence of full-length copies of b-actin, GAPDH, CD18, flk2/flt3, cdk4, CD34 and other mRNAs. Optimal cycle numbers were used to amplify the remaining cDNA. The cDNA was cloned into the pSport-1 plasmid. The AA4.1+Lin−/loSca+ckit+ cap-finder library contains ˜3×10⁶ independent recombinants. Single-pass sequence analysis of random clones from this library indicated that 95% were full-length (based on sequences with an exact match in Genbank). Much more extensive sequencing of numerous clones from subtracted libraries has confirmed this. Other libraries constructed in similar ways include two libraries from AA4.1 cells, and two libraries from AA4.1+Lin−/loSca-ckit+ cells. Finally, a library was constructed from AA4.1+Lin−/loSca+ckit+ cells cultured for 5-7 days in a differentiation promoting cytokine cocktail (IL3, IL6, KL). Competitive repopulation and in vitro progenitor assays confirmed a complete loss of LTRA with a significant retention of progenitor cells. All of the cDNA libraries are large (>2×10⁶ independent recombinants) and of high quality (1-2 kb average insert size). In summary, the most primitive, intermediate and most mature members of the hematopoietic hierarchy were “converted” into representative panels of expressed genes.

Subtractive Hybridization.

These cDNA libraries were used in subtractive hybridizations to enrich for differentially expressed genes. Target libraries from AA4.1+Lin−/loSca+ckit+ cells were subtracted with an AA4.1 driver cDNA library. This yields a population of cDNA clones which is enriched in sequences expressed in primitive stem/progenitor cells but not in mature cell types. In practice, a single-stranded target library is hybridized to an excess of in vitro synthesized biotinylated RNA from the driver library as described . The opposite orientations of the cloning sites in pSport-1 and 2 insure target and driver complementarity. The target library was enzyrnatically converted to a single-stranded form using gene II protein and exonuclease III (Gene-Trapper protocols, BRL-Gibco). Previously, this was done by infection with M13 helper phage often resulting in a bias for small cDNA inserts. Here, driver/target combinations were subtracted two times in order to facilitate the removal of commonly expressed sequences (verified by elimination of “housekeeping” genes such as b-actin and GAPDH). Concomitant enrichment of known, differentially expressed genes is also verified. Generally flk2/flt3 and CD34 probes were used. Both are expressed in the AA4.1+Lin−/loSca+ckit+ subpopulation but not in the AA4.1 population. Following subtraction, the relative number of clones is reduced by up to 200-fold. In some cases (subtractions with AA4.1 material) the number of clones which “survive” the subtraction is on the order of 10-20,000. Because individual sequences may be represented more than one time, this does not necessarily imply that there are 10-20,000 differentially expressed genes. The exact number of unique sequences (complexity) in the pool of subtracted clones must be determined. A more thorough discussion of complexity is found in a subsequent section. These subtracted libraries should be enriched for sequences expressed in the primitive portion of the stem/progenitor cell hierarchy; that is in stem cells and/or in primitive clonogenic progenitor cells. Two other subtracted libraries, potentially enriched for sequences expressed in the most primitive stem cell but not in clonogenic progenitors were derived by subtracting the AA4.1+Lin−/loSca+ckit+ libraries with material from the closely related AA4.1+Lin−/loSca-ckit+ subpopulation.

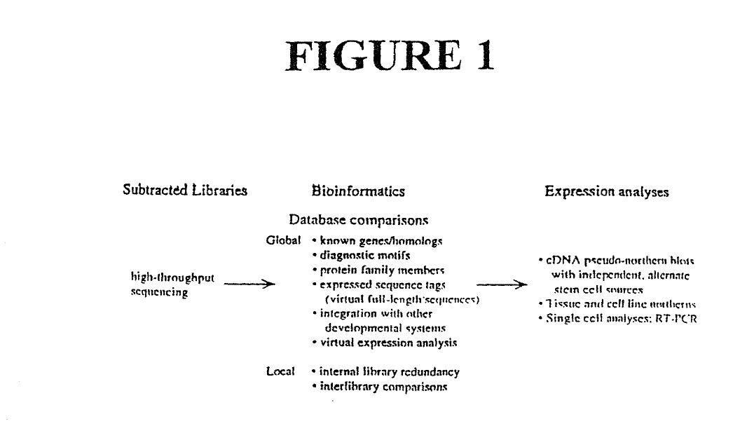

Each subtracted library is arrayed at high density onto nylon membranes. Each clone in the array has a unique address in microtiter plates. A density of 20-30,000 clones on a 22×22 cm. membrane is practical. Because of the inherent “noise” in any subtraction scheme, a positive selection criterion may be imposed in order to focus on true differentially expressed sequences. Details are presented in a subsequent section. To analyze the subtracted libraries, a high throughput sequencing effort was employed using three libraries. These are: 1) two AA4.1+Lin−/loSca+ckit+ cell libraries (standard and cap-finder) subtracted extensively with AA4.1 cell material and 2) a standard AA4.1+Lin−/loSca+ckit+ library subtracted with AA4.1+Lin−/loSca-ckit+ material. An average of 400 bp of 5′ sequence was obtained from about 1000 clones. To facilitate a rational handling of sequence information and to focus attention on a small number of clones for functional analysis a “flow of information” strategy was devised. (See, FIG. 1).

A major component of this strategy is bioinformatics. This can be global (comparisons with outside databases) and local (“in-house” analyses within an individual library or comparison of several libraries). A relevant example of the latter is comparison of genes from murine stem cells with genes derived from their human counterparts. Global bioinformatic analysis provides much information. First, it establishes if a given sequence corresponds identically or closely to an already identified gene in the mouse, human or other mammalian species. Such homologies can be detected at the nucleotide level. This can provide evidence of hematopoietic expression for a previously described gene. Second, where the homologies are statistically significant but not identical, novel members of gene/protein families can be identified. Third, a wider net can be cast over the databases by using conceptual translation of a sequence in the homology comparisons. The Examples provided herein illustrate the power of this approach in revealing similarities to proteins from invertebrate organisms such as Drosophila, C. elegans, and even yeast. In many cases these proteins have functions which have been uncovered by the analysis of mutants. Based on protein homologies “virtual links” are drawn between developmental regulation in invertebrates (such as in germ-line development) and in hematopoietic stem cells. The Notch/Notch ligand pathway first defined in invertebrate cell-fate determination and recently implicated in hematopoietic regulation is a good example.

A number of other putative proteins which share homology with key Drosophila proteins have been identified. Bioinformatics facilitates the recognition of peptide motifs such as EGF-like repeats, Ig-like domains or Zn-finger modules. Fourth, because the databases are annotated, predicted protein sequences can be assigned to cellular processes such as signal transduction pathways or apoptosis. It is also possible to categorize clones according to potential involvement in other mammalian stem cell systems such as the intestine. Fifth, it is feasible to perform virtual expression studies and to construct overlapping EST contigs which can yield virtual full-length cDNAs.

The following discussion summarizes the general murine findings and highlights a panel of “interesting” subtracted clones. A number of full-length sequences have been determined. Bioinformatic analysis summaries on a collection of 863 clones derived from AA4.1+Lin−/loSca+ckit+ libraries subtracted with AA4.1 RNA are shown in Table 1.

The present invention encompasses numerous bioinformatic search and comparison parameters. This is the first analysis of its kind in the hematopoietic system and several important points emerge. First there is a high proportion (˜50%) of novel sequences. Sixteen percent do not show homologies in any EST database queried. A preliminary analysis of a small sequence set from an AA4.1+Lin−/loSca+ckit+ library depleted of sequences in common with the AA4.1+Lin−/loSca-ckit+ subset has indicated an even higher percentage of novel genes. These are higher percentages than would emerge from a random analysis of an unsubtracted library. This was directly addressed by performing these analyses on a similar number of sequences selected at random from one of the mouse EST databases. Second, among the ˜50% of the clones which are significantly homologous to previously identified genes or proteins, the proportion of homologies to “housekeeping” genes is low. This underscores the effectiveness of the subtraction strategy. Third, an internal analysis of the data base has revealed few redundancies. This illustrates the degree of gene expression diversity between the two endpoints of the mouse hematopoietic hierarchy; thus supporting the starting hypothesis. A recent report has suggested that many sequences in mature blood cells are expressed in primitive, non-committed cells (Hu, M., et al. 1997). The data indicate that many of these are removed by the subtraction; thus uncovering a large, previously not described set of genes. Fourth the sequences with homologies to known genes or proteins can be subdivided according to protein families and putative function. Interestingly, a large percentage (32%) fall into the signaling protein category. Examples of these are described herein below.

As part of a more sophisticated bioinformatics approach, automation of database searches, information cross-referencing, and annotation was employed. An illustrative example is automated weekly database queries with the sequence set. New, previously-unidentified homologies were automatically noted and reported. In the mouse studies several genes were encountered whose expression would be predicted to differ between the AA4.1+Lin−/ioSca+ckit+ and the AA4.1− populations. Both murine CD34 and flk2/flt3 were identified in the sequenced population. This provides a good internal control for the screening strategy. As shown in Table 2, a “short list” of identified molecules was generated based on bioinformatics and in many cases, expression-specificity verification. The features of some of the molecules merit discussion.

The SA49P1 clone is homologous to sex comb on midleg; a member of the Drosophila Polycomb group of zinc-finger transcriptional repressors (Bornemann, D., et al. 1996). Polycomb proteins are regulators of homeobox (HOX) genes and maintain the developmental stability of transcriptional states (Simon, J. 1995). There is currently great interest in HOX gene function in the biology of hematopoietic stem cells (Sauvageau, G., et al. 1995; Lawrence, H. J., et al. 1996). The C4-80 gene is homologous to the Drosophila cornichon gene (Roth, S., et al. 1995). Comichon is required during oogenesis for the induction of follicle cells, which provide the environment that supports oocyte development. Comichon is a component of the Drosophila EGFR signaling pathway (Neuman-Silverberg, F. S., and T. Schupback 1996). The LL2-12 gene is similar to the Drosophila neurogenesis gene brainiac. Mutants defective in this extracellular gene product show neural hyperplasia. Brainiac also plays a role in oogenesis (Goode, S., et al. 1992; Goode, S., et al. 1996). Mosaic experiments show that brainiac is required in the developing oocyte. Brainiac involvement in the Drosophila EGFR pathway has been suggested. The LL2-35 gene is similar to the Drosophila germ cell-less gene (Jongens, T. A., et al. 1992). The product of this gene is required for the specification of the Drosophila germ line. The ectopic expression of germ cell-less causes somatic cells to adopt the characteristics of pole cells (destined for the germ line). The homologies of the above clones to Drosophila genes were identified at the predicted amino acid level. This underscores the utility of the bioinformatic approach. In all four cases the cDNAs represent novel unpublished murine genes. Clearly, the involvement of three of the Drosophila genes in the ultimate stem cell system, the germline, coupled with the identification of homologous genes expressed in mouse hematopoietic stem /progenitor cells gives food for thought. Four other cDNAs identified are homologous to the Drosophila genes dishevelled and smoothened (mouse homologs are already described) as well as to kelch and discs large (not previously described in the mouse). DD116 was originally isolated in a DD comparison of AA4.1+ vs. AA4.1 cells. A cDNA clone identified with the DD1 16 probe was sequenced; the predicted protein is homologous but not identical to the murine beige gene product. The beige mutation in the mouse causes bleeding, immune system disorders and a coat-color phenotype (Perou, C. M., et al. 1996; Fukai, K., et al. 1996; Barbosa, M. D., et al. 1996). Murine beige is thought to be the homologue of a gene responsible for the Chediak-Higashi syndrome in human. This novel gene is likely to be the second member in the beige family; it is designated herein as “taupe”. Two of the cDNAs in Table 2 were identified in a DD comparison of AA4.1+Lin−/loSca+ckit+ cells and cytokine differentiated, cultured cells. One focus was molecules whose expression disappeared with the loss of stem cell activity in culture. Cyt28 and Cytl9 fulfilled this criterion and were used to isolate full length clones which have been completely sequenced. Cyt28 encodes a novel, seven-transmembrane domain receptor in the same family as mouse F4/80 and human CD97 (secretin receptor superfamily) (Hamann, J., et al. 1995; Baud, W., et al. 1995). Cyt 19 is a novel putative methyltransferase. In both of these cases protein family assignations were made after extensive bioinformatic analyses. At least in the case of Cyt28, an antibody will be a useful reagent because it may provide a means to further subdivide the stem/progenitor cell population. Such antibodies are provided by the present invention. The cDNA SA61 is similar to a newly discovered molecule called p62dok. p62dok is a tyrosine phosphorylated protein which binds to rasGAP and is likely to be a common target for numerous tyrosine kinases including Ab1 and ckit (Carpino, N., et al. 1997; Yamanashi, Y., 1997). Interestingly, p62dok is also constitutively phosphorylated in hematopoietic progenitor cells from chronic phase CML patients (Carpino, N., et al. 1997). The predicted protein encoded by SA61 appears to be a second member of this protein class. Other representative molecules are listed in Table 2. Two cDNAs were identified as homologous to putative apoptosis regulators (SBSA56 and LL5-68, two cDNAs homologous to genes translocated in leukemias (LL5-03 and B2-67), several homologies to putative chromatin proteins (C4-23, C3-25 and LL2-89) and a LIM-domain encoding cDNA (LL5-96). Most interestingly, three cDNAs (B3-77, C2-48, and LL2-76) are homologous to genes expressed in intestinal crypts or during intestinal development.

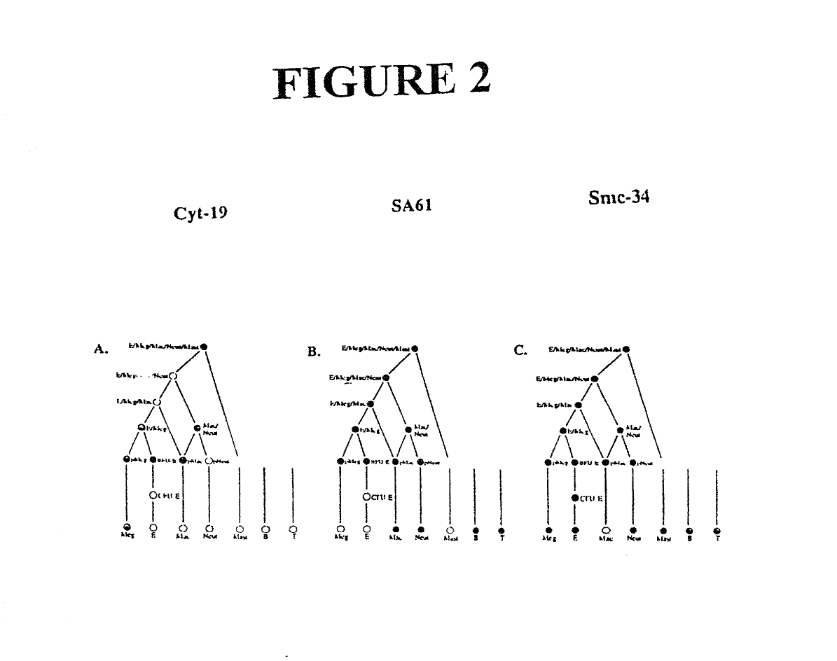

Isolated cDNAs were hybridized to slot blots representing globally amplified cDNAs from pools of individual progenitor and mature cells. Because the developmental potentials of these cells have been measured (by replating of siblings) it is possible to graphically represent the expression of a gene in various stages of the hierarchy. Three examples are shown in FIGS. 2A-2C. The Smc-34 cDNA is a completely novel sequence with a predicted leucine zipper and several potential membrane spanning domains. These examples underscore the value of “interfacing” the above-described approach with a single cell approach. Thus, it was demonstrated that a given gene is expressed in at least multipotential progenitor cells and other members of the progenitor cell hierarchy.

Human Stem Cell Gene Expression Profiles.

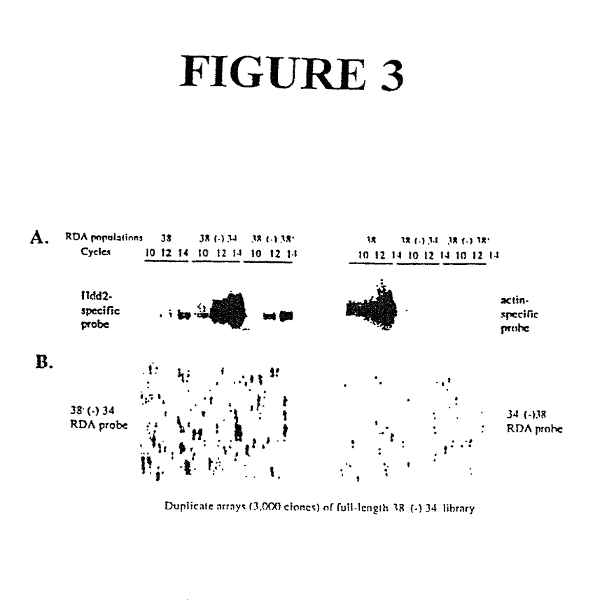

It is also a goal of the present invention to identify genes expressed specifically in human stem cells. It is useful to use a multi-species (mouse and human) approach to define stem/progenitor cell-specific gene profiles. One goal is to integrate mouse and human information. However, rather than proceeding directly to human homologs of the mouse genes already identified, an independent human effort was initiated. The rationale for this is multi-faceted. First, it is possible that some aspects of human and mouse stem cell biology are regulated in different ways. Although it is likely that most regulatory pathways will be conserved, it must be kept in mind that many properties ascribed to the most primitive stem cell population have been rigorously proven only in the mouse. Clearly a human lymphoid-myeloid stem/progenitor cell exists. However, the exact degree of proliferative capacity (the ability to give rise to oligoclonal hematopoiesis) as well as the exact spectrum of differentiation potentials of human stem cells have not yet been accurately measured. Several potential differences between mouse and human have already been mentioned. One additional indicator of differences may be the difficulty of gene-transfer into stem cells from large animals (Larochelle, A., et al. 1997). This is not likely to result solely from the quiescent status of the most primitive cells. Second, while it is usually possible to find human homologs for individual mouse genes by manipulating hybridization stringency, such conditions will vary for different genes. Therefore, to find human homologs for a large pool of mouse genes (i.e. 100) may be more labor intensive and costly than to independently determine a sequence profile of differentially expressed genes from human stem/progenitor cells. Relationships to the murine panel can then be determined electronically where it is easy to manipulate comparative parameters. Clearly, for some individual mouse genes it will be of great importance to physically identify human homologs. In some cases it may be possible to use existing human EST databases to quickly obtain the sequence of a human homolog. Third, the availability of a large panel of human sequences specifically expressed in stem/progenitor cells lends itself to the application of various chip and array technologies. Such technologies will be instrumental in identifying which subsets of human stem cell specific genes are up or down-regulated in the highly clonogenic stem cells from diseases such as Acute Myelogenous Leukemia (AML) and other leukemias. Highly purified human stem cells for cDNA libraries were obtained from normal donor BM. CD34+ cells were obtained from the mononuclear fraction using an immuno-magnetic affinity device (Miltenyi MACS column) and stained with a mixture of monoclonal antibodies (mAbs) designed to identify both primitive and mature cell surface markers. To identify antigens on mature cells a lineage (Lin) cocktail of FITC conjugated mAbs was used . These mAbs were directed against: CD3, CD11b, CD15, CD19, CD20, glycophorin A, and CD71 (CD3, CD15, CD19 and CD20 from Becton Dickinson, CD11b and glycophorin A from Coulter, and CD71 from Ortho Diagnostics). To identify primitive cells mAbs were used which were directed against CD34 (biotin conjugated and detected via a streptavidin-allophycocyanin reagent; Coulter) and either CD38 (phycoerythrin conjugated, Becton Dickinson) or CD90 (Thy1) (phycoerythrin conjugated; Pharmingen). The stained cells were analyzed using a dual laser Becton Dickinson FACStar Plus flow cytometer. Cells of the desired phenotype were sorted into siliconized tubes. To obtain material from cultured cells, CD34+ enriched populations (isolated as described above), were first cultured in serum-free medium (IMDM, 5 mg/ml low density lipoprotein, 2 mM glutamine, human serum albumin, insulin, and transferrin, ) supplemented with IL-3 (5 ng/ml), KL (25 ng/ml), and FL (25 ng/ml). After 1-4 days of culture, the cells were purified as described above. Human libraries were constructed using the cap-finder technology, version 2. Amplified cDNAs originating from several independent BM donors were pooled. Representative cDNA libraries have been constructed from the following sources: (1) BM CD34+Lin− cells (7.1×105 independent recombinants), (2) BM CD34+Lin−CD38+ cells (1.9×106 independent recombinants), (3) BM CD34− cells (1.6×106 independent recombinants), (4) CB CD34+ cells (4.3×105 independent recombinants), and (5) CB CD34− cells (2.9×105 independent recombinants). The average cDNA insert size in all libraries is 1-2 kb. According to the comparative biological properties of the source material used for the mouse and human cDNA libraries, the following parallels can be drawn: (1) mouse AA4.1+Lin−/loSca+ckit+≅human CD34+Lin−, (2) mouse AA4.1+Lin−/loSca-ckit+≅human CD34+Lin−CD38+ and (3) mouse AA4.1≅human CD34−. Thus, in both species, collections of clones representing the beginning, the middle and the end-points of the hematopoietic hierarchy have been generated. Using procedures described above, the human libraries were subtracted and arrayed 3,000 clones from two libraries: (1) BM CD34+Lin− subtracted with BM CD34+Lin−CD38+ material (referred to as: 38−)38+) and (2) BM CD34+Lin− subtracted with BM CD34− material (referred to as: 38−). Before initiating a sequencing effort pilot studies were performed to improve the resolution of the screen. Specifically, in order to develop techniques which would eliminate as much non-specific “noise” as possible from the libraries. RDA (PCR-Select, Clontech) were used . There are several salient features of RDA. First, it is rapid (2-3 days) and applicable to small cell numbers (ie. several thousand). It does not require the generation of cloned cDNA libraries therefore, numerous comparisons can be made simultaneously. Second, it yields short cDNA fragments (generated by 4-cutter restriction enzymes) which are representative of differentially expressed mRNAs. Third, it is based on the suppression PCR technique which prevents overamplification of abundant messages (Diatchenko, L., et al. 1996). In addition, sequences expressed in both populations in an RDA comparison do not amplify exponentially. Differentially expressed populations of cDNA fragments are not obtained by physical enrichment but rather by selective PCR amplification. Fourth and most importantly, it simultaneously yields two populations each representing differentially expressed genes in one of the two starting samples. This bi-directionality is valuable because as discussed below it can simultaneously reveal up and down regulated genes. RDA comparisons were made between CD34+Lin− and CD34+Lin−CD38+ and between CD34+Lin− and CD34− cDNAs. The latter comparison was verified by hybridization. As shown in FIG. 3A (right panel), the control, non-subtracted RDA cDNA population (38−) contains b-actin sequences which are missing in the two subtracted RDA populations, 38− and 38−)38+. A differentially expressed gene (HDD-2, described below) is enriched in the 38− RDA population and at least retained in the 38−)38+RDA population (FIG. 3A, left panel). Two, bi-directional, RDA cDNA populations (38− and the converse 34−)38) were used to probe (See FIG. 3) duplicate arrays of a subtracted 38− library (FIG. 3B). The correct RDA probe hybridizes to considerably more clones than the incorrect probe (compare the greater numbers of spots in the left as compared to the right panel). Hybridization signals for each clone are doublets due to the arraying technique. Hybridization signals with the incorrect probe (right panel) suggest that further improvements to this strategy are worth pursuing. Because the subtractions are based on different technologies, it was reasoned that clones which “survived” the library subtraction and hybridized preferentially to the “correct” RDA probe would be more likely to represent true differentially expressed genes. Preliminary data from the mouse studies had already suggested that this was indeed the case. The arrayed clones were also hybridized to probe populations from the entire starting non-subtracted libraries in order to eliminate residual cDNA clones corresponding to abundant mRNA species.

In one analysis, a total of 77 clones (49 from the 38− subtraction and 28 from the −38−)38+ subtraction) which “obeyed” the RDA criteria were sequenced from the 5′ end. In order to verify expression specificity, a series of pseudo-Northern blot analyses were performed. cDNA populations from independent purified sources of material was amplified. This is of particular importance with an outbred species such as humans. In these experiments additional material from CD34+Lin−CD90+(Thy1+) (three separate 10 purifications) and their CD90− (Thy1−) counterpart subpopulations was also included. Cytokine-cultured samples were also included. These were CD34+Lin− cells and their CD38+ counterparts purified after 1, 2 and 4 days of culture. The collective panel of cDNAs also includes four independently purified CD34+Lin− populations and their CD34+Lin−CD38+ counterparts. All of these amplified cDNAs have been arranged on numerous pseudo-Northern blots. In short, this allowed us to evaluate the expression of a given “sequence tag” in stem cell populations isolated by two different criteria, from a number of independent donors and after cytokine culture. Several interesting genes emerged from the sequence data set. Individual clones obtained from the −38−)38+ subtraction are designated 38 letter/number (i.e., 38A1) while those from the 38−)34+ subtraction are designated 34 letter/number (i.e., 34A1). One clone (38B5) was identified and determined to be human flk2/flt3. A second interesting clone (34B4) is closely related to a gene encoding TINUR (FIG. 4A). Clone 34B4 may be a novel variant of TINUR due to a 25 amino acid, in-frame deletion. TINUR was identified as an orphan member of the steroid receptor superfamily (NGFI-B/nur77 subfamily) (Okabe, T., et al. 25 1995). TINUR has also been implicated in apoptosis. An additional clone (34F4 ) is highly homologous to DAP-kinase. This protein is a serine-threonine kinase which has been implicated in cytokine (IFN—induced apoptosis (Deiss, L., et al. 1995). Clone 34F4 (DAP-Kinase) and 34B4 (TINUR) both exhibit a stem-cell restricted expression pattern. Clearly, the identification of two genes whose products are implicated in 30 apoptosis and whose expression is largely restricted to human stem cells is of interest. In addition, a cDNA (34A5) was identified which is closely related to the MLF1 gene which is a translocation partner in t(3;5) (q25.1;q34) (Yoneda-Kato, N., et al. 1996). This translocation is associated with Myelodysplastic Syndrome (a stem cell disease) and AML. The sequence homologies and restricted expression pattern of 34A5 is shown in FIGS. 4B and 4C. In FIGS. 4C and 4D (and also 5A, 5B, and 6B) there are twenty-one samples of capfinder-amplified cDNA from various hematopoietic populations. From left to right these are: four CD34+Lin− populations, three CD34+Lin−CD90+ populations, two CD34− populations, four CD34+Lin−CD38+ (obtained from the same BMs as the CD38− samples in lanes 1-4), two CD34+Lin− samples (obtained from the same BMs as the CD90+ samples in lanes 5 and 6), three CD34+Lin− populations obtained after 1, 2 or 4 days of culture and finally their three CD34+Lin−CD38+ counterparts. A recent study shows that, at least with some types of AML, the disease can be transferred into NODSCID mice only by the leukemic CD34+CD38− subpopulation (Bonnet, D., and Dick, J. E. 1997). These demonstrate MLF1 expression in normal stem cells. As shown in FIG. 4D, nucleophosmin (NPM), the partner in this translocation was also identified. This is an example of non-specifically expressed “noise” in the screens. The 38G2 cDNA is closely homologous to the LTG9/MLLT3 gene located on 9q22 and involved in t(9; 11) leukemia (Iida, S., et al. 1993) (FIG. 4E). A degree of stem cell expression specificity has also been observed. All of the above cDNA clones are likely to be identical to the homologous, previously identified genes. However, the suggested involvement of DAP-kinase and TINUR in apoptosis necessitates their inclusion in any comprehensive consideration of stem cell apoptotic pathways. Similarly, the expression of two genes associated with myeloid leukemias bears on any speculation regarding the primary transformation target cell as well as the origins of the ultimate clinical phenotype in these and other leukemic disorders.

Other known genes to were found that exhibit identity or very close homology including: (1) GOS3, a fos-related gene (Heximer, S., et al. 1996) (Figure SA) and (2) HLA-DR (FIG. 5B). GOS3 shows a specific expression pattern, while HLA-DR expression appears to be more variable. The expression status of Class II MHC on the most primitive human BM stem cell population is not entirely clear. It has been suggested to be present in a primitive, multipotent progenitor population but not in the most primitive stem cells (Sutherland, H. S., et al. 1989; Verfaillie, C., et al. 1990). If true, this may suggest an additional negative selection parameter for future experiments designed to subdivide the stem/progenitor cell hierarchy. Of the first 75 sequences, 22 have no homologies in the databases or homologies only to ESTs. Expression analyses on these clones are in progress. One gene of particular interest is called HDD-2 as well as 34B5, 34E1 and 38A11. The three latter designations reflect its isolation from both the 38− as well as the 38−)38+ subtracted libraries. The designation HDD-2 reflects its independent isolation in a limited DD “first look” at molecular differences in the purified cell populations. The likely full-length sequence of HDD-2 is ˜500 bp. It contains a short open reading frame of 89 amino acids (SEQ.ID.No.: 71). The predicted peptide sequence is shown in FIG. 6A. The 3′ cDNA sequence contains a poly-A tail preceded by the canonical AATAAA poly-adenylation signal. Neither the nucleotide nor the predicted protein sequences of HDD-2 show homologies in any known gene or EST database. The expression profile of HDD-2 shown in FIG. 6B, demonstrates that it is stem cell restricted. Also shown below (FIG. 6C) is HDD-2 hybridization to a dot blot with numerous human pA+ mRNA samples (Clontech). HDD-2 hybridization is only visible in kidney (the other “spots” are background). It was confirmed that HDD-2 corresponds to a single-copy human gene by genomic Southern blot (FIG. 6D). In summary, the results of this very low throughput human sequencing effort substantiate the overall approach; that judicious pre-enrichments and selections will result in rapid identification of biologically interesting and often novel genes. Most importantly, these studies firmly establish the existence of genes whose expression correlates with the most primitive stem cell phenotype.

Other data were generated using (1) a murine stromal cell line to support enriched human stem/progenitor cells, and (2) tetracycline regulatable retroviral expression vectors. The stromal cell line AFT024 is efficient in long-term, in vitro maintenance of LTRA in purified murine fetal liver and adult bone marrow populations (Terstappen, L. W. M. M., et al. 1991). Additional data demonstrated highly-efficient retroviral-mediated gene-transfer into murine LTRA as well as into primitive in vitro progenitors during the AFT024 cocultures. Further, it has been shown that AFT024 is very effective in supporting ELTCIC. Specifically, the CD34+CD38− immunophenotype as well as the functional capacity of these cells is maintained. The latter was measured in limiting-dilution replating experiments. Moreover, it has been shown that limiting numbers of human CD34+Lin− cells can give rise to both B cells and NK-cells when cultured on AFT024. However, the supportive activities of AFT024 on mouse and human stern/progenitor cells have been indistinguishable. In addition, more than 500 sequences have been analyzed from an AFT024-specific subtracted cDNA library. A number of candidate stem cell regulators have been identified. Three of these are dlk (Moore, K. A., et al. 1997), a novel BMPITGF-b superfamily member and a novel selectin-related molecule. The present invention contemplates identifying human stem/progenitor cell receptors and/or ligands for the AFT024 specific proteins.

In order to facilitate functional studies of stem cell gene products several retroviral gene-transfer vector systems were constructed and characterized. All of these employ the 293T cell retroviral packaging system (Kinsella, T., and Nolan, G. 1996). High titers of virus can be produced transiently without the time and labor consuming effort required for stable producer cell lines. Large cDNA populations can also be converted into virus populations (Kitamura. T., et al. 1995). It is preferable to have inducible (or repressible) vectors which are also selectable. Also, the single-transcription unit tetracycline (tet) repressible vector was modified (Hofmann, A., et al. 1996). This vector includes an enhancer/promoter deletion in the 3′ LTR. The tet system is currently one of the best inducible expression systems available; regulation over a several hundred-fold range of expression is observed (Shokett, P. E., and Schatz, D. G. 1996). Cloning sites were introduced in order to insert cDNAs in a sense or antisense orientation. A green fluorescent protein (GFP) marker was also included in these vectors driven by a thymidine-kinase promoter (TK-GFP) (Yang, T., et al. 1996; Cheng, L., and Kain, S. 1996). The cDNA fragment of interest is under tet regulation while GFP is constitutively expressed in transduced cells. In order to confirm this, NIH3T3 cells are infected with a LacZ virus and GFP+ cells sorted. The GFP+cells express LacZ in the absence of tet, while LacZ expression is undetectable in most cells after the addition of tet. The titer of this model construct is approximately 104/ml., which is suitable for tissue culture studies.

TABLE 1
Known genes (or extensive aa homology): 436 (50.5%)

Grouped by function

| | | |
|---|---|---|
| by nucleotide: 407 | Signaling/receptors: | 133 |
| by amino acid only: 29 | Translational/post-: | 62 |
| to mouse: 230 | Structural: | 49 |
| to human/other: 206 | Transcriptional/post-: | 45 |
| | Cell fate: | 35 |
| | Other | 87 |
| | Unknown function: | 25 |

Novel genes: 427 (49.5%)
Homologous only to expressed sequence tags: 288 (33.3%)
No homology to any known nt or aa sequences: 139 (16.2%)

TABLE 2

| Putative family | Clone | Method | Cells compared | Notes |
|---|---|---|---|---|
| 7-transmembrane receptor | Cyt-28 | diff. display | SC* fresh vs. 7d-cultured | secretin R superfamily |
| Methyltransferase | Cyt-19 | diff. display | SC fresh vs. 7d-cultured | |
| | C3-54 | subtraction | SC vs. AA4′ | |
| Aspartyl protease | SA7 | subtraction | SC vs. AA4′ | |
| Signal transduction molecules | SA61 | subtraction | SC vs. AA4′ | dok family member |
| | LL2.02 | subtraction | SC vs. AA4′ | BTK associated |
| beige-related proteins | DD116 | diff. display | AA4″ vs. AA4′ | contains WD repeat |
| Transcriptional regulators | SA49PI | subtraction | SC vs. AA4′ | polycomb homol. |
| | LL5-96 | subtraction | SC vs. AA4′ | includes LIM domain |
| G-protein signaling | LL4-39 | subtraction | SC vs. AA4′ | cell cycle transition |
| | B1-66 | subtraction | SC vs. AA4′ | similar to BL34 |
| Apoptosis-related genes | SBSA56 | subtraction | SC vs. AA4′ | SMT3A-related |
| | LL5-68 | subtraction | SC vs. AA4′ | sim. to Requiem transcript. factor |
| Chromatin proteins | C4-23 | subtraction | SC vs. AA4′ | yeast HST2-like |
| | C3-25 | subtraction | SC vs. AA4′ | NHP2-like |
| | LL2-89 | subtraction | SC vs. AA4′ | yeast S1S2-like |
| Other stem cells (intestinal | B3-77 | subtraction | SC vs. AA4′ | homologous to A4 gene |
| crypts) | C2-48 | subtraction | SC vs. AA4′ | homologous to C101 |
| | LL2-76 | subtraction | SC vs. AA4′ | homologous to EDPF |
| Genes involved in leukemogenic | LL5-03 | subtraction | SC vs. AA4′ | t(1:11)(q21:q23) |
| translocations | B2-67 | subtraction | SC vs. AA4′ | t(X:14)(q28:q11) |
| Homologues of Drosophila | LL2-35 | subtraction | SC vs. AA4′ | germ cell-less |
| developmental genes | LL2-12 | subtraction | SC vs. AA4′ | brainiac |
| | C4-80 | subtraction | SC vs. AA4′ | cornichon |
| | B4-14 | subtraction | SC vs. AA4′ | discs-large |

The designation “SC” refers to sorted day 14 fetal liver cells having the phenotype AA4.1 ”Lin ‘Sca’ckit”.

### Example 2

### Molecular Cloning and Characterization of AA4, an Early Marker of Hematopoietic Development

In this example, the expression cloning and molecular characterization of AA4, a surface marker expressed on hematopoietic stem and progenitor cells is described. The results demonstrate that AA4 is a 30 kDa type I glycosylated membrane protein whose structural organization suggests a role in cell adhesion. Expression analysis showed that high levels of AA4 are found in lung, heart, and bone marrow. It is not found in undifferentiated ES cells, but its expression is upregulated as these cells differentiate into colonies of hematopoietic precursors and endothelial cells. In the hematopoietic system, expression of AA4 correlates with the expression of stem cell markers CD34 and CD43. Functional studies revealed that AA4 coimmunoprecipitates with CD34 and CD43, suggesting that these proteins form a macromolecular complex which functions in the regulation of cell adhesion, proliferation and/or differentiation of hematopoietic cells.

Introduction

Monoclonal antibody AA4.1 was first described more than a decade ago (McKearn et al., 1984) and since then it has become a useful marker for the isolation and analysis of hematopoictic cells (McKeam et al., 1985; Jordan et al., 1990; Fujimoto et al., 1996). A number of works have shown that the antigen recognized by AA4.1 is present on a subset of primitive hematopoietic progenitors found at various stages of development in sites of active hematopoiesis in yolk sac (Cumano et al., 1993; Auerbach et al., 1996; Yoder et al., 1997), AGM region (Godin et al., 1995; Marcos et al., 1997), fetal liver (McKeam et al., 1985; Jordan et al., 1990; Cumano and Paige, 1993), and bone marrow (Li et al., 1996; Szivassy and Cory, 1993). In yolk sac, AA4-positive cells are first detected at day 8-10 of gestation (Cumano et al., 1993; Sanchez et al., 1996). At day 9-10 of gestation AA4⁺c-Kit⁺ progenitors are found in the P-Sp/AGM region (Sanchez et al., 1996; Marcos et al., 1997), and by day 14 of development, AA4 defines 0.5-1.0% of the fetal liver tissue that contains the entire hierarchy of primitive hematopoietic cells (Jordan et al., 1990). Proliferation within each successive compartment results in increased total number of progenitor cells. Antigen density per cell also increases with developmental progress, which is especially marked for c-Kit and AA4 (Marcos et al., 1997). In bone marrow, HSC are found in both AA4⁺ and AA4⁻ subpopulations, although in adult marrow AA4 is largely regarded to be a marker of early B lymphoid lineage. In addition, recent studies demonstrate that the expression of AA4 parallels the onset of hematopoietic development in differentiating ES cells (Kabrun et al., 1997; Lin and Neben, 1997; Potocnik et al., 1997). Taken together, these results indicate that AA4 plays an important role in hematopoiesis and has to be studied in more detail.

In order to achieve this goal, AA4 was molecularly cloned and characterized. The results demonstrate that AA4 is a 130 kDa type I transmembrane glycoprotein whose structure suggests a role in cell adhesion. Expression analysis showed that high levels of AA4 are found in lung, heart, and bone marrow. In the hematopoietic system, expression of AA4 correlates with the expression of stem cell markers CD34 and CD43. Functional studies indicate that AA4 coimmunoprecipitates with CD34 and CD43, suggesting that these proteins form a macromolecular complex which may function in the regulation of cell adhesion, proliferation and/or differentiation of hematopoietic cells.

Results

Cloning of AA4.

In order to identify AA4, AA4.1 monoclonal antibody was used to screen various cell lines and primary hematopoetic cells. The murine B lymphoid cell line D2N was found to expresses relatively high levels of AA4 antigen (see Table 3). Immunoprecipitation of protein extracts prepared from D2N cells showed that AA4.1 recognizes a protein with apparent molecular weight (Mᵣ) 130 kDa (FIG. 7). This protein was also present in extracts prepared from B lymphoid cell line M2.4, and AA4 positive hematopoietic cells derived from bone marrow and fetal liver.

To isolate AA4, a cDNA library was prepared from D2N cells and cloned into the polylinker region of a retroviral expression vector REBNA (see Materials and Methods). Following production of retroviruses, NIH 3T3 cells were infected with the recombinant cDNA library and selected for AA4 expression by flow cytometry using PE-conjugated AA4.1. After two rounds of sorting, genomic DNA extracted from AA4-positive cells was analyzed by pcr amplification using viral vector primers. This resulted in the amplification of a 3.1 kbp cDNA which was gel-purified and subcloned for further analysis. Infection of NIH 3T3 fibroblast or EML C1 hematopoietic cells with REBNA/AA4, a recombinant retrovirus expressing the cloned cDNA, has lead to the acquisition by cells of high affinity to AA4.1 mAb (FIG. 8A). In infected cells, AA4.1 detects a 130 kDa surface protein which comigrates with the endogenous AA4 from D2N cells (FIG. 8B), thus indicating that the cloned cDNA encodes AA4.

Sequence Analysis of AA 4.

Sequence analysis of the cloned cDNA (SEQ. ID No.: 72) showed that it has a single open reading frame encoding a protein of 644 amino acids (see FIG. 9)(SEQ. ID No.: 73. The deduced amino acid sequence includes a putative leader peptide and the mature protein which starts at position 20. The protein contains a long N-terminal extracellular region, a single putative hydrophobic transmembrane region, and 47 amino acids of the C-terminal cytoplasmic domain. The extracellular part of AA4 is composed of two major structural moieties. The N-terminal region contains a C-type lectin domain (CTL) which has 32% sequence homology to endothelial cell receptor thrombomodulin. This region is followed by a cysteine-rich domain composed of six epidermal growth factor (EGF)-like repeats, three of which are consistent with the calcium-binding EGF motifs. Similar repeats are found in the extracellular domains of a large number of membrane-bound proteins and in proteins known to be secreated (Bork et al., 1996).

Database searches revealed that AA4 exhibits high homology to C1qRP, the human receptor for complement component C1q expressed on surfaces of myeloid cell lineage and endothelial cells (Nepomuceno et al., 1997). Sequence alignment showed that AA4 and C1qR have approximately 68% identical amino acid positions and similar domain structures. Highest homologies were found within the N-terminal parts of the proteins and their C-terminal cytoplasmic domains (see FIGS. 10A and 10B), suggesting that AA4 and C1qR may share functional properties.

The amino acid sequence of AA4 contains numerous potential O-linked and N-glycosylation sites. Although the predicted Mᵣ of AA4 is 67.4 kDa, two protein bands exhibiting Mᵣ 105 kDa and 130 kDa respectively, are immunoprecipitated by AA4.1 mAb in cells infected with REBNA/AA4 (FIG. 8C). When ³⁵S-labeled cells are chased with nonradioactive media, the intensity of the 105 kDa band rapidly decreases, while the intensity of the 130 kDa band increases, thus indicating that p130 is the mature form of the protein, whereas p105 is its precursor. In agreement with this conclusion, immunoprecipitation of biotinyated cells using AA4.1 reproducibly results in the detection of a 130 kDa surface protein (see FIG. 8B).

Expression Patterns of AA4.

Northern blot analysis showed that in adult mouse tissues, AA4 is expressed at high levels in lung, heart, and bone marrow. No detectable expression was found in brain, testis, spleen, and thymus (FIG. 10B). In normal tissues and transformed cell lines, a 7 kb RNA species hybridizes with the cloned cDNA (FIGS. 11A and 11B). In addition to the 7 kb species, poly(A)-RNA from D2N cells contains a minor band corresponding to a 3.2 kb mRNA (see FIG. 11A, lane 8). Similarity search against expressed sequence tags (ESTs) showed that databases contain at least seven different sequences corresponding to the 3′-untranslated region of the cloned aa4cDNA which were isolated from the following mouse tissues: colon (Genbank accession No AA929174), heart (AA435107), lymph node (AA185911 and AA267407), lung (AA220480), mammary gland (AI021507), and spleen (AA145088). Sequence identity with the ESTs abrogates upstream of nucleotide G at position 2481 in the 3′-untranslated region of aa4cDNA, thus indicating that the cloned cDNA corresponds to an alternatively spliced 3.2 kb aa4mRNA.

Expression of AA4 in Hematopoietic Progenitor Cells

AA4 is produced in murine hematopoietic progenitor cells and immature B lymphocytes found at various stages of development in yolk sac (Godin et al., 1995; Marcos et al., 1997), fetal liver (McKeam et al., 1985; Jordan et al., 1990), and bone marrow (Cumano et al., 1992; Li et al., 1996). To confirm that the cloned cDNA encodes AA4, RT-PCR was performed on hematopoietic cells fractionationated using several different techniques. The cells analyzed included AA4⁺ and AA4⁻ fetal liver (FL) cells; AA4⁺ FL cells fractionated into Lin^{lo}cKit⁺Sca-1⁺ and Lin^{lo}c-Kit⁺Sca-1⁻¹ populations; and hematopoietic progenitors isolated from bone marrow (BM) by cell sorting using combinations of different surface markers.

FIG. 12A shows that aa4 was amplified from AA4⁺ FL cells, whereas in AA4⁻ cells it was only present at low levels. AA4 expression was high in Lin^{lo}c-Kit+Sca-1⁺ cells enriched for HSC activity. In adult marrow cells, aa4 was amplified from Lin⁺ cells and Lin⁻c-Kit⁺Sca-1⁺CD34⁺ multipotential progenitors. At lower levels aa4 was present in Lin⁻c-Kit⁺Sca-1⁺CD34⁻ long term reconstituting HSCs (FIG. 12B). In all tested cells aa4 expression correlated with the expression of stem cell markers CD34 and CD43.

Embryonic stem (ES) cells which have been shown to generate progenitors for most hematopoietic lineages during differentiation in vitro (Keller, 1995) were also tested in this experiment. FIG. 12C shows that aa4 was not found in undifferentiated ES cells but its expression was upregulated as these cells differentiated and formed blast cell colonies (BL) and colonies of more differentiated hematopoietic cells (HMT). These results are in line with previous studies which showed that AA4 is expressed in ES-derived hematopoietic precursors. (Kabrun et al., 1997; Ling and Neben, 1997; Scott et al., 1997) and show that this expression is not abrogated upon differentiation of blast cell colonies into endothelial cells (ENT in FIG. 12C).

Ectopic Expression of AA4 has no Mitogenic Effect

EML C1 and NIH 3T3 cells expressing exogenous AA4 did not exhibit morphological changes indicative of alterations in their growth properties. To examine the effect of AA4 on growth phenotypes in more detail, NIH 3T3 and primary mouse embryo fibroblasts were infected with a retrovirus expressing AA4 and maintained in high and low serum conditions. FACS analysis of the transduced cells confirmed that in each case the efficiency of infection was close to 100%. However, examination of growth rates showed that overexpressed AA4 had no apparent effect on proliferation of both cell types as compared to control uninfected cells or the corresponding cells infected with a retrovirus expressing GFP.

AA4 Coimmunoprecipitates with CD34 and CD43

To investigate interactions with other proteins, hematopoietic and fibroblast cells expressing AA4 were immunoprecipitated with AA4.1 mAb and then examined by immunoblot analysis using a panel of antibodies directed against membrane-associated proteins. This analysis revealed that AA4 coimmunoprecipitates with CD34, a membrane glycoprotein selectively expressed within the hematopoietic system on stem and progenitor cells, and CD43 which is a major O-glycosylated sialomucin found on the surfaces of most leukocytes. This result is in line with previous studies which showed that CD43 coimmunoprecipitates with the human C1qRP (Guan et al., 1991; 1994). FIG. 13B shows that in the murine D2N and EML C1 cells, a 52 kDa protein is the major isoform of CD43 that associates with AA4. This 52 kDa protein was found to be reactive with both the N-terminal (S9) and C-terminal (M19) anti-CD43 antibodies, indicating that it is not a breakdown product. In NIH 3T3 fibroblasts coexpressing CD43 and AA4, a 54 kDa and a 170 kDa CD43 isoforms coimmunoprecipitated with AA4 pointing to the glycosylation differences brtween CD43 expressed in different cell types. A 115 kDa CD43 isoform which previously have been shown to be sialylated and thus overly negatively charged (Guan et al., 1994) did not form macromolecular complexes with AA4 in NIH 3T3 cells. Similarly, AA4 did not coimmunoprecipitate with a 115 kDa CD43 isoform found in D2N cells and a 120 kDa isoform found in EML C1 (FIG. 13B).

To examine associations of AA4 with CD34, both proteins were expressed in Rat-1 cells following infection with the corresponding retroviruses. See FIG. 13C.

In Rat-1 cells coexpressing AA4 and CD34, AA4.1 the results also demonstrate that AA4 coimmunoprecipitates with CD34. Studies have suggested a role for both CD34 and CD43 in the regulation of adhesion, growth and differentiation of hematopoietic precursors (Ardman et al., 1992; Bazil et al., 1997; Chen et al., 1996; Suzuki et al., 1996; Wood et al., 1997; Stockton et al., 1998). Coexpression of AA-Fc fusion protein in NIH 3T3 cells stably producing AA4 showed that AA4-Fc forms heterodimeric complexes with AA4. FIG. 11A shows that equimolar amounts of AA4 and AA4-Fc were coprecipitated from these cells by protein A, indicating that AA4 is prone to homo- or heterodimerization.

Discussion

This Example describes the expression cloning and sequence analysis of AA4, a molecular marker expressed on hematopoietic stem and progenitor cells. The CDNA encoding AA4 was isolated from a retroviral cDNA library prepared from the murine D2N lymphoid cell line. Sequence analysis of the cloned cDNA revealed that AA4 is a type I transmembrane protein composed of 625 amino acids. The extracellular part of the molecule contains two major structural moieties, a C-type lectin carbohydrate recognition domain and six EGF-like domains. Similar repeats have been found in a large number of membrane-bound proteins or in proteins known to be secreted. The cytoplasmic domain of AA4, in contrast, bears no structural similarity with known protein families. Instead, AA4 revealed strong homology to C1qR, the human receptor for complement component C1q which is predominantly expressed in phagocytic cells such as monocytes, neutrophils, and endothelial cells (Nepomuceno et al., 1997). Sequence alighnment shows that AA4 and C1qR have approximately 68% identical amino acid positions and share similar domain structure. Highest homologies were found within the Nterminal parts of the two proteins and their C-terminal cytoplasmic domains, suggesting that AA4 and C1qR may have similar functional properties. Expression of AA4 correlates with the expression of CD34 and CD43, two other surface markers normally present on murine hematopoietic stem and progenitor cells. Previous studies have shown that AA4-positive cells are first detected in yolk sac at day 8-10 of gestation (Cumano et al., 1993; Sanchez et al., 1996).

At day 9-10 pc AA4+c-Kit+Mac-1⁺ progenitors are found in the P-Sp/AGM region (Sanchez et al., 1996; Marcos et al., 1997), and by day 14 of gestation, AA4 defines 0.5-1.0% of the fetal liver tissue that contains the entire hierarchy of primitive hematopoietic cells (Jordan et al., 1990). As proliferation within each successive compartment results in increased total number progenitor cells, antigen density per cell also increases with developmental progress, which is especially marked for c-Kit and AA4 (Marcos et al., 1997). A significant proportion of hematopoietic progenitors from yolk sac also express CD34, CD38, CD43, CD44, and Mac-i however show little or no expression of CD4, CD8, CD45R and Sca-. These same markers are present on FLderived HSC which also express Sca-1. Adult marrow HSCs also express cKit, CD38 and Sca-1, but do not normally express Mac-I or AA4. In BM, HSC are found in both AA4+ and AA4-subpopulations, although in adult marrow AA4 is largely regarded to be a marker of early B lymphoid lineage 0. These results indicate that the expression of cell surface antigens changes on HSC during ontogeny and that differential display of theses cell surface markers may reflect relationship between HSCs that contribute to multilineage hematopoiesis and distinct anatomical sites during development.

Methods

Cells and Tissue Culture.

NIH 3T3 fibroblasts were grown in DME medium supplemented with 10% fetal calf serum (FCS). D2N cells were grown in RPMI 1640 medium containing 10% FCS. EML C1 cells were grown in IMDM supplemented with 20% horse serum and 8% BHK/MKL conditioned medium (Tsai et al., 1994). To maintain the multipotentiality of EML C1, the cells were kept at low density (0.5×5×10-5/ml) and subcultured every two days. Cell lines constitutively expressing AA4 and GFP were derived from NIH 3T3 or EML C1 cells by infecting with the corresponding recombinant retroviruses.

Retroviral-mediated Gene Transler.

Retrovirus expression vector REBNA was constructed by substituting the LacZ gene contained within the EcoRI-Notl fragment of plasmid LZRSPBMN-Z (Kinsella and Nolan, 1996) with a synthetic polylinker composed of EcoRl, Xhol, Sfil, and Noti sites. Retroviral vector REBNA/IRESGFP contains a poliovirus IRES element and the CDNA encoding color-enhanced GFP (S. Zolotukhin, Gainesville, Fla.) inserted into REBNA.

For DNA transfections, cells were plated at a density 2×106 cells per 60 mm dish and transfected with 5 ug of plasmid DNA using 20 ul of lipofectamine reagent (GibcoBRL). REBNA-transfected cells were selected in puromycin (2 ug/ml) and grown to confluence prior to collecting virus supernatant. For infections” the culture medium was supplemented with polybrene (Sigma) at 5 ug/ml. The appropriate virus was added and incubated overnight. Multiple infections were carried sequentially, with the appropriate selection between.

Isolation o.t RNA and CDNA Cloning.

Poly(A) RNA prepared from D2N cells was converted into CDNA using Superscript II Reverse Transcriptase (GibcoBRL) and an oligo(dt) primer containing Notl site, 5′-TGGTGTCGACGCAGAGTAGCGGCCGCT18 (SEQ.ID.No.:74). The second strand was synthesized using DNA polymerase I in combination with E.coli RNAse H and E.coli DNA ligase as described (Gubler and Hoffinan, 1983). An adaptor composed of complementary oligonucleotides, 5′-GGCCCGGGCCGGCC (SEQ.ID.No.:75) and 5′-TCGAGGCCGCCCGGGCC (SEQ.ID.No.:76), was ligated to the CDNA and cut with Notl to produce CDNA molecules with NotI and Xhol termini for directional cloning. After size fractionation in agarose gel, cDNAs larger than 2.5 kbp were ligated into Notl and XhoI cut plasmid REBNA and electroporated into electrocompetent DH12S cells (GibcoBRL). Plasmid DNAs were transfected into 293-derived packaging cell line for retrovirus production. Virus-containing supernatants were collected and stored at −80 C.

NIH 3T3 cells infected with the recombinant retroviruses representative of D2N CDNA library were selected for the production of AA4 by flow cytometry using phycoerythrin-conjugated AA4.1 mAb. After two rounds of sorting, genomic DNA isolated from AA4-positive cells was subjected to pcr amplification using retroviral vector primers, 5′CAGCCCTCACTCCTTCTC (SEQ.ID.No.: 77) and 5′-GGTGGGGTCTTTCATTCC (SEQ.ID.No.: 78) (Kitamura et al., 1995). Amplified CDNA was gel purified and subcloned into pbluescript SK and REBNA plasmid vectors. Nucleotide sequences were analyzed using NCBI Blast database search programs and ExPASy molecular biology server from the Swiss Institute of Bioinformatics.

Northern Blot Hybridization.

RNAs prepared using acid guanidinium thiocyanate-phenol extraction procedure (Chomczynski and Sacchi, 1987) were separated on formaldehydeagarose gels and blotted onto the Hybond-N nylon membranes (Amersham). Hybridization probes were derived from cloned cDNAs using Ready To Go DNA labeling beads (Pharnacia Biotech). Hybridizations were performed as described previously (Petrenko et al., 1997).

Cell labeling and Immunoprecipitations.

The ECL protein biotinylation system (Amersham) for the detection of cell surface proteins was used as recommended by the manufacturer. For radioactive labeling, 2×106 cells were incubated with 200 uCi Translabel (ICN) in 2 ml of cysteine and methionine-deficcient medium for 2 hr at 370 C. Cells were washed in PBS and lyzed 10 min. on ice in 500 ul of NP40 buffer containing 20 mM TrisHCl pH 7.6; 150 mM NaCl; 0.5% NP40; 1 mM PMSF; 5 mM benzamidine; 1 mM sodium vanadate; 10 ug/ml aprotinin; 10 ug/ml leupeptin. Lysates were cleared by centrifugation and 2 ug of soluble antibody were added to the supernatants followed by 25 ul of Protein G-Sepharose suspension (Sigma). After 2 to 4 hr incubation at 40 C. with rotation, protein Gantibody complexes were pelleted and washed in the successive changes of wash buffer I (1M NaCl; 10 mM TrisHCl pH 8; 0.1% NP40); wash buffer 2 (0.1M NaCl; 10 mM TrisHCl pH 8; 0.1% NP40); wash buffer 3 (10 mM TrisHCI pH 8; 0.1% NP40). Samples were eluted by boiling 2 min. in 2× gel loading buffer, separated by SDS/PAAG, dried and exposed to X-ray film.

Western Blot Analysis.

Protein extracts for Western blot analysis were prepared as described (Morrison et al., 1991). The antibodies used included goat anti-mouse CD43 polyclonal IgG (MI9 and S-19, Santa Cruz) in combination with HRPconjugated secondary antibodies and ECL detection system (Amersham).

Flow Cytometrv and RT-PCR.

Timed-pregnant mice and 5- to 7-week-old female mice (C57BI/6j) were purchased from Jackson Laboratory (Bar Harbor, Me.). AA4-positive cells were isolated from day 14 fetal liver by immunopanning on Petri dishes coated with AA4.1 antibody (10 ug/ml). Hematopoietic stem cells were purified from AA4-positive fraction by staining, with lineage-specific antibodies as described previously (Moore et al., 1997). Three-color fluorescence-activated cell sorting for lineage negative to low, Sca-1(+), c-Kit(+) cells was performed on a multilaser FACS Vantage with CeIlQuest software (Beckton Dickinson). ES cells differentiated into blasts cell colonies, hematopoietic progenitors, and endothelial cells prepared as described previously.

For RT-pcr, poly(A)-RNA isolated from sorted cells was converted into CDNA using Superscript II Reverse Transcriptase (GibcoBRL) and CapFinder CDNA amplification kit (Clontech). Gene-specific primers for pcr amplification included: 5′-TTCAGCAAGCCCTGACTC (SEQ.ID.No.:79) and 5′GCCACCTTCGAAGCAATC (SEQ.ID.No.:80) (AA4); 5′-GAGCGGTACAGGAGAATG (SEQ.ID.No.:81) and 5′GCCCACCCAACCAAATCA (SEQ.ID.No.:82) (CD34); 5′-ACCGCGTTCTTCTGTAAC (SEQ.ID.No.:83) and 5° CAGCTAACAGCAGGATCC (SEQ.ID.No.:84) (CD43); G3PDH Control Amplimer Set (Clontech) for the amplification of GAPDH.

### Example 3

### In Vitro Maintenance of Highly Purified, Transplantable Hematopoietic Stem Cells

The cellular and molecular mechanisms which regulate even the most primitive hematopoietic stem cell are not well understood. This example details a systematic dissection of the complex hematopoietic microenvironment in order to define some of these mechanisms. An extensive panel of immortalized stromal cell lines from murine fetal liver was established and characterized. Collectively, these cell lines display extensive heterogeneity in their in vitro hematopoietic supportive capacities. This example describes a long-term in vitro culture system, utilizing a single, stromal cell clone (AFT024) that qualitatively and quantitatively supports transplantable stem cell activity present in highly purified populations. Disclosed is multi-lineage reconstitution in mice that received the equivalent of as few as 100 purified bone marrow and fetal liver stem cells which were cultured for 4-7 weeks on AFT024. The cultured stem cells meet all functional criteria currently ascribed to the most primitive stem cell population. The levels of stem cell activity present after 5 weeks of coculture with AFT024 far exceed those present in short-term cytokine-supported cultures. In addition, the maintenance of input levels of transplantable stem cell activity is accompanied by the expansion of other classes of stem/progenitor cells. This suggests that the stem/progenitor cell population is actively proliferating in culture and that the AFT024 cell line provides a milieu which stimulates progenitor cell proliferation while maintaining in vivo repopulating activity.

Introduction

Mammalian blood formation originates in a small population of hematopoietic stem cells. The hallmark features of these cells are: (1) a hierarchical multilineage differentiation potential with the ability to clonally give rise to at least 8 distinct cell lineages, (2) self-renewal capacity which is reflected in the life long continuous activity of few, in some cases single, stem cells and (3) a dramatic proliferative potential which is ultimately responsible for the production of large numbers of mature blood cells. (Leminschka, I. R. 1992; Morrison, S. J. et al. 1995; Harrison, D. E. 1980). During the past decade much progress has been made in providing a physical phenotype for this rare population of stem cells. (Spangrude, G. J., et al. 1995; Lemischka, I. R. 1992; Bauman, J. G., et al. 1998). However, currently, the only reliable functional assay system for the most primitive stem cell compartment is long-term in vivo transplantation. No in vitro system has been developed which adequately recapitulates stem cell behaviors. Therefore, the cellular and molecular mechanisms that regulate the biology of stem cells have remained obscure.

A major challenge in stem cell research is the establishment of culture systems which facilitate in vitro maintenance of long-term transplantable stem cell activity. This is a necessary first step towards a cellular and molecular understanding of the regulatory mechanisms which mediate commitment versus self-renewal decisions. Moreover, the establishment of such culture systems is a prerequisite for the potential expansion of undifferentiated stem cell populations as well as for the generation of stem/progenitor cells committed to selected lineages.

Efforts to develop culture systems for the maintenance of transplantable stem cells can be subdivided into two broad categories: (1) those utilizing defined cytokine combinations as the only culture supplements and (2) those relying on a pre-established stromal monolayer as an additional supportive component (with or without exogenously added cytokines). Both of these strategies have met with only limited success. In the first case, it has been repeatedly demonstrated that combinations of cytokines can exert potent stimulatory effects on stem/progenitor populations. (Spangrude, G. J., et al. 1988; Jordan, C. R. et al. 1990; Fleming, W. H., et al. 1993). In some studies, highly purified stem cells (Li, C. L., and Johnson, G. R. 1990; Spangrude, G. J. and Johnson, G. R. 1990). have been used and the direct effects of cytokines have been demonstrated at the single cell level. (Jones, R. et al. 1990; Uchida, N. et al. 1993). While informative, the vast majority of these studies are limited by their strictly in vitro nature. Thus, it is feasible to expand, replatable in vitro progenitor populations (Li, CL., and Johnson G. R. 1992; Uchida, N. et al. 1993) and to stimulate colony-formation by cells with both myeloid-erythroid and lymphoid potentials, Jones, R., et al. 1990; Uchida, N. et al. 1993; Trevisan, M., and Iscove, N. N. 1995; Ogawa, M. 1993) however, the equivalence of these progenitor cells with the in vivo transplantable stem cell population remains speculative. Several studies have clearly demonstrated a dramatic loss of in vivo repopulating potential as a result of cytokine driven in vitro proliferation. (Knobel, et al., 1994; Peters, et al., 1995; Traycoff, et al., 1996). A small number of studies have shown that defined cytokine combinations promote the maintenance of transplantable activity.(Rebel., et al., 1994). However, most of these are limited both by the use of very short culture periods, the exact nature of the in vivo assay, and the use of non-enriched stem cell sources. (Muench, et al., 1993; Holyoake, et al., 1996; Soma, et al., 1996). This precludes interpretations suggesting a direct action of the given cytokine(s) in maintaining transplantable activity.

A further complication with defined cytokine studies is the inability to ascribe in vivo physiological relevance to the observed effects. It has long been accepted that in the intact animal, stem cells are found in close association with discrete cellular microenvironments. (Lord, et al., 1975; Trentin, et al., 1970; Weiss, et al., 1991; Wolf, 1979). These observations suggest both the existence of stem cell niches and the notion that in vivo stem cell regulatory mechanisms are likely to require cell-cell contact or short range interactions. (Dorschking, 1990). Efforts to understand the features of the hematopoietic microenvironment began with the establishment of the Dexter long-term culture (LTC) system. (Dexter, et al., 1977). In this culture system hematopoiesis is maintained for weeks or months by a heterogeneous adherent cell monolayer derived from bone marrow (BM). While some degree of transplantable stem cell maintenance and self renewal (Fraser, et al., 1990) has been demonstrated, a general feature of the Dexter-LTC is a dramatic net decrease of stem cell activity over time. (Harrison, et al., 1987; Van der Sluijs, et al., 1993). Although much progress has been made, especially in studies of human stem/progenitor cells, (Sutherland, et al., 1989; Verfaillie, et al., 1995; Hao, et al., 1996) a further drawback of this system is the heterogeneity of the stromal cell types present in the supportive monolayer. This hampers the identification of regulatory mechanisms. Studies have been reported where the heterogeneous stromal monolayer is replaced with cloned stromal cell lines. (Roberts, et al., 1987; Kodama, et al., 1984; Issad, et al., 1993; Wineman, et al., 1993). Many of these cell lines can support in vitro myelopoiesis, (Suzuki, et al., 1992; Neben, et al., 1993; Kodama, et al., 1992) B-lymphopoiesis (Collins, et al., 1987; Whitlock, et al., 1982) or in some cases both. Wineman, et al., 1993; Wineman, et al., 1996) However, very few studies have focused on the in vitro maintenance of the most primitive transplantable stem cell compartment. Moreover, with one exception (Szilvassy, et al., 1996) the studies which have focused on the in vitro maintenance of this stem cell population begin with heterogeneous unpurified sources of hematopoietic activity. Wineman, et al., 1992, 1996; Deryugina, et al., 1994). Such populations contain numerous non-hematopoietic stromal cell types. Therefore, it has not yet been possible to assign a direct stem cell supporting phenotype to a given stromal cell line.

It was hypothesized that the rare frequency of primitive, stem cells may suggest an equally rare frequency of stem cell supporting microenvironrnental niches. Accordingly, we established and characterized a large panel of conditionally immortalized, cloned stromal cell lines from mid-gestation fetal liver. This organ was chosen because, during development, it is here that the stem cell compartment is undergoing self-renewal expansion in addition to differentiation. (Moore, et al., 1970). The cell lines were generated as previously described, (Wineman, et al., 1996) by immortalization with a temperature sensitive SV40TAg. (Frederiksen, et al., 1988) The clonal nature of the AFT024, 2018, and 2012 cell lines was verified by Southern blot analysis which detected a single, unique proviral integration locus in their genomic DNA. In order to identify potentially interesting cell lines, we used a “cobblestone area” (CA) assay (Ploemacher, et al., 1989) was initiated with BM taken from mice injected two days previously with 5-fluorouracil (5-FU). It has been suggested that CA colonies which appear after a prolonged culture period are derived from more primitive stem cells, possibly identical to some in vivo transplantable entities. (Ploemacher, et al., 1991). Therefore, a goal was in identifying cell lines which support such late arising CAs. Of 225 lines, 77 (34%) were capable of supporting limited in vitro hematopoiesis, while, consistent with the initial hypothesis, only 2% were able to maintain long-term (>6 weeks) hematopoietic CA activity. Subsequent studies with a selected subset of these lines, showed that the ability to effectively support in vivo reconstituting BM stem cells is infrequently observed. (Wineman, et al., 1996). Two out of sixteen cell lines maintained significant levels of long-term reconstituting stem cell activity for an in vitro culture period of three weeks. Several other cell lines supported low levels of such activity or transiently repopulating stem cells. The cell inoculum, in these studies, was whole BM which was not enriched for stem cell activity. Therefore, it was not possible to suggest that the effective stromal cell lines were directly supporting stem cell activity. This example demonstrates that a single clonal cell line, designated AFT024, can maintain quantitative levels of transplantable stem cell activity present in highly purified stem cell populations. These data were generated using a competitive repopulation assay system, which employs uncompromised competitor BM cells. The in vitro-maintained stem cells satisfy all criteria which currently define the most primitive stem cell population including the ability to reconstitute secondary recipients. This example also shows that the in vitro maintenance of primitive transplantable stem cells is compatible with the concurrent generation of large numbers of committed progenitors.

Methods

Mice.

Timed-pregnant mice and 5-7 weeks old females (C57B1/6J, Ly5.2) were purchased from the Jackson Laboratory (Bar Harbor, Me.). Congenic C57B1/6, Ly5.1 female mice were purchased from the National Cancer Institute (Frederick, Md.). All mice were housed in the Princeton University Barrier Animal Facility, in autoclaved micro-isolator cages on ventilated cage racks. The animals received sterile, irradiated food, and acidified, autoclaved water ad libitum.

Stromal Cell Lines and Culture Conditions.

The fetal liver stromal cell lines used in this study were derived as previously described. (Wineman, et al., 1996). Stromal cell lines were routinely cultured in Dulbecco's modified Eagles's medium (DMEM) supplemented with 10% fetal bovine serum (FBS), 5×10⁻⁵ mol/L b-mercaptoethanol (2-ME), at 32° C., 5% CO₂, 100% humidity. Sera were obtained from Hyclone, Logan, Utah. Other biochemical reagents were obtained from Sigma, St. Louis, Mo. Two of the lines used in this study (2012 and 2018) were previously characterized for their ability to support long-term repopulating activity present in whole unfractionated BM. (Wineman, et al., 1996). The AFT024 cell line was identified as an additional long-term (>4 weeks) CA supporter. Subclones of 2012 and AFT024 were isolated and used in these studies. The AFT024 cell line has remained stable and demonstrated consistent stem cell supporting abilities for over 4 years.

Hematopoietic Stem Cell Purification.

Stem cells were purified from day 14 fetal livers essentially as described, (Jordan, et al., 1990) with the inclusion of c-kit expression as an additional parameter. Briefly, AA4.1⁺ cells were isolated by immunopanning on petri dishes coated with AA4.1 antibody (10 ug/mL). The AA4.1⁺ fraction has been shown to contain all repopulating stem cell activity present in day 14 fetal liver. (Jordan, et al., 1990) AA4.1⁺ cells were collected and stained with saturating concentrations of fluorescein isothiocyanate (FITC) labeled rat monoclonal antibodies to lineage markers (CD3, CD4, CD5, CD8, B220, Gr-1, Mac-1, and TER-119). The cells were simultaneously stained with phycoerythrin (PE) labeled Ly6A/E (Sca-1) antibody and biotinylated antibody to c-kit. The latter was developed with streptavidin allophycocyanin (APC). The AA4.1 hybridoma was a kind gift from Dr. J. McKeam, Monsanto, St. Louis, Mo. AA4.1 antibody was purified by ImClone Systems Inc. New York, N.Y. The TER-119 antibody was initially obtained from Dr. T. Kina, Kyoto University, Japan and subsequently purchased from PharMingen, San Diego, Calig. All other antibodies were purchased from PharMingen. Streptavidin APC was purchased from Molecular Probes Inc., Eugene, OR. Three color fluorescence activated cell sorting for lineage negative to low (lin^{-l/o}), Sca-1⁺, c-kit⁺ cells was initially done on a dual laser Epics 753 cell sorter (Coulter Electronics, Hialeah, FA) interfaced with Cicero software (Cytomation Inc., Fort Collins, Colo.) and subsequently on a multi-laser FACS Vantage with Cell Quest software (Becton Dickinson Immunocytometry Systems, San Jose, Calif.).

Enriched fractions of BM stem cells were obtained from C57B1/6 Ly5.1 mice as described. (Okada, et al., 1992). Briefly, BM mononuclear cells were isolated by density centrifugation over Ficoll Hypaque (<1.077, Pharmacia, Piscataway, N.J.). Lineage negative or low staining cells (lin^{-/lo}) were obtained by magnetic bead depletion (anti-rat immunoglobulin coated beads, Dynal, Oslo, Norway) of mononuclear cells using the same lineage cocktail described herein above. The cells were further stained with antibodies to Sca-1 and c-kit as described herein above. Sorting for lin^{-/lo}, Sca-1⁺, and c-kit⁺ cells was accomplished with the Epics 753 as described above.

Stem Cell/Stromal Cell Cocultivation and Cytokine-supplemented Suspension Culture.

Stromal cell lines were seeded on tissue culture dishes that had been coated with 1% gelatin (Specialty Media, Lavallette, N.J.) and were grown at 32° C., 5% CO₂, 100% humidity. Confluent monolayers were irradiated (20 Gy, ³⁷Cesium source, Gammacell 40, Nordion International Inc. Ontario, Canada) and cultured in modified Dexter (Dexter, et al., 1984) media (DMEM, 10% FBS, 10% horse serum, 5×10⁻⁵ mol/L 2-ME, 1×10⁻⁷ mol/L hydrocortisone). For Dexter-LTC, enriched hematopoietic stem cells were added and the cultures were maintained at 37° C., 5% CO₂, 100% humidity with weekly media changes. The specific numbers of purified stem cells added to stromal cell cocultures are given in the appropriate figure or table legends. In some experiments, week 4 AFT024/stem cell cultures were harvested and replated in limiting-dilution onto fresh, irradiated (20 Gy) AFT024 monolayers in 96-well trays (Dexter-LTC conditions). CAs were scored weekly (as described above), for an additional 5 weeks. Irradiated (20 Gy) 2018 monolayers in 96-well trays were used in limiting-dilution Whitlock-Witte assays (LD-WW) (Whitlock, et al., 1982) to assess stromal-dependent B-lymphopoiesis content of both freshly purified and AFT024 cultured fetal liver stem cells. These cultures were established in RPMI media with 5% FBS, 2 mmol/L glutamine, 1 mmol/L Na pyruvate, and 5×10⁻⁵ mol/L 2-ME at 37° C., 5% CO₂, 100% humidity. 2018 has been identified as a potent B-lymphopoiesis supporting line in W-W conditions. (Deryugina, et al., 1994). Short-term cytokine-supported suspension cultures and short-term AFT024/stem cell cocultures were established in Iscove's Modified Dulbecco's Media (IMDM), 10% FBS, 1% BSA, 5×10⁻⁵ mol/L 2-ME at 37° C., 5% CO₂, 100% humidity. Cytokine concentrations: rmflk2/flt3-ligand (FL) 30 ng/mL, rmSteel factor (SL) 20 ng/mL, rhIL-6 10 ng/mL. FL was obtained from ImClone Systems Inc.; SL was purchased from Genzyme Corporation, Cambridge, Mass. IL-6 was purchased from Upstate Biotechnology Inc., Lake Placid, N.Y.

Transplantation Assays for Hematopoietic Stem Cell Activity.

Competitive repopulation was used to measure stem cell activity present in both freshly isolated and cultured stem cell populations. (Harrison, et al., 1993). This assay was performed using the congenic Ly5.1/5.2 mouse system (Morse, et al., 1987). Enriched stem cells were seeded onto irradiated stromal monolayers and maintained in Dexter-LTC conditions. At the end of 4-7 weeks, the cultures were harvested by vigorous trituration. Single cell suspensions were prepared by passage through 22-gauge needles, mixed with fresh congenic BM and transplanted into lethally irradiated congenic mice (10 Gy, split dose 3 hours apart, 1 Gy/min, Gammacell 40). All purified fetal liver stem cells were from Ly5.2 mice. The competitor BM and recipients were Ly5.1. Purified BM cells were from Ly5.1 mice; in this experiment, Ly5.2 BM was used as competitor and Ly5.2 mice were used as recipients. In order to assess reconstitution, mice were periodically bled by capillary puncture of the orbital venous plexus. Blood (0.1 mL) was collected into heparin-containing (10 U/mL) DMEM and the red blood cells were lysed with NH₄Cl (Mishell, et al., 1980). For the experiments described in FIG. 15 and Table 4., the nucleated cells were divided into two fractions and stained with the appropriate biotinylated LyS antibody and developed with streptavidin-peridinin chlorophyll protein (PerCP) and; (1) CD4-FITC, CD8-PE and (2) B220-FITC, Mac-1-PE, Gr-1-PE. Cells from each fraction were analyzed on an Epics Profile II, Coulter Electronics. For the experiments described in FIG. 16 and Tables 2. and 3., nucleated cells were stained with directly conjugated lineage antibodies (CD4-PE, CD8-PE, Mac-1-FITC, Gr-1-FITC, and B220-APC) and biotinylated Ly5.2 antibody which was developed with streptavidin Texas Red (T.R.). Four color analysis of stained cells was performed on either the Coulter Epics 753 or Becton Dickinson FACS Vantage with the appropriate software interfaces described above. Anti-Ly5.1 was a kind gift of Dr. H. Nakauchi, University of Tsukuba, Japan. Purified and biotinylated Ly5.2 antibody was originally obtained from a hybridoma (AL14A2) kindly provided by Dr. G. Spangrude, University of Utah Medical Center, Salt Lake City, Utah. In later experiments, the Ly5.2 antibody was purchased from PharMingen; CD4-PE, CD8-PE, and B220-APC were also purchased from PharMingen. Streptavidin-PerCP was purchased from Becton Dickinson Immunocytometry Systems. Streptavidin T.R. was purchased from Molecular Probes. Competitive repopulating units (CRU) per 10⁵ were calculated according to the formula: (Harrison, et al., 1990).

$$CRU / {10}^{5} = \frac{\% Ly5 positive cells}{100 - \% Ly5} \times \frac{cell number competitor BM}{number of test cells}$$

Reconstitution values of less than 2% of the test Ly5 donor allele were not considered sufficiently above background for calculation of CRU.

Retransplantation potential of stem cells was assessed by secondary transplantation. Mice from the experiment presented in FIG. 14. were sacrificed 60 weeks after transplantation, BM was harvested and stained with antibody to Ly5.2 followed by streptavidin T.R. Ly5.2⁺ cells were collected by cell sorting (Coulter Epics 753) and used to transplant lethally irradiated secondary Ly5.1 recipients. Marrow from mice in the Control and AFT024 groups was used to transplant mice in both radioprotection and competitive repopulation assays. Ly5.2⁺ BM from the primary 2012 transplants was used only in radioprotection assays. Primary 2018 mice did not contain sufficient Ly5.2⁺ cells for secondary transplantation. Transplanted mice were bled and analyzed by 4-color flow cytometry for the presence of Ly5.2⁺ cells and multilineage reconstitution as described above.

In vitro Hematopoictic Progenitor Cell Assays.

The progenitor content of freshly purified hematopoietic stem cell populations and AFT024/stem cell cocultures was assessed using a variety of in vitro assays. All of the following assays were accomplished with fetal liver stem cells enriched as described herein above. To determine the time course of CA development, enriched stem cells were seeded onto irradiated AFT024 monolayers (300-600 cells/well in 12-well trays). CA development was followed over time and characteristic clusters were quantitated as described above. At different time points of the stem cell/AFT024 cultures, individual wells were harvested and replated into cytokine-supplemented semisolid clonogenic progenitor assays (CFU-C). The cytokine-enriched (rmIL-3 10 ng/mL, rhIL-6 10 ng/mL, rmSL 50 ng/mL, Epo 3 U/mL) methyl-cellulose mixture was purchased from Stem Cell Technologies Inc., Vancouver BC, Canada. Colonies were scored after 8-14 days of culture at 37°, 5% CO₂, 100% humidity according to established criteria. (Testa, et al., 1993). Colonies that reached >Imm in size after 8 days and which contained erythroid bursts and multiple myeloid cell lineages including megakaryocytes were scored as high-proliferative potential-mixed lineage colonies (CFU-HPP-Mix). (Lowry, et al., 1995). Lineage content of typical colonies was determined by Wright's/Giemsa staining of cytospin slide preparation from individual colonies. Colony assays were also done with 103 freshly purified cells. The CFU progenitor contents of the AFT024 cocultures were normalized to an initial input of 10³ stem cells. To assess the ability of the AFT024 cell line to maintain primitive lymphoid progenitors, 4 week cocultures were plated into LD-WW assay on 2018 cells as described above. Resulting pro-B cell colonies were scored after 7 days. The cell number in individual wells (96-well trays, 8 wells/cell number) was normalized from the original number of purified stem cells that initiated the coculture, i.e. stem cell equivalents/well. As calculated from the line of best fit, the cell number at 37% negative wells is the frequency of pro-B cell colony initiating cells in the starting population. (Taswell, 1981). In a similar manner, the frequency of CA initiating cells in week 4 stem cell/AFT024 cocultures was also determined by replating them in limiting-dilution onto fresh, irradiated AFT024 monolayers in 96-well trays. CA were scored as described above, at 20 1, 2, 3, 4, and 5 weeks after replating in Dexter-LTC. The resulting frequencies were calculated as described herein above for the LD-WW assays and are also expressed in relationship to the number of stem cells that seeded the initial cultures (stem cell equivalents).

Results

In vivo and in vitro Assays for Stem Cell Activity Maintained by Stromal Cell Lines.

Highly enriched stem cell populations were used to initiate cultures supported by single stromal cell lines. The focus was on one cell line, AFT024, which exhibited particularly potent stem/progenitor cell support. Two other stromal cell lines, 2012 and 2018, (Wineman, et al., 1996) were included in some experiments. In order to more rigorously establish the clonality of these lines, they were subcloned by limiting-dilution. All subclones obtained from a given cell line contained the same proviral integrant position as the parental cell line. The AFT024 cell line was evaluated both for its ability to maintain in vivo competitive repopulating stem cells, as well as a broad spectrum of stem/progenitor cells defined by a variety of in vitro assays. The in vivo assays focused on the ability of stem cells, cultured for extended time periods (4-7 weeks), to permanently reconstitute multi-lineage hematopoiesis in transplanted hosts. The in vitro assays included the enumeration of CA appearing over time in the initial cultures as well as the quantitation of stem/progenitor cells which can form colonies in cytokine-supplemented replating assays. Cells from four-week AFT024/stem cell cocultures were also assayed by limiting-dilution for the content of progenitors capable of initiating secondary CAs on AFT024 or B-lymphopoiesis in Whitlock-Witte cultures supported by 2018.

AFT024 Maintains Quantitative Levels of Long-term in vivo Repopulating Stem Cell Activity.

One line of investigation inquired if and at what levels in vivo transplantable stem cell activity was present in four to seven week-old cultures initiated with highly enriched stem cells and supported by AFT024, 2012 or 2018. Purified day 14 fetal liver cells (AA4.1⁺, lin^{-/lo}, Sca-1⁺, c-kit⁺) and adult BM cells (lin^{-/lo}, Sca-1⁺, c-kit⁺) were used as sources of stem cell activity. Both of these populations are about 1000 to 1500-fold enriched for stem cell activity, as measured by competitive repopulation. (Harrison, et al., 1993). The Ly5.1/Ly5.2 congenic system was utilized for all competitive repopulation studies. (Morse, et al., 1987). The data presented in FIG. 14 demonstrate that the cultures supported by AFT024 contain stem cell activity at levels quantitatively identical to those present in the uncultured purified populations. In this experiment, individual Ly5.1 mice received 10³ freshly purified Ly5.2 cells or the cultured equivalent of 10³ purified Ly5.2 cells. Each mouse also received 10⁶ Ly5.l competitor BM cells. The percentage of Ly5.2 positive peripheral blood cells was approximately equal in both groups of recipient animals. Moreover, the cultured stem cell activity is as effective as freshly purified activity for in vivo periods of greater than one year. The data in FIG. 14 also show that the 2018 cell line is completely ineffective in maintaining highly purified stem cell activity while the 2012 cell line supports intermediate levels of repopulating activity. The data presented in Table 4A. provide quantitative competitive repopulating unit (CRU) value calculations as well as the results of multiparameter lineage analyses. The extremely low levels of reconstitution by 2018-cultured stem cells precluded lineage analysis. The CRU values of the AFT024-cultured and freshly purified populations are nearly identical. Moreover, both fresh and AFT024-cultured stem cells reconstitute myeloid and lymphoid cell populations to a similar degree. In order to further access the supporting activities of AFT024 and 2012 we utilized a 10-fold lower number of fetal liver stem cells from two separate purifications to initiate the cocultures. The cultures were continued for 4-7 weeks, harvested, and used in competitive repopulation studies. Each recipient received the cultured equivalent of 100 purified Ly5.2 stem cells plus 4×10⁵ Ly5.1 competitor BM cells. A total of twelve mice were transplanted with AFT024 cocultures (four each after four, five and seven weeks of LTC). The parental AFT024 line was used in the 4 week group and two different subclones were used to support the five and seven week cultures. The AFT024-cultured Ly5.2 stem cells contributed to 20-30% of peripheral blood cells in these recipients while cells cultured on 2012 demonstrated more limited in vivo function (Table 4B). The 2012 cultures were done with two subclones of the parental line and were maintained for four weeks prior to harvest and transplant (four mice/subclone). The data utilizing different cultures time or sublcones did not vary significantly from each other and are presented together in Table 4B. An additional experiment was undertaken using enriched BM which was cultured on AFT024 and 2018 for six weeks. In this experiment, BM was purified from Ly5.1 congenic mice. Each Ly5.2 recipient mouse in this study received 100 freshly purified cells or the cultured equivalent of 100 purified cells. Both groups received 10⁵ Ly5.1 competitor BM cells per mouse. Data analysis, for the presence of Ly5.1⁺ cells at 4 months after transplant, is presented in Table 4C. For 6 weeks of culture, AFT024 cells maintained quantitative levels of reconstituting activity present in 100 purified BM stem cells. The 2018 cell line failed to maintain stem cell activity.

These studies were extended to include secondary transplantation as an additional assay for primitive stem cells. BM cells were harvested from the primary recipients of fresh and cultured fetal liver stem cells (see FIG. 14. and Table 4A.) and the Ly5.2 positive, fetal liver-derived fraction was collected by cell-sorting. Secondary radioprotection and competitive repopulation transplants were performed. The data are presented in Table 5. The secondary recipient repopulating activities are nearly identical for the AFT024 cultured stem cells and the non-cultured controls. Lineage analysis of the Ly5.2 cells in the secondary recipients revealed similar numbers of myeloid and lymphoid cells derived from both AFT024-cultured and non-cultured stem cells. Some level of secondary reconstituting cell activity was observed.

Additional experiments were also performed to determine where the levels of stem cell activity present in long-term AFT024 cocultures were compared to those present in short-term cytokine stimulated cultures or in short-term AFT024-supported cultures (FIG. 15). Purified fetal liver cells were seeded onto an AFT024 monolayer and maintained in Dexter-LTC conditions for 5 weeks. Simultaneously, the same numbers of purified cells were cultured for 5 days with; (1) different cytokine combinations or (2) on AFT024. The transplantable activity in the cultured cells was then assayed by competitive repopulation. Each mouse received the cultured equivalent of 600 stem cells together with 4×10⁵ Ly5.1 competitor BM cells. It is evident from the data that the levels of in vivo repopulating activity present in the long-term AFT024-supported cultures are much greater than those remaining after a short-term cytokine-supported culture period. Of interest also is that short-term AFT024 stem cell cocultures do not maintain significant in vivo reconstituting activity. In fact, these levels of stem cell activity are identical to the levels seen in the cytokine-supported cultures.

In vitro stem/progenitor populations are expanded by AFT024. AFT024/stem cell cocultures have vigorous hematopoiesis throughout the entire iii vitro culture period. This is reflected in the large numbers of relatively mature hematopoietic cells which are produced throughout the culture period. In addition, CA colonies are observed throughout the culture period. FIG. 16 shows a time course of CA appearance with purified fetal liver stem cells (3 separate experiments). After 28 days in culture, approximately one in every twenty input stem cells is capable of proliferating into a CA. In addition, CA appearance over time follows a biphasic distribution, with many CA observed early in the culture period. In order to enumerate the various classes of stem/progenitor cells present in AFT024 cocultures, we performed a series of in vitro replating experiments. These included the quantitation of: (1.) progenitor cells capable of colony-formation in cytokine-supplemented semisolid cultures (CFU assay), (2.) progenitor cells capable of initiating secondary CA in limiting-dilution AFT024 cultures and (3.) progenitor cells which can initiate B-lymphopoiesis in LD-WW cultures. In all of these experiments the primary cultures were initiated with purified fetal liver cells. All data presented below are normalized to an initial input of 103 purified cells (CFU assay) or the actual number of initial input stem cells (limiting-dilution assays).

Shown in FIG. 17 are the numbers and types of cytokine responsive CFU progenitors present at various times in the AFT024-supported cocultures. Production of CFU is evident at all time points. However, the content is especially high after four weeks, representing a 5-7 fold increase/expansion when compared to the content in the freshly purified populations. The content of more primitive progenitors (CFU-HPP-Mix) is increased by 12-fold. These HPP-Mix colonies often reach a size of 2 mm in 8 days and contain large numbers of erythroid bursts and megakaryocytes. Interestingly, there does not appear to be a correlation between CA number and CFU content at different culture times. This is most apparent at day 6, when CA numbers are at their peak but the progenitor content is similar to that observed in non-cultured stem cells. Furthermore, there is no correlation between CFU content and the absolute numbers of maturing hematopoietic cells present in a given culture.

Next, the content of primitive B-lymphoid progenitors present in the AFT024/stem cell cultures was determined. This was accomplished by plating cells from the four-week AFT024 cocultures into LD-WW assays over the 2018 stromal cell line. Two experiments with freshly purified stem cells and AFT024-cultured stem cells showed that the frequency of pro-B cell progenitors is expanded 10-fold in AFT024 cultures compared to the frequency observed in the freshly purified input population (day 0 frequency 1 in 11.0, r²=0.98; day 28 AFT024-cultured frequency 1 in 1.1, r²=0.97).

In order to measure the content or frequency of progenitor cells capable of initiating secondary CA, 4 separate, four-week AFT024/stem cell cocultures were replated in limiting-dilution onto fresh AFT024 monolayers. CAs were scored after one week. The data are presented in FIG. 18A. Large numbers of secondary CAs were observed. When normalized to the stem cell numbers used to initiate the primary cultures (stem cell equivalents, see Methods), the frequency of these progenitors is 1 in 3 to 4. FIG. 18B. shows data from one of the 4 experiments presented in FIG. 18A., where the quantitation of secondary CAs was extended for 4 more weeks. The frequency of CA decreases slowly over time (1 in 19 after an additional 4 weeks), approximating the frequency seen in the primary cultures at four weeks. In summary, our in vitro replating assays collectively demonstrate a significant expansion of primitive progenitor populations in 4 week AFT024 cultures. In these same cultures there is no decrease in the levels of transplantable stem cell activity present in the total hematopoietic cell population.

Discussion

In this example, it was demonstrated, that the AFT024 stromal cell line can maintain quantitative levels of in vivo repopulating stem cells for at least 7 weeks of in vitro culture. Highly enriched stem cell populations in low numbers (100 cells) were used and cell activity was measured in a stringent, competitive repopulation assay system. The cultured stem cell activity satisfies all in vivo criteria normally ascribed to the most primitive stem cell compartment; (1) long-term engraftment ability, (2) multilineage potential and (3) the ability to repopulate secondary recipients. In addition, the studies with the low culture initiating stem cell numbers for both BM and fetal liver, imply that AFT024 stromal cells exert their supportive effects in a direct manner. These studies represent a clear example of an in vitro system capable of directly supporting the most primitive stem cell compartment.

Other data revealed that stromal cell lines isolated from a single tissue source are heterogeneous with respect to their abilities in maintaining long-term repopulating stem cells. (Wineman, et al., 1996). It was speculated that the rare cell lines which were effective in supporting in vivo reconstituting stem cells may represent immortalized components of in vivo stem cell niches. However, the data argue for the necessity of using purified stem cell populations in order to support such a hypothesis. Specifically, the 2018 cell line maintained transiently reconstituting activity present in unfractionated BM. However, 2018 fails to maintain measurable repopulating activity when cultured for 4-6 weeks with highly purified BM or fetal liver stem cells. Similarly, in experiments using purified cells, there was a failure to show robust levels of reconstituting stem cell activity in cultures supported by CFC034, the most effective cell line in the whole BM studies (Wineman, et al., 1996). The 2012 cell line which was reported (Wineman, et al., 1996) to be effective in maintaining long-term repopulating whole BM derived stem cells is only partially effective in the present studies. Moreover, only some subclones of 2012 display such activity (in spite of identical proviral integration positions in all subclones). Recently, studies have shown that the S17 cell line which consistently supports the stem cell activity present in whole BM, (Wineman, et al.,. 1993) is not similarly effective in the maintenance of purified BM stem cells. (Szilvassy, et al., 1996). Taken together with these current data, the previously observed stem cell supporting stromal cell activities may reflect the action(s) of indirect mechanisms and therefore do not permit the identification of cellular stem cell niche components. One previous study has shown that the Sysl stromal cell line can maintain high levels of transplantable activity present in purified BM. (Szilvassy, et al., 1996). The competitor cells in that study were compromised by prior serial transplantation. Moreover, the culture period was extended for only two weeks and effective maintenance required the addition of exogenous leukemia inhibitory factor. In contrast, AFT024 is a cell line that provides a direct-acting long-term stem cell supporting environment without the addition of exogenous factors.

In addition to recovering net input levels of transplantable activity from AFT024 supported cultures, significantly expanded populations of primitive progenitor cells were also obtained. CFU-HPP-Mix progenitors are expanded by 12-fold after 4 weeks of culture and the numbers of stromal-dependent pro-B lymphoid progenitors are similarly amplified. This suggests that the AFT024-mediated process of stem cell maintenance is in reality a dynamic phenomenon. Specifically, during the first portion of the culture period, the majority of transplantable stem cell activity may be lost, through differentiation or cell death. The remaining primitive stem cells may expand to yield input levels of transplantable activity as well as increases in the numbers of more committed progenitors. One hypothesis is that short-term AFT024 supported cultures should contain reduced levels of transplantable stem cell activity. FIG. 17 support this hypothesis. The standard Dexter-type media used in parallel long-term AFT024 cocultures was not used in these short-term cultures. However, in another short-term experiment, utilizing Dexter-LTC media, a similarly dramatic reduction in stem cell activity was observed after 4 days of culture on AFT024.⁶³ These observations are intriguing because they suggest that the AFT024 cell line is able to facilitate some degree of ex vivo transplantable stem cell proliferation and expansion. Indeed, in other studies it was shown that AFT024 can support colony formation initiated by single purified stem cells with B and T-lymphoid, mycloid and erythroid potentials. Moreover, the data suggest that is possible to efficiently introduce retroviral markers into transplantable stem cells at various times during AFT024 cocultures. Extension of such marking experiments and an analysis of proviral integration patterns will be necessary to rigorously ascertain if self-renewal replication is occurring during these coculture periods.

The ability of AFT024 to maintain the most primitive stem cell compartment while generating and expanding at least some less primitive members of the stem/progenitor cell hierarchy raises interesting issues regarding the nature of stem cell niches. The present invention suggests that microenvironmental niche models which postulate distinct cellular entities responsible for stem cell self-renewal and other cellular entities which support the generation of committed progenitor cells may be overly simplified. (Uchida, et al., 1993). Quite clearly a single microenvironmental cell type represented by AFT024 is sufficient for keeping stem cells in an undifferentiated state as well as allowing commitment and progenitor expansion to take place. A hallmark feature of a stem cell niche is the ability to facilitate the generation of the entire stern/progenitor cell hierarchy from very primitive cells. Therefore, the main functional role of such niches may be to provide an environment which permits the production of the correct numerical balance of more and less primitive stem/progenitor cell entities. This model contains several testable hypotheses. The most important is that in vitro stem cell maintenance should not be interpreted, literally, as the maintenance of quiescent cells but rather as a phenomenon which results from a balance of self-renewal and commitment decisions which occur during stem cell division.

A cytokine cocktail of IL-6, SL, and FL is not effective in maintaining fetal liver stem cell activity. It was shown that RNA transcripts for these and 10 other cytokines are present in AFT024, but they are also detected at similar levels in non-supporting lines such as 2018. (Wineman, et al., 1996). These observations suggest the existence of novel AFT024-derived molecules which may act on stem cells. Indeed, using a subtractive hybridization molecular cloning strategy, a number of candidate molecules have been identified. Two of these molecules contain EGF-like repeat motifs that are most closely related to those found in the Notch/Notch-ligand family. Interestingly, one of these molecules appears to have activity on primitive stem cell populations. (Moore, et al., 1997). (See, Examples presented herein above.

Table 4. Multilineage stem cell activity, high CRU levels maintained in AFT024 cultures

A. With 1000 Purified Fetal Liver Cells

| | | Lineage Contribution, % Ly 5.2 Cells | | | | Total | |
|---|---|---|---|---|---|---|---|
| Cells | Week | CD4 | CD8 | B220 | Myeloid | % Ly 5.2 | CRU/10⁵ |
| Control | 5 | 3.3 ± 1.5 | 4.4 ± 1.7 | 48 ± 5.1 | 32 ± 6.0 | 28 ± 1.3 | 386 ± 24 |
| | 12 | 27 ± 10 | 24 ± 8.7 | 63 ± 8.9 | 43 ± 9.2 | 44 ± 3.2 | 800 ± 102 |
| | 24 | 42 ± 6.9 | 34 ± 4.1 | 71 ± 4.4 | 62 ± 1.4 | 48 ± 4.8 | 984 ± 171 |
| AFT024 | 5 | 7.7 ± 6.2 | 6.0 ± 3.5 | 58 ± 6.6 | 36 ± 4.7 | 39 ± 2.1 | 641 ± 54 |
| | 12 | 44 ± 7.1 | 38 ± 6.4 | 70 ± 5.6 | 53 ± 3.6 | 48 ± 1.4 | 942 ± 51 |
| | 24 | 56 ± 3.8 | 49 ± 3.1 | 72 ± 2.4 | 64 ± 3.7 | 56 ± 1.9 | 1270 ± 96 |
| 2012 | 5 | 0.8 ± 0.5 | 1.4 ± 0.3 | 6.8 ± 3.6 | 14 ± 11 | 7.4 ± 2.3 | 52 ± 8.5 |
| | 12 | 9.1 ± 8.4 | 10 ± 6.9 | 14 ± 9.3 | 23 ± 20 | 14 ± 4.5 | 194 ± 68 |
| | 24 | 17 ± 3.8 | 18 ± 2.0 | 23 ± 5.9 | 32 ± 6.4 | 21 ± 4.4 | 196 ± 71 |

B. With 100 Purified Fetal Liver Cells

| | | Lineage Contribution, % Ly 5.2 Cells | | | Total | |
|---|---|---|---|---|---|---|
| Cells | Week | Granulocytes | B-cells | T-Cells | % Ly 5.2 | CRU/10⁵ |
| AFT024 | 15 | 33 ± 4.6 | 41 ± 6.0 | 25 ± 5.3 | 28 ± 4.6 | 1888 ± 315 |
| | 26 | 17 ± 4.4 | 18 ± 4.8 | 18 ± 5.9 | 24 ± 5.9 | 1825 ± 571 |
| | 46 | 22 ± 5.7 | 19 ± 6.6 | 15 ± 6.7 | 20 ± 6.5 | 1595 ± 628 |
| 2012 | 15 | 32 ± 7.8 | 28 ± 7.2 | 20 ± 5.2 | 24 ± 6.6 | 1390 ± 382 |
| | 26 | 10 ± 2.8 | 9.7 ± 3.2 | 9.7 ± 3.2 | 14 ± 3.9 | 699 ± 176 |
| | 46 | 11 ± 2.3 | 6.7 ± 1.6 | 8.9 ± 3.2 | 9.0 ± 2.0 | 409 ± 80 |

C. With 100 Purified Bone Marrow Cells

| | No. of | Lineage Contribution, % Ly 5.1 Cells | | | | Total | |
|---|---|---|---|---|---|---|---|
| Cells | mice | CD4 | CD8 | B220 | Myeloid | % Ly 5.1 | CRU/10⁵ |
| Control | 8 | 38 ± 7.3 | 30 ± 5.8 | 43 ± 6.7 | 31 ± 4.8 | 32 ± 6.5 | 583 ± 172 |
| AFT024 | 7 | 20 ± 9.8 | 18 ± 8.8 | 20 ± 8.7 | 24 ± 11 | 22 ± 10 | 490 ± 296 |
| A. AA4.1′ day 14 fetal liver cells (Ly 5.2) were further purified for a lin⁻¹⁰, Sca-1′. ckit′ stem cell surface phenotype. Fresh purified control cells, 10³, were transplanted with 10⁶ Ly5.1 competitor marrow (n = 6 mice). From the same purification, 10⁴ cells were cocultured with stromal cell lines for 4 weeks. Subsequently, 10% of each culture was transplanted per mouse (n = 8 mice/stroma) together with 10⁶ | | | | | | | |
| #competitor BM cells. The contribution to each lineage in peripheral blood is expressed as the percent of the total specific lineage population that was Ly5.2⁺. CRU/10⁵, relative enrichment of competitive repopulating units. Data are presented ± SEM. | | | | | | | |
| B. The lineage and CRU content of low numbers of enriched fetal liver stem cells maintained on AFT024 and 2012 were determined. 500 stem cells were maintained in Dexter-LTC for 4-7 weeks over irradiated monolayers. 20% of each culture was used to transplant groups of 4 mice (i.e. each mouse received the equivalent of 100 stem cells that initially seeded the cultures) combined with 4 × 10⁵ competitor BM cells. Data are presented as ± SEM. | | | | | | | |
| C. Ly 5.1 BM cells with a lin⁻¹⁰, Sca-1⁺, and c-kit⁺cell surface phenotype were purified. 100 fresh cells per mouse (Control) were transplanted with 10⁵ Ly 5.2 competitor BM cells. One thousand of the same purified cells were cocultured with stromal cell lines for 6 weeks. The cultures were then harvested and 10% of each culture was transplanted per mouse with competitor. Data are from peripheral blood samples | | | | | | | |
| #taken 4 months after transplant and are presented ± SEM. | | | | | | | |

TABLE 5
LTRSC maintained on AFT024 are able to repopulate secondary
recipients at levels comparable to non-cultured stem cells.

% Ly 5.2^{+ peripheral blood cells}

| | | | Comp. |
|---|---|---|---|
| Group | Weeks | Radioprotection | Repopulation |
| Control | 6 | 21 (1) | 2.6 ± 0.6 (4) |
| | 22 | 54 (1) | 5.6 ± 1.6 (4) |
| AFT024 | 6 | 13 ± 0.2 (4) | 1.3 ± 0.9 (8) |
| | 22 | 44 ± 15.1 (3) | 4.3 ± 0.9 (8) |
| 2012 | 6 | 6.7 ± 2.9 (4) | ND |
| | 22 | 14 ± 2.2 (4) | ND |

The retransplantation potential of LTRSC in primary recipients of stromal cell cultured stem cells was studied in secondary recipients. 60 weeks after transplant, primary mice (see FIG. 14, Table 4A.) were sacrificed, BM harvested and stained with antibody to Ly5.2. Ly5.2⁺ cells were collected by cell sorting and used to transplant secondary recipients (congenic Ly5.1 mice). Control and AFT024 groups were transplanted with 1.5×10⁶ Ly5.2 cells/mouse for radioprotection (4 mice/group were transplanted) and 7.5×10⁵ Ly5.2 cells +7.5×10⁵ Ly5.1 cells for competitive repopulation (4 mice for the Control group and 8 mice for the AFT024 group). 2012 mice were transplanted with 3×10⁵ Ly5.2 cells/mouse (4 mice). Weeks are the times after transplant that the mice were analyzed. (n), number of mice surviving/group; ND, not done. Data are presented ±SEM.

### Example 4

### Hematopoietic Activity of a Stromal Cell Transmembrane Protein Containing Epidermal Growth Factor-like Repeat Motifs

Primitive hematopoietic stem cells are closely associated with discrete in vivo microenvironments. These “niches” are thought to provide the molecular signals that mediate stem cell differentiation and self renewal. The fetal liver microenvironment was dissected into distinct cellular components by establishing an extensive panel of stromal cell lines. One particular cell line maintains repopulating stem cells for prolonged in vitro culture periods. A subtraction cloning strategy has yielded a cDNA which encodes a cell surface glycoprotein with a restricted pattern of expression among stromal cell lines. This molecule, previously identified as delta-like/pre-adipocyte factor-1, contains epidermal growth factor-like repeats which are related to those in the notch/delta/serrate family of proteins. The potential role of this molecule in hematopoietic stem/progenitor cell regulation was investigated. The delta-like protein displays activity on purified stem cells by promoting the formation of “cobblestone areas” of proliferation. These cobblestone areas contain both primitive high-proliferative potential progenitors and in vivo repopulating stem cells.

Introduction

The positive and negative regulatory mechanisms that govern the proliferation, self-renewal and differentiation of primitive hematopoietic stem cells are complex and poorly understood (Ogawa, M. (1993) Blood 81, 2844-2853). Numerous cytokines have been identified that, when used in vitro appear to act directly on purified stem cells by promoting proliferation and differentiation. However, attempts to demonstrate the in vitro maintenance and/or expansion of transplantable pluripotent stem cells using defined cytokine combinations have been largely unsuccessful (Knobel, K. M., et al., 1994; Peters, S. O., et al., 1995). Moreover, it is unclear to what extent any currently identified cytokines reflect mechanisms that are responsible for regulating normal, in, vivo, stem cell behaviors. It is widely accepted that in vivo, stem cells are intimately associated with discrete microenvironmental “niches” (Wolf, N. S. 1979). Such niches are likely sources for the molecular signals which collectively mediate the differentiation and self-renewal of stem cells. Indeed, it has long been possible to demonstrate that preestablished stromal cell monolayers derived from hematopoietic tissues can support long-term hematopoiesis in vitro (Dexter, T. M., Allen, T. D. & Lajtha, L. G. 1977). The long-term nature of these cultures, together with the continuous production of committed progenitor cells suggest that both self-renewal and commitment decisions can occur in vitro. At the cellular level, the hematopoietic microenvironment consists of numerous distinct cell types. Previous studies have shown that this cellular heterogeneity reflects a similarly broad heterogeneity in terms of hematopoietic supportive abilities (Deryugina, E. I., et al., 1994). Some cloned stromal cell lines can support stem cell activity in vitro, while others are ineffective. Similarly, distinct stromal cell types appear to influence the outcomes of stem cell differentiation processes (Friedrich, C., et al., 1996). Recent studies have shown that stromal cell lines that efficiently maintain long-term transplantable stem cells in vitro for prolonged intervals represent a small fraction of the total stromal cell population (Wineman, J., et al., 1996). A fetal liver stromal cell line, AFT024 was identified which maintains high levels of transplantable multilineage stem cell activity for extended in vitro culture periods (Moore, K. A., Ema, H. & Lemischka, I. R. (1997) (See, Example 3). The stem cells used to initiate these cultures are highly purified. It is, therefore, likely that in this system the mechanisms that mediate stem cell maintenance do so by acting directly on primitive stem cells. A different fetal liver-derived cell line, 2018, fails to maintain long-term repopulating stem cell activity. A PCR-based RNA expression analysis of 13 cytokines reveals qualitatively identical expression patterns in AFT024 and 2018. It wastherefore, hypothesized that the hematopoietic supportive ability of AFT024 is, at least in part, mediated by novel gene products not expressed in 2018.

Using a subtractive hybridization approach a number of cDNA clones were identified which were specifically expressed in AFT024 but not in 2018. The sequence of one cDNA was identical to a molecule that encodes a transmembrane protein that contains six epidermal growth factor (EGF) repeat motifs. The EGF-like repeat sequences of this molecule, variously known as delta-like (dlk) (Laborda, J., et al., 1993), preadipocyte factor-I (Smas, C. M. & Sul, H. S, 1993), and stromal cell protein-1 (Genbank, D16847), are most closely related to those present in the notch/delta/serrate family of signaling proteins. In Drosophila and Caenorhabditis, these molecules are required for correct cell-fate specification decisions in a variety of tissues (Rebay, I., et al., 1991). Vertebrate homologs of the notch/delta/serrate family have been identified (Ellisen, L. W., et al., 1991; Bettenhausen, B., et al., 1995; Lindsell, C. E., et al.,1995). While the exact functional relationship of dlk to the activities of this family of molecules is unclear, in one in vitro study, it has been shown to block adipocyte differentiation (Smas, C. M. & Sul, H. S, 1993). No studies have been reported that demonstrate a hematopoietic function for dlk. Expression analyses and these observations show a limited temporal pattern of dlk expression during murine fetal development which coincides with the time period of hematopoietic stem cell expansion (Smas, C. M. & Sul, H. S, 1993).

Functional studies were undertaken to determine if dlk can act as a hematopoietic regulator. This molecule affects highly enriched stem cell populations by promoting “cobblestone area” (CSA) colony formation in dexter-type stromal cocultures. These CSA colonies contain an expanded population of primitive, high proliferative potential myeloid-erythroid progenitors. These cultures also contain stem cells capable of in vivo engraftment at levels equivalent to those present in parallel AFT024 supported cultures. It is proposed that dlk represents one molecular component responsible for the hematopoietic supportive ability of AFT024. As such, dlk may define a novel molecular pathway of stem cell regulation by the microenvironrnent.

Materials and Methods

Cell Lines and Culture.

The fetal liver stromal cell lines used in this study were derived as previously described (Wineman, J., et al., 1996). Cells were routinely cultured in DMEM containing 10% fetal bovine serum (FBS) and 50 uM b-mercaptoethanol (2-ME), and maintained at 31°-33° C., 5% CO₂. For long-term cocultures with hematopoietic stem cells, confluent monolayers were irradiated (20 Gy), placed in modified Dexter media (DMEM, 10% FBS, 10% horse serum, 50 uM 2-ME, 0.1 uM hydrocortisone) and maintained at 37° C., 5% CO₂ with weekly media changes. NIH3T3 cells were obtained from ATCC.

dlk Expression Analysis.

Total RNAs from stromal cell lines were poly A⁺ selected, Northern blotted, and hybridized to ³²P-labeled probes according to standard protocols (Sambrook, J., Fritsch, E. F. & Maniatis, T, 1989). A 600 bp dlk-cDNA clone from the AFT024 subtracted library was used as a probe. cDNA templates for RT-PCR were prepared according to manufacturers′ protocols (GIBCO/BRL). Oligonucleotide primers were: sense 5′-GACCCAGGCTGCCCC-3′ (SEQ.ID.No.:85) and antisense 5′-GGTACTCTTGTTGAG-3′ (SEQ.ID.No.:86). For analysis of dlk expression at the protein level, antisera specific for dik was generated by immunizing rabbits with a Flag-dlk fusion protein (described below). Resultant antibodies were purified by affinity chromatography. Cell surface expression of dlk in stromal cell lines was accomplished by flow cytometry. Cells were incubated with dlk antibody and a similarly prepared irrelevant control antibody. Specific labeling was developed by donkey anti-rabbit-fluorescein isothiocyanate (Jackson ImmunoResearch). Stained cells were analyzed on a Becton Dickinson FACScan using Cell Quest software.

dlk Fusion Protein Preparation.

The expression plasmid pCD4-Ig contains cDNA for the extracellular domain of human CD4 fused to genomic sequences of the human immunoglobin heavy chain (Zettlmeissl, G., et al., (1990). cDNA for CH2-CH3 of human IgG, (Goodwin, R. G., et al., 1990) was cloned into EcoRI and NotI sites of pcDNA3 (Invitrogen) to give the plasmid KB52.3.2. cDNA encoding the extracellular domain of dlk was obtained by RT-PCR with primers BP 151 and BP 152 using total RNA from NIH 3T3 cells as template. The resulting PCR fragment was cloned into KB52.3.2 via HindIII and EcoRI sites to obtain the soluble dlk-Ig expression plasmid. pdlk-Ig or pCD4-Ig were transfected into NIH3T3 cells together with pSVNeo and stable clones were isolated. Soluble CD4-Ig and dlk-Ig fusion proteins were harvested and then purified by affinity chromatography on HiTrap Protein G-sepharose (Pharmacia). Primers: sense BP 151, 5′GAGGGTACCAAGCTTCGTGGTCCGCAACCAGAAG-3′ (SEQ.ID.No.: 87); anti-sense BP 152, 5′-CTCAGATCTGAATTCGGCCTGTCCCTCGGTGAGGAG-3′ (SEQ.ID.No.: 88).

Flag-dik fusion protein was used to immunize rabbits for the production of dlk antiserum. The protein expression plasmid pcDNA3-Flag is a modification of the plasmid pcDNA3 (Invitrogen) and contains the coding region for the Flag peptide (DYKDDDDKI) (Hopp, T. P., et al., 1988) as well as a BglII restriction site. A cDNA fragment encoding the extracellular domain of dlk was obtained by RT-PCR using RNA from NIH3T3 cells. Primers: sense BP 155,

5′-GACAAGATCTCAGCTGAATAGCGACCCACCCTGTG-3′ (SEQ.ID.No.: 89); antisense BP 154,

5′-GCATCTAGAGCGGCCGCTCAGGCCTGTCCCTCGGTGAGGAG-3′ (SEQ.ID.No.: 90). The PCR fragment was ligated into pcDNA3-Flag to yield pFlag-dlk. pFlag-dlk was transfected into cos cells. Purification of the Flag-dlk protein from cos-conditioned media was performed according to manufacturer's directions using the Flag monoclonal antibody, M1, immobilized on agarose (International Biotechnologies).

Plasmid Constructs and Stable Transfection.

Full-length murine dlk cDNA was obtained by RT-PCR with primers BP 151 (see above) and antisense BP 200: 5′GCATCTAGAGCGGCCGCGAACGCTGCTTAGATCTCCT-3′ (SEQ.ID.No.:91), using total RNA from NIH3T3 cells as template. The product was subcloned into the vector pCRII (Invitrogen) and then cloned into a retroviral expression vector (Kitamura, T., et al., 1995), (G. Nolan, Stanford University), via the primer-encoded HindIII and NotI sites. Supercoiled plasmid was transfected into BFC012 stromal cells together with the pZeo (Invitrogen) selectable marker and selected in 50 ug/ml Zeocin (Invitrogen). BFC012 cells also were transfected with pZeo alone and selected as above. Clones from both selected populations were isolated and all remaining colonies (100-200 per dish) were pooled and expanded as populations.

Hematopoietic Stem Cells and in vitro Hematopoietic Assays.

Hematopoietic stem cell populations were derived from wild type, Ly5.2-C57B1/6J (Jackson Laboratories), day 14 fetal liver, enriched for the AA4.1⁺, Sca-1⁺, c-kit⁺, and lin^{lo/-} phenotype, by immunopanning and fluorescence-activated cell sorting as described (Jordan, C. T., et al., 1995). Adult bone marrow (BM) was used directly after density centrifugation and immunomagnetic bead depletion or was further enriched for Sca-1⁺, c-kit⁺, lin^{lo/-} cells by flow cytometry as described (Okada, S., et al., 1992). Cell sorting and data analysis was accomplished with a Becton Dickinson FACS Vantage using Cell Quest software. Stromal cell/stem cell cocultures were initiated in 12-well trays with 300-1,000 enriched stem cells per well. Cobblestone areas were quantitated by inverted-phase microscopy as described (Ploemacher, R. E., et al. , 1991). Clonogenic progenitor assays were performed with either freshly purified stem cells or cells harvested from the stromal cocultures. These were cultured in cytokine-containing semisolid media according to the manufacturer's recommendations (Stem Cell Technologies, Vancouver, BC). Soluble dlk and control fusion proteins were added to semisolid progenitor assays at concentrations of 0.1, 0.5 and 1.0 ug/ml and also to BFC012 stromal cocultures at concentrations of 0.1 ug/ml. Fusion protein was replenished weekly in the stromal cocultures.

Competitive Repopulating Transplantation Assay.

Cultured cells were harvested, combined with fresh unfractionated BM obtained from congenic C57B1/6 Ly5.1 mice (National Cancer Institute) and transplanted into lethally irradiated (10 Gy, split dose 3 h apart from a ¹³⁷Cs source, 1 Gy/min) Ly5.1 recipient mice. Each mouse received 2×10⁵ competitor BM cells and a fraction of the cocultured stem cells. Mice were bled by capillary puncture of the orbital venous plexus and 100 ul was collected; red blood cells were removed by NH₄Cl lysis. The nucleated cells were stained for the Ly5.2 (CD45.2) allelic marker using either fluorescein isothiocyanate-labeled directly conjugated Ly5.2 monoclonal antibody or a biotinylated form developed with streptavidin conjugated to Texas red. Cells also were stained with directly conjugated antibodies to lineage markers. All antibodies and chromogens were obtained from Pharmingen. Flow cytometric analysis was done on a Becton Dickinson FACS Vantage using Cell Quest software.

Results

Genes expressed in AFT024 but not in 2018 were identified by a subtractive cloning approach. Sequence analysis identified one of these AFT024-specific clones as dlk. Expression studies, (FIG. 19A) show high levels of dlk in AFT024 and subclones isolated from this line, but undetectable levels in 2018 and BFC012. The latter two stromal cell lines do not support repopulating stem cells. The PA6 stromal cell line and NIH Swiss 3T3 cells both show expression of dlk and were the cell sources for identification of SCP-1 and dlk, respectively. PA6 cells have been shown to support in vitro hematopoiesis and long-term, in vivo repopulating stem cells (Kodama, H., et al., 1992). Swiss 3T3 cells are also capable of supporting multipotent hematopoietic stem cells in vitro, promoting CSAs and maintaining in vivo spleen colony-forming units (CFU-S) (Roberts, R. A., et al., 1987). Interestingly, an additional fetal liver stromal cell line, 2012, which has some degree of stem cell supporting activity (Wineman, J., et al., 1996), and its subclones also express dlk. Furthermore, an RT-PCR analysis (40 cycles) of an additional 10 fetal liver-derived stromal cell lines and several other lines, shows detectable levels of dlk in only two additional lines (FIG. 19B). These two cell lines (CFC032 and CFC008) can maintain some level of long-term transplantable stem cell activity present in whole BM (Wineman, J., et al., 1996). A correlation between a stromal cell line's ability to support stem cells and the expression of dlk was suggested. Therefore, functional studies were undertaken in order to delineate if dlk can act on or modulate hematopoictic stem cells.

Soluble dlk protein was added to progenitor cultures in semi-solid media. The soluble protein consisted of the dlk extracellular domain fused to the Fc portion of human IgG₁. The stem cell sources in these assays were highly enriched fetal liver cells (AA4.1⁺, lin^{lo/-}, Sca-1⁺, c-kit⁺). The influence of soluble dlk on hematopoietic progenitor colony-formation was assessed. As shown in Table 6, no differences were noted either in the number, sizes, or lineage compositions of colonies. Identical results were obtained at dlk concentrations ranging from 0.1 to 1.0 ug/ml. In addition, no differences were noted in similar studies using enriched BM cells (Sca-1⁺, c-kit⁺, lin^{-/lo}).

Evidence for a positive effect of the dlk protein on stem/progenitor cells was observed when the soluble form was added to dexter-type cocultures. For these studies a stromal cell line (BFC012) was used that neither expresses endogenous dlk (see FIG. 19) nor maintains significant in vitro hematopoiesis. In four experiments, two each using highly enriched adult BM and fetal liver stem cells, we monitored the appearance of CSAs over time. These colonies provide a convenient, quantitative estimate of hematopoietic activity initiated by primitive stromal dependent stem/progenitor cells. As shown in FIG. 20, the addition of soluble dlk (0.1 ug/mL) results in an approximately 2-fold increase in the number of CSAs initiated by purified fetal liver or BM stem cells over a 2 week time period (P=0.001 for dlk vs control and P=0.01 for dlk vs no additive, Student's t-test). There was no difference in the numbers of CSA observed in BFC012/stem cell cultures with or without control fusion protein (mean of no additive/control=0.96±0.11).

In order to assess the activity of the normal transmembrane form of dlk, a full-length dlk cDNA was transfected into BFC012 cells. Expression of the introduced dlk was demonstrated at the RNA (Northern blot) and protein levels using both Western blot and flow cytometric analyses with rabbit anti-dlk antibodies. The flow cytometry data are presented in FIG. 21.

Initially, dlk-expressing transfected populations (BFC-dlk) were compared to a negative control “mock” transfected population of BFC012 cells. A 4- to 6-fold increase in the number of CSAs was observed in two separate experiments. The maintenance of CSAs was transient, lasting less than 2 weeks. No further hematopoietic activity was observed during an additional 2 weeks of culture. Once dlk-expressing clones had been identified from the transfected populations, they were studied for their ability to support CSAs in experiments designed to more precisely identify the time course of hematopoietic activity. Ficoll-separated, lineage depleted BM was used in these experiments. Five different negative control, non-dlk expressing BFC012 cell groups (parental BFC012 cells, two “mock” transfected populations, and two “mock” transfected clones) and 3 dlk-expressing BFC012 cell groups (one transfected population and two clones) were studied. The data are presented in FIG. 22A. Neither the negative control BFC populations nor the “mock” transfected BFC clones supported high numbers of CSAs. In contrast, the BFC-dlk populations and the two individual dlk-expressing clones supported significantly greater numbers of CSAs at all time points studied (P<0.001 days 3,4, and 5; P<0.01 days 6 and 7, Student's t-test). As observed previously, all the CSAs were transient. This experiment also indicated that the dlk-promoted hematopoietic activity peaks early, at 4 days, in this culture system. Three additional experiments using purified (AA4.1⁺, lin^{lo/-}, Sca-1⁺, c-kit⁺) fetal liver stem cells were performed using two individual clones, BFC-dlk-5 and a “mock” transfected negative control BFC-Zeo-1. The results are presented in FIG. 22B. There was a dramatic and significant difference in the number of CSAs observed in the BFC-dlk-5 cultures compared to the control line (P<0.001, days 4, 6, and 8, Student's t-test). As before, the effect was transient and the CSA declined in number over 2 weeks. AFT024 was included as a positive control and, in each of the three experiments, verified the quality of the input purified stem cells. In the first week of culture the numbers of CSAs observed on AFT024 were similar to the numbers in the BFC-dlk5 cultures.

In order to address the “primitiveness” of the cells that give rise to the CSA observed in the BFC-dlk cocultures, a series of in vitro replating experiments were performed. Individual wells were harvested at various time points of coculture and the cells were plated into semisolid cytokine-containing media. The numbers and lineage compositions of the colonies were scored after 8-12 days. As shown in FIG. 23A, the CSAs obtained from day 4 BFC-dlk-5 cocultures contained numerous progenitors capable of extensive proliferation and multilineage differentiation. The total number of progenitors from the dlk-expressing cultures at day 4 was significantly expanded compared to the content in the freshly purified uncultured stem cell population (P=0.01, Student's t-test). The number and lineage composition of colonies derived from parallel day 4 AFT024 cultures was nearly identical to BFC-dlk-5 derived colonies. The content of CSAs replated at day 6 from the BFC-dlk-5 cocultures was devoid of multilineage colonies, although CFU-granulocyte-macrophages were maintained at high levels; the progenitor content in the BFC-dlk-5 cocultures continued to decrease when next sampled at 10 days. in contrast, few progenitors could be demonstrated in the BFC-Zeo-1 cultures (P=0.001, BFC-dlk-5 vs. BFC-Zeo-1, Student's t-test) (FIG. 23A). Taken together, the data strongly suggest that dlk acts to promote stromal-dependent colony-formation by primitive cells capable of yielding large numbers of committed progenitors, including those endowed with a high proliferative capacity and multilineage differentiation potential. The lack of CSAs and significant progenitor maintenance in the BFC-Zeo-1 cultures argues that expression of dlk in the transfected BFC012 cells is responsible for both their ability to support CSAs and to generate/maintain primitive progenitors.

In order to determine if the CSA-containing cultures supported by BFC-dlk-5 contained stem cells capable of in vivo engraftment, portions of the same day 4 cocultures that were plated into progenitor assays also were used to transplant mice in competitive repopulation assays. Shown in FIG. 23B are the results from two independent experiments analyzed at 10 weeks after transplant. The same BFC-dlk-5 cultures that contain CSAs and primitive CFU-high proliferativ potential (HPP)-Mix progenitors also contain repopulating stem cells at levels equal to those maintained in parallel AFT024 cocultures. In addition, a significant difference exists in the levels of repopulating stem cells derived from dlk-expressing cocultures compared to non-dlk expressing BFC012 cells (P=0.05, Student's t-test). Multi-color flow cytometric analyses also demonstrated that both myeloid and lymphoid Ly5.2 cells are present in these animals. A subsequent analysis of these animals at 22 weeks demonstrated lower levels of repopulation with Ly5.2 cells derived from the AFT024 and BFC-dlk-5 supported cultures (data not shown). Most significantly, no repopulation was observed at any time point in mice that received cells cocultured on the non-dlk-expressing monolayers (FIG. 23B).

Discussion

As part of an ongoing effort to understand the biology of the hematopoietic microenvironment, a panel of stromal cell lines from midgestation fetal liver was established and characterized. Among these cell lines, the AFT024 line has the ability to maintain nearly quantitative levels of transplantable stem cell activity for extended in vitro time periods (Moore, K. A., Ema, H. & Lemischka, I. R.,1997). Because these cultures are initiated with highly purified stem cell populations it is likely that the AFT024-derived molecular mechanisms responsible for this ability act directly on the stem cell population. Other stromal lines that fail to maintain stem cell activity were also identified. These observations facilitated a subtractive hybridization approach aimed at identifying potential candidate molecules whose collective actions may be responsible for the AFT024 stem cell maintenance activity. This effort has identified dlk, a transmembrane molecule containing six EGF-like repeat motifs. Although lacking the DSL motif indicative of the notch ligands delta and serrate (Tax, F. E., Yeargers, J. J. & Thomas, J. H., 1994), dlk is most closely homologous to delta/notch/serrate when compared to other EGF-like repeat containing molecules (Laborda, J., et al., 1993; Smas, C. M. & Sul, H. S., 1993). The predominant role of these types of molecules in cell growth and differentiation led us to investigate the potential role of dlk in hematopoiesis. Constitutive expression of translocated human notch (Tan-1) is found in a T-cell leukemia (Ellisen, L. W., 1991). Moreover the expression of Tan-1 in primitive human stem cells has been demonstrated (Milner, L. A., et al., 1994). Nevertheless, a functional role in hematopoiesis for the notch ligands Jagged (Lindsell, C. E., 1995) and Delta-like-1 (Bettenhausen, B., et al., 1995) has not been described. dlk expression is highly restricted in a panel of stromal cell lines. Two lines, AFT024 and 2012, which maintain repopulating stem cell activity in vitro, express dlk, whereas two non-supportive cell lines, 2018 and BFC012, do not. Interestingly, the S17 stromal cell line which is considered to be a potent stem cell supporter (Wineman, J. P., et al., 1993) does not express detectable levels of dlk. The S17 cell line was derived from adult BM (Collins, L. S. & Dorshkind, K., 1987)). The other lines described are all derived from fetal sources (AFT024, 2012, and NIH 3T3 cells) or from newborn calvaria (PA6 cells). It is therefore possible that dlk acts in a developmentally regulated fashion. An extensive analysis of dlk expression in adult BM stroma is currently underway. Taken together, the data suggest that, at least in fetal stromal cell types, there exists a correlation between hematopoietic supporting ability and the expression of dlk.

The potential activity of both soluble and transmembrane dlk protein, on highly purified stem cell populations were directly measured using in vitro and in vivo assays. Initial experiments designed to ask if dlk can enhance colony formation in cytokine-rich semisolid assay systems were negative. These results may indicate that: (i) progenitor cells capable of colony formation in semisolid assays do not respond to dlk, (ii) the collection of cytokines present in the semisolid cultures may “mask” an effect(s) of added dlk or (iii) that the soluble form of dlk requires a stromal monolayer to mediate its effects. The first possibility can be addressed more extensively in delta-type assays, where stem/progenitor cells are first cultured in suspension in serum-free media containing various cytokine combinations, with and without dlk, and then replated into colony assays (Muench, J. O., Firpo, M. T. & Moore, M. A., 1993). The second possibility can be addressed by more extensive studies using subsets of the cytokines present in our initial studies. These experiments are underway. As a first step to address the third possibility, we added soluble dlk to preestablished BFC012 monolayers. Using both purified BM and fetal liver stem cell populations, a significant increase in CSA colony formation was observed in the dlk supplemented cultures (FIG. 20). This was a surprising result, given that dik is a transmembrane protein; however, before its cDNA cloning, a soluble form of dlk was identified as FAI or fetal antigen 1 (Jensen, C. H., et al., 1994). A role in hematopoiesis was not indicated in these studies, but expression was detected in stroma of placental villi, in yolk sac blood islands and in fetal liver (Jensen, C. H., et al., 1994). It is of interest to determine if a soluble form is produced by the stromal cell lines that express dlk. An additional explanation, for the effects observed with the soluble form added to stromal/stem cell cocultures, is that they may be facilitated by the Fc portion of the fusion protein. It is possible that Fc receptors expressed by some of the hematopoietic cells in the cultures are able to sequester and present the dlk-Fc fusion protein more effectively. This possibility can be addressed by using a different type of soluble dlk protein. These studies have been initiated. Alternatively, the soluble dlk may be sequestered and thus presented by the stromal cell extracellular matrix. In order to further address the third possibility, an intact transmembrane form of dlk was introduced into the BFC012 stromal cell line. Initially, dlk transfected BFC012 cell populations were compared to BFC012 cells transfected with the selectable marker alone. In these studies, the dlk expressing monolayers were more effective at promoting CSA colonies. As with the soluble dlk experiments, the CSAs appeared early and were transient. When cells were replated from the BFC-dlk supported cocultures onto an AFT024 monolayer a reiteration of the burst of CSAs was seen that was maintained for 3 weeks. In addition, replating of these cocultures revealed a high progenitor content (˜1 in 10) that included a high percentage (43%) of multipotential colonies including HPP-Mix. In the BFC-Zeo control populations neither replatable CSA nor CFU progenitors were maintained. Further experiments with individual clones from the transfected populations confirmed and extended the results obtained with the populations, demonstrating highly significant differences in the numbers of developing CSAs (FIG. 22). However, in two experiments we observed that one dlk-transfected BFC012 clone, which expresses a very high level of dlk, supported fewer CSAs than non-dlk-expressing control cells. These cultures also suggested differentiation phenomena, as indicated by the number of rapidly accumulating nonadherent cells. Experiments utilizing this cell line were not included in our analyses. It is possible that there may be a threshold level of dlk expression necessary in these cultures and when it is surpassed the cells differentiate and die rapidly in the culture mileau provided by BFC012 cells. In addition, it is possible that an aberrant form of the dlk protein is made by this line. Further studies are necessary to clarify this issue. Nevertheless, in FIG. 22A, dlk-transfected BFC012 cells (one population and two clones) show a significant enhancement of CSA formation compared to controls.

The observed low level maintenance of competitive repopulating stem cells in short-term dlk-expressing cocultures is of interest even though the activity diminished over time. These studies show that the ectopic expression of a single molecule (dlk) in a previously nonsupporting stromal cell line restores or enables hematopoietic support. This is demonstrated by maintenance of three different stem/progenitor cell compartments: (i) CFU-HPP-Mix, (ii) CSA, and (iii) short-term in vivo repopulating stem cells. It is also of interest that both qualitatively and quantitatively similar stem/progenitor cell compartments are maintained in short-term AFT024 supported cocultures (Moore, K. A., Ema, H. & Lemischka, I. R., 1997).

Two mechanisms underlying the effects of dlk are considered. First, it may be that some level of dlk expression is sufficient to retard potent differentiation signals provided by the BFC012 cell line. Second, dlk may provide a proliferative stimulus not normally produced by BFC012. A more direct and perhaps relevant assay will be to eliminate the expression of dlk in AFT024 cocultures, thus maintaining other components that make up the culture “milieu.” Studies to evaluate potential neutralizing antibodies and various dlk antisense strategies are underway.

The failure of BFC-dlk-5 supported CSAs to persist for periods longer than 1 to 2 weeks also may suggest the existence of other molecules in AFT024 that facilitate hematopoiesis. In this regard, it is interesting that our subtraction screen has yielded several other clones with expression patterns very similar to dlk. Eventually, with the addition of dlk and other AFT024 specific molecules it may be possible to reconstruct a supportive phenotype. This should lead towards a further understanding of the ill vivo hematopoietic microenvironment. In summary, it is proposed that dlk represents one molecular component responsible for the hematopoietic supportive activities of the AFT024 cell line. As such, dlk may define a novel molecular pathway of stem cell regulation by the hematopoictic microenvironment.

### REFERENCES

The following is a list of documents related to the above disclosure and particularly to the experimental procedures and discussions. The documents should be considered as incorporated by reference in their entirety.

1. Andrews, R., J. Singer, and 1. Bernstein. 1989. J Exp Med 169:172 1.

2. Ardman, B., et al. (1992). Proc. Natl. Acad. Sci. U S A. 89, 5001-5005.

3. Artavanis-Tsakonas, D., K. Matsuno, and M. E. Fortini. 1995. Science 268:225-232.

4. Ball T C, Hirayama F, Ogawa M. Blood 85:3086, 1995

5. Barbosa, M. D., Q. A. Nguyen, V. T. Tchernev, J. A. Ashley, J. C. Detter, S. M. Blalydes, S. . et al., 1996. Nature 382:262-265.

6. Baud, W., et al., 1995. Genomics 26:334-344.

7. Bauer, D., et al., Nucl Acids Res 21:4272-4280.

8. Baum, C. M., et al., Proc Natl Acad Sci USA 89:2804-2808.

9. Bauman, J. G., et al., Acta Histochem 36:241-253.

10. Bazil, V., Brandt, et al. (1997). Stem Cells 15, Suppl. 1, 13-18.

11. Beavis, A. J., and K. J. Pennline. 1994. J Immunol Methods 170:57-65.

12. Bettenhausen, B., et al., 1995 Development 121, 2407-2418.

13. Bhatia, M., et al., 1997. Proc Natl Acad Sci USA 94,5320-5325.

14. Bonnet, D., and J. E. Dick. 1997. Nature Med 3:730-737.

15. Bork P, et al. Q Rev Biophys 1996 May;29:119-167

16. Bomemann, D., E. Miller, and J. Simon. 1996. Development 122:162 1 1630.

17. Borrow, J., et al., 1996. Nat Genet 12:159-167.

18. Bradford, G. B., et al., 1997. Exp Hematol 25:445-453.

19. Brady, G., and N. N. Iscove. 1993. In Methods in Enzymology, 225: 611-623.

20. Brady, G., et al. 1990. Methods in Molecular and Cellular Biology 2:17-25.

21. Brandt J, et al., J Clin Invest 86:932, 1990

22. Braun, B. S., et al., 1995. Mol Cell Biol, no. August, 1995:4623-4630.

23. Britten, R. J., et al., 1985. Hybridisation Strategy. In Nucleic Acid Hybridisation. B. D. Homes and S. J. Higgins, editors. Oxford, Washington, D.C. 3-15.

24. Broxmeyer H E, et al. Proc Natl Acad Sci USA 89:4109, 1992

25. Brugger W, et al. Blood 81:2579, 1993.

26. Carpino, N., et al., 1997. Cell 88:197-204.

27. Cashman, J., et al., 1997. Blood 89:4307-4316.

28. Cheng, J., et al. (1996). Blood 87, 479-490.

29. Cheng, L., et al., 1996. Nature Biotechnology 14:606.

30. Chiang, M. -Y., et al. J Biol Chem 266:18162-18171.

31. Chomczynski, P., and Sacchi, N. (1987). Anal. Biochem. 162, 156-159.

32. Chubet, R. G., and B. L. Brizzard. 1996. BioTechniques 20:136-141.

33. Collins L S and Dorshkind K: J Immunol 138:1082, 1987.

34. Collins, L. S. & Dorshkind, K. (1987) J. Immunol. 138, 1082-1087.

35. Craig, W., et al., 1993 J Exp Med 177:1331-1342.

36. Cumano, A., et al., (1993). Proc. Natl. Acad. Sci. USA. 90, 6429-6433.

37. Deiss, L., et al., 1995. Genes Dev 9:15-30.

38. Deryugina, E. I., et al., 1994 Exp. Hematol. 22, 910-918.

39. Dexter TM, in Wright DG, Greenberger JS (eds): Long term bone marrow culture. New York, Alan R. Liss, Inc., 1984, p 57

40. Dexter, T. M., et al., (1977) J. Cell. Physiol. 9, 335-344.

41. Diatchenko, L., et al. 1996. Proc Natl Acad Sci USA 93:6025-6030.

42. DiGiusto, D. L., et al., 1996 Blood 87:1261-1271.

43. Dorshkind K Annu Rev Immunol 8:11, 1990.

44. Dosil, M., et al., 1996. Blood 88, no. 12:4510-4525.

45. Elledge, S., and J. Harper. 1994. Curr Opin Cell Biol 6:847-852.

46. Ellisen, L. W., et al., (1991) Cell 66, 649-661.

47. Fairbaim, L. J., et al., 1993. Cell 74:823-832.

48. Fleming, W. H., et al., 1993 J Cell Biol 122:897-902.

49. Frampton, J., et al., 1995 EMBO J 14:2866-2875.

50. Fraser C C, et al., Blood 76:1071, 1990.

51. Frederiksen K, et al., Neuron 1:439, 1988.

52. Friedrich, C., et al., (1996) Blood 87, 4596-4606.

53. Fukai, K., et al., 1996 Am J Hum Genet 59:620-624.

54. Galy, A., et al., Immunity 3:459-473.

55. Goode, S., et al., 1996 Development 122:3863-3879.

56. Goode, S., et al., 1992 Development 116:177-192.

57. Goodwin, R. G., (1990) Cell 60,941-951.

58. Graf, L., and B. Torok-Storb. 1995. Blood 86:548-556.

59. Guan, E., et al. J. Biol. Chem. 266, 20345-20355.

60. Guan. E,, et al. (1994). J. Immunol. 152, 4005-4012.

61. Gubler, U., and Hoffman, B. J. (1983).

62. Hamann, J., et al., 1995. J Immunol 155:1942-1950.

63. Hao Q-L, et al., Blood 88:3306, 1996.

64. Hao, Q.L., et al., 1995. Blood 86:3745-3753.

65. Hao, Q.L.,et al., 1996. Blood 88:3306-3313.

66. Harper, J., and S. Elledge. 1996. Curr Opin Genet Dev 6:56-64.

67. Harrison D E, et al., Exp Hematol 21:206, 1993.

68. Harrison D E, et al., Blood 69:1021, 1987.

69. Harrison D E, Stone M, Astle CL. J Exp Med 172:431, 1990.

70. Harrison, D. E. 1980. Blood 55:78.

71. Harrison, S. M., et al., 1995 Development 121:2479-2489.

72. Hawlely, R., et al., 1997. Cancer Res 57:337-345.

73. Hawley, R., et al., 1994. Gene Ther 1: 136-138.

74. Heximer, S., et al., 1996 DNA Cell Biol 15: 1025-1038.

75. Hofmann,A.,et al., 1996 Proc Natl Acad Sci USA 93, no. May:5185-5190.

76. Holyoake TL, et al., Blood 87:4589, 1996.

77. Hopp, T. P., et al., (1988) Biotechnology 6, 1204-1210.

78. Horan, PK., et al., 1990 Methods Cell Biol 33:469-490.

79. Hu, M., 1997 Genes Dev 11:774-785.

80. Huang S, et al., Blood 83:1515, 1994.

81. Hubank, M., and D.G. Schatz. 1994. NucI Acids Res 22, no. 25:5640-5648.

82. Issad C, et al., Blood 81:2916, 1993.

83. Jensen, C. H., et al., (1994) Eur. J. Biochem. 225, 83-92.

84. Jones, R., et al., 1996. Blood 88:487-491.

85. Jones, R., et al., 1990. Nature 347:188-189.

86. Jongens, T. A., et al., 1992 Cell 70:569-584.

87. Jordan, C. T., et al., 1995. Exp. Hematol. 23, 1011-1015.

88. Jordan, C. T., and Lemischka, I. R. (1990). Genes Dev. 4, 220-232.

89. Jordan, C. T., et al. Cell 61, 953-963.

90. Jordan, C.T., et al. 1996. Exp Hematol 24:1347-1355.

91. Kabrun, N., et al. (1997). Development 124, 2039-2048.

92. Keller, G. M. (1995). Cell. Biol. 7, 862-869.

93. Khochbin, S., D. et al., 1990 Cytometry 11: 869-874.

94. Kinsella, T.M., and Nolan, G.P. (1996). Hum Gene Ther. 12, 1405-1413.

95. Kitamura, T., et al. Proc. Natl. Acad. Sci. U S A. 92, 9146-9150.

96. Knobel KM, et al., Exp Hematol 22:1227, 1994.

97. Knobel, K. M., et al., (1994) Exp. Hematol. 22, 1227-1235.

98. Ko, M. S. H. 1990. Nucl.Acids Res 18:5705-5711.

99. Kodama H, et al., J Exp Med 176:351, 1992

100. Kodama H, et al., J Cell Physiol 118:233, 1984.

101. Koller M R, et al., Blood 82:378, 1993.

102. Kulessa, H. et al., 1995 Genes Dev 9:1250-1262.

103. Laborda, J., et al., 1993 J. Biol. Chem. 268, 3817-3820.

104. Lansdorp PM, et al., J Exp Med 175:1501, 1992.

105. Lapidot-Lifson, Y, et al., 1992 Proc Natl Acad Sci USA 89:579-583.

106. Larochelle, A., et al., 1997 Nature Med 2:1329-1337.

107. Lawrence, H.J., et al., 1996 Stem Cells 14:281-291.

108. Lemischka IR: Dev Biol 4:379, 1993.

109. Lemischka I R, Cancer Surv 15:3, 1992.

110. Lemischka, I. R. (1997). Blood 89,4337-4347.

111. Lemischka, I. R. 1992. Curr Top Micro Immunol 177:59-71.

112. Li S H, et al. J Biol Chem 1994269:4431-4437.

113. Li, C. L., and G. R. Johnson. 1992. J Exp Med 17 5:1443.

114. Li, W.-B., et al., 1994. BioTechniques 16:722-729.

115. Li, X., et al., 1995. Cytometry 20:172-180.

116. Liang, P., et al., 1994 In Methods in Molecular Genetics, 5: 3-16

117. Lida, S., et al., 1993 Oncogene 8:3085-3092.

118. Lindsell, C. E., (1995) Cell 80,909-917.

119. Ling, V., and Neben, S. (1997). J Cell Phys. 171, 104-115.

120. Lord BI, et al., Blood 45:65, 1975.

121. Lowry, P.A., et al., 1995 Exp Hematot 23:1135-1140.

122. Lyons, AB., et al., 1992 Cytometry 13:809821.

123. Mackarchtschian, K., 1995 Immun 3:147-161.

124. Marcos, M. A. R. et al. (1997). J. Immunol. 158,2627-2637.

125. Methods in Mol Biol 5:303-322.

126. Matthews, W., et al., 1991 Cell 65:1143-1152.

127. Matthews, W., 1991 Proc Natl Acad Sci USA 88:9026-9030.

128. Mayani, H., 1993 Blood 82:2664-2672.

129. McKeam, J.P., et al. (1985). Proc. Natl. Acad. Sci. U S A. 82, 7414-7418.

130. Mehta P, et al. 1997 90:2381-2389.

131. Metcalf D: The molecular control of blood cells. Cambridge, Mass., Harvard University Press, 1988

132. Milner, L. A., et al., (1994) Blood 83, 2057-2062.

133. Milner, L. A., 1996 Proc Natl Acad Sci USA 93:13014-13019.

134. Mishell BB, et al.,: Selected Methods in Cellular Immunology. San Francisco, Calif., W. H. Freeman and Company, 1980, p 23.

135. Moore et al., BrJ Haematol 18:279, 1970

136. Moore, K. A., et al., 1997 Proc Natl Acad Sci USA 94:4011-4015.

137. Moore, K. A., H. Ema, and I. R. Lemischka. 1997. Blood 89:4337-4347.

138. Morrison, L. E., et al. (1991). Oncogene 6, 1657-1665.

139. Morrison, S. J., et al., 1997 Development 124:1929-1939.

140. Morrison, S. J., and I. L. Weissman. 1994. Immunity 1:661-673.

141. Morrison, S. J., et al., 1995 Ann Rev of Cell Dev Biol 11:35-71.

142. Morse HC, et al., Immunogenetics 25:71, 1987.

143. Muench MO, et al., Leuk Lymphoma 16:1, 1994.

144. Muench, J. O., et al., (1993) Blood 81, 3463-3473.

145. Mulder AH, et al., Exp Hematol 15:99, 1987

146. Muller, C., et al., EMBO J 14:61276135.

147. Murray, L., et al., 1994. Blood Cells 20:364-369.

148. Nakamura, T., et al., 1996 Nat Genet 12:154-158.

149. Neben S, et al., Exp Hematol 21:438, 1993.

150. Neering, S., et al., 1996 Blood 88:1147-1155.

151. Nepomuceno, R.R., et al. (1997). Immunity 6, 119-129.

152. Neuman-Silberberg, F. S., and T. Schupback. 1996 Mech Dev 59:105-113.

153. Nolta, J. A., et al., 1996. Proc Natl Acad Sci USA 93:2414-2419.

154. Ogawa, M. (1993) Blood 81, 2844-2853.

155. Okabe, T., et al., 1995. J Immunol 154:38713879.

156. Okada, S., et al., (1992) Blood 80, 3044-3050.

157. Okuda, T., et al., 1996 Cell 84:321-330.

158. Orkin, S. H. 1996. Curr Opin Genet Dev 6:597-602.

159. Osawa, M., et al., Science 273:242-245.

160. Patanjali, S.R., et al., 1991 Proc Nat[Acad Sci USA 88:1943-1947.

161. Pawliuk, R., et al., 1996 Blood 88:2852-2858.

162. Perou, C. M., et al., 1996 Nat Genet 13:303-308.

163. Persons, D., et al., 1997 Blood 90:1777-1786.

164. Peters SO, et al., Exp Hematol 23:461, 1995.

165. Peters, S. O., et al., (1995) Exp. Hematol. 23, 461-469.

166. Petrenko, O., (1997). Oncogene 14, 1671-1680.

167. Petzer, A. L., et al., 1996. Proc Natl Acad Sci USA 93:1470-1474.

168. Ploemacher R E, et al., Blood 74:2755, 1989.

169. Ploemacher, R. E., et al., (1991) Blood 78, 2527-2533.

170. Ploemacher, R. E., et al., 1989 Blood 74:2755.

171. Porcher, C., et al., 1996 Cell 86:47-57.

172. Randall, T. D., et al., 1996 Blood 87:4057-4067.

173. Rebay, I., et al., (1991) Cell 67, 687-699.

174. Rebel V I, et al., Blood 83:128, 1994.

175. Robb, L., et al., 1996 EMBO J 15:4123-4129.

176. Roberts, R. A., et al., (1987) J. Cell Physiol. 132, 203-214.

177. Ropp, J., et al., 1995 Cytometry 21:309-317.

178. Roth, S., et al., 1995. Cell 81:967-978.

179. Sambrook, J., et al., (1989) Molecular Cloning: A Laboratory Manual (Lab. Press, Plainview, N.Y.).

180. Sauvageau, G., U. et al., 1995 Genes Dev 9:1753-1765.

181. Schmid, I., et al., 1994 Cytometry 15:12-20.

182. Shalaby, F., et al., 1995. Nature 376:62-66.

183. Shaw, G., and R. Kamen. 1986. Cell 46:659-667.

184. Sherr, C. 1993. Cell 73:1059.

185. Sherr, C. 1996. Science 274:1672-1677.

186. Shivdasani, R. A., et al., Blood 87:4025-4039.

187. Shockett, P. E., et al., 1996. Proc Natl Acad Sci USA 93:5173-5176.

188. Sieweke, M., et al., 1996 Cell 84:49-60.

189. Simon, J. 1995. (1993) Cell 73, 725-734.

190. Soares, M. B., et al., 1994 Proc Natl Acad Sci USA 91:9228-9232.

191. Soma T, et al., Blood 87:4561, 1996.

192. Spangrude G J, Science 241:58, 1988

193. Spangrude, G. J., et al., 1990, Proc Natl Acad Sci USA 87:7433.

194. Spangrude. G. J., et al., 1995 Blood 85:1006-1016.

195. Spangrude, G. J., 1988 Science 241:58.

196. Stockton, B. M., et al. (1998). Immunity 8, 373-381.

197. Sutherland, H. S., et al., 1989. Blood 74:1563.

198. Suzuki J, et al., Leukemia 6:452, 1992

199. Suzuki, A., et al. (1996). Blood 87, 3550-3562.

200. Szilvassy S J, et al. Blood 87:4618, 1996.

201. Taswell C, J Immunol 126:1614, 1981.

202. Tax, F. E., Yeargers, J. J. & Thomas, J. H. (1994) Nature 368, 150-154.

203. Telford, W. G., L. E. King, and P. J. Fraker. 1992. Cytometry 13:137-143.

204. Telford, W. G., L. E. King, and P. J. Fraker. 1994. J Imm. Meth 172 1-16.

205. Terstappen L W M M, et al. Blood 77, 1991.

206. Testa NG, Molineux G: Haemopoiesis: A practical approach. New York, New York, Oxford University Press, Inc., 1993

207. Traycoff C M, et al., . Exp Hematol 24:299, 1996 208. Traycoff, C., et al., 1994. Exp Hematol 22, no. 215.

209. Traycoff, C., et al., 1994 Prog Clin Biol Res 389:281-29 1.

210. Trentin J J: Regulation of Hematopoiesis, vol. 1, 1970.

211. Trevisan, M., and N. N. Iscove. 1995. J Exp Med 181:93-103.

212. Trevisan, M., X. -Q. Yan, and N. N. Iscove. 1996. Blood 88:4149-4158.

213. Tsai, S., et al. (1994). Genes Dev. 23, 2831-2841.

214. Tsai, S., et al., 1994. Genes Dev 8:2831-2841.

215. Tsai, S., et al., 1992 Genes Dev 6:2258-2269.

216. Tu L, et al. 1996 J Immunol Nov 1;157:3995-4004.

217. Turner, C., et al., 1996. Blood 87:3237-3244.

218. Uchida, N., et al., 1995 In Hematopoietic Stem Cells: Biology and Therapeutic Applications. Merteismann, editor.

219. Uchida, N., et al., 1993 Curr Opin Immunol 5:177-184.

220. Uckun, F. 1990. Blood 76:1908-1923.

221. Van der Sluijs J P, et al., Leukemia 7:725, 1993.

222. Verfaillie CM, and Miller JS: Blood 86:2137, 1995.

223. Verfaillie, C., et al., 1990 J Exp Med 172:509-520.

224. Visvader, J., 1992 EMBO J 11:4557-4564.

225. Wang, Q., 1996. Proc Natl Acad Sci USA 93:3444-3449.

226. Wang, Z., and D. D. Brown. 1991. Proc Natl Acad Sci USA 88:1150511509.

227. Watson S R, et al. J Cell Biol 1991 115:235-243.

228. Weilbaecher, K., et al., 1991. Blood 78:945-952.

229. Weiss L, et al., Blood 78:975, 1991

230. Whitlock C A, and Witte O N Proc Natl Acad Sci USA 79:3608, 1982.

231. Whitlock, C. A., and C. E. Miiller-Sieburg. 1990.

232. Wineman, J. P., et al., (1993) Blood 81, 365-372.

233. Wineman, J., et al., (1996) Blood 87, 4082-4090.

234. Wolf, N. S. (1979) Clin. Hematol. 8, 469-500.

235. Wood, H. B., et al. Blood 90, 2300-2311.

236. Yamanashi, Y., and D. Baltimore. 1997. Cell 88:205-211.

237. Yang, E., and S. Korsmeyer. 1996. Blood 88:386-401.

238. Yang, T., L. Cheng, and S. Kain. 1996. Nucl Acids Res 24:4592.

239. Yang, Y., et al., 1996. Exp Hematol 24:605-612.

240. Yoneda-Kato, N., et al., 1996 Oncogene 18, no. 12(2):265-275.

241. Yonemura, Y., 1996 Proceedings Natl Acad Sci USA 93:4040-4044.

242. Yonemura, Y., et al., 1997 Blood in press.

243. Young, B. D., and M. L. M. Anderson. 1985. In Nucleic Acid Hybridisation. B. D. Homes and S. J. Higgins, editors. Oxford, Washington, D.C. 47-71.

244. Young, J. C., et al., 1996 Blood 87, no. 2:545-556.

245. Zettlmeissl, G., et al., (1990) DNA Cell Biol. 9, 347-353.

246. Zhang, L., et al., 1997. Science 276:1268-1272.

This invention may be embodied in other forms or carried out in other ways without departing from the spirit or essential characteristics thereof. The present disclosure is therefore to be considered as in all respects illustrative and not restrictive, the scope of the invention being indicated by the appended claims, and all changes which come within the meaning and range of equivalency are intended to be embraced therein.

What is claimed is:

1. An isolated nucleic acid derived from an isolated hematopoietic stem cell, the isolated nucleic acid comprising the following characteristics:
specifically expressed in the hematopoietic stem cell; and
encoding a hematopoietic stem cell-specific protein.

2. An isolated nucleic acid of claim 1, the isolated nucleic acid further comprising the following characteristic:
capable of hybridizing under standard conditions with a sequence selected from the group consisting of SEQ.ID.No.: 1, SEQ.ID.No.: 2, SEQ.ID.No.: 3, SEQ.ID.No.: 4, SEQ.ID.No.: 5, SEQ.ID.No.: 6, SEQ.ID.No.: 7, SEQ.ID.No.: 8, SEQ.ID.No.: 9, SEQ.ID.No.: 10, SEQ.ID.No.: 11, SEQ.ID.No.: 12, SEQ.ID.No.: 13, SEQ.ID.No.: 14, SEQ.ID.No.: 15, SEQ.ID.No.: 16, SEQ.ID.No.: 17, SEQ.ID.No.: 18, SEQ.ID.No.: 19, SEQ.ID.No.: 20, SEQ.ID.No.: 21 , SEQ.ID.No.: 22, SEQ.ID.No.: 23, SEQ.ID.No.: 24, SEQ.ID.No.: 25, SEQ.ID.No.: 26, SEQ.ID.No.: 27, SEQ.ID.No.: 28, SEQ.ID.No.: 29, SEQ.ID.No.: 30, SEQ.ID.No.: 31, SEQ.ID.No.: 32, SEQ.ID.No.: 33, SEQ.ID.No.: 34, SEQ.ID.No.: 35, SEQ.ID.No.: 36, SEQ.ID.No.: 37, SEQ.ID.No.: 38, SEQ.ID.No.: 39, SEQ.ID.No.: 40, SEQ.ID.No.: 41, SEQ.ID.No.: 42, and SEQ.ID.No.: 43, SEQ.ID.No.: 45, SEQ.ID.No.: 47, SEQ.ID.No.: 49, SEQ.ID.No.: 51, SEQ.ID.No.: 53, SEQ.ID.No.: 55, SEQ.ID.No.: 57, SEQ.ID.No.: 59, SEQ.ID.No.: 61, SEQ.ID.No.: 63, SEQ.ID.No.: 65, SEQ.ID.No.: 67, SEQ.ID.No.: 72 or a portion thereof.

3. The isolated nucleic acid of claim 1, the isolated nucleic acid further comprising the characteristic of encoding a protein capable of modulating hematopoietic stem cell activity.

4. The isolated nucleic acid of claim 3, wherein the activity is selected from the group consisting of hematopoietic stem cell differentiation and hematopoietic stem cell replication.

5. The isolated nucleic acid of claim 3, wherein the protein is selected from the group consisting of a growth factor, a transcription factor, a splicing factor, a capping factor, a transport protein, a translation factor, and a replication factor

6. The isolated nucleic acid of claim 1, wherein the nucleic acid comprises the nucleotide sequence of SEQ.ID.No.: 72, an analog thereof, or a portion thereof.

7. The isolated nucleic acid of claim 1, wherein the hematopoietic stem cell is a primitive hematopoietic stem cell.

8. The isolated nucleic acid of claim 7, wherein the primitive hematopoietic stem cell is selected from the group consisting of an umbilical cord cell , a bone marrow cell and a fetal liver cell.

9. The isolated nucleic acid of claim 7, wherein the primitive hematopoietic stem cell is selected from the group consisting of a AFT024 cell, a 2012 cell and a 2018 cell.

10. A composition comprising the nucleic acid of claim 1, wherein the nucleic acid comprises one selected from the group consisting of SEQ.ID.No.: 1, SEQ.ID.No.: 2, 20 SEQ.ID.No.: 3, SEQ.ID.No.: 4, SEQ.ID.No.: 5, SEQ.ID.No.: 6, SEQ.ID.No.: 7, SEQ.ID.No.: 8, SEQ.ID.No.: 9, SEQ.ID.No.: 10, SEQ.ID.No.: 11, SEQ.ID.No.: 12, SEQ.ID.No.: 13, SEQ.ID.No.: 14, SEQ.ID.No.: 15, SEQ.ID.No.: 16, SEQ.ID.No.: 17, SEQ.ID.No.: 18, SEQ.ID.No.: 19, SEQ.ID.No.: 20, SEQ.ID.No.: 21, SEQ.ID.No.: 22, SEQ.ID.No.: 23, SEQ.ID.No.: 24, SEQ.ID.No.: 25, SEQ.ID.No.: 26, SEQ.ID.No.: 27, SEQ.ID.No.: 28, SEQ.ID.No.: 29, SEQ.ID.No.: 30, SEQ.ID.No.: 31, SEQ.ID.No.: 32, SEQ.ID.No.: 33, SEQ.ID.No.: 34, SEQ.ID.No.: 35, SEQ.ID.No.: 36, SEQ.ID.No.: 37, SEQ.ID.No.: 38, SEQ.ID.No.: 39, SEQ.ID.No.: 40, SEQ.ID.No.: 41, SEQ.ID.No.: 42, SEQ.ID.No.: 43, SEQ.ID.No.: 45, SEQ.ID.No.: 47, SEQ.ID.No.: 49, SEQ.ID.No.: 51, SEQ.ID.No.: 53, SEQ.ID.No.: 55, SEQ.ID.No.: 57, SEQ.ID.No.: 59, SEQ.ID.No.: 61, 30 SEQ.ID.No.: 63, SEQ.ID.No.: 65, SEQ.ID.No.: 67, SEQ.ID.No.: 72 or a portion thereof.

11. The nucleic acid of claim 1, wherein the nucleic acid is selected from the group consisting of DNA, RNA and cDNA.

12. A vector comprising the nucleic acid of claim 1.

13. The vector of claim 12, wherein the vector comprises viral or plasmid DNA.

14. An expression vector comprising the nucleic acid of claim 1 and a regulatory element.

15. A host vector system which comprises the expression vector of claim 12 in a suitable host.

16. The host vector system of claim 15, wherein the suitable host is selected from the group consisting of a bacterial cell, a eukaryotic cell, a mammalian cell and an insect cell.

17. An isolated hematopoietic stem cell specific protein or a portion thereof encoded by the nucleic acid of claim 1.

18. The isolated hematopoietic stem cell of claim 16, further comprising the following characteristic:
capable of modulating hematopoietic stem cell activity.

19. The isolated hematopoietic stem cell of claim 18, wherein the activity is selected from the group consisting of hematopoietic stem cell differentiation and hematopoietic stem cell replication.

20. The protein of claim 17, wherein the protein is selected from the group consisting of a growth factor, a transcription factor, a splicing factor, a capping factor, a transport protein, a translation factor, and a replication factor.

21. The protein of claim 17, the protein having substantially the same amino acid sequence as one selected from the group consisting of SEQ.ID.No.: 42, SEQ.ID.No.: 44, SEQ.ID.No.: 46, SEQ.ID.No.: 48, SEQ.ID.No.: 50, SEQ.ID.No.: 52, SEQ.ID.No.: 54, SEQ.ID.No.: 56, SEQ.ID.No.: 58, SEQ.ID.No.: 60, SEQ.ID.No.: 62, SEQ.ID.No.: 64, SEQ.ID.No.: 66, SEQ.ID.No.: 68, SEQ.ID.No.: 70, SEQ.ID.No.: 71, and SEQ.ID.No.: 73.

22. A nucleic acid probe capable of specifically hybridizing with the nucleic acid of claim 1 under standard conditions.

23. An antibody capable of specifically binding to the protein of claim 17 without substantially cross-reacting with a non-stem cell specific protein or homologs thereof under conditions permissive to antibody binding.

24. A cell capable of producing the antibody of claim 23.

25. A method for identifying the presence of a primitive hemopoietic stem cell in a sample comprising:
(a) contacting the sample with the antibody of claim 23 under conditions permissive to the formation of an antibody complex; and
(b) detecting the presence of the complex formed in step (a), the presence of a complex formed indicating the presence of a primitive hemopoietic stem cell in the sample.

26. The method of claim 25, wherein the antibody is labeled with a detectable marker.

27. The method of claim 26, wherein the detectable marker is selected from the group consisting of a radioactive isotope, enzyme, magnetic bead, dye, flourescent marker and biotin.

28. A method for generating a stem cell/progenitor cell from a primitive hematopoietic cell in a sample comprising contacting the sample with the protein of claim 17.

29. A method for identifying the presence in a sample of a compound that modulates hematopoietic stem cell activity comprising:
(a) contacting the hematopoietic stem cell with the sample;
(b) determining the hematopoietic stem cell activity;
(c) comparing the hematopoietic stem cell activity determined in step (b) with the activity determined in the absence of the compound an increase or decrease in hematopoietic stem cell activity indicating the presence in the sample of a compound that modulates hematopoietic stem cell activity.

30. The method of claim 29, wherein the activity is selected from the group consisting of gene expression, replication, differentiation, transplantation, and self regeneration.

31. A compound identified by the method of claim 29, previously unknown.

32. A method for identifying primitive hematopoietic stem cell-specific nucleic acids comprising:
(a) creating a primitive hematopoietic stem cell cDNA library and a non-primitive stem cell immune cell cDNA library; and
(b) subtracting the two libraries, thereby identifying primitive stem cell specific nucleic acids.

33. The method of claim 32, step (b) comprising:
(i) contacting the nucleic acids of the stem cell and non-stem cell libraries with each other under conditions permissive to hybridization, thereby forming hybrid complexes;
(ii) separating the hybrid complexes formed in step (b) from the nucleic acids which did not form complexes;
(iii) isolating the nucleic acids which did not form complexes, thereby identifying hematopoietic stem cell specific nucleic acids.

34. The method of claim 33, step (ii) further comprising amplification of the nucleic acids.

35. The method of claim 33, step (iii) further comprising ampliciation of the nucleic acids which did not form complexes.

36. The method of claim 33, further comprising displaying the amplified DNA on a chromatography gel.

37. The method of claim 32, step (b) comprising differential display of the two libraries, thereby identifying primitive stem cell specific nucleic acids.

38. The method of claim 32, step (b) comprising representation difference analysis of the two libraries, thereby identifying primitive stem cell specific nucleic acids.

39. The method of claim 32 further comprising cloning the stem cell specific nucleic acid.

40. The method of claim 32, wherein the stem cell is selected from the group consisting of AF024, 2012, and 2018.

41. A nucleic acid identified by the method of claim 32

42. A composition comprising the compound of claim 31 and a carrier.

43. A pharmaceutical composition comprising the compound of claim 31 and a pharmaceutically acceptable carrier.

44. A primitive hematopoietic stem cell specifically expressing one selected from the group consisting of: SEQ.ID.No.: 1, SEQ.ID.No.: 2, SEQ.ID.No.: 3, SEQ.ID.No.: 4, SEQ.ID.No.: 5, SEQ.ID.No.: 6, SEQ.ID.No.: 7, SEQ.ID.No.: 8, SEQ.ID.No.: 9, SEQ.ID.No.: 10, SEQ.ID.No.: 11, SEQ.ID.No.: 12, SEQ.ID.No.: 13, SEQ.ID.No.: 14, SEQ.ID.No.: 15, SEQ.ID.No.: 16, SEQ.ID.No.: 17, SEQ.ID.No.: 18, SEQ.ID.No.: 19, SEQ.ID.No.: 20, SEQ.ID.No.: 21, SEQ.ID.No.: 22, SEQ.ID.No.: 23, SEQ.ID.No.: 24, SEQ.ID.No.: 25, SEQ.ID.No.: 26, SEQ.ID.No.: 27, SEQ.ID.No.: 28, SEQ.ID.No.: 29, SEQ.ID.No.: 30, SEQ.ID.No.: 31, SEQ.ID.No.: 32, SEQ.ID.No.: 33, SEQ.ID.No.: 34, SEQ.ID.No.: 35, SEQ.ID.No.: 36, SEQ.ID.No.: 37, SEQ.ID.No.: 38, SEQ.ID.No.: 39, SEQ.ID.No.: 40, SEQ.ID.No.: 41, SEQ.ID.No.: 42, and SEQ.ID.No.: 43, SEQ.ID.No.: 45, SEQ.ID.No.: 47, SEQ.ID.No.: 49, SEQ.ID.No.: 51, SEQ.ID.No.: 53, SEQ.ID.No.: 55, SEQ.ID.No.: 57, SEQ.ID.No.: 59, SEQ.ID.No.: 61, SEQ.ID.No.: 63, SEQ.ID.No.: 65, SEQ.ID.No.: 67, SEQ.ID.No.: 72 or a functional portion thereof.

45. A primitive hematopoietic stem cell specifically expressing a nucleic acid identified by the method of claim 32.

46. A method for treating a condition in a subject comprising administering to the subject a therapeutically effective amount of the composition of claim 43.

47. The method of claim 46 wherein the condition is an immune system condition.

48. The method of claim 46 wherein the condition is leukemia.

49. A method of introducing an exogenous nucleic acid into a hematopoietic stem cell comprising contacting the stem cell with the composition of claim 42.

50. A method of ex vivo expansion of hematopoietic stem cells comprising contacting the cell with the composition of claim 42.
